SEQUENCE LISTING (72 sequences)

SEQ ID NO: 1 - DNA, 170001 bp, Homo sapiens
  variation (80217)..(80217): S = G or C
  variation (130613)..(130613): Y = T or C
  variation (130625)..(130625): Y = T or C
  variation (139968)..(139968): K = T or G
  variation (142199)..(142199): R = A or G
  variation (142460)..(142460): R = A or G
  variation (145782)..(145782): S = C or G
  variation (146008)..(146008): R = A or G
  variation (146012)..(146012): R = A or G
  variation (148109)..(148109): Y = C or T
  variation (148199)..(148199): W = T or A
  variation (148333)..(148333): W = A or T
  variation (151403)..(151403): M = A or C
  variation (153127)..(153127): W = A or T
tgtattcttcagcaaaacaatctttccatgaagtttccacgaataattcccaggtagatgcattaaaaagggagtcttggcacatgaaaaatgcaatatgaacctagccaagcaagagtcagatctgagcctaaaataaaactggaaggtaatttcataaaatgcataaaaacattcaccatagattattcgggtttttttttaagcgggtatttcagattagctttctatatttggctatgtaatcgtgtccaaaaatgaatcagtgcagcatgccaagataatgatcacagtgacacaattagttgtggaactgctgcttaatgaacatagcaagtattcatggccactctcctggatcccgcgcctgccttgttccttttccagaagcgtctggatcacagtcaccgtgtcccgtgtgcacagcctgggcggagcagcacatgagagggtgatgctcagtggtctaagcaccgtgatgtttacaccactactcttgcttcctgttgaatattcccatagctaattgtgaaaatactgaagtttgtgaaaatactgaagttttacagaataaacatttggaatatattgcaatagcaaacacagatggcccgcatagtggaattcaaaaacttctcatccttttcagaagaacattaagcttcaaaaaaaagaatcctgaagattttcagaaatggattcacttgacttggcagactaaagtctaccaaaaagttatccctcttaatacaatcactatatttaaaacacacactaaaataaaaccactattcaaaaataactatccaacaggaaatatctggacataaagggaatcgagaatgttcccacagctaaaccttttcaaaacacatatacacaatcaaaaggcatattggatacgtttttacctaaataggatatcttgtaattagaacatttccagaaaatgggtgcctcttaaatatatttatacaacctttatctcagtctcttaaatgtacagcataacctcaacattgcctcttcacaatagaaataaatagaccagagatgacacacatactttcaaaaaagaaaaacaaaaaaccaaaaaataaacagaggtagtccagttatttaggcttctgcagtcaaagtactcaactcatttccagaacctttcatttcacatccaagcaatatgatatcagagagccactgccaaatgaagcacaggtaaatgcattttatggctatttaatcaaaatgcgctttacttcagagaggcattaagagacacgattaaggaggctttccaaaaaaaaaaaaaaatcaagaaatgttaagagtgaagaaacagtaacttttattaaactaaagattcagcaatgaggttttgatgggcttatccacacaggtgccctgcccatataggacacattaaatactgaccccgggccgggtttggtggctcacaactgtaatcccagcactttgggaggccaaggtgcaaaggttgcttgagtccaggagtttgagaccagcctgggcaacatggcaaaaccctgcctctacaaaaaatacaaaaactagccaggggtagtgatgcatgcctacagtaccagctacttgggatgctgaagtgggaggatggcttgagcccaggaggcagaggctgcagtgagctgagatcatgccactgcattccagcctggatgacagagagagaccctgtctcaaaaataaatgaataaatactgaaccccaagaaggccttccttcctgggagctaactccaggggatggtggcaccagtcttggaatattacgctatcaataatttagggggtcctgagaaaataattaagggggtcctattttttcatgagatcatttgtagccattatgtgaaatgcacaggactttagggaactgttgctagagctcttataacatctcccaaaggtggtgatattatccgcttacactgtaggtttatcatctctctgcaaagcactgaaagctagtaggagtaccaaaacctcctaagacatcccattacatggaaaatcctcttggaagatacaaagcgtgtcgctcctgactatcacagttctaggtgtttggtaataactcatccacaaaacgaacgtagtgaatgcttatcccctcaataagctaccaagcgatttcctgaactcaaggtactagaccctgggctacctttgctctgttattgtgactagagtaattatcacaatcattttagggattttcccccctcattgtcaaatgtggggagggtatgggctatagaattagatgatgggccgggcgcggtggctcatgcctgtaatcccagcacttttggaggccaaggcaagtggtttacctgaggtcaggggtcgagggaggtgtattacctgaggtcaggagttcaagaccagcctgaccaacatggcaaaaccctgtctctactaaaaatacaaaaattagccaggcatggtggcgcgtgcctgtaatctcagctactctggaggctgaggcaggagaagcgcttgaacccgggaggcggaggttgcagtgagctgacattgcgccattgcactccagcctgggcaacaagaataaaactctgtctaaaaaaaaaaaaaaaaaaaaaaaagaagaattagatgatgatctcattaacacgactctcttccacaggatccctcaattcattaatctccaaaaaagtaatctgtttagaagattgttgaaacagccttgagtcacttaagatgatggcataatgatcccctgagactcgcccccatatgaaatccttccacaagaagtatgttttccctagtctttaagcttccctcctcacttcttccacgtctgcagtattaaattctataaagatcagtcatccaactcagacggtgctactcctagcataagatcagaaaatgggacaagtactcagggtttatgttgctgtctctggtgccatcgcctcaccctcgactctcaatcttgagtgtgggctgctaaaccaaaggcagggatggcagggcctaatacctttcgcttgcctctaccttccgcccttctgcagtctgctaagggggtcgagccgctcctatcaatgcacctcctgctcttccttgcttcagtgggagcccagatgggagatgcagggagggaggctagtgaagacatggtatttgttccctggcaagctacgtggggatggctgtgtccttcagtggcacatggctgctttctcaaggtggcgcaatctgactttttcttcctgggttctaggaacgcctcccttcccttgtcccttcaggctgtgttaaatgttcagctgctactagcatcaagctgctgcctattcctccccattcccctctgctaatggttctgacactgtagcacttatcgtcagcacccgaagggcttattaaagcaggtggctggccccaaccccaacgtgtgggattcactaggtcttgggtggggcccagacattggcatttctcacaagtctgaggccacctttgaaaactgctgctgctttatagacacctttgtttacaaggaatataggcagagagtggatggcaactgctgccaagaaaccactcccacaattgccaacagctggtttgggagagataattaataggcctgcttgtatcagttagggtcccaactgcaaacagatggtgcacttcaaataggataactccctacagccagtcagtgaatgtgcaaacctctcaaggccctgaggttttgtactgaagacctaagcacacaggtaagaaattgaactctgcccgtcttcactcattgaaaatagctaacacagtcaggtgaccaggcgacatggatctgcaggctggctaaaaggcctaagcaggtattccagctgtaccctctgcaacttctgtttttttctccaaaagcacttgatggccttttcctcttcattgcactaattcatctttactcaaagcatgactcttgtgattttaagcatctagaaaagcttttttgtgatggataaaacttttatcttaccagctgatgtatagcccatttctctattactcacgataacagctcttctcacttccacacatgtgcagagcattttccatttctaggctggaggttatatccattcttgctactctgtttcttcatatacttgtcaaaagagtgttaaactgacacactgatagctttcaggttacagatggtctcgatgtggccctgtccaattttccagcattctccttttatttatttttatcttatttatttatttatttatttatttatttatttatttattttttgagacggagtctcgctctgtccagcccagactggagtgcagtggagcgatctcagctcaccacagtctccatcttccgggtttaagtgattctccggcctcagcctcccaagtagctgggattacagacgcccgccaccatgtccgggtaatttttttgtatctttagtagagacagggtttcaccatgttggtcaggctggtctcgaatgcctgacctcaggtaatccacctgccttggccttccaaattgttgggattacaggcatgagccactgcgtctggccttgttttgttttgatgttagatattttaatagtgacaaatattttaaataacatataaaatatttttaagttatgtacaaaataaataccagctactagaaacccatttcccatctcaggactagaatatacccccttcctgcgtatctctcctcatccttttaaaaaacgattatggggctgggcgcacacctgtaatcccagcactttgggaggccaagttgggggatcacccgaggtcgggagttggagaccagcctgactaacaaggagaaaccctgtctgtaataaaaatacaaaaaaaaaaaaaaactagctgggcatgttggcgcatgcctgtaatcccagctactggggaggctgaggcaggagaattgcttgaatctgggaggcagaggttgcggtgagccaagatcatgccgttgcactccagcctgggcaacaagagtgaaactctgtctcaaaaaaaaaaaaaaaaaaaaaaaaagcaaatatgctatctcttctgtatatttccctaagcaacatattgtttagttttgctggtttgggcacattttctgcaatttgtattttccactccctcatagttctaagaattattcatattgttacatgtaacaacagcacgttcattttccctgtgtaaagttcataatgggaatatggtataaattatttattcattctgttgctagatacttgagtgttttccagtttttgttattatcaacaatgctgctgtgaattttcttaaagatatttcctggtgtacacgttttagagtttatcttgggtctcctaaaactagtagtagaattgctaaattataagatacatgcatctttatgttatattaccatctttataaggtaatatcaaattgttttccaaagtaattgcaccaacttccagtcctaccagtattgtatgagctcctgttgacccatgccctcaccacctgtgatatgacatcttgatttttttgccagtatagtttttgtaagaggatattacatttctctaatagctagtgaaattaattatctcagccagtcatggtggctcacacctgtagtcccagcactttgggaggctgaggcagatgggttgcttgagctgaggagtttgaaaccagcctgggcaacatagtaaaaagccatctctacaaaaaatattagaattagccaggcgtggtggcatgcacgtatagtcccagctactcagaggctgaggcggaaggatcactcgaccctggggagctggaggagctgcaagtgagttgtgattgggccgctgcactccagcctgggtgatagggcgagaccctgtcttgaaaataaatgatttattcttagacactatattagtttctcagggctgccataacaaatgaccacaagctgggtggcttaataataacaaaaatttattctctcacagttctggaggctagaaatccaaaatcaaggtgttggcggggctgtgcttcctctgaaggctctcagaagaatccttgcttgcatctccacctcttgctggctccagacgctccttggcttgtgactgcatacctcagtctctgcctctgcctgcaggtggccttctcctccatgtgtctctccccgggatggctcttctaaggatgcttgtcattggatttaggacccacccaaataatccaggatgatctcaagattcttaattacatccgcagagaccctgtttccacaaaaggattcacaggttctggggattaggacatgtccctatggtagtccccactcaacccactgcatgtacaatccctattcatatttcctcttcctgaaatataggttcatgatttttttccatttttctatctaatctctgcctgtttttcttttcttttttttttctttttttgagatgaaatctctatcgcccaggaaggagtacagtggcacgatctcggctcactgcaatctctgcctcccaggttcaagtgattctcctgcctcaacctccccagtagctgggattacaggcacatgccaccacgcccagctaatttttgcatttttagtagagacggggtttcaccatgtcgtccaggctgatctcgaactcctgacctcaggtggtccgcagtattcatacaataatatagtattgcttgaataatacagtattgtatgaatattctcccagtctgtatattttcacattttaatagaggttcttaatttttatgtaattgaatttgttaatcttttctttcattttaaaaacactttgtgttttacacagtaaatccttccatagtcccaaatacattattttcttatattttcttccaaatcatttgacaattggccttacagctttacattttaattcatcaagaaaatgtttatgtttatagtgtgcagtaaggatgcaatttttcttttttacgtgagtggagataaccgatgcccctcatcccgccctgatgaatgtctagagcagctgtggttaccttgtaataatgtgactgcaagcttgagaataaaggcatgacttgacatgactgagggatgggatgccgtcaacactgaagtgaccattagctttattacaaagcacaactcaacatatatcattgtattcccagcactttgagaggctgaggctggcggatcacctgaggtcaggagttcaagtccaacctggccaacatggtgaaaccccgtctctactacaaatacaaaaattagctgggcatggtggtgggtgcctgtaataccagctactcaggaggctgaggcaggagaattgcttgaacccaggaagtgaagtttgcagtgagccaagatcacaacactgcactccagcctcctgggtgacagagcgacactccgtctcaaaaaaaaaaaaaaaaaaaaaaaaaaaaaaaaaaaaaaccaactaaaaagaataagcaaatctagatcaacctccctactctgataacagcatgaaatacgtgctatcctataagctctaatacaattttgcatacactgaagtctatctctagtcaagtttagctataaccaccagtaacttagtgccattcaagctgtattttgtgttgcatatttgccagatgacatcattgaataaatagagtgtggccaacaagccctcaggtttgaagtcgttctcatccaccaggtggatgaatcagcagtgcccggtgtggttaaggaaactgggcacagttgcattgcatgcctcttcaattaatcatgcaaattagccagttgggtgagacagtccactgaatccataatttcatgtctgataaattgaacatgtacatacaaagagaaatctgaggtctcagaaaacccagttttacaaaatgcatagttcttttttaaaccaggaaggaaataattgttcaatggaactcaatccatcacttctgggcctcataaataacacaagtcttcctttagaaaagtcacaatgggggaagaaaacaggcccacaagctgtatgtgctgtattctcactgaccagattttgcaagtcatttgttggcttagaaagtctctagacctgctctgtccaaaatggtaggcgttagcctcatataactgtttaaatttaaatgaattaaaattaaataaaattaaaacttcagttcctcactttgcactagccacctgtcaggtactcagtagtgccacccagggcagcatagatagaacattcccatcatcacatagagtctttggatagaactattctatacattttcttccagtctatgcacagggttctaacattcgtggtagtccttaatggtttaccaagtgttttctcatcctcataacattattggtagaaagccaataatattttattaatagccaataataatttattccaaatttctttatcaattgctgagtagcagattccctcaaaacatagcaatttaaacaacaaacatttgttaactcccatcatttctgatggtcaggggtctgggtgtggtcggctgggtgatgtggctcaggggccctcatgagggcacaagtcaagatgtctgctggggctgcagtcatctgaaggtacagctagggctggggacccactttctagctcactcatgtggctgttagaagaagccttccattcctcatcacgtgggcttctggctttccccagagtgaataacaggagagactatgagagcgagagcgagagagagccaggagagacctcggtgtcttttatagttgctgagggttgaatgtgtcccccagagctcaggtgttgaaaacatgatccccaaggtggccgtgttgggaggtggggcctagggagaggtatttgggcatggcggcatcaccctcatgaacagattaaggctattatggagggagtgggttccttataaaaacacaagttcaatctcctcttgttctttctcttgccttctctcaccctctcaccttccaccatggaatgctgataaagagaaggcccttgccaggtgctgggccctcagtcttggacttctcagcttccaagggcgtgagccaagcaatttctgttcattgtaaattacccagtctgtgctattctattacagcagcacaaaatggactaagacaataacctaatctcagaagtgacatcctgtcctttctgtattattctattacacagaccctggtggataagagatgggggagatgacaccaaagtgtgaggatcaggagttaggatccctgaggctcagcttagcgtcttcgctaatcacattttagctaatcccccattgccacttaagaagccaagtaatttgcgaagtttacacacagcccgtaaatgtcagccgcagaacgctcacctcaatcctgacttttagctccgtttctctacagtactccacctttacccatctagataccaccccaacccccttaaacaatttggcttctaagaaatcgatgacacagatgaataatgttaacctgcaaatgttacagctaaggttcaaaaggtctcagaagcttgattaataacaaatgtccctctcctttcactgctttttcaggacaaaataatcataagacagtaggaaaaaaacagaaagagcctgctactgacaaagattttctcctgagccaaacttcagtcgggctcttgcaagccccccaggctgtgaccttggtgtctgtccttgttgcacctgcactgaccacttgcagcaagagtcctgtgagtcactttaaccagaactcctctcactcgtgatgtatcgttgtagttgttttccatccacagaccctccccactttgctccctggctgtaaatccccagttctcctacttgtgtttggagttgagcccagtctctctccccaccgtaagaccccactgcagtgccttacagctgtctcagtagcactgaacacatagcctgcctttccagcttcactctactctggaacaaatgtctagcagagggaaaaaatcagggaaaggttgttatttgttctgggtctttcatacacctaaccacacaggagaggcaatatttcacaaattgttgcatagaaaaggatggggtggggcagggacaaacgatgttccttcctaaacatgcaaaacaaccaaacccacttaaagaatcttatccttaactcggcacggtcttttttattttttttggtggtgggggacggagtctcgctctgtcgcccaggctggagtgcagtggcgcgatcccggctcactgcaagctccacctcccaggttcaagcgattctcctgcctcagcctctcgagtagctgggactacaggtgcaagccaccacgcccagctaattttttttttttttttttttttgtattgttagtagagacggggtttcaccgtgttagctaggatggtcttggtctcctgacctcgtgatccacccacctcggcctcccaaactgctgggattacaggcgtgagccaccgcacccggccaatatggcaaaagtcgtaattttttttgaatgattctgtttcaaataaaggtcaaagtgtgaacaaagtaggccatatcaccaaatataagctgacttcacttttcgactcgaaatttgatattgacagttacaggtacaaagaaattcaaattccaaatgttcatagatttgctgacagtcggtgtttccagctgactatttatcctctggtaagtattggatatctgagtttggttaatattcacttccgggagttcgtttacacaattggaaaatatactagaaggttttgggtacttgaggaaagagcagtgggagcagagaaaaatcttctggttgaacaaataaaatttcatgttcttattttgtctgccattatgatagaacaatagtctgcaagactgaatagttttgcatctgaaaacttctttctcactgttatggactgaatctttcttccatctccccagttcatgtgttgaagccctaaccctcaatgtgactgtatttggaaacagggcctttaggaaggcaattaaggttaactgaggccataagggtggggccttaatccaacaggacttgtatccttgtaagaggaagacaccagagcttgctctctctgtaggcacggagcaaggccatgtgaggatacacagagaatgtgtccacctacaaagcaggaaaagaggccacaccagaaacgacactgtgggcaccttgatcttggattcctagccctcagaaacatcagaaaatacatttctattatttaagccacccattcgatggcattttgttatggcagcccaagcagattggtatactcaccattaagagaaagcttcaagaatccttcaatcttatcattggagttaggcccaattcctgctgaccaatgagctgaagtacatcaatttattttttctttaaaataattcctaggatattttaaaagggactgcttagctgaaatacatgatgtcaactgcaagtaatgctaatcccaacacaactcagattaaacaatatggaaacctatgctctcccattaaaaacaaaacaaaacaaacaaacaaaaaaaacacatctttagtttgcgtggcctacagggatggttcattcaatgtcatcaaggcccttcgtctgccttccttggtttaggcctcatcctcactaatcaagataactattgtagtcccaaccatgacatccttatttttgtctgccattatgatagaacaatagtctgcaagactgaatcattttgtatctaaaaacttctttctcagataatatctcttgtgattctttcttaggagggaggaattttttttccagaaacctccaagaaacttcctttaatatctcactgactaccattgatcaacatgcccatgacaagaaatagagaactaccctgatgggtttagatagatgatctgttagagaatggatgctgagaaaatatcaccatacctcattaaagtttctatataagattagcttttgtgactaaaaaattctaccaggaaaagggatgagatttaatagtaaagaagtatagatagagggaatattccaagggtaaaattacttgttgtcatatgtaaaacagaaatggagagaaaggagaattggagagagaaaaaaacaaaacactagtggttccagtccaggaaagagaaaagcaaggtctacattgaaatgctgatcatgaaatcatgtgacatagtccttgtcagaattcattgagaatcattggtgtgtatttttctgctgttcagcctaaacaagttaaaagccagactataatttttgaattgaaaagcaatgtgtctaacagtatgcatatatatacatgaaaatatgtaatccaaacaagtaatggatgtgttttatgtatgtattcctgaacacaatcactgatgttctcactctgattaaaggacctacttttacacaattatttatccaaactcgtagctgctacaagagagcattttcctgcagctattatttgatagttttttgctcccctcctaggccaattcttaaaccacaacactgctgacctttagtgtaaaactgtcatggttcctgaagggtgcaaatgaggtgcaggctatgaggagctagtgcactatagcaggcaagagtgtttgctctggaggcagccagccttagggttcagtctgaaaggtgacccagtgtgttatttaatctacctcaaacctcagttttcatctgtaaaatcggggtagggcattaattgctaatgcttcccaggattttgtaagaatgacatgagataatgcatataaagaaggtggaaccaagtccggagcataactggcagtaaatacatgttactggccattggttctacagagcttcatagctgagataggagtggcagggtgagacacagtaggtaaatttgaggagactaatgatattgtagttacaattattctttttttttttcagatggagtttcgctctggtcgcccaggctggagagcaatggcgcaatctcagctcactgcaacctctgcctcctgggttcaagtgattctcctgcctcagcttcctgagtagctgggattataggcacacaccaccatgcccagctaatttttgtgtttttagtagagacggggttttgccacgttggccaggctggtctggaactcctgacctcaggtgatccacacgcctcggcctcccaaagtgctgggattacaggtgcaagccactgtgcttggccacaattattctttactggtatctctcacaaccagtgagggagattttgtgtgtgtgtgtccattttagagatctatctttatagaggaaagtgagattcccaaaattcagttataaccaaataaattcaaactatatatgtcaaagacaagattcaaaacaggtcaaaacaggtcttctaacctcaagtgtaatgctctttctactgcatacttctattgagaaagtagcttcaatagtaagttgccagatgacttgttgcctactgttcaaaaggttagaatctttcaatcactgagctggtaaagcatcacaaaaattatatttatcttgcaatatctgttcccaaaccacctggggatgtttatcttatatttgagggacagaatgtctacggccaccctactccactatgggtgtgtctttaaaatgaaagtatactttgtaccttttttttttaatggttagccattagtaggtgatttgtaatacctccaaatactttcccaagtgactggttatggcatcagaaatttcatgttttaactatagctttaagaaagcccttgctataaggcactgggaagggtggaaggatattattataacatactcatacccatagttaaccagcactgattcccatcagattgactaggtgtctaatgttctttttttttgagatggagtctcgctctgtcaccaggctggagtgcaatggcgccatctcagctcactgcaacctctgcctcccgggttcaagtgattctcctgcctcagcctcccgagtagctgggattacaggcacccgccaccatgcccagctaatttttgtatttttagtagagacggggtttcaccatattagccaggctggtatcaaaccctttccctcaggtgatccacccgcctcagactaccaaagtgttgggattacaggcatgagccaccgtgcccagccaggtgtctaatgttctaatggctccctctgagagtacaagctgattccggagaagagctgtatttgaatgcaaagggtaaaataattccaaggtgcaggggaagtggagagggctgatatttcctctcacctccaggaaggggaggccgcaatgggcacacaggcagatgagaaagcagtgaaagcagcagagagggtgggatgccaggagtcagtcaagcctacttagaagtatttggtcctagggctattccaaaaaatgtctcagaattgtttcctgctttaaatctatgatttcccctggttttatgagtcagcaaagacggaaaacaactaaaaggccatgttaatatttcttaatcactaaacacacctacctcattaaatcttgggtcttacatattttcacccagcatattgttggtacttgataaatatttgctgaatgaatgaatccttccatgattctgggtcctcccttatacttcagaggtgactcagtattggacacactgaaattttaatgcttgatgtagccttccataaaatagcagtgtttttaaaggtctagaccaaggtgtccagtggaactctgtcatgacgaaaatgttctttgatctgctgcgatcaatacaattgtcactagccacacatggtcactgaaactcggaatgcagctggtgagactcaggaagtgccttttaatttagtttaactggaagtagccacagggggcaagtggctgcccactacattagcacagctctagaggttctgttaaaagttttctgcccatctcactttgaaaaccagctattttgactaatatcatcaattgttattcataagttcctgtcatttgtgatttttatcttttttaagtaatttggtttatttattacaactttaaagtgagtatagcaaaccaaatggacttcagcgagaagcctctcggggtgaatttattgtgaatcattttgaggacttgcaatatgtggtcaatttctaatttcaactattgacacaaaaggagagagcctgtgtagttgaaacagggaagcaggagaagtccgctgggcactgacagactggcctctcaagccagcctcctccctccttaggaatctggcaccctctagctgccctcggggtctgcaggagctctaaaatcccttcctatatccctccgtgtcctagctccctggatcctctcctcccttccaaatcaaaatgtaccttgagggagaggcttgggcaggaggatctcttgagcccaggagacctgacttgagtctggagacctgcctggcagaatctccaatcagtggtgatgatgctgattttatggattaaaaactgagacactgccaagaagtgagggaagggagcgggggagggcaggaaaactaatattgtttaagtacctactatgtgcgtgctagacacttcatatgatgctcagtaaaaatgctttgcctgcatgaataactgttatctccctggcaagtagagtgcagtatgttttttttttgaatggataaacacatgaatacatttttaccacatgaagcagatattagtcccatatcacagatgaggacactgacatttagacaagttatgacttcaaggctaaacacacacaacattccaatcacctaatcttgttctctccttactgtttagctccctgcccagtttcctctgtgctcagaattttgctcttgacctttgtaaaagaacgaccagctaaactgatcaacatatttataccaacggatgcaaatatgatagaaccgcccccagcccccttttgcaaggatcttgaatgttaatgacaatcttctggggacaatgtcaactcacagaaatggctccctcggaatttcacgctgttcggggaggtattttggtactttagtggcattaaaaatatattccacagcagatattttcctggaggctggacacttacataaagacgacgcattctttataaattaaacctcaacagtgtttgcatttatctaaatggatattatttcgaggaggagacagtgatttgcgtattagcccctatcactagggatgacttgtaagacacattaggaactggctcctcccctatcccttcgccccctcctctcagaaccttataagcttcagagtacggtcaagacaagaggagcattgggaaagaatttgaaaataaaaatacagctccgtgattttcaacacaaagaccacgaagagcataataataagtcgtgaaaaaaaattcacatccagaagttggagggccacaaagtacaagtcgtccaggccagtctgcagagactgttcatgcttccgcccagcaaaaatctggcaggcgacacatcgcgacgaatttcggctgcagaagaggaagagcatcacctgcagtccgcggcgtgggaaacgccgccggaggggctccgcgagccggggaagtgcgcgcagcggggctccaggcggctccaggcgctgagtgcgaggaacgcccgcgctcgccggcgcccgggagacgcagccgaaggaggagggcgggcgtttcacccgcgccacagctgcggccggccagagagaccgcagaggccgggacccccgcagcctccacggccgctgctgccactgtcggggtggcgcgcccagccggagaatcccgtccgcgcccggggcggggggcgaggcggggcgctgacgtcacctcccctcccccaccgcctccaagggggagggagtggggaaagaggcagcgagtcgccgcagccggcggccgcaactcctcaactcggcggctggctgcctgggcacgtcctgacaaacttggctgctcccgggccctcgttccggccccgcgcactctcttttcgcctgcgtttggtgcttaagtttgccctggtcggaagcctccggagatgcggccgcctgggtcccggcggcacggacgttggcactctggttagggacatgctgaagatcccggggaggcaaagttggtacttcaaacccggctcttaacacatatctacaccgcgaggggatcacggagctgccgaggactttagagcccagggccgaggactcggctagactggtgagttctttgttcaaatcgtgcatgccttttggcatttaagtttaaacgttaatgaaattacgtggggtgtgtggctctaagcgagtgcggggaagcgtgtgcgccctacgggttctcggctccaggagctgcgcatccctctggctttgtgcggatcggcgatgaaagattcgtatgcatagctttgggagttcataaaatacatatagagcactgttaaaaagaacttgagaggcgatgctgtttgtgcattattttgaatgtaacttattatagcaatggtgcctagggagggcgcatcggggccgcgagatttgcaggggcgcagcgccctctagtccgggcacctggcgaggcgcgctccttgcagggttctgtgcggaggcgaacctggacctccccggcgctcggcgacgtgggcgcttgctcaccccagtcttcactgaccacccacccgggcccattctttatatgagttacaaaaatcagcttgggtggactggatgtagaggccgtgctggaggaaagcgttttgtttctggaaccgtctccccaaccccctccctaatttccacgagcctttggggacagggttagggagggtcgattatttaaaggacaaatgtggaacttgaaccacatttctctctctcatataaaataattcgatttgtggtagctaataggcatgtccatttcgttctgttggcgcttaaggatcagtagaatcgtggcagagtcctaagtctgaaatctccatccctggctctcggcggctcctgtggcgacgaagtgcaacttcttccaggcagatagatttctcagggagcctggaaaatcgggattcccagacagatgtaaaacatctagtaagccctaggaagcgttattacttattaattaatggtaatcttctccaggaattctcttgtgccatttaaaaaaaatcccgtgattttacgtcagggaatggtatctttgtactttttttactgttcttcttttccccttttagcattactcccgcttcctcgctcccaggaaacggctggagttccggtgtgtttgggttttgctgagatttacaattcggcaagcggcacaagccggttgggcttggagatcaggtcacccactttcgaggtgttccatgtatgaaattgacctgtaagcacttggagcaattatggccgggacaaacacgtctttggatgatggagtttgcatttgctggatgtgtgaatctatgtgtattccaactggtgggcagggaatatagggctacgaatcggcacactcatcttgcaggcattatttttccttcccagccacaacctgctagacacctttattattattattttttccggtcacctctaagatggcttataaggtgactgcgttaactttgaacccaggaaattctctttcacacttgactgccctgccgttcccctccccgccaagctcccctcaccagtcccagggaaatcggatctctttgtcttcccagtgttaaaatgcctgagtcccacaaactttccgagatcaagtccgccctcttgtccgggctgcgtcccagcgcctccggccctccgggcttcggccgagtccgccctgcgccgagcctgcggctggagccctgcccgcaccggggatgggctccgcgcccgggacgctcaggctgggcgcccggcgcggggagttctcggagcgggcttctcggcgctggggcttcccctctgcctaggaacttgcagttgtcggcctggggtcacgctttggggcccctggggccgactcccgggctaggaagcccgcgggacgccgcgactgccttttgtcccacaggcctgggcgtccggggtgtcagcgggggaggccaggcgcccgcggcggggagggcctggggtctaactcggcagaaaaaggtcgcgggggaagcgtttccattttctggggattgaaggaggaagtagattccgacctagctgcattcctttggagacctgaaggacgacttcagcccaccaaaccttcgcggacccagagccgcgccattcaaatgcagattcaggggctcccagaaggctatggaaaagctaaagggatgcctctgggaagctttctcattaggccccagccttttttttttttttttttttttagctaagctaacgtttccctttcttggcagggagctttccccaattccttgcacagcgcctgtggctggccgtaactaacctcggcatcacaagcctcgccctgcccctcggagccgcgctagaccccgctgggcgccttctcgtctcccagcacggacctggtgttcgcagggactggggctctttgggtccgggcgggactgcgtggccgagcccgcggtcccagagccggcagccggggaaagtgggctcgagtttccgcgcccgagaaatgagcgccggcgccggcgggcggggccggggcggagggatctcctgcgctcggggagcgtgaggcgccggcgccgagctgggcggccgggcgcggggagagggcgcgggagcggctcgtgcggcaggtaccatgcggacgcgcgagcccggcgagggccccggcaggcccggtccctgctcgggggcgcgctgagacggcgggtgagctccacgagagcgccgtcgccacttcgggccaactttgcggttagttcgctggggcgcgggccggcgggcgggggcgagagcgtgcagcgcgctgcggtgcggactctgtgggtgtccgcccggtccccggctcgggctccggcgctgccgaaaacggatgagcgcgcgacttcccggagggtggagtgactcggtccttcccagttgggaaattcagcactggctaggggggtcccggcggcggagcgggaaaaggaggcgaccgcgcgggaccgatccggggccattgctggggtccgatcctttctgacacgccgccttccctccgggagctcgccgggctgcgccggcctcgcggtctcctggggcatcccgccgtctgcgaaagacccgctcgctgagcggtgcacgcgggaggcctgcgcggggctgccgggtccctgggtctctgggtgtttgcgccctgcaggagcgctgcccggaggtgcagatgcaaccggtggactgcggagtgaagggctgcgaggctgcatttcggggctgaacatctgcacaatgtgcggtttgtcagtgttttctgtcttctctggaggaggaacttctaagatctttttaaaagaaagagtttttggtccttcattgcctttaaaaaaaaatgctttcttttagagagccagctttctgctgtgtgttagatttttttgttcccgctgatgtgaattgcaaggctttggaaacaaggacgcaattgtatgagttaggaaagtaaactcataaatattttctatgcagttaagtttttttttttgtctgaactgtggtttcattaagtggcttttgttaagcaattcccgggtgcctgatagaggcgattttttaaaagtggatattaacagtgattattagtatgagaaaagattgaaaagaaaatgaccttcagatattcatttccttttcaaaagcagattcttaatgcttcgtatattcagatacaggtttcgatagtttaagaaggtatctgtgtgtgagaactaggtaatggtgatgtttttcacactattcttaatgagtttggttatgctgtccaacacatccagttgttacagggacgtttgaaaaggtgtttctccagttaatagttaattgtgatatttgtctttgtctttttgtcttaggaagacagctctgattccacagttagaattattcctttgtatttaagatgtaaaaatacttaaagcactatgtaattttttaaaacaacaattggtataagccttgagtgatgctaaacatggcatttaacaagctcaaggtaaggtttgatttgaatgaagtttgcatttgtataaccattttttgaaaaatgaacaccttaaatgagccaaattctttggcactctggaaagaagtgtggttgttagaggactttataaaatcatttcgggccacctgtatattcatttgtctcttttgaagtatccccgttgggttctcatcatgttagtagatcatttaaatccatggtccggacaggtgccaacagaaaaatgtttaaaaattatggccgggcgtagtgcctcacgcctgtaaacccagccctatgggaggtcgaggcgggcggatcaccggaggtcgggggtccgagaccaacctgaccaacatggagaaaccccgtctctactaaaaatacaaaattagcggagtgtggtggtgcatgcctgtaatcccagctacttgggaggctgaggcaggagaatcgcttcaacccgggaggcggaggttgtggtaagccgagatcgtgccattgcaattcagcctggggaaaaaaaagaaaagaaaaagaagagaagaaaaatctttaaaaattatgcacaaactaattctgttttcattcccctgatcatcctctacctttccattgcctttagactttgctttgaggtccctttgaagcagtcgtttctttgaatgttagaggtggataggatcttagagattatgttttaacactttacccgcttgctttccacccaagtttggctgattttacgtagaatcaatcagccaaatcaaattagtacattattattgcattagattctgtctcagtggcataatttgtagcctaaaggacctgatttaatagcagggagccgtaaggctggatgtgctgtttaatctttatgagtgggatttgcgctagataattgttcttttgaccaaattatgtagaagttgaagccaaactctatcttttggtctcacttaattgttcagtttgcaagtgcttatttatccttagacactggagaagagtttttgaaatgagtgatatacatcagttaagatcaggaagttgccattttctcatacctgaggggaacttttcatttcaaaaatatattctgatgaggaatctgcatatttcactcttttaagttttttaagtcatcttttactgtaagtcccagatttaaatacattgcctgaattcttgcttaattccaagggttcattatgtatgtgaagataagtctataaagtaaaaataataaaggcttctatacactgcttaggaaatattagtcatgatttaatctgtatccttcatagaatattaaggttagagctttgtgataatctgcaacacagacactgatctggcattatctatagccaattttgaggcagcgtaaggtgggtggtgacctctttctgagctattggaaatgagtcctctacagagcagccacctgcagtgttgttaccagcctcgtggttgtgcctggattttctgttaatttgagaaggatggatgttttattttcctatgttatccctctctatatcccctccccaacacagtcctgcctcacatttctcactcttaacgctttattcatctgtccatttaccaatccaacaatagtggacctcctctgtgtcaaacattgggccagacacaacatctagtgctgaacaaatcgtgtttcctgccctcaagcagctcgtagtctgataagggagaggacaagtacacagtctcttatagaccagtgtgataagagatccattcaggtcaaggtggtccatagcagcacctcagtgagctgggggatctggggcaggtggatttaggataatgtgagttctgagggaagtgtgaatcagaggggaacaattcaagcagaggaaatgatgtgtgcaaagcaagttatagaaagcatagtgtggcttgtggtgtgatgagtggagagaaattctgaagacctcaccgtaccatgttatcttaggaaaaggattggcttcttttttttgtctaggtagaacatagagaatcttgactatgacttaaataatcttattaaaaaagggaggattgtttttatgaggcagaggttaaggagagagaaaaagaaagaaacgttgaagagaaaggtgaaaacaaataagctgtatgtgaggagtggagggcaggaaacaaagaaaatagtaatgtttgcattatctttcggaagggaagaggtcaagatgttacattactttagagccttcaccactaggtggtgttattggctcaagacagcttggttactgaacttgttgcaagtctgggaagtatgcctttttgttttgaaataagaagtagtgttgggaaattaggaaattagttagaggttagattatcaaaggtctttaatgcaaggttaagaatcatatacttagcaggctaaggggaaatactgaatattttcaagctaaagaataatatcacaaagaagtttgaataggttgactgggattgggggcagaaagcaggaataccaaaagactgtaggaatactctgcatgaacaaaatacccattctaactaggtggtagcagcagaaatgcaaagggaaccattttgagaaattgagaaagaaatgtcaagtttggataacttttgagaaatgaaggtttgagagccaggaggctgtaaagattttagtccatccttcaatttctcgaatacatagagtcgaaaggattaagcaagttccttgaattcacacagttgattagtgacaaagctggagctaggttccacatttgatgttctcccaaccccttcctttttgtttgatttgttagtttgcaaacttggttgatttgggaagtggtgagattggaaaataactggaagagctgttattttgcaatggcgaaagggtgaaaagtctatatagttgtcccttggtctctaagggaggtttgggattttatacccaaatctgagaatgctcaagtccctgatataaaatggggtactatttatatttttactgttttattgtttattttaaagacgttctcaacctgaggttggttggatccatggatgcagaactcttatatagtgaaggccaatcgtataacttaggtttgatggtattttaggaggaatgttacgaggtgtttgagagaggttagagctgaaattgcagatttggtgcactgatccttcatgatttgaggcagagtagatgagatctttaagggtggtaataaatgcatgtggaggaagagttggaagacaagtttgtaatgccagagttgtaggtgagtcagtgtagagtataaaagagtatggaagagaatcaggagaatgttgtcattcatttttcatacctgtggattgcctctttgttttgggggactctgtgaccgaatgaaggatcaggagtgttggggaggggagtgagtggtgtcaaaggtgtcatgaaatcaaggaggatgagcactgagaaaaggtcattggtttgacattagcttgtgatgcgggaacggctgttctgccagggtggacctgcaggatctctgtggagcagcgtttggcggagagggagttctaaggaagccagggcggtgctgtgaaactccttttcttgcgatctttgccagtataaaaaaagagaagaataagagtgtttgtttgaaagaagcaaaggtttgaatgaagtttattttattttattttattttatttttttgagacggagtctggctctgtcgcccaggctggagtgcagtggcgcaatctcggctcactgcaagctccgcctcccgggttcaagcgattctcctgcttcagcctcctgagtagctgggactacaggtgcatgccaccacgcccggctaatttttgtatttttagtagagacggggtttcaccatgttggccaggatggtctcaatctcttgacctcatgatctgcccacctcagcctcccaaagtactgggattacaggcatgagccaccgtgtctggccaagcttattttttaagtaggagaagaacctgtgtgtgtgtatggattctgcacaaggtgtcaggagtgggaggagctgtagaagaggcaggattttgtggtatcagagtatctttatttgcagctagggtgggtggcatctcttctgaggtgtaagaagagagaacaggtgaagagaacattttaagaggaggagggaagatggacttttctccctgaagatttccgttttcttgctgtacatggaacattcactttcctggtctgtcatttagtgaagggttgaccgaatctaattatacagggagcccaattcaacgtataaaataaagtcttacttaccaacttgggagtggaagaagtggccattgttgattattggctactctgccaagatgtcatttaaattacttctgtttgtggagaaaaatacattagatgagatgatctccattcacaagaggtcaacagtccattgagcttctccttgggcaaagtttgtcctggaatgtggatgaggagaaagtggttggaggataaggaatatatagagaggggaaagaagagaatagcacaggaatgcctcaccctgccagaaatagacacacagccgctgcctacctgtcctgtcttgtcgttcccaggagcacagaacaactagaacccgtcatcttcgtaatgccaacagacatgttttataggttcatgtagataactaggttttatatagaggctgtttgaaaaataggacatctgtttatctttcttaagggcaggaacacttcaggaatctattttggattgattaaatcatgtacatggtgaagttttctgaaaaagccagattgtagttccacactgtcattgtcagtaaagaccattctcagggttaaattggaaccggtggttgatcatgtgaaggcaatatttaaagtatgtcttgctagagttaccagattattgcagccccagtcaaagataacaacatataaatggagttagcagaagtgattaaatgacatataccaagcctaaggctgctgctcaatttctagggtcacagcttcctccgttcatgctgaaaatcatatgaagatgggttgaaaagaattagtgtacacccagaaaataaacaggatctactcattagtatttcagtgtaaggatgaaagcttttaaaagtaccgacatggctatgataattgaactagaaaaatagagagttttttttttcctgttgtaaattttatttggaatgtctgaaattttagaaatcattcaacattctttttaatatacagtatttttaaaaagccatttacgctgggtgcggtggctcatgcctgtaatcccagcactttgggaggctgaggtgggtggatgacttgcagccaggaattcgagattagcctggccgtctctactaaaatacaaaagttagctggtcatggtggtgcatgcctgtattcctagctactcaggaggctgaggcatgagaatcgcttgaacccagaaggcagaggttgcagtgagccacgattgcaccgctgtgctccagtctaggtgacagaacaagactgtctccaaaacaaacaaacaaacaaaaaaactcgtttacaattcataatctcttgacaacttcatgatcctgctttgaaaatgtgtttgccggccaggcccagtggctcacaactgtaatcccaacattttgggaggtccaggcaggtcgatcagttgagctcaggagttcgagaccagcctgggcaacatggtgaaaccccatctctacaaaaatacaaaaaattagctgggcatggtggtacgcccaggtaccccggaggcggaggcgggaggatcacttgagcctggaaggttggtgctgcagtgagctatgatggtgccactgctctccagcctgggagatagagcaagaccctgtctcgggggaagaaagatgcttgcaatttagagatctcttaaaaatactttaaaaatgtgacactgaactgaactcgaaattccaaattttaatcatgagttaaattctactgacgctttcactatgttgatagaaaatgagagtggagaagagcacagtgagggagaacacagagttctcaacttaataattttgtgtttttatcataacatgtgaaatgaaaagtttaataatgtcctgttccataaaggctagaatgactacttgtgcttgtaagaattgtgaccgctcagtaataaatatgcggcgtttaacagaattggtgcattgtgtgatgctgtgtctcacctaatgtcctgtctctgtggcgtgtgaaacccttcgaagacttgagcttgaggtcatctaaacacttgaatatatgcagttaggttttcagcacaggggctgtgttctgttttgtgaagaatgtgcagtcacagtaattgatggaagcattttacatgaaggtctccctaatgaagaaatatgaagatattaatatctgatttttactgcaaaagttatttttctgccgtatttatagattttcagagatctatgctaaagatatttaagctttagactgggagcccttctccttcaaggctagactaagtaaacactctttgttttggcttcggaaaatgcattttagaaatcccaaattgcagtgagtttaagtagtgtgatcaccttctaaatatcaaagtatagcccttggctcagatggtctttggagtttaatttccataagcaaaggagtctaggcgataaggtcctttttagatgaaatggtgttggagtctctccccgcttcagctttactgtttgttttgcatgagagccacagccattgttttgatcagggtggtttttatagtgaaacctgaattcgtggttcttggttacattactgcacggttaacaatgtcttccaaaaagtgtattttttcatttaataatttattctactcacagtagcattctgttagccaagtttgcagagtacattcaggtttttcaaagatttcagttaggaatgtggtccacaagtccgtcccagctgtattgtgtcccgtgagaactgctgagtaggatgtggaaggtaattgcatgtttgggaatccagttggggatgagtgggtggtggtgggggctgtggtgggactgagcacctgggacagcatggggagagaacagccaaagcagtctgacgtgttggccatggtctgtgtgtcttaggggatggaacagtgccagagccttctggtggggggatggagggggaagcagcgtggcgcatcagcaagtacttttttttttgtttttgagacagagtcttgctctgtcacctaggctggagtgcagtggtgcgatctcagctcagtgcaacctccgtctcccttgttcacgcgattctcctgcctctgcctcccaagtagctgggactacaggcgcacaccaccgcacccagctactttttgtatttttagtagagacagggtttcaccatgttggccaggatggtctcggtctcttgacctcgtgatccgcctgcctcggcctcccgaagtgctgggattacaggcgtgagccaccgcgcccagctggattagtgagtatttatattatatagaactccaagttctaatgttaggcttcagctgtagaaaaatctttatttactcactgtttcttatcacaccactatgctgtggttccctttcctccctcagtggccgacatctcccctgtcagggcctcccgtttggacccttcaaatcttggggttctgctcttggattttttttttcttctgcctgctctcttccttggtgatctcattcagttctgtggttttaaataccgtctttcatatttagacaactctcaaatttctatctgccggctggagtctcccacatatctgctgcccactcacaatctttacttggatagcccgtagctgttcccatcttaccctgtccaaaactgacctcccgatctgctccctgaggctgcttctcctcctgtctccctctcggttcctggcccccttgttctctacgctgagcagtctttggcatcttctctggtactgttctctcctcctttccctccctctccccactccagcatcaggcaaccttgaaatagattcacaatctgccccccacccccgttgtgccccagcccacacgcgctccacctgtgttgtggctgcagcctctgactgctctggtcgccctccagcttctgccctggctgcccacaggtttttccattttactgagaccgtggcactcctcggttcagaatccgctgggggacttctcatttgattcatactaaactctaaagtcctaaaaattgtcctttatccatgagtccagacggatcttgagaaaaaaaaagattcaggttggatgcagtggctcacacctgtaatcctagcagttcgggaggccgagttgggcagatcgcttgagcctgggtgtttgagaccagcctgggcaacgtggtgaaaccccgtctctataaaaaatacaaacatgagccaggcgtgctggtggttcctgagtatgtaaaattctgtcagtctaaatctacagacgcacctatagtcccagctactcaggaggctgagacgggggcccggaaagctcaaagcttcagtgagccgtgatcgcgccactgcactccagcctgggtgagagagcgagactctgtctaaaaaagaaaaaaaattgaataaataaatggaggaagatagacaaatcagtttctagaagaattataaatattttgagtagcgaaactctctgtctcttaagaatgggctacatgcagtgacttccttaccaagaatacagtacagggaggggtgaaagtaacctgactgtgaggaaacctggcagatactacctcagtcacgtggtcaggagcagcgtcaacatcgataaatcgtgttgattttacccgcttgctacggtgtggtggaaacgactctacctctgtggcttttctccccaaaacagaaccccaatctaatcaggagagaaacgtcagattccagttgagggacatcctacaaaatacctgactagtacttctggaaactgtccagatcattaaaagcaaggaaaatctgagaaacgtacattccagaggagccttaggagacatgacagctaaatgtaaggtggtgtcttggaagggattctaggatggagagaaggccttgggtaaaatgagcaaatttgagtgaagtatggactgtagttgatgatgtgtcagtactggtttgttagttgtatctagtataatgtactaaagtgagatgttaacagtaggggaagctctctttgcaacttttctgtaagtctaaaagcattctaaaataaggtttatttttaaaaagaacccccttcatgataggttctctcccaggatgtgccccggccttaccgctgtccctcaaccgctgtgctccagccacgtggtctctttctggcttccctaatctagtgcttctctgcggggtgtgactttctgccctagagggtgtttggcaatgtctcgagatagaggtatttttagtcatcacagctgggtgatgagggggatgccacaggcttcatgctggtggggaccaggaatcatattgaacactctacgtttccccaaagaatctaagagttgcccagcatgtcactagtgtaagccctgaccctttggatttagggttccctcttttccttcaggttcctgaccctggtctctctttgccctgctttagttttctccttagcacttaattatcagcatcggatatactctgtttacttgtctgtctgtgtctggccaggaaatgtatcttccatcaggacttgttggtaatttggttcacttttgaatccccagcgtctagaactgcacctgatacatagtagatgctcattaaacctatgtaaaaggaattaaataattaatgaaaggggtgagtggatgataaagacaacacatactcatgctaaaaaaaaaccacagtagagaaaaatgccacagtaatcattccctcagctacctgtttctctaccggttctccagaagttaccactgttaacagttgcttgtctgcctagacttttctgtccattttaaagtatatccgtgggtacattgtatctaccccccttctcctctcttttaaataaacataaataggatcagcttcgtttttccagtttatatagctagacatatgtcaacatacagagtgctatcttttaatggctgcatgataactttatacgtagaattaagcaaaaatacttctagtacttgcaatagaatcctggtataatattttgttgtacaatggatgatggtaaaaatgtctcctaaaacataatgtgactcctaaaacaaaagacactgcttgctaaacatcatccaagtgtttctggttttgtttttttgtttttgagacagagtctagctctgttgcccaggctggagtgcagtggcgcgatctcggctcactgcaatctctgcctcccgggttcaagtgattctcctgcctcagcctctggagtaactgagattacaggcttgctccaccatatcctgctaatttttgtatttttagtagagatggggtttcaccatgttttggccaggctggtctcgaactcctgagctcaagggatccggctgcattggcctcccaaagttctgggattacaggtgtgagccaccgtgaccagcctctttatatgtatttttatttctagtttgtaaatcccttgggattgggatctttgtcttttattccttaacaccatgcaaggttctgtctgataaggtcctccatacctgtaggttgaactttttatttgttttgataaaattacaagattattttataatattcaaatttccctgagtattagaactgttgactaaatggagatacttaaagagatagttttgggagaaaattgttttcttattttgtttaaatgataattgttctcctagaagccaaaaatctgtctaatttctgactttatctggagaggagaaggcatcatagctaaagaacaagttagggtggccaacttgaacgtggttcaatatcaattaagctttaattgctttaagcacatcaggtctttttttccccctaaggcaatgacttatggggcagagttggcagtagctaagttaagatactataaattaatgttgtcgtaccaatttcaactgggctgtcaaacccaccctccagtttgctcttcatttacctctggtagcattcacattttgtgaatattaatttgagagtcactttgcaagatgacagtgctgtcctttgtgaagctgtgttgatttacaagatgaatttgttggcaaaggggaacagttgatctgaaaataactgtcctgcttccctggccagacctcagcccagtggacttcgcccaccttcccatttgtcattgttaaaggcaacgttttcttcatttcttcagtttctctcttttctccgccagtcacccagggactgtcctgcagagtctgccaccatgtattaatagactgatttgacccttgacgtgtgttagcttagaagatttagcttttgtgtgtgtgtgtcatcttctcatggaggggcccagggtgacaggttgttcgatgccctgaagataggagacattctgtgaacaactggttgttgacagcagtggtgataaaacattgatatagttggtaaagtttgcttcactgacttagagaggaaaaatgctgtttgacatcaggctgctgaattctggtttttcttttgtaaattgcctttctttggggcttatgttgctgataagtattatacttgatataattattttaatacttttaagtagttacatagagcgaaatgagacaataaacccttacattatgtagggagtgaacttaaccattcttatacagaaataatataatttcaggatacattttgaaatgagattacaagctgttcttatggagcgccatgtaaaaccactgctggtttttgtttttttctgaattagttgtcagaagctttgcattctcacaatactgtaggggcattatgtttgccagcttttgatcatttaattcattgtatgtaagtaaatgtaaacaatgttcattaatttttaacagttgcttatatttgccttaacgatttaaatataaaaatataacttagttgttacagttagtagcctttcatttgatactcatgaagatttttgagtcgtttaaatatgaaagagaggaccttgtcgttcaaaaatgaaaaggaagtttttgttaagcttaataattctagacatgttacaagaatgttgatacttatattggaaaacatgaaaaacatcccaaaccgttgcatttatttatattccatgtgtatggaaaatatttttttaatgagaagtttaaaacaatgtacacacttttgcttgttttatttccccaaggtaacgattctgatttcttctgtttcagattcccgacagttaagcaatggggagacatttggctttgctcctgcttctgctccttctcttccaacattttggagacagtgatggcagccaacgacttgaacagactcctctgcagtttacacacctcgagtacaacgtcaccgtgcaggagaactctgcagctaagacttatgtggggcatcctgtcaagatgggtgtttacattacacatccagcgtgggaagtaaggtacaaaattgtttccggagacagtgaaaacctgttcaaagctgaagagtacattctcggagacttttgctttctaagaataaggaccaaaggaggaaatacagctattcttaatagagaagtgaaggatcactacacattgatagtgaaagcacttgaaaaaaatactaatgtggaggcgcgaacaaaggtcagggtgcaggtgctggatacaaatgacttgagaccgttattctcacccacctcatacagcgtttctttacctgaaaacacagctataaggaccagtatcgcaagagtcagcgccacggatgcagacataggaaccaacggggaattttactacagttttaaagatcgaacagatatgtttgctattcacccaaccagtggtgtgatagtgttaactggtagacttgattacctagagaccaagctctatgagatggaaatcctcgctgcggaccgtggcatgaagttgtatgggagcagtggcatcagcagcatggccaagctaacggtgcacatcgaacaggccaatgaatgtgctccggtgataacagcagtgacattgtcaccatcagaactggacagggacccagcatatgcaattgtgacagtggatgactgcgatcagggtgccaatggtgacatagcatctttaagcatcgtggcaggtgaccttctccagcagtttagaacagtgaggtcctttccagggagtaaggagtataaagtcaaagccatcggtggcattgattgggacagtcatcctttcggctacaatctcacactacaggctaaagataaaggaactccgccccagttctcttctgttaaagtcattcacgtgacttctccacagttcaaagccgggccagtcaagtttgaaaaggatgtttacagagcagaaataagtgaatttgctcctcccaacacacctgtggtcatggtaaaggccattcctgcttattcccatttgaggtatgtttttaaaagtacacctggaaaagctaaattcagtttaaattacaacactggtctcatttctattttagaaccagttaaaagacagcaggcagcccattttgaacttgaagtaacaacaagtgacagaaaagcgtccaccaaggtcttggtgaaagtcttaggtgcaaatagcaatccccctgaatttacccagacagcgtacaaagctgcttttgatgagaacgtgcccattggtactactgtcatgagcctgagtgccgtagaccctgatgagggtgagaacgggtacgtgacatacagtatcgcaaatttaaatcatgtgccgtttgcgattgaccatttcactggtgccgtgagtacgtcagaaaacctggactacgaactgatgcctcgggtttatactctgaggattcgtgcatcagactggggcttgccgtaccgccgggaagtcgaagtccttgctacaattactctcaataacttgaatgacaacacacctttgtttgagaaaataaattgtgaagggacaattcccagagatctaggcgtgggagagcaaataaccactgtttctgctattgatgcagatgaacttcagttggtacagtatcagattgaagctggaaatgaactggatttctttagtttaaaccccaactcgggggtattgtcattaaagcgatcgctaatggatggcttaggtgcaaaggtgtctttccacagtctgagaatcacagctacagatggagaaaattttgccacaccattatatatcaacataacagtggctgccagtcacaagctggtaaacttgcagtgtgaagagactggtgttgccaaaatgctggcagagaagctcctgcaggcaaataaattacacaaccagggagaggtggaggatattttcttcgattctcactctgtcaatgctcacataccgcagtttagaagcactcttccgactggtattcaggtaaaggaaaaccagcctgtgggttccagtgtaattttcatgaactccactgaccttgacactggcttcaatggaaaactggtctatgctgtttctggaggaaatgaggatagttgcttcatgattgatatggaaacaggaatgctgaaaattttatctcctcttgaccgtgaaacaacagacaaatacaccctgaatattaccgtctatgaccttgggataccccagaaggctgcgtggcgtcttctacatgtcgtggttgtcgatgccaatgataatccacccgagtttttacaggagagctattttgtggaagtgagtgaagacaaggaggtacatagtgaaatcatccaggttgaagccacagataaagacctggggcccaacggacacgtgacgtactcaattgttacagacacagacacattttcaattgacagcgtgacgggtgttgttaacatcgcacgccctctggatcgagagctgcagcatgagcactccttaaagattgaggccagggaccaagccagagaagagcctcagctgttctccactgtcgttgtgaaagtatcactagaagatgttaatgacaacccacctacatttattccacctaattatcgtgtgaaagtccgagaggatcttccagaaggaaccgtcatcatgtggttagaagcccacgatcctgatttaggtcagtctggtcaggtgagatacagccttctggaccacggagaaggaaacttcgatgtggataaactcagtggagcagttaggatcgtccagcagttggactttgagaagaagcaagtgtataatctcactgtgagggccaaagacaagggaaagccagtttctctgtcttctacttgctatgttgaagttgaggtggttgatgtgaatgagaacctgcacccacccgtgttttccagctttgtggaaaaggggacagtgaaagaagatgcacctgttggttcattggtaatgacggtgtcggctcatgatgaggacgccagaagagatggggagatccgatactccattagagatggctctggcgttggtgttttcaaaataggtgaagagacaggtaaatatgtttttattgctctcatttgatgcccttttccattttttttttttttttaaccatctgccctgtggcagcttcttcaaaattccctgttgctctttgggctcggtatagaagtgcgtgtgaatattttttttggcccggctgctgtattgtagatgtgtttattcgtagtaagccatgaagatggaaacaaaagggcctgagtgatttaagtggcgtaaagtgagtaaaagaccagggatacattgaaattgttatcactgatactgatctccaaactctattttgaactctattatcaaaagcatcattatggttttgttctgactttgagaagtctttccaacttttttcagtcagtgtatttgcaggctcatacttagaacaactactgctaccatctggcgtgccattcagaggttgttgcacaccggcaaagttacccagtgcagagatgatgtgccaaaacttctctaatgtggtcttcacttagtacctcgtctaggttttggtcgagaggcagaggcggggtagtggcatgctgaactgagaagacctggcttggaatcctggctcttctagttggtaattggtgatcctggacaagcaaatgacttcttagaactaactttttcctcttctacatggggacacttagaaataccggaaggtttaagtgagagtaaagtgaaataacacgggaaaactcctgacgtgtggtaggtttattcatggtagcagtgctttgttgtaatactgaaggttctgaaaccgggacctccaaggcagcagtccaagctagaaagtgcgtgtgttgcactttgtacatacctcagtcttgctgcgcagatattgtagttatatgtcaatatgtcctggtggagtgtgagcctttcggggcaggcttttagcccctgcatcttctgtgtttagcacctagcaaaaagtgcctggcacatagaagctggtcaatcagtgttaaaagactgcaggagtaaatgagcgaatgtgttggcttttcatgtcctgccctctgagaatgatagtggaaatagaaaaggggtgatcatagcactcatcagcaataggtggggctcaatttgtttactaatctcacccatgtgtactttataaatctgtgcattttaaaagggacctttttaaaattacctttaaactttatatattcagtaataccacacacacaaaaaaacaagagagtcttcagtcaacacccaccctgggatccatgccatctgataggtgtgtagcacatgaaaaatgtaactgctgtttagaaaaacattattttgggctgggtgcgatggctcacgcctgtaatcgcagcactttgggaggcgcaggtgggaggattgcttgaggccaggagttcaagaccagcctgggtaacagcaaaacctcgttcctaccaaaaaaaaaaaaaaaaaaatgctggggtggtggtacacctgtagtcccagctacttgggaggctgaggtgggaggatggcttgagtccaggaggtcgaggctggagtgagctgtgattgtgccgttgcactccagcctgggcaacagagccagactatctcaaaaaaaaaaaaaaaaaaaaagttattttggaagttgtaatatattttttgtgcttctattaatgcaaggaattctttctctgttggttatgtgccactagcatatggcctatgtattaagtctacagactatatatcactatctaaaatacccataattagtttatttcaagagaaactagcataatcaacagcatttgataatcctctgaagagtagaatgtcttttttatcattttatctgtccagtataagttagaattttcaaatactgtttcaacatagaaaaataccccaaatttaacaactgaagctgagtcaaactgtttttgtaaaatttttgaagaacagttattcctactttttttccttctccaaagtaggtttcaaaagcctataaagaactgttaaaagttaaagttgagacaaaatggaaccaaaaccttaatttaaattctcttcatgactttgtatgaacactgccttttgggttttttctttcaagtgaagaagagtatttttattgttgctttttaaggaatcatttcgaaactagtatttttcacctacacatttaagagattgggatatttttactaccaagaaaaaaaaaataaggaaacaaccagccaaacgaggtggtgtcccaaaatagatttatggtattttccagaaagggcagtgccagacttttccagtagtaatttagtgggaggcggggaggtgttgggattgtgggcagtgtggttggtaagtatataagaaactgtgtgtaaatattgagatttaaaggtgagagagaagctggaaatttgtaggtcatttattctgtggtccttgctttacagagaaagtaagtagtgtacagtagacttacgtttatatgcttttggaaatgaacaaaagtgatagaataatatttacacattggacctcgtattcattacaagatacattttttggcagaagaattatcagcccaagttatttcttcagagattcctggcaggacaattggtatcacagtgtagaaactcctttaggaaaaatcagtatattttgttggctaaaattaggcatgctaattattcacttggagtgtgtcttaactaatttctagggcatgaaggaagtgtggaaagatgcgtgtcaggtgaagagatggaggggcagttgaggaacacccgtgggggctgccagagactgcactacagcttggtgtgccttggcgtgcacccggcccaggccaggctggtgcttgctggaacttcgctccttgcccattgcccatgtccaccagggtcttctttgcctaactcgggggcatttaatattgcgctaattaatatgagcctgtgcaggagtgctgtggctctggggaccaacaacctgtccacgtaactgtttgcttcttgtctttgacttaaagctactagtgttggttgagtattataaccaaatttttgtcatattttacctatttcaacctagtcaaatatgtgtatcatttatatacttttcacagaacagagaacctatccagcaagctgaatgaagttctattttaagatctttccttttcttctctttaaatctgcttaaattggaaacatcctctgaaacgtagttttctgtttttgtagaagtagaaattctatggactacctaaaaatctgtcaccaaacggacagtcctgttagctctgtgttcttagctccgtggtttgtacatgttctgcaaatggctttgcagcgagagaaagcgtagtggcagagtggggatggcgggcgtggactctgcaggtctcacggtatcatctgcccttcctggagaggcctctggatgttggtgaactgttctgacagatccatgccacgtgactgcccttccatgtgaggtctggaaggggagagggccatgtgaacaattatttggaaagtcggtctcgttagagagccagtttccagagctctaattaagattctcctcctcagattgctatctgtaaggtctacagtgaggattataaagtagatttaacaaagatgtttagcggagtaaagatctttgaagcatgtgtaatcccacattcagggttcggtctgcctgccatgcgtctcagcatccaaccccctcttctacttcagatccattctctgttgctttgttgtaagcttcaacaggttttcctgtaatttaagcagaataaaatctggagcatccctcttgtgggtgtgtggaataaatttggtgtagtgttcttagctgatgtgtacttagcaagttaattattacttgagcaccaatgaaatgctgcagctttttgttcatttttaaaactaagggaatgtctagaaaaatgcctggccagaagattttgtagaaattgtgaatagaagttggtgttgaagcagtgttgggttgaggtctgttccctgatgtgtgtattgcgtacaggacgccctggaatgtgtttgaccttcagtctagagtagtttggttctctttttggctgttaatgttagtatacaaagcagcccaggacgagtgtttccccttctgactctacttatttggggagcccgtgtttttcattaagataagagcagatctctaaacatattactgctgtgttacttttagcatcccaatcctggtttcaggattgaaatttacgttttgatgaaaagactgggaatgaattaatatggaagtacagccgttgtcacaacttaagtcagcattcatgaaatacaggaagggaaatgaggacatttttcttaagagaaaatggctgcattcacacgatggagagtgttggagcaccatccctgtgtcacctgtgtggaggccggctcctggcctcatgtctgtgtcacctgtgtggaggtcggctcctggcctcatccctgtgtcacctgtgtgggggtcggctcctggcctcatgtctgtgtcacctgtgtgggggtcggctcctggcctcatccctgtgtcacctgtgtggggtcggctcctggccgcccggcttaggtgcacacaggtgagtgcgtttctacagtgggaacggaacggaattccggcccaggctaacctgattgttgacctcagtcagggaaaatgtttctggagcttctgtctggttctgcagaactctgaatgtttcagctatttattattcattcaagttagaaacccttttcctcatataaagtaggatattttacttacatcctgggatccctgctcctgctgcacgtcctcctgtgacaggagaaggaatgtggaggaggctgacgggccccggatcctgactaaagggtctgcaagtggctgagagctgggtggctgctggaagcctggcctgctgtggtgggacctgatgccctgtgggagctggggctagaattcctggcagcaggaacttgattagagacgggtgggaggcccggcagagagttaaaaaatgtgctggtgaaagggcttttgtgttgctggctcctccctgaagtggcagcagctgctgtcctcttggctctttatgttctcctcctaggaaagcttaagagaaaagaagctcgtttcccagccacagggagactggaagttgtgtcttggcagaaatgatgttaccacagtgtgttgttgctgtgttctgggataaaaatcttgtgtgtgtgtgattctgagtcaagcagggcgtggtaacaatttctaccaggggaagggtccttctttgaagcaggaagatgttaaggtgaaaataggatctagtcgaaatcctaaggaagttccattttcttactaaaaacaacagagattcagcttttctttagttatttgtatgatgtttaccacgttgatgcgttactgactgaacaagtctagcctggaaaggctgtggatgggaactcgggggaccgtgactcagggccgtcccacgttgtggagcagcacgcagtgacgcttaccgtgctgtgatgtaattctcagagtgtgcaggggccgggacctggaggtgtggggtggaaacagcttacgtcagacaggccggctttgtcctcatgctgtcagtggtaatgacactggtggtgagcaccagtctagtaataaccacgacactgagtgtcacagccgggcactgggcccggcattctgtgtgtgccagtccatttaattctcacaaccaccgggtggactctgttatccctacattacaggatgaaacgtcaatgagaggttaagtaacttgcccaagacttcttgggtgctaagtgccagaaccgcattttactcctcttactctttgaagaatgatctacggatgctgcgagggagcctgttcaaaaacgactagtgaagtgggtttctgtgctaagctgggttaggccaggttgcagccttgaatcctagattcgatgcttggtcctgaaggagtcttgaatgtaagaatcttccagcatttttactctgaatgacatattgctatctagcacaattaggaggacaggagtttgaattccgtcgcggtggcagtcagcaagtcaagatcaggccagcgaaggcgtgggtgaatccaggtgagtgcctgtagcttcccttgtctcattggccattgaagagatggacaggtgtcatatttgtgtttgcagttgtaaaagtgtgatttaatcggtaaaaatgctgcccatctgaactggacaggacgttaattgttggaaggcttttcttgtggaaaccatgtttaagatggtggttaggtcccagctttcaggcctgcacgccaaagctaacgtgatttgtacctccctctgcatcgagttacatgaggccatcgttctcttgccttgtcctccttggtgccttggcatcagcagctgtcctcttggccatctagtacggggcaagaaagccagagtaagcttactcaccgtcccccttgccagggaactgttttttgtcctaaggatgtttaaggattgggtgttcgtaatttgttgagtgacctgtgtgtgtagaggtttaatgttcacttaatacctggtctgttctttccgtattacctaccgtgtacaactatgaaaattgaaaagattttaagctttaggtgttagtttaaatgttggagattttcttaccatttagttcccattgtttttactttttggattattttagttcgtagggctggatttgtcacgtttttatttattagtgtgtgttttgtgtactgttattttgcagttgcctttctggcatttttttttttttttcaaacagcagaccatgctgcttataaatagtgcatgctccatacaagacatagtgccacaaagcgcaatcttttctgtctttgttactaggaataaatggacttggaaagattgccgttcaagggcacatgcctgtacttgttgaattggcagttctccagtttcacagcagtgacagtaagtctaggaaactattcacatatatacttcaagggccagtcttatcaattaacttgccttgccagttcaacagtaccatatctcctcgaatatctgataccatgttgaactcggtccattgctaggcctccgagtggctcaacctgttgaatgggattctttagaatgtcttactcatttcatgttggggagagtctactaatacagtctttttagttttaaatcaattggttttttgtttttttttaataaaagttactcagctatttatgtagtttctgaagactgctttctggtttgtgataatttaaccatattcttatcacaggtaagctttttcttcttaatcatcatcagcatgtaattatagttaacgtttagtgagtacttgccgtgtacaggccctattttatggtctttccatagactgtctcactagattgtcacaattagtgagtaaaattacgtggtgttatactctgctcatggagttcatgtctgtgccaaattataaaaacacgacaaatgatggaaatatgatgcacgtcgacttagtgccattgaattttgaaatattctgttgcattttttaaaggaaagtagcgcgtgccagttgatgtagtttatggtgtgatgcgttaggaggcattctgacaatacagacgtgattcttgagctgaggtgttcatctgtcacggaatcctccttaacccgtttcactgtctcccatcgcacggggtccagtctctccgtggcggagccgcattgccctaacaggcctccctgctctcccatttaacatcacccgtgcttccctcactggctcctgtgcagccctggtccctcagcccctgccctgtgcagccctctacccctctgtgcctcgccgttttctgtctccctgcctttgggcatacagtggtcagctcgtattatctcataactctacttaataaagctttcttgacagccctcttcgttacgtggagctgtttcttatcttttatgtacttaaacatatgactgcagtatcactctagtgtaattgtttatttacacgcgcctttcctctagattgtcagctctttgtgggcgcacgtaagtctttgaatgcttggtgtatataacaggcactcaatgcataatggttaggctggcctggtaagccatcgtggggtctccggggaaacctttccttcaagccaggtagcctttgcttctcttcgctgcgggggcacttcctgtgtgttcctctgttgtagtgtatgactcagtgcgcggcctgtctgtgacttcagtccgtctccatgaactcaaggacacagtccagcgtaatcatctgtctaccctcttctaagcacagtggctgatagagcagtaagtagccctcatttgttttgtgagcaaatagattgtaacacacagtaattgcagttgtgaaggttgagcgaagcttcttttctgccaccctggacgcctatttgtaggctccaaaactgtggttcttgatggttttggcgtcacagaaactttgagaatctgtggaaagctgtatattctttcttcagagagacagtgatttgattcaataccaaggggctcagtgaagtgccgaacctgtgttcatgcactgaccgtcttcttggaatcctggttttgagatcctctgttctggaggttccagggttgtcagtcctcacaaattctcataaaggaggaccagcgggtaaggataatggtgatgatgatgatggcagtgatgacagtgacagtgacaagcatttttataatgccttacaacctgcaagtatttctacatacattatatggtgcttaatgtaatagagaaataaaacctgtataaaaattataaatgagctagataaaccttaacaatggacaggtcacttttctatatatttgtacatgtgcctgaaatgatttaggagaaaccgtcattcaaggctgtttccaagtgaaataagaacctgtagttctttatacacctgtttcatcagatgttctgtctccccattgaggcatacggcacaaataaaacagatataatgaacagtgtgcatcatccctcggagagcatggggatcggtttctggaccgtccgcgcacacccacatctgtgcgttctgaggtcccgcagccggccctccacagacgctgcgctttcgcgtgctgtgagtactgtgtttttgatttgccttcagttgaaaagagttcatgtgtaaatgggcctgtgcagttcaaactcgtgttgtccaagggtcattgatacctcgtttaagttaagcagctttctaggccaggctttggtctttagttttgccagctcaccatggaaaacccttaatgccacaccactagtattcctttacttattaaggcaatctgtcaggaaactcaggcctctaatgataaagttagctcccttggcagacagaattaattaattaattaattaattattttctaagacagagtcttgctttgttgccaaggccagagtcgagttgcagtggctcaatctctgcctgctgcagcctgcacctccaagttgaagtgattctcctgcctcagcctcccaagtagctgggattacaggtgtgcaccaccacacccagctaatttttctgtttttactagagacgagtttttgctgtgttggccaggctggtctcgaactcctgatctcaagtgatctgcccaccttggcttcccaaagtgttggaattacaggcgtgagccactgtgcccagtccagaatttttatttaaatggacttgccgtatgttctttgacctctatggaaaatagccctgccataagttcttagttaaaagattctccaaaatgcatttgtgggtcctggtttgcttttagctttctattaatttttttacttgcctctttattcttagggctgcttgacattactctgtctcagacgtccgtgttcctcttatcacagtgcttggcacagggctggtaccgagtagctgcctgctcagggggtgttcatcttttcatagatcctgtgcatgtttgtcccatgatcctcttctttcgatttgttgcccagctgaaggtgtttacatatttaagtgaaaaaaggcgggtttgtcatgtaaataccaagttagtctgttggttgaggcaacaaaatggaaatcagttattttttaaggatctaaaaaattttaaaaggaagtgctttttcttaatagtgtggcaagctcatttagaagatggaaactgattaggaaagtatgaacatgagtaatatccatatgtggttttaattggcagaatgttgatttcagaaagaagtcacacgaagtttcatatctacaaagtatcaagattagagtttaaggtcagtaatttttaaaataagtgttctagggcctatggccgggcgctgtggctcccgcctgtaatcccaacactttgggaggtgaaggcgggcggatcatctgaggtcaggagttcaagatcagcctggccaacatggtgaaaccctgtctctactaaaaatacaaaattagctgggcctagtggcgcacacctgtagtctcagctgctccagaggctgaggcaggagaattgcttgaatctgggagctggaggttgcagtgagtcgagattgtgccactgcccttcagcctgggggacaagagtgaaactccgtctcaaaaatgtgtgtgtgtgtgtgtgtgtgtgtgtgtgtgtgtgtgtgtgttttatatataatacatattaaaaattattttagcatgtttaaaaaatctgcttttgtcatttgacttccaccagaatataaggctgaaactggactctgtccgtgtctgtatcacttacttaggaactgactggcatctaggcatccagccaatactggttgaatgcgtaaaatttaaaatggaaatagattgtaacaaaatactaaagggaaacgcttatctaaataactattgttgagaaagcccttttaaatttgtcctttttaatatagttaaggctgctcttccacttgagacaccctgtctcccataccttccaaaattacaagaataagaacaacaaagcagtttagtagcaatgcgggttcctctttgtttctgctttgcttacctaggagcagcctcctttgtgagattgagtatctagattctgaggctggccttccaggtgaatatacctgagttacgtgttgtgtccttgggctgtagagtagtgctattgcctgtatcatcctctggattgaaaagtatttcttttgttttaagtataaaagatgagtatccattgccattgcacattattcagcaaaaacttgaactctaaaaaacgttcagattagtaggatctatttttgttagtatcattattgatgaatttcttaaaacttaagaaaatagttacttctagagattcacagattatttttgaaactgttggttttctggattaaattgagctcacccaactatcctttgaaaaagctgttttgtatttttgttttgttttgtttttgttttgagatggagtctcgctctgttgcccaggctggagtgcagtggcacgatcttggctccctgcaacctctgcctcctgggttcaagcgattcttctgcttcagcctcccaagtagctgggattacaggcacccgccatcatgcctggctaatttttgtattttttgtagagacggggtttcaccatgttggctagctgatcatgaactcctgacctcaggtgatctgccttcctcagcctcctgaaatgctgggattactggtgtgaaccactgtacccagcctgttttgtattttaagtgtcttggttacttctgtggagaggtgcaatttacttaaaaccaaacactttcttttaaagggtggttgaataatggggggaaatttagttagttaccagactcttttaagaaaaataatatgtatgtaaactaaaagtagttttcttattttagtaactacatctaagtaaaagttttttaaaaactcatttgttctttgtcaactcttcttgctacttaggaactctgattctaaaccttccactcatataaatagaaaattatcagtagctagttcagttaacttcattattctatttaaaagtaactctttaggatgattaaaggaaggaaaggaaaccgttgtggaagttgttcactgtgtatttgagtagaaatctgttcatagcaagttgcatttgagagaggttgagcgttatgagaaatggggagatggtggttaggttaggtgagaggcaggttgagtttgggagatgttgagtttcgttagaaataggaagatggtggttggattagtagatgagaggcaggttgagtctgggagaggttgagtgttgtgagaaataggaagatggtggatgggttagatgagaggcaggtcgagcttgtgagatgttgagttttgtgagaaataggaagatggtggttggattagtagatgagaggcaggttgagtttgggagaggttgagtgttgtgaagagtggggagatggtggttgggttagatgagaggcaggttgagtctgggagaggttgaatcttgtgaggaatgtggagatggctgttaggttagatgaaatacatgagaaactcaagtggaataagtgtaggcaaatccagagctgaactgaaacgttttattgtctttgggccaatattaagtattgtgaaggaaattacagagaatggcatttagccttttgttgggaaacaaaccatcccaaaacctcatagcttagaacagcctccactcatttgggtcaccgttctgtgggtgggcgatttgggctgggctcagctgggtgtttccttggctgttttccacgggctgactcatgtttgatcagttgctgttgagctcgagggccctgctcccaggaggtggctgggtgttcgctgcaggcagcagctcctaggccctgtgtctgtgatcatcccgcagcctgtcctggacccgtttacatggtggtctcggggttctgggtgctcgaggaaagcagcaaatccgctgggccaaggctcagaactcctccactgtcatctcccacgtgctgtgacacagttcgttcatccagccacaggtagggaggtaggctaaccttgctgagaggagctgccgagtactgtgttcatgttcctacttccagaggcacttttacccgattgcgtttccctttgagggaatcccttctgaatgttaaatgtgcattactatataaaggcttgtgaaaattgtgaaaatatcaatatggtcttaaaacttttctgtcttacataacgatactttaagtttgtcatactgttcaaacatgtaatggcattacagtatatcttcaagttatttttagcacatttccatttaaactagaacgtaagcagagataccagggggatgcttgaagtctgaagataccaaagcaatgctcctccatcgtcttcctacttcattttctatcacaaagatctctgaagtttcttacccccaaaggaattggagaattccagttatcgtgtttctaaagtgaaaggcggagagatggagtgtctggtaaagggcagcaaggggttcttcacgatagactttggcctgcagatgttcgtttttctcattagcaccacttcctgctaattaatttgtcacaaactatctcattgatttattgctgtcccgtagagaatggaatacttggggaaatggaattggaagtccatggaagactggggggcgtggaaatagacaccgaaatgaggagagaatataggagtgttgatttggtatctttaggtttcatgcagccctctgacatctctgcttaagtttcagcttaaatggaaaagttttaaaaataacctaagattcccagcactttgggaggccgaggcgggcggatcacgaggtcaggagatcgagaccatcctggctaacacggtgaaaccccgtctctactaaaaatacaaaaaattagccgggcgtggtggcgggcgcctgtagtcccagctactcgggaggctgaggcaggagaatggcgtgaacccgggaggcggagcttgcagtgaaccgagattgcgccactgcactccagcctgggcgacagagcgagactccgtctcaaaaaaataaataaataaataaataacctaagatgtattttaataccacttagtattttgctaattttagcttatattagagttgtttgttacgggacgtatgtgcaacagttaataggaaagtgctctgtggtaagttgaaatagaaatcttaggatttgaaatagaaacgtgtaactgtattgactcactaagcattttgacattatttctgtttggaaattttgggagtggatgtctttgaacacattgacatatacatgggggggaagaaaaacaaacttgtggccttctttaacagagatttttagagaagtagtaagtcccagctgccattcttctcctcaaagagattagaaaaagaaccacataaaaataatcattattataggtaaaaaaaaatagtagccacagtaaagcatacatgtttattgtttactgcttgtagaaaggttcagtgatcattaaatgtcagaacgaatcgtcttgtttctgttttttttaatattttattttgtttttatttagagatgggatctcactttctcagactggagtacagtggcacaatcagggcttactgcagccttgaactccggggctcaagcaatcctcccaccacagcctcctgagtagctgggactacaggcatatgccaccatgcccggctaattttttttactttttttgcagaggtggggtcttgctatgttgcccaggctggtctcgaactcctcagctcaagtgatccatcctactaccttggcctcccaaagcgctgacattacagatgtgagccaacatgcctgtcacgcctggctcagtggccttgtttctaacaaaaaattttctcatgaaatgaaatgaagattctgtaacaagttagttgacaaactttgttagaaacctaatgtagagatggtgacttaaaatgtgatattctcataaacagtaagtcttggatatagtatatagaagatcatgttagcgaagagaataaaagtcttaaattgtttcttttggtgttagagatgaatctgcactagaggcacctaatcagcacggtgtactctgtataagctaaaacaaaacacaacagtgattcatgtcccagagtagaccatgaaaccacttattagaaatattccaaagtttactgtttttagatacaggtgctcatatgtgaaaggtgaactgatttttacaatttatattaacaaaattgtgcaaattctgccactttttaccgttactcgttattttttaaatcaaatgaattagtataacaggtcaaaagcagcactgcccaatagtactgtctttatttgtaattttatatattttagtagccacaataaaaaagaatataacagatgaaattttaataatatattttatttagtccaacatatcaaaaagtacaattacagcatgtgattagcgtaagtattaatgagacctttaacattcattttttcctatgaagtctttaaatcagtgtatattccacccttacagcaaatttcagtttcgactagccccatttcaaatgcaagtttagagacttatgtagagttcaactgccttaaagatatttgtgactttaaatttggatagtgggctactagattttttttttttgaggcagagtctcactctgtcgcccaggctggagtgcagtagcgtgatctctgctcactgcaactcctgggttcaagtgattctcctacctcagtctcccaagtagctgggattacaggtgtgagccaccacacctagctaaatttttgtatttttattagagacggagttttaccatgttggttaggctggtcaccaactctggacctcagatgatctgcctgcctcggcctccaggtgctgggattacagacgtgagccaccgtgcaggctattagattttcttctgtgaatgacgtacacgaaataagtgacctagagctacgtctctgatcattcactttggttataatttttttgcaaattacctatgctggaatcttttggggttggtgggaatggttttgtttctgattgtttttacttcctcaactgtcctaagattaccagcattctaatgaatagtaaacttgagataacggcttcatgcctaacaagtataatttatctctgtagtctcaagtgtttgctcttctttttaaaatgtttctttttagagaaatacagctttgaattctactacacttttctgtaacgcaatgcagtaacatgttttttgtttatttgtagctatcctatagggatagggaaaatgtaattgaatagtaactgaggaaggggctactaaaacctgagaaaaaatgataggaaagttactctttcccttcatttgggtgatgggaaaaagtaggtatgagatcttctaatgggcttgtgagtgcggtagtctttttaaagagtcacatggtgtgttaattttcctctggatattaagagatcaccaccaacaaagcaaggcttgatcgcccaggtgaggtggtgtgcggagtcgagtcgtgtggcgggtgtgcatgcccttgtgggaaaggaagtggaagagttaagtagttctgcctgttgaattagcctggtgtgggtgggtgccagataccctgggctaaggacattcaatgaggatgttaaaagcctattacactggtgttgctgaaaatttatttgttgaagaggctggtggcctagatcctggcatggctttaaaaatattacagtgaagcaattcttgtgatgttaagcgagtgtgtgagctcattttggagtcttttgacttggactctctctcgctttggttaatgagtgtggaaaaacagcccagaagttaatttaaaatcagactttgttttcaattaaaaattgtccatgatgggtcattggatgtgaatgcaaaaagaaaaaaaatgtccagtgatagcttttgtaaaattccgagtaaatcatgtgtctttttgaggataaacatttgttattgaagttccctcatttaaatagctttttggggggcataatttggatttgtagcataattctaatttgatttgacgtacaagatacaaatcttctttatgtgaaagtatatgacatcttaaagtgatatttttacagagtgattttccaaaagatttatttagaccaggtccatcatttccaagtgaagtgaaatctttattcattaactgtaaagtttctgtgggcttccacaattgctaacatgtataattaattcaaaacccatgcttttcacacttctttttgaaaagtagttgctttctgtaaacttgacctttctgttgtttcaatggcctgtttattcttttcagagttaaaaaggtcattttcaggtttatggaattttccatgtaaattttgggatctgcatgctggggtgattttctgctttatatttattgcggtactttttgtgtccttgggtttaaacgaatacaggttatgaaaacctaccctgccacattgtggctgctgcctttgtatctcacagatacacctgtttcatttgggcttcatcagtttcacgtcaagagttgtgactgaagcttaagtaaatatgtttattttgtgccggctgctggctggaaagtctactgtagcagctgaagggtgtgtgtttttttgggggggggcggggggggtactcttgctgggtgaaccatcattgtgacaggtaaatggaattcgtcaataaagcttgaattgcgtcccccttcatttcttccgagctgttgaatgtagggattgtcttgcttggaaccctattgattaggtcacagcctggtctgcagaaaacagagctgcccaggggccatctcactgcaggggatttgccgaagcccctgtgaaaggaccacatggggtccgcgattaccttatgaacaggaatcagttcagggagcagatacatattaaggacctattaacttctacaggcttctctttgacttgagaagcactggagagtgaagattccttcacacataatgaaaggaaattattgccaaagccaccgaaggtgcatcttgccatatctctgtgggtgcctctgttgagttgcaaatttatttcctgctggggcatctgcttagctttagaagtcccatttaaggtcatgaaaagggaatttggaaatgattacatagtgttggctttttttttttttttttttcagctttgagtctccatagcatttggacagaattagcgaactcccctcattaaaaaaatgatctttatttccaataaaaaggcagtatatagtcattgtagaagcctgggaaaatgtaaactactggaaaggagaaaacatgactaaaattttactacctagaaccctggtgaacttttcccctaactcatatgtacctgcacaaagatgcctttccagtgccaggtgcgggatttgtaaaaatgatgaacagaaaacatttactgaacgtgtagttttgttgactgccttctgctatatccagaacattttctggttggtaggtattcttaaagaatgtgatttttagaatatagagtgccagcacagagacagactaatagaagttgagcatctttatctgaaatacttgggactagaagtgttttggatttatttggattttgagatatacgcattataccagttgacatcccacatccagaaatctgaaatccacaatgcccagcgcaccatgttggtgctccaaagtttcagattttgcagcatttcagattttttgatttgggacactcatcctgtttgtatagtaatactcaatttttgaatatgtaagttccttgtttgtgtgtatctctgttgccttagcgtaaataacaggaagtgaaattgctgggtgaaaggtaatgaaatttttaaaagctgttctttttttttgtttgtttgtttttatgagaactagacatcccatcagcagtttgtgagtgcccatttctctgcattcttgctgccactgaatatttggatttagaaacagaaatcaaaatgatggcagatttatgagtggaaaatactaattcagtcagggaactatcataattgaaacagtggttgttagttttgtggctgtggttctggttgtgtgtttaaaaattctgatttttataacccaactttttgggatcatattctctgatttaacttaccttttctcgatctttgtccccagtgcatattgaggcttatttcttcacattaattaattgtttaaaaagcgtctcttgtatgccagataaaggctaatgagggatggacagactgtaaaacatgctttcactcttcatttcattggccagatgctacttcgtaaaatcagaagtagttgaatttttcgttttcgctgctatcaggcttgtcaaattaaatttaaaaatgtaattttgctgttgaaatcaagaatataagttctcttttccttcccaagtatccctatgagttaattcactcacctgtgtccatagtgtataatcagcagtgtagtgattggatccaaacagatagactgtttcatgggatcatctgagaacacattatcaaggatgtgatactaggcaataagcagttccttggcagctattaattcatgttaagaacttggaaaagagtatgaggcggataaaaggcacagggttctgttcacaatgcagttgggaaaagaacagcttaaaaaaaacagcgctaagcaaaccagtttaaagttgctgactttaggcaatagattcaataattaaatattaaaatgagatttgtattgaggcagagggttgcatttcaacagagcaggcccagatgttgagattactatattgatttaattatgccaagatgctttcattgagcccttggaaaaaagaacactcattcaaaaattgacacatattttattcttcattagcttagtgttgcatacttacataattctggtatctcataaaaactgcaatatagagtctgatttaccaataaaatgatgaatgtggaggaagaatgcaggaaaaaaatcgctacatgttttcttcagaatgtcgggggggggtgaaaattctcaaataattatggtgcacaaactttctgccttcctgcaccatggggaactcttctaaaggcagcagtaccaaatgaatgcatctttaatcatctggaagtaagaaatccattatttttctaaaccagacagtagttgtctgttttaatggaacagttggttgattacagctcttcaggcctttatttgctgactgcccatagatatttgagttccagcttccaggattgtcataagtgagtcagagtccaagaatttagagtaggaatctgaggttgatgaatttactttggtgtagagagtttaactgagggaagtagatttccttatacagattctcacaaccggtgtgcttacagcttgctggctgcttagagacggactcatctacaaatgcaacctaccagatagagcaaagctcaggacctgccagtattctcttttttagtagagagagcaatgctcacctcagttgaagggaatacgagtctcttacagatgtcttgactgtcttttacagtaaccaaatgtatgttggttttatagaaatcagagttatgttttaattgtagaataccatatgtagaaagcattatgcaggttgaaaataaccactggttaagtgctttgaaaagttaaaggaacctctgagatgcagtcaaaggaaaagttaaccagagtcaaaaaatactgactgcgtaatccttacctaaggaatataatcagaaacactaaagcaaccccaggccacatatgctggttagaaactctgctgtgaattttagaaaatcttagtgaagatgtttcatttgagataggaatttttaaccttcatatattttatggtgttactgaaaaatagaggggtttggctaagttgctcttattgtggtggtggtttaacgttctctacggttccgtatcctatcagctattggacattttatttaaggatatagaaatgtttcgttacagcttattcattcatgaaatgtaagacttattcttcctttccctgtgagaagagcctttgcctttctggaatgctttgtttgatctcagtgtaatgcccttggagtgcccttcctgtcatgaaagaaaggtgaggtgaatcaaagtaacgccagataaggcaggaatgtgcgctcctggttactcctgtgtcactctggctttgagactgtggctgtatactttacctttcttgcccaagggattagcgagggcactggctgactttcttccctttagataatgtttcatgcacaaatcagacttgcctgcttccttcctgaattcatgacttggttgtggcataaaaggagttgccgaaataagaagagatgagcatttccagttagtcaaaactaaacagagtagcaaatgaaaaccacgcctcctctgtttaatatgttacgccctatccagaggtgaccgtttttgtttaaatttgttaaattaaaacaattgttaccctgtttgtattacgtgtctgaaagtgtttcactgtgctgcgttaatactggtggggtaaagggagtttgggtttttttttttttccctcttatcccccaagttagtgattaacatccagtcattcacttgtgagaggaatgttagttgctttagaaaatacatggtaattatttaaagtgtcctaaagaaatgtggtagctgtttcattctgttgaaaacgggccgagcgtcagttccattttctgtgcatggcagtgttcgggcggaggcaggtgcatcccggggaggagcacagttcatggtcctacgtctcctacgagagccgacaggaaatctgcaagagtaccttcagcccctcgcagatactgtcctggcccttctaaagcatccccttcccagggtccagcacggaagcactcctataactgtgcagtgagaccttcatctgggattggatatagttataattggagaggagattcagtctagatgagacgattaaacaacaagagaatgtagtttaatattgtcaaatacccatagttcataatgacctgagttggcagttaagactcctatgtaatgaagaatttcttacagatttaatacattcttaaatacagtaaacaagaaaaatcctggcttattttattcagtaggtaagcatatgtttatagttctatttaaactgttaatttacttattcaattatttggataagtgaattatgtaatgcctgtgtgttggctcggtggatttaatgaagtagcaatcttactgttttgtagctattgaaaaacagctgaaaaactcagttttgttaactattacatgcaatgttttgtgattaaaaaaaaattccctctgaatctttttggaaggatcactgagggaatttaaccacttttacatttttcatagggatctctgatacccaaaaggattaataataactccagagggagcagaatgaagctctccattctggtgatccttgatgggtccatgaccacagggagaagtcgtgtattggactagtctagaaccctaggaggaggttgtggggaaatgagagtgggagggagaagagtggcaagtgtgcctgctccaggggccgcaggtcagcagctgcaagtcctgctgtaggtcctggaaggaaaaaggctttggagaaatcacgagtggcctttgaaagcaaaactttcggaaaatttcacagaggcaagatttgagagggtgaaagaaggcttgggtgaatgcccttccatgcattcattcatctggtagttgacgtttattgaccgcctctagaatgccagaacctgctctaggtgcccaggactccacagtttgcaaggtagacggtgctgtgcccttcttggagcccaggactccacagtttgcaaggtggatggtgctgtgcccttcttggagctattcttctggagagaggagacacgagtcaacacagagatgaggagaaaagaacgcacagtttcagctgagcggtgctgtggaggaaaacagggcatgtgttaaggatttttgcagggtgtgcatggcctccttagatggagtggtcacctgaatgacaagaacgaagagccaactatttgaagacaatttttaatcagagggaataatagccctgaaacacaagcttgtatggtggaaggtctgggatgaggtagttaaggtaggtgcgtggccaggacatgaggctgtggtctgtggctgaagggaatggtaggaataccagggcagtcagtccagggccccttacacgggagtgaccctgccttaccacaggagaggactccgttttgtggcacacactcttcctttttcctgctgctgactgtcctgctgtcctggagtcccatcgctaggttttatcccagtccaggctttctgcctggttcccccttggggccctcagtggtgtttgtagtcactgatgacatggagaaaagaaggaaaatcctcctctgatctgccgggctgcctctttaagtggccatgtcctgccagacccagtgtcctctttgctaacagactttcttttggatgtaaacccttgaaataagattgctgttttaactactttaaaaacaaatcattaaaagttgttttctttcagaagcatgtttttagttcaaaaagtctgtcttcatgcatacgcatcatgttagaaacatttacaactttcttttttttttttttttttgagacagagtctcgctctttcactcaggctggagtgcggtggtgtgatcttagctcactgcaacctccacctcccgagttcaaggaatcctcctgcctcagcctcccgagtagctgggattacaggcacatgccagcacacctggctaatttttgtatttttagtagagatggggtttccccatgttgaccaggctgttctcgaactcctgacctcaggtgatccacccgccttagtctcccaaagtgctaggattacaggcgtgagccaccgcgcccggctaacatttacaacttttagcaattcagaagataagtaataatattttgaaagaaatcttaggaggtgcctgattcctagtcctgaatctgggtggcaacctaggttttcttcccagctgttagataaggagatcatccaacgtcccttatcgcctccatatgacaactaataattttatatatttggtgactaaacacgaactattatgctttacttttattttggtaatcatgatatcttctagcaatgacttcttttgaacttggttagtctttacttatagatccgtaaccagttctgagaaagtagttgtgacaaaatgtaacgaaaataagtggggaaagatagttatggaggcaaaggtggactaatgttcctttctgtgtcccagaaattcctaatttgacttttcctggtttttattgtcctttcatgtttgaaatgaaactttgaaacgttttataaaagtagctcttcatttggggctggagggaaagtttgtggcagacctcactgccacttcagcggtattgatgcatactgtttcaggttaaaaatgctttacgctgtcagttgaaataactcatcgatgtgtctcagtgaacaacgttgctgtggattctgactttcttctgtgctgcacgtatgtgtgtatttcactttagagcactgctaatgccggtcccttgtcttgataaggaatgtcacctgggctctacaataagtgattctaagttctcttccgaacaccgcttccatcatcgtgtgatttctgttagtagccagtttctgtcttgaataatagcatccttggaaatacagaggttagggattaaggtgtcatacctctttaagaatcttcttaaatcttggttttgctttagtttctcaaacatcaaaatagattcaatagattaaatacctttatttttatagtaattcttataacccatatatctcccacttttaaataagaggaataataaatattgatataaatagtaaatatagagaggtataataaattgccaaagtcacacatttattaaaatgctgagttgggacagttaacaggcatgctgtaaggatggttgattataaggccaatactttgaagtaacagactgagaataaaacctacattcactcttgtttccctctccaattccgaaataattcggtgatactcttgaccagtgattgagatgtgtgcctaggtcaaagtcactgataccatgttaaggtaaggccgattattttttaagctttcttagggtttggttggcagatcattaactctgagatagtatcattgacttttgcttttcctaatgtgagccatttccctcaccaatcagaaaagccgatttcagtcttatgagatttgtgttaatgcaagcgtgcacctctctactaggtgccactctcgaacgtagactgagatttcaagagatttgggttccagttatggctttgattcaagctctgcaacctagtaacttgtgaagtcatttattccctggacttccttgtctgtaaaattagagggttgcactagatgatctttactgctctcttttaactcttgttctttgctgattcgttggtttgattatttaagataaccaaacaccatgctttagaacttacggtggtcttggagctttatgaggtagtgttcatgtaagaggtcggctgcccaccacatgtgcctttgtaaaggtatttgggagtgtttttcttcccttccagaggggcaggggaggagtggtgcctgaagagagaaatgttacagaaacggtcgtgtgtaattaaagcctattcagtgttttcacagtggtgtcaccaacaatgataaactcagtcatagtcagatttcatgtttgtgttatatgctatattactggaatttgttagttttatcttattgaagaacataataggatggcttagcctttgcttctcaacactggtacccctttgcgtgtctcttttgaacagattgtgttagtctgttgagttgtatttcttttttaaggacaataattaaagccagggatatgccagcattatttcaaaataggcttatcaagggagcatctgtcaaatgtcttcagtattaggttaaattaatcgaggctttaaatttgcagtcgtgttgggatatttgggatatttccctgttccaattaggtaatgaaagcattggaagacactcatggtcttctattaatgggtacagttagcagtaaacaggttagtttactgggcctgtgaattatggttgattgagaatttataaaattatgaagtagttctttagatggcacgttataaaaaattcttgagtttcagattttaacaaatagcctgctacagcaaaccaaccaatgtagtaaaataagtgggaaaaaatatacgattgcttcaccacctctttcataaattgctgaatgaaaaggcaatctcaatccaagagtttagcacaaagtcatttttcaaaaatcatcttctaaactgtagacttgataacatgcttttccttttgaacccatatatctgatactgtcatgagctggaaatatttgagctttaggaatggcaacagtgttgtaagtactatgctatgtttttttttttcccatgttgaatttgtatttaggttttggaaaaagcagacagttttctaaataagatgtgagatacttcctgcttatggatagaaaacaaactcagtttcctgaattagactctcagaacatgcttctgacaccagatgtgtggggttcccccaacccccatttttttttttttttttttttttttttgagacagagtctcactctgtcacccaggctggagtgcagtggtgcgatctcagctcactgcaagctccgcctcccaggttcacgccattctcctgcctcagcctcccgagtagctgggactacaggctcccgccaccacgcccgggtaatttttttgtatttttagtgttatttgtttagtgttagtgtttttttgtatttttagtgtttttgtatttctgtgtttgccaggatggtctcgatctcctgacctcgtgatccacccgcctcggcctcccatagtgctgggattacaggcgtgagccaccgtgcccagcccagcctaaatatttttttaaacaaaaggtacttttactgttccaatcactgaagaaattacagggcacatgtaattcctttttgtgactgcccagtattccatcgagtatgtgtaccacattttctttatctagtctatcactgatgggcatttgaattggttccatgtctttgctattgtaaatagtgctgcagtaaacatatatgtgcatgtgtctttatagtagaataattaatattcctttgggtatataccaagtaatgggattgctgggtcaaattgtatttctggttctagatccttaaggaatcgccacactgtctttcccaatggttgaactaatttacattcccttcaacagtgtaaaagcgttcctgtttctccgcagccttgccattcccctgtaatttcttatatgcaggcctatgtatatatatttatgtatgtaatatgtatataaaagtaataatatattaataacatatataatacataatatcaataatatacaataattgatattaatgacaatatatgtaatatatttacatagctatacatgtataaaataaagtgataatagaatatatttgtctataatgacattgtataatgttctattacattaatttacaataataaatatttatttattattatattataataaattaatacagtaggccaggctggcctccacctcctgggctcaaggatcctactagattggccttacaaagtgctgggattaccagcatgagccactgcgcctagctatttatttatatttttatcttctccgatacgtatattttagtaaccaccctgctacttaattacaactagtgtgttataatgaacacttgtgtgtttttgaatttgttttttctagtggttagtttgaatatatgcttttcaaataccatcaactgcttgatatccctgtgatgtttattttcatattctacatcttctgttatcactttattttattaatggttagtattaaagaagaaaatgtcactttaaaatgaaatgtttattattactttgttaaagaacagctctcttaatgaacaactttgctctcagagaaagttaaggacagagataatataagaactactgcagttattctctatttttcattttttcttgtttcagttctgtgctttaatttttagtaatgtagatacatgacagttatttgagacttgccaatctattcttatatagtcttttaatgtctccagcaagatttgtcttcatagtctctaaagtgatgaattagcagtaatccgttttcctcctgttttaattctctttcaaagtactttatgagatttgtttcctgtttgcataaaatagtgcatattaaatacttcatttgtctgccccacagttaccactaagaaatcttggttaagattgttcagtttctgagcatatactgggtattgtaggaggtttgttgagttttgattgcttacctctcaattttagaatcctgatacatacaggtgtgccctcttttgtgatttcaaaacatatataaaattagaaatcttccgttatatgatacaccgttcttcattaaaaaaaaaaaccctgaatttagaaaacgggagttatgggattaatatgcatttacaaattgatgttttgccttaacgaagattcttagccaggtttaacatcaaatcttgcagtgcatttagattattagatagggctgtttgatttaatatgaagcgttagcataaaaggacatatctgaagtttctcctaggttatcagtgtccgtgaatcacttcctgattaggttagcttgacctaacttaagcttctgagttcttacaggtattggttttggcaacaaattgggtattagaagtagctgaacactgcttgtaagtagttcgtcttcagtttgcaatttttaaagtttaaagatttgaattaaaatttaagaactgctttgtaattgaaataaaatttaatagaaaaagaagtttctaaaaatttgataaaagtggaggctggagtaaaggaaaatgctacttgggaatggatgggcaggggggcagcttatacatacagtccatgaccacagggaccaaaaagtcaatgggacttttatttgcttcagtcagcattcagttcatgtaaaacacaccttcctttttgtagaactgaattgcatatgattcttagggtttattttgagagcaatagttgcggttttctgtatttataatctgtttacaggtgcctaaacaaaccttagaaacaggaaaactggcccttccaccagctgtgtgcattctaggactccagagacaactgtctctttggagagagttctgtttatacagagaccaatgtagtgcttgactgtaacaggaggaatctcattccattccttctttagcaagggagtgctttaaggagtttggcatgaggcttagtcatttcaactgacattttaatcattgaatagtgccttgtcttcttgctggaacacactttgcagtacaagagctttaggcagttgttattcagcctatgaaaacctgtaagtcactcttaacttttaaaggttgcttttacatttctgaaattccttaagttctccttatatgcttcagattaaccctttatacacgttggtttaaaaaatatttttatataatacatgtatatttcttctagaaacattagaaaatattaacaggaagaagataaaattaactcatccgtataactcgccatttttcttgcatatcctcttctccctgtttttttttgtttttttttttcacaaaatgaattgacatttatttttttaaaatcactttattacatgttgcattataaaattcaagcaggaactcataactgaagaaaggatttgtttggtctttgtgtaaaagatacttatacaataaatggaaaataattattaaagtacctagttgttaaaatgctgtttcccttctcaaacaggtgcccttcttttcctctctctctgctggtccaaggaataagttactcttttatttaactgtcttctcataggacctcattttgctcttctttttgtttcttttgcaccttgtagcatacctggcatgtggtaggaactcattctgtgttgaatgaattaaaacaggtgtaaatcaataaagccaggttccagattaagatccaagccagtacaaacaacattaagtcccttgttgaaaaataaggtacagcttttaaaaattaattgtgtgtgtgtgtgtgtgtgtgtgtgtgtgtgtgtgtgtgtgttttagacagggtcgtgtgtatgtgtgggtgtgtgtgtgttttagacagggtctcactctgtcacctgggctggaatgcagtggtgtggtcatggttcactgcagcctctgtgatcctcccacctcagcctggagctacaggtgtgtgccaccatgcttggcaaattttttgtatttttagtagagacagggttttgccatgttgcccagcctggtctagaactcctgggctcaagtaatcctcccacgtcagcctcccagagtgcaaagtggagaaattacaggcatgagccactacatctggctggtttttctttctttttttctttttatttattgcccaggatggagtgcggtggcgcaatctcaactcactgcaacctccgcctcctgggttcaagcgattctcctgcctcagcctcctgagtaactggtactacagggcacaccaccatgctgaggctagttttttttttttcttaatgagatttctttcctaaagagggtttttccttaaaacgtttctgttgatctgccacaaaacttttacagctgatttacacttcagagctctacatctgccacccaccactctagtttttttatagttaataacagtattcaagactcagttataatattctcgtagttagattcagtattgttagtataatgccatatacctacttacacctagtatatttcagttaatttagcctatgcaggttttttgtttttgtttttgtttttgagacaaggtctcactctgttgcccaggcttgagtacagcagcatgatctcggctcactgcaaccttgacctaccaggctcaagcgatgcccctgcctcaggcacccaagtagctgggactacaggtgcctgccaccatgcttggctaatttttgtattttttgtaaagacgaggtttcgccatgttgtccaggctgtactcgaactcttgggctcaagtgatccacccacctcagccttccaaagtgttgggattacaggtgtgaaccactgtacctggccgccttttaatacataaagaagatatgataaacacagtaacttcctaaatagagagcagtgttggattttttttaatatccttttttcttttttatcctaaatgggcctggtcacacggagttcttgaactctatacagggatctgtttggccttcctttagttgttataacttaagttccatcgctcctcccaccacaggcaagtgctggtccaccttggtggatgtgttgcctgtttttccactaacagtgattgattgatgagagtcagacctggctgttgaatcacaactctgcatctcatggagatagtaccctctggttttatgtgtggactctggagccagtcagccaggtttggggttctggcttctctgctaccaaagttggataactgaggatgacttccttaacttttcaggctaagttgttagatgtaaagctctaagatgactaactggtaaggtaatccattaattcattggttactttattacggagtaaataacttcagaccagtgacttttgagccttaagcctctgcttctgaatatggaaaatggggagagaccactgggctaattgtgaagattaaacaatatggcatattaaagcttttactaagtctgcatccagtccgtataaggggagaagagtaatcaacactgaaattgcgtatcttctgaaatttgaaacaaacttccagaagcattgccattggtaggtaaaatatattctctgtatcttaatgcttctgaaggctgtcttgtggatgtagttttatccaattaacaggtgtccagatgataagttttgatacccaagggtgatcctctttaacagtagagcttatccacaaaagccgtcattgacaatatcagtgattctgacagggaagtcagaacttggaatcattcctcaacaaagcgggacaacatggtttagcagagcttaccctgggaatgactctttgcagagctaaagaaatccctgggcttgaaacatgatgcttaaaatctgattatgtacttaattaatactacgttttagtaagtttgcaagtcagaaagcttcctcagctaattttgaagttattgcagcaattttaattaaaagaattgataggtatatgttttaattaaaatgatttaaataggcaatctaaataatgcaaacagtgacagttgtgtttttgcttttccttttgtcattgacttatgatctcattctgggcaatgtgattatagaatttcctagacctaaagctttgtgtaagtagttacctacataaatacttcagtgttagttatcgaagctgccagaatatccattgtcatatatctactcaaatgaaacagaaatacttaaaaatgaaagtacatctaagacatagacagaaaaataaaacagggaggttctgtacataaaaccttctgatactgggctgcaggtattgttttaattgacttttattgtatacattttaaatatagatttttaaatccatggttacagaaatttacagattaaaaaagtttctgttcactggatggatttatctgctgatcaccttgtttatttcagaccttttttctctgcacatataattgtgtatgtgtgtttaaaaaatttttctgaacagcagtagcatcatattttatatacttttatacgtttatatttttctctcaatttctgtatcactgacattcttctgtgtaattaatatgcatgtcttcttttttgaagctgtgtatgtaccatcatataataagtccattccttttccctcccgatacaatgaaattcgctttagcccagattttcaaatgttgggaaaacggcatctggcagctacatttctggattgcattcaagtcacaacttctctggtgaaatttgcaggttgtgttcagttgccccttaatcttccctccaattgaaggacaggcagggactccaaacatttagcacgattgtctggaacctgacttttagggttgaatcccagttccccactaatggactgtgtgacttctggcaggttgcttgtactgtctgtttcctttctgtgaaataggatatgataacgttacctacttcttagggttgctgtaaggagtaaatgagctgatgtataagaagttcttctctgctatatcttgagtccatgtatgctgttatgttgccatccctgtcagtcatcatcaggccactgagtttgctcaaaatacaaatggcttctcacaaaatggcactcatggcacatcactggttttgaaagattgggtcatggtggtcagtgtcatggcttcagttaaactggagagaaagaaattatacatgatttactgtatttttatctgtaagggggcagagaggcacctgtacggtacactagtatactgcctagcagtttgattggcccacgagggatgcttcttggttgacttactttcttttacttattttgactttcttttattatcaaaaacataaaatatttatagaagtggtctctagcaattaaaattttatttattaatatatgtatacacacataatagaacattgtttttaaaagtactagaacttgtttttttccttcttctgcaaaataaaccattaaacattggtctttacatattccttatgttttatgattagcaacaccagtgaagtaaatagtaatatcttataaacttcaggtgaaaggaaagtaacaaatgttgattttagttctgagagcatttcctgtgtcattccctgagaaagtagactgaatagccattttagaatcttaaatgccttcagcccaatagactcctcctttttcatttcttgttctattttagtccacagtagtaaataacataaatttgccaactgatggcagaagcaagaacattaaatgtatatttacacacacacacaccacacacacacacgatctactgtattgtttctaaaagaattcaaaggtttcgatttatgagataatactgcatgtcaaactagtatcgttatttttatttttatttttatttttttttgagacggagtttcgctcttgttgcccaggctgcagtgcaatgacacgtcttggctcaccgcaacctccacctcctgggttcaagtgattctcctacctcagcctcccaagtagctgggattacaggcgcctgccaccacgcccagctcattttttgtatttttagtagagacggggtttcaacatgttgggcaggctggtctttaactcctgacctcaggtgatctgccagcctcggcctcccaaagtgctgggattacaggcatgagccaccatgcccggcccagttattttaaagaaaggctaaattgcagaaattttgctttttatattaaactcgttttacagcttttctttgtatataataggcaaaacaagtaatatttttttattattttacagtaaattatattttgaattagattgttatttaaatctttagcctttaacatgtaaagtttcttatacacacattaggctctcaaggtatcatcaagatgtggaaatattaacatcagtctaaatctagtatttaatgaaaacgagattaaaaacaggtaagttcaaaaaagtttttaattgaaggaaggaaacaatctacatattatgagctctcatagacaactttttaaaaaaataaaacatttgcagaaaaatggagcagaaagtagaatgctcacataactccctgctccctgccccagcttctcacgttgttgacatcttacatgagtatgtaagatgttacaattaaagaatcagtattggtacattgttactaactgaagtccataatagatgcagattttctgagttcttacctaaggtgccttctgtggtccaagctcccacatgacatttagttacgtctctctggtctcctcttgcctgcgactgtttctcctcctttgcttgtctgtaatggatatcattttttataagcactcactattgacccggccttgaaccaaacacgtaacaattctttgtccttaaaaagtttgcattctaataggaagcttacaatctacttttaaaatttaattttaaggaagaattgatgtttttagtgattaacatttaatatgacaattctgagatgtttagtctttgcataattagcaggaaaaattgaataaccgtttatagattaattgcagcaacagagatattggctctctagtgtgttgatggtatgtctcttctttttccgtgagtattgttttcatatttgggaaaatgcttattttcaaaagagttaccctgtttacgctgagggtattgaattggagaaaagagtttctgtctgttgttgcctgtagggaaaatatcgttaaaattatatctcacatttaagtttttcttgtgattgaacaaagcagcttttaatacaattgctgccttaagcagttgtatgaaaagtttaaaacattaagtatgtgacatatcagaaaggagggtagcgtttgcaaatttgtgttgctgggttgttgctgatgataggtatttctgagagtcagaagtaacgaggctttgttgaaagccttggcattaaggcttgattgggggccaagacctgttaaaataggaaaattctaggaggaaaggtgttctgtggccatgggaatttgaatcagttaaattatttttggcaaacatcatagctataactttatttttgccttaaagattactgtgttgcaagagtcctgttccacctgtgtcagcgggagggaatgctggcctttcctgatcaaggactattccttaacagcagcaggggccagcagtggcggggagtaggagttattattggaaactgtgatttacttttgaaccgtttattggagcaaccctataacctttgaataatcaggctgtggaaagacaacgtcagtatccgtttagttaagtaagtggacattaatgaatccttccatacagaaattttgctgtgagctctattttcaactatcttggttaagctcttttagaaattacatttttcccccttcagtctcgttctccactggatgcatatttaaagtgagtttccatagtgacatctggtggtggttttgaaatcctggagagtatgtttggaatgataaatattcttacttaataactccaaatttccggcttctttcggatttttcaggttattaacaacttcactgtattctcgaggttggaaagctaggctttcattccttccgtaatgcttcattcaggtaaagccgtgatttctgatggcactgctgagtggcacacgtaacaagcgtgtctcaacctgtctcctgtgctccaaatctgcgtcatagcatcacgcagcagcatcacgaccgctctcactgcgtgtgcacggcaacctcctgacgggagtggcgacagagtttccctgtcacccctctcacggctctgttgctcccggcatctggctggagggaatcctgcggaaggcctgggctgcagccctccctcctcacagcctgggcgcaggcaccttctggccgcatgctgtgtttgctttcagaaaggtagaaggaggggagcgcagaggggaggagatgccgaagacaggagggagaccgcagagaagagtttgggggtggatatacataggtgcccaaaaaggtgaggtcgtgaatatgtgtaaatagaaagatacctgtaaaaactatagtttttgttggttccttttgtctccagggtgagtttataacccactatataaaaaagtacttattgaaagttgataggtagacattcatggataaagagaaatagacagggatagaggtttgagagtgaccacagtttgattcacatccagtgatgactggatgtctttagttttatttttgtttttctctgtgttaaaggcactaattagaagtcttcccaatggaaatgtgaaaatttgtttctctctatggaattgaggtaactgctaaagttacagaagaaatcaatctgtaatctgtaaatgccagtcgtggctttaggaaggactggtgaaagcatgatgaagtgtaggaacaagaactgctaatgacttaatgagtaaaggcagcttcggtcatacttggagacagctggtgtgtcactccctgcagaaaccaggactcccttaaagaatggtttggatcgtgatgaacccctgattaaggaagtccttcactctggattctgaactacacctgtttagcttcttggagttggacaaatcatttcaacgttctttaagaacaagggagttgcactggagggttttgaaggcgttcccagtctaatgcttgattttgagatggcgaaggctagctctacccctggtctttctttgcacattatctagactctcactcagccatcctggaccttcgtcttgcctctgattcaagatgggaagcttcggggttttgacctagagatctcgggctagtgccataggtccactttggaagtttctgcccagggttcttgaggagtgtatcatgcaactgggctgacagattaaaaagcggaaaacaaaataacttggcacgcttaaggcttccttccacccactttgtgcagtgcctataatagcgcctaggtactgaaccccaaaactctgagcggaatcctcagatcgtctcccgtctgttgtcctgcatgcatgaggaagacccttccttccacactgggaaactccttgtggcctttcctcatggttatagccccaccaggtccccagcacctttggtgtccgacagtgttgctgttgattgatagtggcaacagcccaccgctcagtggagaagaccctgaggcctggagtcagactgtccggctcagctccggatcccaccagttagctgttgaccctggccttgtttcctcatcactccagggcttaagcaagtttcttaatggatctaagcttctctttcctcatgtgtaaagctgggatgaaaatcatgcaacttccatgttctttcaatgatttaatgcacataaagcatctagtgacgtgattggtggacatgagggatctgaggattttggttagaagataagattcttcggtgtgccctcctctccctttggcagttgaaatttactttatagttctccgtaagttgtattcagtgattttttatcttacaaagaggtaataattaaaatgcatcgaagcagaagcttatctcagcaatcacgataggcattataaaagtacacagtcatgaattcatgaaataggaacatccagcatctcactcattcatacttactattttaattgcgaattacacaatctccgatatttgaatagagtcgctttgttgcctcagtcttaacctttagtaatatccgtagattaccggggaggccgacaatcccctcctgatggacatgactgtcccccaccccctggttgtacagctgatgggtcggtggggaccagcagatgccatctcagaattgcacactgcagtctggacgcttacggcccgaactagaaataaggcacgacggaaaaatcatagctgtttctatctgtaattgcaaacctcagatgtgagggtgatatacttccatttcataattcttttttcccttgagattagagaaagctattgtgtatgagtgaacatttaatacattcctgctgaatactgacatgtagaacaggtggatgaaataggaagtttgactcatctttcccaaattaccacattaagttttgtttactcttttgtcataacagccgtaggactgacaaggatattgctggcgctgaagatgagatttgtggtattcacactccagctctgccattcactgtggccctgggaaagtgcctcatttctctcaatctgagtttttcgttagtgaaatggagttaatagcatcacatatctaggcaggttggatgtaaagattatataagataatgcatgtaagacacttatcacagggcctggcatttagtaaacactgagcaaattgtttctgtaattatcactgtttgtaaatgcctctagtcacatttagaggggcacattttaaaacagtagatggaagcatgaattgttggaggattagaactcaaaagaggtagataaaagcaccacgtacctgtcttcttgagattttctaataatacttcacctgtcaagtgaattttattgcattttaatgaagtagcagcacacaggaggagttgtgtacattttgttaatgtgtaatcctcccggagagaacattctgttatcccctgacgtctttgtatctgtggatagatgtgatgaatcagaggaactgggaaaggttattatttgagcctacagacagatataattttgctaatttagcagaccagttgagtgtgtgtgcattatgcttgggttagagaacagggaagcatgtttgagttcatcttcagttgtctgctgtacggaggaaccagttagctcatttttatttggttctgaggtttattttattttatttcatttttttttttttactatcctttaagttctagggtacatgtgcacaatgtgcaggtttgttacatatgtatacatgtgccatgttggtgtgctttacccattaactcatcatttacattaggtatatctcctaatgctatccctcccccctccccccaccccacgacaggccccggtgtgtgatgttccccaccctgtgtccatgtgttctcattgttcaattcccacctgtgagtgagaacatgcagtatttggttttttgtccttgcgatagtttgctgagaacgatggtttccagcttcatccatgtctctacaaaggacatgaactcatccttttttatggctgcctagtattccatggtgtgtaggtgccacattttcttaatccggtctatcactgatggacatttgggttggttccaagtctttgctattgtgaatagtgccgcaataaaccaaaagcaatggcaacaaaagccaaaattgacaaatgggatctaattaaactaaagagcttctgcacagcaaaagaaactaccatcagagtgaagaggcaacctacagaatgggagaaaatttttgccatctactcatctgaccaagggctaatatccagaatctacaaagaactcaaacaaatttacaagaaaaaaacaaccccatcaaaaagtggctgaaggatatgaacagacacttctcaaaagaagacatttatgcagccaacagacacatgaaaaaatgctcatcatcactggccatcagagaaatgcaaatcaaaaccacaatgagataccatctcacaccagttagaatggtgatcattaaaaagtcaggaaacaacaggtgctggagaggatgtggagaaataggaacactttaacactgttggtgggactgtaaactagttcaaccattgtggaagacagtgtggcgattcctcagggatctagaactagaaataccatttggttctgaggtttcttccatgtttctagaagcagtattataccccctttctgaaggttcagtatgaaataatgaagcaataacaaatcattgtaatgcttctgaatggcaataaagattcattttatgataatttcacattttatttagctaagttttctattttcaagttgtgagtccttttgggagtattagatataattatcattcattcaaaaaatgagtttttctgatattaacctcgaataagttaatatttaatttcgtgtgatcaaaattacgaattgcaacagtgtatttttctcaaccaaatgtttcattgctgagcataggtaaaattggtaattggttatttttctcacagtagtttgcgtttaacgggaaacttcagtgattctagagcttcagcatttccagagtccagcctgtaccatggtgtagaagaacactgaaagtatctttagtcccgagtgatcaatccatgagaaaaagtgcagtgatatgtttttggtagacaaagggcgagattgcaccattgaataaaaaggtgggacttgccaaagagaaatggatatttctacttcttatccccagaggaggtaaatcctggcttgaagttaagttttgactggtttcagtggctggaccaaaacattacagatactctaaccctcttctttgtaatcatataatttcatctcttgctagttgacttactaagaaatgtatcctagtttgggtcccagttgaggacttttctataccaaccctgattaagatacttagatatttcacttgtgtatttaatacgttattgtgctttgtgtaaggtgctgcggatactggtatttaagaatgatgtggcctctgcccttttggagtttatagtgtagtttatatttttatagcatgctggttgtcttcccatttgtttgaggcgattgaaagaaataaaaggacctcaacactcaggagatctgagcagccccttattgtggactttgaaggttgcagtttctttccttagccactgtgagaaggggtgtgtgtgcagaccgagctttctggaggatgctgctcctgcctttttccttagacagtggccgccacagggatgcagtgtggggagcgggggtgctggactgtggggtctcagatctgggccatggctctggtgttgtccctgtcttcctgagtagcctccaggaaagtcagcacgtctctcaaccaactataaactttacctatggaaaaatgaagtgttgggccattagatgatctcttagtgctcacctattttgagacttcagtgagataatatttttaaagtgctccatttagtgcctgacacctgattctaataacaaatacttttatagcctatattatgtgccaagtgttctaatcattatatgcatgttagttgaatatactattattgtctctattttagaagaaaggaagctgagtcacagaaaggctggttaacctgctcaaggttatgcacctagtaagagacatataaagctctcagtaagccgtagttgttgacactttctgaagctggcagttttggtcgttgatgtgctgctttacgcttttcttttttctctstaggtgtcatagagacgtcagatcgactggaccgtgaatcgacctcccattattggctaacagtctttgcaaccgatcagggtgtcgtgcctctttcatcgttcatagagatctacatagaggttgaggatgtcaatgacaatgcaccacagacatcagagcctgtttattacccagaaatcatggaaaattctcctaaagatgtatctgtggtccagatcgaggcatttgatccagattcgagctctaatgacaagctcatgtacaaaattacaagtggaaatccacaaggattcttttcaatacatcctaaaacaggtatgtgttaatactgctataggaaatgtttatgcttatgcttttctgttagggggaaaagtaagaattagcatttatctgttacatacttttgtttcttgataaattgttttccttttgaattattcaaaataagcctttattttggataaaaatttagcataaccgggcacagtggctcacgcctgtaatcccagcactttgggaggccgaggcaggcagatcacgaggtcaggagatcgagaccatcctggctaacatggtgaaaccccatctctagtaaaagtacaaaaaattagctgggcgtggtggcgggcgcctgtagtcccagctactccagaggctgaggcaggagaatggcgtgaacccggggggcggagcttgcagtgagccgagatggtgccactgcactccagcctgggcgacagagtgagactccatctcaaaaaaaaaaaaattagcttaaatgtgctttttaaactttgagtttgcttttataaagctattttacgattatgctcattagtagggtttcactttcaggttaaaaagcaaacattgttatttcagtaatagctcctaaattagtgatatatcttaaacttacagcaaattctactgctgctaagaatttgaattgtcctaagtcggctttttgtaaagagtattaaaagagtttatttcacagttggctagttttttaatgttttaagtgtttcctatagagcagtgctgtatctgcaggtggtaagggattgggattgaacagttttcagattggaagtggttactttcctgaagtgacaccccaggccttttgcttctaatatttttttccatcgatgtctgtgactgacttccctgcttccaagaagatagctccatgacctcaaaatcctcctccgaaatttttataaacttctaatctttgttcctttactgcacaagtggtgactgcgttcaccctccttgtgaaataaagcaaaagtgagctaagaactaagtactgaaaaattaaaggcattgtcaaacataagagaaaagtcagaggtatataagaattgtgaggatgaatttggagtccagaatgtatgtatgcctctctagtggcggtcccagacctccagagagagaaatctagaagtaaaagccttgattgacacaatgtgagtgtgaacaaaaaccctcaaagagatgttttcctaccctcttcccaaacctcaacaatcatcatcacagaagacttctgggaccaagtgtgtgtttggggggctggccggggggcgggggggaggatttcctacacaccaagcagcggacaccttgggagtcctctcattcaattctgacatttttttacctggagacagcctcagattctgcaggttaagggctcagttcccaagagtgttcacttcttcccaccagttgcaagccgaggcctctgaaactcctgacggaccagctccaagcgagggttcccataacctctcttcaagcccaggcctccgaaactcctgacccaccagctccaagtgacggttcccatgacctctctttgggttctgttactttattggagcggctcacagaacccagggaaacacttaacatttacctgtttattacagaggatattttaaaggatgcaaataaacagccagatgaagagatacacaggggcaaagtctgcaagggtcccacgcacaggcatttctgtcccggtagagtcagggtgtaccaccctcccggcacctggatgagttcttgttcaccttcccgtagcctccacgtgttcagcggtgagaaactctgaactctgctttcttgggccttttatggagacttcattgcctaggcatgattgacgcacggacagccatgtcaacatgtggttgacaaaggcctgtctcttgagattgttcttggcctctgtctgcagcgtttccgcctccaggatatggagcgggaccctctctggaatgaggggcttatgacacacagtcagattggagtcctgccttggcaggtgaaaggagagtggcaggtggcccggagagagattctgtttcctgaggcaaaaagtgccacaacattataaccaaagactaacaggggttctgggagttaacgagccaggaaccatggacagaaacaaatatgtacccctccccacaacacacgtatatgtcatatctcaggtggcaattcaagaccagaactacccctatagtaatttgtttgatgaattagactaagcagtgttttcaattttcttgtgtcttctgtctctcttttagattagagtaagtcctctgtgaagagatagatgagcaagcaagaaatatcctttgggatatctgagcacatggcataggtctgggcataatatatgtttgaggagtaggaggagaaagaaactgaggagattatatagcttagcattgctgtggggagccttgaaaacccttggttcttcctgggaccccagaaatgatgatgtgtctgctatggtgttatgtcttgttgtagaaacatgattaatatggcattgcctctcccttttaaatctctctattctgacaactctaaagaaaaggtgtttgcagagatgtctgtgctgttgtcttcagaaggaataaaccctctccaggtgtccacagccacaccccagccccggcgggagcacaggcactccgctggcagggcggcaggaggaaattcagtgcaatgcacacagaggctttctttccttatgagttgaagtcaggtaatagtggaggataccatagttagtacacagcagtatttgttgtgaagtatgttaaaaactctgtggttagtaaagaagtctcaacattccacagatcacctaaagtctctgaagatgaagaacagtattttagcatggaattattaaataatatagtaaatatagcttcattagatctgtttctagatgcttctcttcccccacctcagcagaaagttggggtgtgatcctcagggcctgaagtacacatgcctcgtcttcttacaacctttaaagcagactgttcccaggtaggtcagtatcatggcggcctgattgtgaaggctgatgaaagtccgtccggtaggaaatagtgcttgggagtcactgacccatctcgccacctggagaccctaaatcgtgttacttaatgattaacggctgagcaaggaaatcactgggctggaaatccagagatccggaccatagtgaggccttccctctgggatcttggggcgaactgacttccaggtccttcgattttgtgattcatggtgtttctgtcccctgttgaggctcgtgtgtgtagagtgcaggagcgcccagcgtcgttcaggggacaggtgccacagccggcttgtgtgtgtagagtgcaggagcacccagggtcgttcaggggacaggtgccacagccggcttgtgtgtgtagagtgcaggagcacccagggtcgttcaggggacaggtgccacagccggattgtgtgtgtaaagtgcaggagcacccagggttgttcaggggacaggtgccacagccggcttgtgtgtgtagagtgcaggagcgcccagcgtcgttcaggggacaggtgccacagccggcttgtgtgtgtagagtgcaggagcgcccagggttgttcaggggacaagtgccacagcgggcttgtgtgtgtagagtgcaggagcgcccagcgtcgttcaggggacaggtgccacagccgcttcctcttgcgggtgttacggttgctctgttaatgaatgtcagacaatcaagaatctagtagactgataaacttgtgcctttggataaacacatttgctagaataaagtgggcccacttgtttcttagaaacatgaatttgggaccctggtgattatcctaaacgtccagcagctagagaatacctacagttatcatcatttaaagcatttgtttatgcatcaagtacagtcatgggaggctgaatgccagggatgcattttgagaaacgtgtcatcaggcgattttgtcgttgtgtggacaacgtagagtgtacttacccaaacctagatggtctagcctactacacacccaggctgtatgggatggtctattctagtctatagacctgtatgccgtgatcctgtaccgaacactgcaggcagttgtaacacatttgtgtatccaaacatagctaaatgtagaaaaggtgcagtaaaaatgtggtattaatctttcacaaccacggtcgtatgtgcggttcgatcgtatatgcagcccgtggttaactgacacgtcgttatgcagtgcgtggccgtgtttatttaccgaactatgaaatttttttaccaaatctttgaaagttcgtcatctacaaaagtgaaataaccaagaagaacagatgtgtgcagcagtgtggcacaggctctcacgggtccggtggaggcgtggcaggggccctcacgggtccggtggaagtgggaaactggcagtggttatgttgggaagatgagccccttggaacgggtactgggctgattgtgaagatggtaacaatagattgatggagaggaggtaggagatgttttcatgtcggcgacaatttgaaggaatggtgagttggagaccaagaaataagtgtatgggagacggctaccctcagtggaaaacctcgaatgccagaataagggttaaagagaatatttgttatttttattaataaaagtgacctctttttgagtcagtataaactgccttgcaacattttggataaacttaaataaaagtctttttggaaaagagggagacatctcctcaatatctttttctccttccccttttgtcatagtgtaacaaaatattttaaaagtcacaacaagaaagctttctaggagtgggtttagtgagtgagcttatgaattttgtgttctaaaccacaagtttgagtgaacttttttgaatgttttttctcttcaaggtttttgtgcttaattatggtgccaagaactgtctgtctgcatttcagacattcttattatattgtttgcaagtaggggggcagtgggttttcatttttaggagatacaagtgaaagaccgcatccattctctttttgtgatatatgcacttttgttgacattgctaatttttataacattttttgtttgactgtgtccatcatcagttacagggtaaatgtgcttcttttaacttaagttgagcacttgctcaaggtggaagagagacaggagcatagaactcagattggagccttggcagctgcagcgcctgcaaacaaaagcatcacagctttgtggtgtgtcaaggacagagaagaaagggagtcccaaacaaaagagactagtgtgagcttctcatgactcattattcacgaacaacctcgggaaatcgacaccacccttgcaaagagtctttttagttaaagttattgctttgaacttgggggagttgatatgcaacttacaattggcatttattaataattttgaagtgttacttttagacatgtgtgtcctttagaagagaccatggtcttgctctcttatctgtatgtttgatatttttcacgtttttgctacaaaccaagtattgatctttatttttaaaaagcttttttgaggtgtaattcacttacagcaaattgcacatatttaaagtctacaatttgataagctttcacatagcttttacacctgagaaaacatcaccactatcaggcagtgaccacacccatcattcccaaaggtctcctcacacttctctgtattccccctcccaaccctcccacctccatctctagtcaaatgctggtgtgctttctagcactaaataatagtttgcatttcctggaattttatcttggtgaactcacatggcatatactctttttggtaaggcttcttatacttcgcatgtttattttgagactcatgcatgttttggcgtgtatcaagactacgtttcttttcattgctgtgtatcctttatatagatagattaatattataatttgtttatccactcacctagtaataggcatttgtattgctgccagtttttggcagtttgtaaatccgctgtgaacattgagtacaagcctttctgtggacacatacttccatttctcttggacatcacataggggtgcatagctgtctggaaactgtctcaaggcagaaagctgctgcgatcgtgggtgtcccctcatctgtttctctcagggatccttgtctttctttgcctgatgtctagtgtcctttcaactgccagtgcattcattttgtctgcttcttggtggttttatgttaaagcataaatctgatcccttactagaagtggaaggtcagctattgcttttaaatcagaaaaagatgcctttttaagtttagaagtgccatcaatatgtatgtactgtaaacacctcctttaataaacagtgaatcttcttaaccagggtttctgtgtcttggagtaaaaactgaatttctaggtttattttaaatcgaaacttagcaatatcctggttatgtttctggagtttgtggaaatacagatatgtatgcactttttgtccctcccaaattgtgctgttgtgttgtacccagtagccggaatggcacttctcagtcctccttccccaggtaccattcatcatcagtttaccctgctcagcaaggccggaaggcagatgtgtgtttgtgacgtgtgtttgtgatgtatgtgtatgtgtgggtagcacacctcataaattctatcacttgttttaaatgggtaatgcaatcacatggcacaacgtgcaaatagtacaggaagatacccagtcaggttttactttcacccagttctccagacactccgttcccttccttagtatattaactctcttttcttattttctgtatcctgaatgctgtggcatctgggccctcattgagtggggaaggactgccccttccaggactcgggatagcagatgactggtgcaggaccgtgcttttcatatgcaagctgaccagtccagagtcccactccacaccaccatctgtgtcacgcccaagtcatatcagagccagtgctagacaactggggacagctatgccccagagcctttgaaattacttaaaccagccagtcctgagcgtctcaccttgcctttctgtgagatcctcagtcaagctttcccggttccttccacctccagaccaaccctggcatggtcccgtatggcccagcacggccttcggcctttgccaccttcagtggcggtggtatggcctcctcttggggaagtaccaaactgcctttgcaggtactttcaggtactggcggtcgtacctgatctgttgcctcaccatacctgaataataaaatctacttagaggcaactagtgtttgcattttctttcacttgtatataagcaagaaagaatatgttggttttttcctcatctttttatacagatagcatactgtatacatggatatgcatcttgggtttttccataacagtattggatgctgtgcttttaagagcaccttggctgggtgtggtggctcgtgcctgtaatcccagcacttcgggaggctgaggcttgtggatcacttgaggtcaggagttcgagaccagcttggccaacatggcaaaaccccatgtctgctaaaaacaaaaattagctgggtgtggtggtgcacacctgtaatcccagctacttgggaggctgaggcatgagaatagcttgaacccaggaggcagaggttgcagcgagccgagatcactccactgcactccaacctaggcaacagtgagattgcatctcaaaaaaaaaaaaaatcaccttattgtggttactgatgaacttgccacattattataggaatatatgtgcatatccatgtacctttcactgtccatttcactggtctactgattttatctaaccaatgtttactttttacctcttctcattcatcttcctctttgagaacaaatacagtaagaaagaaatttatttatgactaaaaataaatttatcagaaaagtttagtagttaatggtacagggcatcctctttgactatatatgtatgtttgtgatttctgtgtgtctatatttgcatcttaaaaggttttaggctctttgacagctgctacctgtatttctgtcttcctgagattttaaatacataatgcaatcacatagtacacaattcaatatgtatataccataaaaacctcatttgataaatagtgaatcttcttaatggaggttttgtgttttgagtttgtttcagcttaatgtttcttaggttttccttcctccatcctgtagagtgaagttttttttgtttttttttttgtttttcgagacagtgtatcactctgtcgtccaggctggagtgcagagatgccatcttggctcactgcagcctcaaactcctctgcccaagttcttccttcctcggtctcctgaatagctgggatcacagacctgcaccaccacgaccagctagtttttatattttttgtagaggcaggggtctcaccgtgttgctcaggctggtctccaacttctgatctcaaacaatcctcccatcttggcctcccaaagtgggatttttataggcatgagccactgcgcccaacctgtagagtaaaaatttcatgtattttggagagtgatccttactcaaaatcccatctcattattgtggagcacattgcactgttagaatcttgctggtcctgctggtccatcagctgtctcatgcctgttcccttggagagctgagcttgaaggagatagcccaagggaaaccagcaaagcctaattttccttgttttgcccctttctgtctcattctctgaagtctttttttataccactcttccacccgtctactgtgtcttgccatcacttttggatagggctggaacagaactttcgttaggagtcagaggaacattgacaagtgtgtctcatctattacgagcagcttccacagacttcatctttacaagagccctgtgagatggtgactacagataccaatagcctgctgtgctccttatttacattttcttcattttacaaataatctaagacttcacagaggtcagagctaccagaggtcaggcaatgtgaatttcctggctgccgcttctcagtgcatggtgccactcccagtccctgtgcctttgcatttgggtgtaaatgtggttgtggagccagtttttctgtctcttatctactctttgcattgagggactctgtttcatataatagttgcttttcaggctaatcttattcctatggtgccatcagaccaaaaatgggaaacttttaagtaacactagatttttaaacttagttttatgaagcatgtaccgcaggggtcatcaaactgtttctataaagggccacacggggcttttgggtttgtgggccatacagtctctgtcacaccactcagccgtgtcactgtagcccagaagctgctgtagatgatacggaaatgagtgagcaccgctgtgttccagtaaaactttgtaaataaacgaaataaattcaataaaatttgaattttctgcccttttcatgtatcacaaaagtttttttcaaccatttaaaaatgcaaaagccattcttcactagccagccacacaaaaacaagctactgtgctttcctgactcctgatgagtggacgtatttactggtgggactagttaaacaatttcgttgattgctgatttttaaaattattttgaagggatagacttttatatttctgaagtaaaaattgaatccctcccgttgctctaagcaagagtgcttagaaaggatgtttgtatttcacttctcttggttgttaataacaaaattattctttaaatagagcaattagagtatcagtatcaaggttgagatctcagatacgattttgcaagttggttagtagactgttagatctcttctacattgggaagtaattgtttctcattagaagataaattttcctagattttgaggttttcctactttttctgtgttgatgtgtcctgttaagttttcagagctcccaatttcagctcaaacagttggctctaagtatacttagcaggtctttatcatctcccccattttgtttattctcttattctgtgtcgacatgttatactcaaattactcagtttccatttcatatgctttttagaggacggcgttatacagtgttctgttttttattcgcttctttacatgtaaaatcctgtatttagtatagaattattttatgtttactataggaaatgtgatttgtgattgcagactgggcagcgatgtatagtttatgtgtggtaatccctaaagcacgcagtaaagttacaaaatggtagaatagctggtaggtatactgggtgctcatcatgtgttagtccagtaacactttacaactatttttttctgaaccagagtaaatggcattttattgtggctggaacgaatactgaacaaactgcaataggaaatagctaaagctgcaaacaacttccaggggtttccggtaacatttgatagctgcaaaggaaggaaaggacgggccagtcgcatttcaatggcaggaattcatttaaaaaaaaattacctatttgcagtctttttaccaagatacgcagatattttaaggaggaatctgtagatttttttcctatctctttctcatgctcattattattttataaaataagtgaagtatatcatccctaaagtttgcaagaagtcaaatgttattcagtacatttctgccgatgttgatgatgacaaactttttcctccgcaaattggttaacttgaatctttgtgataaattctgtcagcatttggtttttctatagctgctgaatattatttgctaaaattatagttaagattttgcatttgggtgcctgagattagtctttttatgtcaacatgatgttaaccttataaaatgggttgtgtaccctatcctcttcttctgaccctgctatatctgacatttataggatcactgaaggcaattccctgtaaattgagttttgctgtcgatcttatgttttaatgccatcaattttcaacaagaaacagctagtaaggtagaattgaaagcagaagagacgtctgaagactagggtatggggtccctgtatgactccccatgtgactttagactagtcgtttgatccattttattagtgttttctttggaactctgaaagtttgaactaaatgagttctgagctgctttccagcctcgccagtctatgaatagaattccatttttggctccacgcctaatcctgccggcaccagtacttcagggacaaacaatagctgctgagacggccacagctgccgaaagagcgcgcagccggctgtagtgacgtgtggtcttaggacaccacctgagtaaagacggtgcggtgctgcggagatctccgagctcacagtccagagctctgaggggctgggtgctgagtgataaagtgaaggagaagctgcgtgtttgctcgtggccagcaaggcaatctgtgttttgcagaagcggaggacgtgacgcctccctgccggtggactagtcggcttgtcggctctgcatgttcagtgttggctctggtgtgtgtttcggggggatccagtgttggctcaggtccttatgggccaggaccaccttcattcattattgtatccaccacagaaccaggcgtgatactcatgaaagatttttaaaagcagccacatttgaaggattttccctttcacccattggaagaggaggcatagcagttcacacagacaaatgctgcccttgatgtagtttctgtcactcccgctcacttgcgccttctgtccacccattgtacagctctcaggactgtgccatgatctctaagtcacatacacagctttgttctggggccgtttcattttatttaagctggttgggtacagatccgtagactagaatgttccatggatgacatgtttacaaagggacagtcaggtgctatctggagttccacagttacagtctgttatttctgtagctcatggtgctttattaatttattattaataaatttattattaataaataaatttagtaattatttgttaattattaatttatgaataataatgaagcatcaaataatttatcaattattaataataaataaattatttaataatagtaaataaattatttaattatttaatacttcattgtaaaaatcaaaataatgaccaattgtgttgccctctcttttccgtgcagttggttgaccctggaccagtggtgttgccctctctgttttccatgcatctggttggtccctgtagttctttggtagcttacctaactcagagcaagctctctttcaacactgaaccatggacatttaaaacaatttttaggagcccaagatttgtaacacatgtcctgtaaagttctaaagcacttttacttaaatctctgacctctcatatcttccctatttatgtaatttatgttcatttttcctggtattaggcctttttactgttctttcttagctgctgtataagagaagcaagaggtacggttaaaatcgaaaggacagtttgggtaaacacagggtgagcacaggtaagtttttctgactaatgataacattaggcaaattagttttgtaaaaaaaattttgattaacttattgtaccctgaggcccatgttgctggtgaagtgaactcttgaggacagagaggctattttgctttactcagaatcaaagagtctgttctgtcttggtccttagttgaaacagctttagagttggaacttttgtgtgttacaggcatgaagctagaatgtataattattaatagagttactaagtcaatatttagggagttttttttctgttatggagaggaaagaatatgagaaataatatactcagaactgatgtataatttgtgtcatattttgtatattcataactgaaaacaaaagacttagctgagttgtggttctcagccagagatgttaaggaagtcccattccagagccgaggcatcctgctctctcctcctcctgagggaaagcatgactccacttgggaattttgtttcatcccagatcacctttggagctgagttacttgtttaatcctctgctttttgtctcgtgtgtagagtgataagcctaccaagccataaaacatttcaagtgaagacactaacaaaatacagaaggtaataaaagtggttatctctgaataatggaattgttcaccagtgttacccaattcttttagaaaatattttttcagtactttcatttgctatccaaataaaaaggtgtattagtatgtatatccagactcatcagattgtatatgttaaacatatgtagtttttggtatatcagttatacctcaataaagccattaatttttttaaaaaaggtttatttccctaaggctactttccataaatttaaacgacagaggtggattctgtcgcggttctggaggccaggagtttgaattccaggtgccgtgagggcctggctccctctgaaagcgctaaggaaggacctatcctcgctcctcccagtctccaggaacctgctggcttgtgacggaaccactccagtctctgcatctgtcatcagacacctttcttccctgggtctctgactcaaatctcctctacctttctcttaaaaagactcttgctattgaatttagggtccaccataaatccaaggtggtctcatcttgaggttcttaattacatcttcagggatgcttatccacatcagttcacactcacagcttccagggttgtatgtatcttttgggcaggagccaccattcaatccaccacaaaggtatttccaagaagacggaaaatatgctgttcaatggaaatgtatatagccatttagattcctcttaatttttttaatgttgtaaagactacatttcatttaacttttgttctaacaggctttaatgatttggtcggtagttgcagacttgtcaggaagattctgggtgatgttttaggacttaataagcagttgaagaacagatggaatcttcaggtgggctctcccggccacttctttgttgcattttttgcaatgttaattatgcttcttatggtacttaagaaccatacttgagaaatactgtgtatttcttatatgtccatagtcctattgtgtttctctgtatctcaaatagtattttaaagaaatagataatatgctttgttccagaatatgtccagtagtaattattataatgactgaaaacaaaactgttaaaaaaagagtgaatttttttcaacttctgtcagcacctgtactgaggaggtcagcatgggatggaatcaccgggaaaccttcatgagatggtacaggctgggtcctcatgagtggagagtgtgatttggtggctctggcatggagcgggaagaaggggggttggcattcacaccccatgatcccgatgcacacctctgcaggaaaaggctcgagcagtagagccttctgaacactttccttggtaggaaagtttctcctgctgactcagaccttcaggaaattattccacatgcctagacctcatttcctcatgttttaaggtacaggatttgggctagagcatcgtgaaagaccctctggctttagcaaactttatttttacagtaagaggatacttagcacccgtgatccatttcattaaaaatttgctgcaactcaaaaacattagaaaaagtcatataggatatgccacttcaatatttctaaggagtttgagttttttatatgtgtggccatatattagtgtatataaggcttaaaaaataattgatttgagattttccagcctaagtttagttttcagccatcaaatatgtctatattttcactctgagctttaaaatgttttaatatttaaatttatttgagtaatatgcccattaaagattaaagatggccacaataaagtagtgtataaaatgaagttctcctgtctcctctcttccctcctctctcctctcttccctccagctacctgttccatttcccaggagtagccagaagtaatatgtcagggttgaacgtgtcacagcttctgtgcacaggagtttgtgcacacattacatccgtacacacatatgcatgtctcatcagccccttgccttgctggtttttgtagtgctccttgtctaaaagttacccaaacagtgtaagtttcacagtccgcccatgagtgtattacaggtgttttattgtaattgtccttaatgttgctgattcttaagcacactgaatgtccctgggagcagtccctgccacagtcaggattgaagggtagaaatctaacttccggatcctggtagtaacaactcctgaagaaattacactgaagaccctaaaacttaaactagacacttcctttaccaccaaaaatactatgattacagttcttaaacttagtatcaagtagaatatatgaaagtgtttcagagacatcatcgggtgaaaatggtatgtatctgaagtgcgtgcctgcagtcaagggtttgtcagaataatgctatgattaatacaacatcgtgagaaagggatggagcccagaggactccttcctgcaaaaggagctgggcttggtggagagaacattctaatcctagaagaaagtgagatcactgaataattttgttgtataatcattatacatattaaatatgattgatacatttagagttgtctatgtgaattaagagcttggactctggaatcagacagacttggggtcaaatctcagctttgcatttagctggcaatgattttacttctctaaacctcagatatcttaaaaataaaggtagtgtgtattggtagctcccttctagggttgctgtgagtatccagtgagagtgacgcatataaaatgcctaccccagagctgggtcttgggaaacacataattagtggtggctaaaaagcgaactgtgagaggacagtttaatattcacctctgccttttacatgtggtagttgtttccttaaataaattgtatgcctttgaggacaagagccttgtcttaggctttcgtatatgtctctcatggtgacatttgggaacagcattctgagcagttgaatgacagtgcacagtgaacaagtgggtgactgagatcgtaaacattatgacattactactgtttatgaacatagatttaatttagtcacttttagtgcttaggaaaaagaagcaaaaacaaaacaaattagtagagtcatgattctccaagtcacagctaagcacagagcctcttgattggtacgtgttcaggtgaagtagtacaccagcaattctaaagcaggcctcttcgtcgcccgctgagccaagtggagactcatagcagagaacggggagcaggagcatggcagggcagtatcagcacagtcaaaatctaggaaagttttactttttttttttttttctatgtaggtttaagacagatgtctacaaaaatttggatttagataaatattttttgcttcaaattttaaccacagaaaaggattttgaatattgcagatgttaaagtttttagttttgacaggcagcatttgaaattggagatgctataaaggatcagacataattggtgactatttaaatttaaattatttaaatttaaaattcagttctatggtgacattagctctgtttcaagtgcatagtggccacatgtggccaggtgctaccatattggacagtgcaaattatagaacacacaaatcatcacagaaatttctgttggacagtgcttttctagaacgtgtatggctgtggttcatgaattattgtaaaataaaagggcggaaaggccaaaatcattgcagggggtagaggaaaataatcagagtaaaaggaaggtatcgggtgaccagatttttaccttttttccttgatctttttcccttcctgtgcttattctctagtagagtaattgaaaagtgttccaagtaaatttcctgccgggtgaggtgaacatagggctgcagagattccttcctttagagccaggaccctgtctttcagtgtggggtcatcgatgactcgtactacagattgcaagtgcttgctctcttgttgcagttggacctgaatcctattcagaaatcctatttttttccttttctagctgccatctttcatgtgtgtgtgtgtgtgtgtgtgtgtgtgtgtgtgtgtatatattttttttttttttttttgtgagacagtctcactctgtaacctaagctcaattgcagtggtccgccctcagctcactgcaacctcggcttcccaggtccaagtgagtctcatgccttagcctcccaagtagctgggattacaggtgcacgccaccacacccagctaatttttgaatttttagtagagatggggtttctccatgttgaccaagctggtctcgaactcctagcctcaagtgattcacctgccttggcctcccagagtgctgggattatgccatccatttttatccactgtgctgatgttgaaatttattttttacagaaccacagtcttcagtccctccctggtttctttcacttttaatgtagacttactagatttattgaacccccaacttcaatactttgctaaatgacgttgagtaagcaacttaatctctctaagcctgtaaaaatggagatatcttcacatatttacacatcatatgtgaagatatacacatatacatatgtggatatatctcagttttttacaggcatatatatatatatatatatatatatatatatatatatatatatatatatatatatgtaatgaagtatctattaaagcataataccttggctaacaaactgtcaaaattgagtggcttgaaatagctgtcataatttagtttagggttctgctgggcagttcttccgcccttgaaataactcataatttagggttctgccgggtgcttcttccactcttgtgcaggcttggctggtggccagctggtcctcggtggcctcactcacatgtcaggcaatggtggagcagcacgaatgactagaccacgtgtcttctcttccaaccaagtccacacttttctggaacacagagttgctggcaagagccttcctcctgcttcctgttttctgtgtgtagctcctgcactgtgttttctgtcagtgccctacaatctatcctgtcttactgtttctcacatgtaaagtagtctttgccaacttctagccttcatcctggtttcttgcaaacaagactgtcttatctccactttctgagtggacagtacaacacagttgactgaatgcattcatcgctccagctgtgtggaccatgtcacatgactgtcttcacctactagtcctttgatgttcacttttacgtctatctgcttcgcgaagtctactttatctcttgaattttgagaagtttggagatggttttgttactgatttataagaaatgtcaaagagcacaattttatattgaaacatagctgatctgacattctctctgttctaaatgaactcatctctaaatgtctaagtggcgttgaaaggccatgagtggtttttaatgatgttcctcaagtgccataaactttgttactgcattgtgcatacatttctggggtgaactggtaactgctcattggtgagaatatactgtgtctgctgtctcagcccacctacatttatcatagttttattatatagaatgcagtgtttttctggaccaaggaaagaaaaatttctagtttttcttcctttctatttttaattatcagtgcttggcagaagaggaagatgaaacacaaagttacaaaaagaaaacagttgcatatacgtttttctgcttttgcctcttttatctcctaacttgcttttttggatctattaaatgactaatgatgagtcattcctggttttttcgttacagtttatgaaagtcacagttcagagttcatggagtgttgaaatactataatacagcaaattacgcagtcaggtgtgatagggagaaaaaaacgctcatctgagcctcaagagaacgaaatcctactcctggctttgttaatgacttactagtggccttggaaaattttaacttctctttggttcaggcagctcaacttgataatcttaaaggtttctgtctagttctgaagttctgtacagtttcctttaagtatatatctttctgggatttggcaaaaaatattgcagatgagcacagatgtcgaaaatgaaagaagtggacatacttactaggacacttactacctactaggttaataacatgtagtgggtatatggagcagttgtactttagaattatgtttgaagtcgatgtttcaagtgggtgggggtgggggggggatgaggagcaggagtgatgctcacagaggcgtgaatgagtgtgaacacagaacacactcggtagattgtcttgtatggccagagtgcagggggttcgggtagcgttccccggggtgttaggcactcagtgtggttgattgctgtaaggtaaagcgcacctcgaaaagacacccagaatgcctagtattaaattacatcattaggaaattctgttttcattttgtatgaaaatcccacaggaagccctaattttactgtaatttttcgcagagggggtgcatcacctggtgccacccccataggacactcatgcacggcctctctctggagatggcattagggaaaagcactacggcagccttgctaagattctttttgctaagtgttgtgaatttagaaagaaagtttgttgttgttatttgtttgtttttgtcagtctgaaatatcttacgtgttctccgcagaaaagtaaacccaccctgcagggtttcttgcaggatcttgatttgtgaattatgcctcctgagtagaccggtgccgtccagcagagcttcctgccgtggggagcgcgttctccatctgccttgctccatgctgtcgtcaccggcccctgggggctgccgtaccctggaaatttggcttagtgtgattgagaaactgaagtgtaaactttactgaattttaattaatttaaattccactggtctcaggaggctagtggtgactgtatttgttagtggaagtctacatcttcgtcttctctctagtttacagttacatgtgagacaatctcgtcttcgtaaaagtaatacttctcctggtctacttcctgcctcactttctcttttcttccaggctcctccaaagtacgtcctccttaaaatcgtctgcctttggcttctgggacacgaagcttctcttccagcagattgcttcctaggttgaagatcggcccgctattttctctaccgtttttataggcccactgttctctttagtattatgctcacttctgtgggtgtggtctgagtcacacctgtctgtatgcccacagcttcttccctgcgcctcgggtctgtgcttctaaatacccgcagggtgccgccagcccctagccacccatcgcagcacgtagccacccattgcagcacctagccacccatcgcagcacctggcacacgctgcctataaatatgtactaacgcattctctgttggctcacttatgggctgtggatttgtatcattttcaggtttacagtagtttatctctctcactctagcacatatttttaattctgctcattcatagttttaagaaattcacatgttcacacgtttaagtgtttcttgaggatgacggagagatagctatgtgagcaagctatgcaagaaagacagccactgtcctcgtgagttttccagtacagaaaagaagactaatttttaaatgaggggctttaatactgtggagtaatattatgatagaagaaatattagaacttggagaatgcagcagcaggaacccacgccaggccctgcagtgagtttaaaagtttaaacccgcttcagtttaaactgaagggtgaggcagtgactcaggctgaagtggaaagaaaggggctaatattaccattcattgcattgggatgaaattaaagctccatctgagttaacggaggagaattaatttgaggagtatagaacatgcaaataatttcagacccaggaaggaggtggtcaggtagccttttggctttatgtggattttgtttttttttttttgacagagttttgctctgtcgcccaggctggagtgctgtggcgcaatctcggctcactgtggcctcctcttcccaggttcaagcttttcttctgcctcagcctcctgagtagctgggattacaggtgtgtgccaccatgtcagctaactttttgtatttttggtagagacagggtttcgccatgttggccaggctggtctcgaacctctgagctcaggtgatccgcctgcctcagcctcccaaagtgctgggattacaggtgtgagccaccatgcccagcctttttatgtggaattggtagtatggacacaggtacagctttccttagccttcttgtgtgctctaagtaaatcgtgaagtggggagttgtatttgctcataagtatttgcgcagtgctgttggtgagtgattaagtgctctgcagaagaatactgttttcctgggaacgtgtcaagctgctattccacgtgatgttaccatcaatcacaacttcttggtggacacattacaataattagatgtgggactaatcaaggggctccttgtcgttcctgaactatttacaaccacagttaggagaggatggtacatttttgattggggaaaaaaagagattaacttaaaaggtttttgactaatgaaaattaagcttgtgccacattcaagcgtcatttttacacccgtaagcactgtgaagcatttccctttagcaccttttaatctatacctatgatctgggaaaaccccagtgtggaggtatgatatagccttctgttgcccttgttaaaaagagaacactgtttcgttgaattgtgaggaaaataatgttgatttagattcttagtaactacttaaggaaaaaaacctttgaattaagtgaaaacacaagcctttcacttattgctgcagagaaacatgttgaatatcccttaagtgatagttctgtgaaattagtaaagattatgggagaaaataaattagattaataatgtctattcttaaatgtgaattatttaaggtatttccatatgagaaaaaagccgcagcactgggaggagaacgttcagccctggtgattgtcctgccacacaggctccacctggttttaaggtcggaggtgcagatcatcctgcatgcacctgttcaccatcaacacagattggggacgacattttagtgcttaggggagttttgtttgtggttttggtgaagcataagaatacattttataaattgtaattgccttctagttccaaaatatgcagacagttgagcagttattaaagttctcaagagaggtcaaaactcatagatattctagtttgataggaaatttgttttctagaatacagaatttgaggatcattaattttcttttatgggaaattaaatgagcaagattacctggaaaattacattactttttataacagttctttatttatgagagtggataattcaaaggtaataaattagagttaacactttattttcaatagtccttattaatatagttatagaagtcatttctgtcaactctcagttatctcttgagcagttaaaggagatggatgagattgaaatcctggataacttcatctcacttttaaaaaatcttttctgacaaaatgtatattgaaaccactaacaaatagaaaatgtaactgatttgagtttttccactttcttactatttgtcatttatccaatatgagtcattaaagtgagaaagtataaagtatgttgttgtttgtagacaaaataaccgtacctggaaattaaatttctttagtaagaaatttctttcttctttttttcttgagaagaaatttgctcagacttttacaagagtatggttttgttttttattgtatttttgagatggagtctcactttgttgcccaggctggagagcattggtgtgatctctgcaacctccacctcccaggttcaagcaattctcctgcctcagcctcccaattagctgggattacagtcacgcgccaccatgcctggctaactttttgtagatacagggtttcatcatgttggccaggctggcctccaactcctgaccttaagtaatccacccccctgggcctcccaaagtgctgggattaaggcatgagctaccgcacctggccaagagtgtgattttaattgcagtattttgtaattgaccttattttgtgaggattaatgaactgttatcattttacaggtctcatcacaactacgtcaaggaagctagaccgagaacagcaagatgaacacatattagaggtaagttttttttttatctttaatacttaaagattcgtagttacaagtatcatgggaaagaacaatcttgttcccattaaccagtattcttagctattaatattttgttgtattaatattatttactggttgttaggaaaaaggaatataaggaaattgttaatgaatatagctcttgtcccctttatccctcaataccattcccagaatacacagccactgtcatgggccggattcagatctttgtattttatgcttgcgcacacacagggggcgaacagatctataaacagtgtcatgtggaattcatttgcatttacataagtgatatttttctgtatacatctttttttccccttactattatgtttttgagatatatttatgttggtatttatagatctgtttcaacctttttaactagaggtaatttgtttccagattcatttttttgctcttaaacagttgcattatttttgcgttttaaactatgaattacttagtttgtgcttcttaaatacctcaaaagtaaaagtagattgaagtctcagatgtgctcttctagaaagttctaataaggcttctcattctcatggataatttacaatgccttttagtccttagaaaacccacagcttgttatcatgtattggtcacctggtttttggctaatagtaaatacattgcagaaaaaggccgcgagtaatttggagagggaaggtggagcgtaacatggacatcaggttgatgagatctctgggccgtgtcgtcaggaagcggctgagagacaggcagaccgcagagaggggcgtagagacatctctctgtcccgtgtggaagactggtgttcattatcggagccagccttggaaaaggccacctgcctttgtttaattgagttatcagtgctcttcttccactctaacagtataagtgacaaaattctaaactgtaacttttgaaaatttcttttaatattgctgtttataatgctgcctgcttaatttcattttatccttggtcaaattttcttaacgttgtcagtaaatacttagtttttatagttgtgcagtgtatgtgtgtgtgtgtgtgtgtgtgtgtgtgtgcattgggaactgggaataccctctggaaaaggtggtttcacagccacagtgctttccccattaggagctgcttcatgtattacatctgagtttttagttcatagggtgtaaagttcgcttgaattcattttctttatgatagagcaaagttttgaattttcattctgtcactacaaggattgacagaaggggcaaatgcagtcactgacaaagataaacagatgggaagataggagagcagtagttacactgacattttgctccagtcctttcaaatttcacattaattgcatacgtcactccagagtcctcttttctccgtgaatatcaaaggctaggtggaagcagataccagatatatagcactttgaaagtcatatttggacatggatgggaaataaattttcccttcaacatacataattagtgtatttttcagtagggtccttttggggtggtatatttgagagccagtgctggcattgatttggctgtcttaaagatagtgtctccattacatgttgaaagctgtgactagctactggttcttgaacccaaagttaggaacaatttggtgatgaatactaaaaaacatagtgcttgcttgtatttctctttcttgtttttctaaagctcatatgcaaactgatgaaaaggaaatgtttcgagagtttaaaattaaaatagtaatgttttattttgaaaaagcttaggaaggtaaataatataaagcaactacttaaatacttttgacaggcaccatcgggatatagtcacactttaataaacagggtttacaactgccagacttcagtctccttgaccgtttttagtgttttggtaattcagttgggaagaaggtaggggtgtggataggggagaataagcattactaatgtcgttatatttaatgtgcccattgacgtaaacgtgaacatttgatttaagggtgaaatggtctcttagggacatagaaacagtcatcagtgcagtcctcaagactctctgatattttttcctaaatagaatataagaatatatggatagtttacttaaaatgaaagttattttaccgaaaatcttatttaatgagttggtcttaaatttaaatgtttaggtccactcctataaaaatctgtagaggaaaacagccaaaataaaatggtgattactggtattaaaaggtgtactatttatttaacacatactttaaaatgacaatttagcatattttactgatgaagtttaacatttttaacatgatatatggaaattttgtttttaagtatgtagagtgtgttgtttttaagaagactctttaaatgtgtaaagcagaatcttcacgtatctcttccatggcctaaaggtattaagtttttggtagcttaacatttgggtgaggttcactttccacttaaactaatgcgagaagagagcctgcgtggtcaggaaggcgcgcaggggtgcagctgtttctttcttagccatgcccggccccctgactctctccctgtagagatgggaggtgctgcctcacctccttacagccctgttgttggaatgaagatctgctgtgtatgttgtcatcgtgccctgcaaaatgcagagctttacacacatgcggagtagcatgaggaagaagatgctcactttttatatagtatatcttaacatgttaatctgttaactttaaaaaaacaggttactgtgacagacaatggtagtccccccaaatcaaccattgcaagagtcattgtgaaaatccttgatgaaaatgacaacaaacctcagtttctgcaaaagttctacaaaatcagactccctgagcgggaaaagccagaccgagaaagaaatgccagacgggagccgctctatcacgtcatagccaccgacaaggatgagggccccaatgcagaaatctcctacagcatcgaagacgggaatgagcatggcaaatttttcatcgaaccgaaaactggagtggtttcgtccaagaggttttcagcagctggagaatatgatattctttcagtgagttaagattgtagtactgtaatttttcatatgttgtgactataaaccttattttgtattttttctttagtaactttatttctattttgtaaacggctgaagtgtttgtgacctgctggccccattaggcaatgccggataacgtttgagaatcagggtgcttattgtttatgttgaatgtggtcatgaaatcattgttcttctgtcaggcattccatacacagaatacaaatgtgaaatgtttgaaattggctcagtttaagaccaaaccagtctgtgcttcattcatttctgggttgtaactcctgatttgctgtttttaatcccctctgcccaaatgttgtagattaaggcagttgacaatggtcgccctcaaaagtcatcaaccaccagactccatattgaatggatctccaagcccaaaccgtccctggagcccatttcatttgaagaatcattttttacctttactgtgatggaaagtgaccccgttgctcacatgattggagtaatatctgtggagcctcctggcatacccctttggtttgacatcactggtaagcattccccctcattttcgttgtccgtatgggttacactgaggagaacctgcatacggtttgatgagttttgggcacaagtcaggatttaaaattgggaataagatcgacaggtggagaatttgctttcaactataatattttgattgcaaaatttttcacaatatatagcatatgttaaacttgcttaaaataggtgctctattctaccaagttattgatataaaaataaaattttgtaaaagtttgtcaaagagttacagacaaaagccttgaatgttttggtccagaagcacagcttcttactgtttatttattgatgcataacctatggcacattgttatttttattgatgcataacctatggcacattgttatttttagtgaatatgattggaacatttaaagtaactatgcctgtgtgactgtcacatgtcaatatttaatagggaataagcttacaaattttgattcataccagcttatggtacaaagttatggtaccagctatgggtttttagttttatattacctcattttcaacaggtaggaggattcattagattatttcatcctccagtaaagtttacttgctaaagccatttcttcttcttatttgtcatgtagtttagataagacattgaatgtaataccctattcaacgtattttttaaatttcacattagagtttgcatcttagatttttttttattgtttcctcacattttagttacaagggtatttaagaacaagagtctctgttgcgattcatctctctgggggggactttatgtccatttttccccttctgccccttagtcatgctaacccgaagcccccaaaagttgcagctgatggacccagaccactgggcatgtcaggccaggccaggccttacctttggaactttcatgctaaatgctttggctgttttaccaatttggtgattgtgtgaaattgatgtcatcagcacctgtgaaacccttacagcatactttcaagggaaggggaaaaagagactattttcgtagttaacgatgctcaaaaccgtttgtttgctttctgttaacttttatcacatttccagatcaacaggctgctgctgtgcagtacctgacctcgctgtgtaaccttgctgtgcttggacctggtttgattgattgcttttattctgtgcttgctttccatgcgcgcttgcttctttttggctgtgccaggagacacgcttgctagagaagttttttacatctttcagatgacttgtgacccgacctgtacagcagaaggtatctgctgagatcacataatttgcctcagattatgagatctaaaataaaagccttgttttgtttttgtttttttttacttttttatttccagtgactatcttccatcgctgttttttttttcatagtgtgcgcatggtcacccatcccactcgccagcgtgttgccacgtaatgctcacctagctccgtggttttaggtttctcttaagacgtcgtgtgtgttgttttgtttcttggttagatttagtagtagaatttcatcatgacattaaacctctattttcatcccatttcattttatatctaggtcgatggatttgaaaagagagttttatcaaaaatatttggataatattaataggatagaaacttttgtttcattaggctttgtgtttgtcaaaaatgagaaaaaaaattcttttcaaatagtttatagtagccaggactaattaaagttaacgaccgctattttttttagagttaacattgatttttaatagctattttattaggtcaatttgctcaaagtatcagaaagttttgttagtaagtaagcttttaaatggcccagtgatggtatctactgagatacaaattgaagaggaattcagaacccctgaaaaagcaaattgaaagtaactcattggcaatattagcaatgtctttgttttcagagttgctttcttgctttctttcagttagttgcttcctggacaattagggttcactttgccttaacaagcattgtctttctaattggccactcttttctttcaaaatgctcctccttggaagctctttgctagtatattagaagatttttcagtgccattaagtatgagaaagccacagaataaactacagagtgttatcctgtgacctgtttttacttttttttccccaaattaggtggcaactacgacagtcacttcgatgtggacaagggaactggaaccatcattgttgccaaacctcttgatgcagaacagaagtcaaactacaacctcacagtcgaggctacagatggaaccaccactatcctcactcaggtaagtgaattactaatgacacacttgtcagaggaggatggagataacgttctgacttaggggtccacgggcaatatgagcggtggattctgccctgtgagggtctaaaatcagaagtttacacacatgcacactaagaatttttatttagggaaagaaaattaattccatatcccaatttgggttttgcatataattaagcagttttgagaattttgagagaagacttcaaaaaccaaaccaaaccaacgttataccatcaaaatcttcgaattttacactcattttaaactatgttaacatcaagtacatcactgttgagaatgaattgtttaaatcattttcagtaaaactggttaaagtgattaatcttctaaatcacttttttttggattcctcctggctatataaaataactaataaaattgttctaataattcctttaccacctttatttgttcctatctggtaattgagatggccagattatggggatttcaaacttaagtgttaaaatcctgtaagtgaaatttaaattctgttctttcttattacttttctttggttctcagtgtgggccgtttggagtgtacagaaggaatattcctagatagaagaagcatcctgaggtttcaggagcaagcagagtgtcattcactcatttatttaccaagtatttgttgaacacttgttacatgccaggcaccattctggaaggtgaatagtaggaaacagaagaggcaacaatcttgccctcttggagcttacactgggatggcagaagctaaaggcttgtttataatctactgtggcctaaatgaaaaagaaagaagttaaatgtcagagaacaaggtgcatctgagtttgaaattttctagcgttagattatcctgttttaagtactaagcaagacagatcatctgctaactcatttagctgcattaaaagataaaaattatagtcggtgatgtcttttcgattaataaatgggtagataatttaaagccatttacaaaaatatttctttctccaaatgcttgtgatttttagtgtatataaaattgatacatgtatgttatatgcttttggggtcattttgagattgtcttgtaaaagttctagtatggtgtcaaaatttcaatttagttggtagctgttctcattctagagacaagcaaaatctattgattttctctctttaaacataagcccttgtaaaattttagagcacaaaaatgagaataaaaacatcataaattgtaataaatctaataggataattctgagagaaggatttaaggggtataactgtagaccaattcacagaatcgactacccagaaatctaacaaaaggagcttcataaacagtggagctgtccctctttgccaagcattctcattactaaagaccctacgtctatgaataattaacagtgcacctctttcccaataattatatttttaattagtgcagttaaaagtttagcatgaaatatcacgtgcaccaactaaaatccctataaaacccaggaagtgtattctagttttctttcttggaaacacaaaatatcttaaatttcattgcttcctttatacttctgtaacttatttttgggccattgtcataggctgtagatgctatcctaatcgctggcagagaaaggcagtcggactataagagaacagtagcccaggttgacctgttaaggtgtgggacaaagggaatgatttgtgtttagattatttttcctctttagctgagaacctggaaatatgaatttgggtgggggcgggaggggggtgcagcagctgccacctggagaagagcacaggaatcagctggacagtgagaggcaccaggatacagcggggatggattgaaaagcagagggatcagctgggcggcgagacactaggatactgtggggatggattgaaaagcagaggagtcacctgggcagtgagacactaggataccgcggggatggattgaagagcagaggagtcagctgggcggcgagagactaggataccgcaggaatgggttgaagagcacaggagtcagctgggaggtgagacactagggtactgcagggatggattgaagagcagagggatcagctgggcagtgagacactagcataccgcggggattgattgaagagcacaggaatcagctgggaggcgagacactacgataccatggggatggattgaagagcagaggagtcacctggacggtgagacactaggataccgcggggatggattgaagagcagagcagtcagctgggcagtgagacactaggataccgcggggatggattgaagagcagaggagtcagctgggcagtgagacactaggataccgcggggatggattgaagagcagagggatcagctgggagatgagacactaggatactgcggggatggattgaagagcagaggggtcagctgggaggtgagacactaggatacctcggggatggattggggcttggaagtaacactcagaagtcaacagagcagccattgtgtatccaggtcaggggcagcagatgccgatttgagaaggtttaggtgagccgttggtgagtcaagcgggcagcctccagcagaggtaggcggcaggacagtgggagaactgccagtgccctgcggaaacagctcttctctggctttcctgtcctgcgtgtgtggcttaggatgctgaccgccattgattccagtcacttggctttatcttgtatctccaggtaacttaccaaatccagttaccttttttttctttcattcaaacaactttgctttcatatcttctttaactttctctcgagcatcagcatcgtggcctctggagcagatcacttggtttgaatcccggctccacctcttgtactcctttccatgggcaaatagctgtaactctctgtgtgtccatgttctcatctacaaagtgggggtggtgataatcgtccttttggaggtcttaggaaggttgatgaggtgacccgcagggtgcttgttcttcacgttctgagagcctgtcacccagtaagcacgcagaaagccatagtcttgttgcacatcagcactggtttgggctcttgtcccaccaacactcaattcaccttccagtcgccattcttcatccctgccactcatgttggaagcgagtttcttctgaccaaaaggagctgtgtagtcctcccacatcagttctggagtcgaggctctgcgtatttgactctgccttcttcttgctcctttactgtatcacccctgagttccaagtcctctccagcctctgaaaggcaggatgttccgtcccactgcttgccttcatgtcgttgacgcagcccttcctcatacttactcacctgccaaccaaaaatgtacccaagaaagcattatctccttaagagctttctgaatatctcagccagtctctggactcttaaaaactttacatgataatatatgagacatggttaggctgtgcatgtgcagaacattgcctggtaagcctggtggctctatgagggcagagaccattgcatgctgatttcacacccaggcaagcattgggcatggaaacgcacaatccgtgtgtttcaattgaattgctttgacttccagttctttctacttaataatgcttgagcttggagcaggattaaacttctctgctcagtacccactacatgggaaaagtaatttttaaatttatagtggaaatgaatatgtaatctaatctatggtaattgcctttatagccatcccttctggatcatcattgacctgtaaaagtgaggcatctgtagttttaaaggtttacatagctttcacatgcaacctttccgtggtttggtggatgcatctggaattcatacaaaatgtaatgttacgttttatttaactaaaacatccttgatattttggtcttctgtgagaacactgatgtagccctcatggaaggaaggcctctgttcatggcctgcaccatgtggttgaccgctctgcctgggaccctggccccctgtgctgcttggaccagcagatccctttgttccatatgcccccacgaagcgcagttttcttcttcttaaactctaaagcatgttttataattggcagtgatttccgctgtgccaaatccatacgtcttttcattatggaaattaatcctcttactgtcaaagttttgtttgtttctaatttagcttagtaccaagtttcttaacatttgcttttttcctctaaaggtttttactttatgtagacatagttaagagagtttattttttcaatctattgactaaattggttttagggagtaaacatatttactacatagatgtaggtttatgatcacagaaatacagtgatttctaaacacatttcttattttttctttttacgtattaaaaaaacctatgatcaggggcttgggatgtcaaagatgagaatgagaaatttggatagtgtaatcatctgtcttacctcagtgggttggtagttgcagatacaaatctgatttaagaaattagtcttctataaatcctggtaaattgttatccgtacatgagtgacctacagataccttctctgtggaggcgtaagagaagttaagcccacgtaaagacgtggcaagtgtgaaaatcaatacatattttgtcccagagttagttataagaatgcagaaaggtcatcattatcttactcatatttagataagtaatataccaatgagaaatgttttaagaaatgtatgattctaggtttttgttgcctttctcgtattttattttgccacagtatcgtttcaatatacatgactttcgttcatggttctttaagtattaaaacattcataattggaaatattgtatgagtcattttgtcatccattacaggtattcatcaaagtaatagacacaaatgaccatcgtcctcagttttctacatcaaagtatgaagttgttattcctgaagatacagcgccagaaacagaaattttgcaaatcagtgctgtggatcaggatgagaaaaacaaactaatctacactctgcagagcagtagagatccactgagtctcaagaaatttcgtcttgatcctgcaaccggctctctctatacttctgagaaactggatcatgaagctgttcaccagcacaccctcacggtcatggtaggcgctctacccgctctcctcccaccatttggtcctgaggcctttgtgtgttcgagcgtgatttttaattagttataaggaaagcaccaatgggattgataatgtgtcaattctttttaggtacgagatcaagatgtgcctgtaaaacgcaactttgcaaggattgtggtcaatgtcagcgacacgaatgaccacgccccgtggttcaccgcttcctcctacaaagggcgggtttatgaatcggcagccgttggctcagttgtgttgcaggtgacggctctggacaaggacaaagggaaaaatgctgaagtgctgtactcgatcgagtcaggtacttttggagccttcagtggcattttaaatttgcagttgttaaaagtctgtttctacttttccaaaatcagttctgggtattgaagcatttctatgtatctgcaaaaatggcttaaattgatctctagatacaattttacatgttaggagaaaggaatttttttttttttaacttttagcccatcagagagtttgggggaagttctgggggattcacaatattagcccagttcaaggctctgagaagccacgtagtagaggaaactttatttgaccaattcagtatttctgaaattattttaaccacagtttcttctgtaacattggttaaattaaacacacttaggaagctgcttgcctagtaatttaacgtttcacttttttgtcagagagtattttctcttgagcagagcaggaagctccataacagtgttgactcatctcattgtaattggctgaaacctcagaaagaggtgtcgtgcccctcccaccccttccacatttacctggatttttgccactcgcctttaacaggtggatttccctcatggaattgggttgaggactagaccccagagctggggcctgctggggtggggcttgaggcagcaagggcagcaaaaaaaacagctggagctgcggtcctggacggttccgggacccagaggccaagagatccagagtcgggataacaagggggaccaccaggaagcagtcctggcatagcaggatgtttttggtctgttttccactgccattccctcattcattcattcattctgtgggctgatgaagctcgttttatgtgtcaggtgccatgttggtcactcattcattcattctgtcggctgatgaagctcgctttatgtgccaggtgccatgttggtccctcattcattcattcattcattcattctgtgggctggtgaagctcactttatgtgccaggtgccatgttggtccctcattcattcattcattcattcattctgtgggctggtgaagctcactttatgtgccaggtgccatgttggtccctcattcattcattcattcattcattcattctgtgggctggtgaagctcactttatgtgtcaggtgccatgttggtccctcattcattcattcattcattcattctgtgggctgatgaagctcgctttatgtgccaggtgccatgttggtccctcattcattcattcattcattcattctgtgggctggtgaagctcactttatgtgccaggtgccatgttggtccctcattcattcattcattcattcattctgtgggctggtgaagctcactttatgtgccaggtgccatgttggtccctcattcattcattcattcattcattctgtgggctggtgaagctcactttatgtgtcaggtgccatgttggtccctcattcattcattcattcattctgtgggctgatgaagctcactttatgtgcgaggtgccatgttggtccctcattcattcattcattcattctgtgggctggtgaagctcactttatgtgccaggtgccatgttggtccctcattcattcattcatgcattcattctgtgggctggtgaagctcactttatgtgccaggtgccatgttggtccctcattcattcattcattcattctgtaggctgatgaagctcagtttatgtgcgaggtgccatgttggtccctcattcattcattcattcattctgtaggctgatgaagctcgctttatgtgccaggtgccatgttggtccctcattcattcattcattcattctgtaggctgatgaagctcactttatgtgccaggtgccatgttggtccctcattcattcattcattcattctgtaggctgatgaagctcactttatgtgccaggtgccatgttggtccctcattcattcattcattcattctgtaggctgatgaagctcagtttatgtgcgaggtgccatgttggtccctcattcattcattcattcattctgtaggctgatgaagctcgctttatgtgccaggtgccatgttggtccctcattcattcattcattcattctgtaggctgatgaagctcgctttatgtgccaggtgccatgttggtccctcattcattcattcattcattctgtaggctgatgaagctcactttatgtgccaggtgccatgttggtccctcattcattcattcattctgtgggctggtgaagctcactttatgtgccaggtgccatgttgggagtgttcctcctgggcatggtacgtaggcgttgccattgggtccacaatgcgtgtcttgtaaaagttccaccccgatagaaacagggctctgctttagagcatacagtctgtacaatcagaagtcacgttgctttaggatgttcctgctcatgccagatcctctgaaagtgatgaaatgttgggaagacagtttacagttttctgttttcttcagtgccattgttagtacaaggaatatagtccctttcagtcttcttcataatgagtttagagaattaaaatattccatttaacttgttttcttttttaagtctgtagctctacatatttagaaattgtctctcctaggcattagaattggtacaggtatagattaattatagatcatgtaataaaaatttgaggaaatggagagtaattttaaaaaacttactgatgtgttctaggtggttaaactatgtttatttaacatatttgaaatttcatttgtttcccttaaaacaaccaaatcgagttcattagtcacaaataagatctcatacactgcctgtgattgcagtgttaaaatgtagtgtagttagtgccttgagtaaagatgaagtttttttccactggttgagaaatttgcagttagaaaaacagtgcattccatttgtgcatgcctgcgtacttcaataaagtaattctgtcatagacaaactgactgggtttatgtaggagaaaatgttaaacttccccttagctttcacataattgtcaacaactgggtggttaaaatatgattgttggtgtctgagtagaagcatgtgcattctttgaatgcttctaatgactgacaggattaggaatctggtattcagagaatggaactccctcctccatgaatagataataccaatggcctattctggcatagaattaggacttttaagatgctatttttatagttttggtaatataaaaattatgctcagcagttactttgtaggagaccactaccattaaaatgcagtagaattttgtggaaggggtgtgtgcatgtgttggagatggaggtggacatctgttggaatagtacgaactataaaatgttcatacaaatgcctttcatttaagtttattcataattatctgcttctaatttgtgtcagattacatttaaagttcaaaaagactagttacaaattcagtacgtaacactgcatgtttaattagcagcaattaattgagtaaaaggtttggtctgattaggagtattcagggaaaaaggagttaattaagagatattttcagtaaccattgagttatatgctggagacatgtcagtgccatactctggtctgtgtgttttcttcttacatctttttaaaaaagtagattattagagcactacttctaaaccattttaatacctggaaatagaagatactccataaataaatatacttattgattagaattggttttcctccccaaacttaagtaattgccttgagagtcaacggagggtgccactctgccagtcagtggggccccagctgaacatccagtgtgtgtctacaggggaagaaacagactccccttcacagttctgtgtaatatacttaatagcttaagagtaaaaattagcctgtttgtctagataattgccctgttcccatacagggaacaatttaaagttgtttaaaaatcactcatattgtagctaaagtcagtgaataagcccccagcctcgtacagaccttttctgccaccttaggaagtcgttcaacagcaccagtgcatctcagtgtctgtgaatgaacctgttgttaggccaggcaggggcactttctaccactcagcagccctcattgtttcttcacataaacacgatgctgctttcccacctgcaaatctgatgatttctgccttaatctatatggatgtgggattcatgtatcccatcacgagctgggttcctgtttaaccttgttatcagtgacatgctccaagctgtgttcaccagagtcatgatgccccagataatagaggatgtttctgaatcttgcatggccgataaagagcaaggttgtaatccatgctgtaaagaaaacacagggcgatgagacagaaacagtaagaatagcgggaggagactcttccaggagaggatgcatcagacactggggctctgagatgagtgtgctctcctgattgaggacaaagagaatgcccacttggctggagggcagggagcaagttttgcagaaagcagtgtgttctagcagcaccagcctcctccacagcagatagtctcttcgttcctccacatgaggccttgcatatttgaagcttcatcggcaagataagattatcctgtacaataagagagaagtggatatagaagaatgaatataaaatatggtaggtcagctggtgagtacagacatagggaagtctgaggggcagggatgaagagagtcaccatggctgccccagtctcctgtgtcaggccaggcagtcagtcacaattttagacagtgagggtgatgtacaggtgggatgagaaaaggatcttggcgttctgacgacggccaaaaatgcaatcaggaaggacctgggatcactgactcataagtacagacaggccggaaattagtgtctcaaaagcctggcagaggcagcaggacccagttctcggccagggttcagctcagagcgtcagggcctagagaaaccctgggcctccaccctgttgcagactgaggcactgccttggggctcggatgcagagccagtaagacttaatgaaaagcaggctggatctggtctctaaaaaatggccgtggatttttggaaaatgtatgaaaggaaggaagtgaagactgtagaggaagtgtttcctgttaatgatgtggagaacaaagtgcttggtgaatccctggtgtgcagctcacactgagcagttctggtctgtgaaatgccacactcgtggcaaacctagagcaagaatctgagaaagcagttaagtggcaagtgagctttgttttgggtaagtataatagttcatttggggaaaataaattctgtgctatgacagggatggtgaattctgggtgcttgttcatgaaacagaaaaaggatgggctcgtggatggattaaacctgagaacctgcaacgccaccctgatttatccaaaagggctgtagagaactgcttgtttctttgaagattcagtcgatgatacaaaattatacaatattctggagttgtggttgcagctaaagatacatacagtatagcctgaaaaggtccaaagaaaaggaggtaaaacactctatctttgagcagcctctcagcttcagcactgctgacatttggaaccagatatttttggttgtgtagggctgtcctgtgcgttgtaggatgcttagcaacacccctggtctccacccacgagacgccaggagcaccctcttccgacaagtagtgacaaccaaaaatgtctgcaggcattaccagatgtctcctagggagcagaattgcccccaggggaaaaccactgacctagaggataagttgacctgaaaaagataagaagggaatgttgttgcaatcttcaaaacctgtaaagtggggtgggttacacacatttatccatggacaattgtctcttgaagcttgatgtcaatttcttactgaatcgaaatctttcgtaatgaatacatatcttaagaatttgtaagtcataaccgttgaaaatacaaatgtggtccagaagtgttttgagggactcctggaaagtgactcactcaactgctttgtggaaggatagccttgtctttccaccacacattcccagtgcagccgagagagggagttttggcttcgctaagagcagagtgctgtttcttctgttttttgtttttcatcatttatggacaaaattctgtaaagtatacctggttttaggtggataaaaataaaacaagttgaaggttccattcaaatattgacatcccctgagtttctgcttaatttcagaatataatttttaaggatactttgtttctgtttctctaactactactactactgccccttgtgtttttctgtaacatgtattttttcctctccttccaggaaatattggaaattcttttatgattgatcctgtcttgggctctattaaaactgccaaagaattagatcgaagtaaccaagcggagtatgatttaatggtaaaagctacagataagggcagtccaccaatgagtgaaataacttctgtgcgtatctttgtcacaattgctgacaacgcctctccgaagtttacatcaaaagaatattctgttgaacttagtgaaactgtcagcattgggagtttcgttgggatggttacagcccatagtcaatcatcagtggtgtatgaaataaaagatggaaatacaggtgatgcttttgatattaatccacattctggaactatcatcactcagaaagccctggactttgaaactttgcccatttacacattgataatacaaggaactaacatggctggtttgtccactaatacaacggttctagttcacttgcaggatgagaatgacaacgcgccagtttttatgcaggcagaatatacaggactcattagtgaatcagcctcaattaacagcgtggtcctaacagacaggaatgtcccactggtgattcgagcagctgatgctgataaagactcaaatgctttgcttgtatatcacattgttgaaccatctgtacacacatattttgctattgattctagcactggtgctattcatacagtactaagtctggactatgaagaaacaagtatttttcactttaccgtccaagtgcatgacatgggaaccccacgtttatttgctgagtatgcagcgaatgtaacagtacatgtaattgacattaatgactgcccccctgtgtttgccaagccattatatgaagcatctcttttgttaccaacatacaaaggagtaaaagtcatcacagtaaatgctacagatgctgattcaagtgcattctcacagttgatttactccatcaccgaaggcaacatcggggagaagttttctatggactacaagactggtgctctcactgtccaaaacacaactcagttaagaagccgctacgagctaaccgttagagcttccgatggcagatttgccggccttacctctgtcaaaattaatgtgaaagaaagcaaagaaagtcacctaaagtttacccaggatgtctactctgcggtagtgaaagagaattccaccgaggccgaaacattagctgtcattactgctattgggaatccaatcaatgagcctttgttttatcacatcctcaacccagatcgcagatttaaaataagccgcacttcaggagttctgtcaaccactggcacgcccttcgatcgtgagcagcaggaggcgtttgatgtggttgtagaagtgacagaggaacataagccttctgcagtggcccacgttgtcgtgaaggtcattgtagaagaccaaaatgataatgcgccggtgtttgtcaaccttccctactacgccgttgttaaagtggacactgaggtgggccatgtcattcgctatgtcactgctgtagacagagacagtggcagaaacggggaagtgcattactacctcaaggaacatcatgaacactttcaaattggacccttgggtgaaatttcactgaaaaagcaatttgagcttgacaccttaaataaagaatatcttgttacagtggttgcaaaagatggagggaacccggccttttcagcggaagttatcgttccgatcactgtcatgaataaagccatgcctgtgtttgaaaaacctttctacagtgcagagattgcagagagcatccaggtgcacagccctgtggtccacgtgcaggctaacagcccggaaggcctgaaagtgttctacagcatcacagacggagaccctttcagccagttcactattaacttcaatactggagttatcaatgtcatagctcctctggactttgaggcccacccggcatataagctgagcatacgcgcaactgactccttgacgggcgctcatgctgaagtatttgtggacatcatagtagacgacatcaatgataaccctcctgtgtttgctcagcagtcttatgcggtgaccctgtctgaggcatctgtaattggaacgtctgttgttcaagttagagccaccgattctgattcagaaccaaatagaggaatctcataccagatgtttgggaatcacagcaagagtcatgatcattttcatgtagacagcagcactggcctcatctcactactcagaaccctggattacgagcagtcccggcagcacacgatttttgtgagggcagttgatggtggtatgcccacgctgagcagtgatgtgattgtcacggtggacgttaccgacctcaatgataatccaccactctttgaacaacagatttatgaagccagaattagcgagcacgcccctcatgggcatttcgtgacctgtgtaaaagcctatgatgcagacagttcagacatagacaagttgcagtattccattctgtctggcaatgatcataaacattttgtcattgacagtgcaacagggattatcaccctctcaaacctgcaccggcacgccctgaagccattttacagtcttaacctgtcagtgtctgatggagtttttagaagttccacccaggttcatgtaactgtaattggaggcaatttgcacagtcctgctttccttcagaacgaatatgaagtggaactagctgaaaacgctcccctacataccctggtgatggaggtgaaaactacggatggggattctggtatttatggtcacgttacttaccatattgtaaatgactttgccaaagacagattttacataaatgagagaggacagatatttactttggaaaaacttgatcgagaaaccccggcggagaaagtgatctcagtccgtttaatggctaaggatgctggaggaaaagttgctttctgcaccgtgaatgtcatccttacagatgacaatgacaatgcaccacaatttcgagcaaccaaatacgaagtgaatatcgggtccagtgctgctaaagggacttcagtcgttaaagttcttgcaagtgatgccgatgagggctccaatgccgacatcacctatgccattgaagcagactctgaaagtgtaaaagagaatttggaaattaacaaactgtccggcgtaatcactacaaaggagagcctcattggcttggaaaatgaattcttcactttctttgttagagctgtggataatgggtctccatcaaaagaatctgttgttcttgtctatgttaaaatccttccaccggaaatgcagcttccaaaattttcagaacctttctatacctttacagtgtcagaggacgtgcctattggaacagagatagatctcatccgagcagaacatagtgggactgttctttacagcctggtcaaagggaatactccagaaagcaatagggatgagtcctttgtgattgacagacagagcgggagactgaagttggagaagagtcttgatcatgagacaactaagtggtatcagttttccatactggccaggtgcactcaagatgaccatgagatggtggcttctgtagatgttagtatccaagtgaaagatgcaaatgacaacagcccggtctttgaatctagtccatatgaggcattcattgttgaaaacctgccagggggaagtagagtaattcagatcagggcatctgatgctgactcaggaaccaacggccaagttatgtatagcctggatcagtcacaaagtgtggaagtcattgaatcctttgccattaacatggaaacaggctggattacaactttaaaggaacttgaccatgaaaagagagacaattaccagattaaagtggttgcatcagatcatggtgaaaagatccagctatcctccacagccattgtggatgttaccgtcaccgatgtcaacgatagtccaccacgattcacggccgagatctataaagggactgtgagtgaggatgacccccaaggtggggtgattgccatcttaagtaccacggatgctgattctgaagagatcaacagacaagttacatatttcataacaggtaaaaaaaattcataccaaacggtttaattaatttaaaatgatttttagaagcataaagtataagatctgtattgtatggaaattggggatttaagggatgattttagttaaaaatatctaaaataatacgtagaaataacatctgttgagcctcatttcaagcttgttacctgagcaaagctaccagttgtttcattagactgtttttgatttttgtgactcattcttttgaaccttatacttagtgtataataaatataattaaaaacatctatagttttaatgcaggctaaatgattagtagtcttaatgctatgtttatgcattttaatgctgcccttgtttttgtaaattggaaggctaatatatgccaaattaattcttttaatactcaattcttttaaactataacattatacatcttttgtacagtttattttttctgttacttatttttatcctttcaacaaatttgattattatcttcctccattgatttaataggaggggatcctttaggacagtttgccgttgaaactatacagaatgaatggaaggtatatgtgaagaaacctctagacagggaaaaaagggacaattaccttcttactatcacggcaactgatggcaccttctcatcaaaagcgatagttgaagtgaaagttctggatgcaaatgacaacagtccagtttgtgaaaaggtaagcagctcttcccttacatttgtgtgttatgaatttcaatttcttaattctttcaatgggaccatcatttggcgctgtggtgtaattcacatgtgttttatgatcacttactgattaagcagaggtgttaaatggggtaagacttggaaaagcttcagaactatttgcgggaatgctcaataaatcaaaagcctcattctagaaaactaaattacggacaggactccagttgcatccaacactgaaaatgctctgtaaccttttcatgaggggattctcttaatatggggatacagctgttttgggggaaggatctgacgggaaacaaagtgccgacgtgccgtgagacactgttttgggggcatctgctcttctgcacgctggagaagctcagaggtggacagcgtgcttcctgcatctgcgcggctcaccattcagtgggaactaaaagtagacgtaggaagggctgctccctccaaggttagatgagggcatgtggctcgagcaggggtggtccctggggccagagggtgagaagggctgcccagagaaagggctggctcagcttcggggggcagtaagtgggtcgtcaggagtgggtggggaggggccatggagtgtgaggagggattgtgggagtagccactgagtttgccagaagtggggagtctgcactgagagtcttggaaaagggtgaactggattacagtgggcagtggcggtcgttccaagggatttgaactcgatcctagagaaaatagggactttggagccttttatatcggtaaggtagagatgacatgagacgagttcttaagcttagtttggaagcatcgtgcccattatttagacagggaggagtggatgcaagggggccctggaaaggcagaggtggggaaggagtgaaaggaccaaagccaccaccgaggaagaccatctggtgtgacagccagccagcatgccatcgagcccgactttctacatgagtagggggagcgaccaaggccgtggcaaagaggagtctgagccgggggctggttttagaggagttccgtgttgagttccaattccagtttgctaaagtaatgagacatacagttgaccatgttttttagacattgagagatggcgagatgttggatgggtattagggaaaggtgtggaaacttggcgttgtctctggatagctgaaaattgacttcatttgagaggatactaacaaaagggaaaaagaagagagagctatttccaaagggagaagggagctgagctggaaggaagaggaggacaccagtgcagtgccaacctgggttggggagaagaggactttggggaggcagggtggcgagtggagctgggatgtttgggatgcgaaacagaccatttgggtcccgtgatgctgtgtaggtggcggtcttggtagcaggacgtggatggatgcctgatggcagatcagagagtgaagtgatgaagaaatagacctgcccagggaagaaggctgagagtgacagggaggaggaatgtgctgggccaggagaaggctgggtagagaggaatgtgattgtgaagaggatggaggagttggaacccaccacaggttctgtgagtctgagatgcttgtggctgtggaaagtgatagggctgttctcagggaaagacttaagatggaagaacccttatattcagtcatgaggggaaggggaacttctgagggatgaggagttaattccagttcttatttgtactttatattttcagaaggggtgcgacattctctctctcttacagcaactgtctttggttgttttttagccctttgatattatttcagtacaatactctatcatcttcaggaataagactgggacactagatcctttaaattcatcctattatgaaataccctttttcctgagtcctttttcatggagtacttttccgtgagtacttcactggtagttgagttcagattaagaaaatcaacagaatcttaatctttttgtcttaggttttaatttatggcatggatttctaaataattggaactggaaacaagtgttcaccgtgtttgagcatgagggcagatgtgaggagagagggcaccaggatcacgcagggcagtagaagggcctcagccttcatcagagtcccttctgtggccttcgagaatcttcgttttctcccataacatgaaacttgcttctctctcaactgcagttatttatgttaggaacatttgcaaaaaataaattcctgtatttggaaatgagcagatatgatacgacagatatttattctctgcagtttttaggtaaatacggagcactacttacatttttgattagtacttacgttttctcataaggatttgaatatgaaatcagcttaataacatataatagcattttgtaggttaaatgtcaggccaggctctttcagggatgtggagctggtggctgctccacggctcctccttgtttcacatgtgttttatgttttatgagaaaaatcgttttcaaagctcttgggattttatctttagtcatttcccatgttttctgtggtcaaagatgaatgatttcttttccattattcccttgatgtttgtgctgcctgttaatgcttttatgtaacgtttttgtttaaactctttgcagactttatattcagacactattcctgaagacgtccttcctggaaaattgatcatgcagatctctgctacagacgcagacatccgctctaacgctgaaattacttacacgttattgggttcaggtgcagaaaaattcaaactaaatccagacacaggtaatgatggagtttgggacaaaataaattgtatattccaacagtatcatatatgtgggtgattttagatcacacacaaaatttgaaattacattttgcccttttcaaatgtattctgatgctccgaagttgacatctcttagaaatcttaccagcaatttagtgcgtaaaattcaaggtgagattttttaaaaaacagcaagaatatcacctaattgaaaaaaggatttaaaatttagaaggcgtttataccacttgtttaaaaaaaacaaccctcagaaattaaaatataactcaagctttctgctttttatccattttatttaaatagcatattttcttgaattggtgaccataaatttataacttagcataatttagaattttcataaacaaaatgatttatgggattttatttattttaaaatagagttgttttccttttgtattttggtaaaattgggatcaactaagtttcctgttaagcctttttattttcttcctaaagaactaattgtgaacatgtgaatctaatttttgagtcaatttaaacattttttaaattttggtaaatttggtagctgtttcacctgtgcaatcttatacacaaggtgagaggccctggcctcagacctgcaggacttaaagagctagcccctggcagggttggagacctggagctcactgcctcttgcagctgttgaatctgtcattgataatactggtctcaagaggaaggctgtgaatactcagactaagtaattgaaagacaaaaggggaggaaataagatgtacaactgcaagtcacgtcttaaattacaagtgagtccatttgtttttgtttgtaggtgaactgaaaacgtcaaccccccttgatcgtgaggagcaagctgtttatcatcttctcgtcagggccacagatggaggaggaagattctgccaagccagtattgtgctcacgctagaagaygtgaacgataaygcccccgaattctctgccgatccttatgccatcaccgtgtttgaaaacacagagccgggaacgctgctgacaagagtgcaggccacagatgccgacgcaggtatctcccgggaatgctcttgcctgacgctgctcactgagatcctaggagcctctgtttcaaaacacaccccagattcagcctgtgccactcactccacctcatgcatgttccatagcccttccttgtggaaggaaatctttagaataattaattaatttttaatgaatatttaaagttccaaagttgtgcccagtgaacctactgctgtgaggtctctaaaattctggttactgaatattaactgatcttcacatcccattgaattaatcagtgatgtgttattttgtcaataaaccattgaggacagagagagcttttagcctcattgcaacataatggaagcaaatgctttatgataggtttacaaacctttcaaaatttttttagagaaaattggactatagaatgtatatttgtagcatttgatagcagagaataacttcttcagtcataccacaggttgagtattccttatttattatagttggaactagaagtatttttggatttttggatatttttcagattttggaatatttgcattatgtacttatcagttgagtatcccaactccaaaaatctgaaatccgaaatgctccaatgagcatttttttttaaagcatcggttctgcactcataaagttaatagattttggagcatttcaaatttcagattttcagtttagggatactcatgctgtgattctgagaagtttcagaagaatagcaaaagtgttgcttttcagatctttgtcaccaaagctcaatgataaacagatgtgatttgtaaaggtttgttaggtactgtattattggctcctatacgaaatagtctaagaagccagattcccatgaaattatatttacatttgttttaacagagtaagttgtttaccttttaaaggtcatagtcatacaaatttgcgaataatgtatattagcgtttacccttctaaaaaaattttgtttggtttttaaaaaaaaaaaaaaaagcgttggccgggtgcggtggctcacgcctgggatcctggcactttgggaggccgaggcgggtggatcacgaggtcaggagttcaagaccagcctggccaacatggagaaaccccgtctctaataaaaatacaaaaattacagtgtgtctgtaaccccagccactcgggaggctgaggcaggagaattgcttgaacctgggggacagaggttgcagtgagccaagatcacgccactgcactccagcctgggcgacagagggagactccatctcaaaaaaaaaaaaaagtgttaaacttttttatttaaaatcttttgacatgtttttatgtcttcataggattaaatcggaagattttatactcactgattgactctgctgatgggcagttctccattaacgaattatctggaattattcagttagaaaaacctttggacagagaactccaggcagtatacaccctctctttgaaagctgtggatcaaggcttgccaaggaggctgactgccactggcactgtgattgtatcagttcttgacataaatgacaacccccctgtgtttgagtaccgtgaatatggtgccaccgtgtctgaggacattcttgttggaactgaagttcttcaagtgtatgcagcaagtcgggatattgaagcaaatgcagaaatcacctactcaataataagtggaaatgaacatgggaaattcagcatagattctaaaacaggtaacctaccatgtatctgagaggaaacccttcattccaccaagaaaactagatttgcttaaccacgttgccttgtattaagtgaccctgtgttgtcagtagatgttctaaaaaaggcaaatcaagttttcttttagttatttacagtgggattgaatgttgtttggaaggcttcagattatagttctacttaatacattttgggcatgttcattataaaatacaagcaaattaacttaaaatagaaaatcaaagcagcatgttctattagtttagttgagatgtaaggtatagcaagttagtaaagataagaataggggatagtagaggtccttatgtaatcagactcatggatggtaacttcccaagtgaaatagtaattccagtcttttttattttagtttctgtagtgggcttctgataatatattaagttgacttcgttatcatgctagttgtctataacagttggagtactttgaatcacagttagggaaagaccgccaacaagctcaaaaggcataggaactgctgactcctgctgcgtgaatgcttcatcatcttgctaacctgtggcctaaaatagtcctaagtcttaaaacagtgtagaaagttgctccattcaaatttatatttatataaattatataaatttatatttatataaattatataaatttatatttatataaattatataatttatatttatataaattatataatttatatttatataaattatataaattatataatttatatttatataatttatataaatttatatttatataatttatatttataataaaattatttttagggctctttaggactagaatgatgtgaaagatgttaaaaggaattgtttaaaaagggatttttaaaaatgagaggatgtaaataaagggttatctagaagttaaaaggatataaatctccattttaaaaatatgagaaggaaaatattttaaaaagatgagacaaaaaatgaaaccgaaagacacaatagaatttttacgtttttattttgttgattaaattagagtgaagtcgagtgagctatggtatggaagggatgatacaatcatcatgtttataataagcagggatgtaggtggtgttgactggaaaagcaatttaaatatcatgaagaatatcatgaaaattaagagaaataaagatccagggatgggcagattcatagtttttaattgttgaattaagaaaataggaaacatacgtaaatggtaagtaaacttttgaagtgaaaactaaatgtactcaaaagcattcatctatttcagtcatagtgggaaaatgtggtggcattgcagaatgctggaagtaattttgatttctattcaggggccgtatttatcattgagaatctggattatgagagctctcatgagtattacctaacagtagaggccactgatggaggcacgccttcactgagcgacgttgccactgtgaacgttaatgtaacagatatcaacgataatacccctgtgttcagccaagacacctacacgacagtcatcagtgaagatgccgttcttgagcagtctgtcatcacggtgtgtattttttccccattacagtgaaaactgtgtatcttctggatctttgcaaaagtttttagtaaaatcaatctagtttttgtagcggcctttgtaaatattttggagatttgaaagtgtcttttgttaggccaactgattgctgactttgttatgaataattgtgttaaaacagctaagctaaccctgtctctactaaaaatacaaaaaaaaattagccaggcgtggtggcgggcgcctgtagtcccagctactcgggaggctgagacaggtgaatggtgtgaacccaggaggcggagcttgcagtgagccgagattgtgccactgcactccagcctgagtgacagagcaaggctccatctcaaaaaaaaaacaacaacaaaaagaaacagctaagctattaatatcacaaattttgtgtaataggtttaggcctcttcttagctgtctcctaaacaggagtgtggtgtgttctaggttatggccgatgatgccgatggaccttccaacagccacatccactactcaattatagatggcaaccaaggaagctcgttcacaattgaccccgtcaggggagaagtcaaagtgaccaaacttctcgaccgagaaacggtaagtgaaaaaggttgatttacgttgtttacaaatcaccacatcaataactagaaatgccttgtgctcttgtctgctgggcgtggtgtgagcaggactgaccgcctcaagaactgacggcaacatgtaaacactcgaaaagcaatgacactatgaagatctcaatgtgagaaatgtgttccatgctaggataaagtttggataataaagctttgcattcaggaaggaattactgttggttccagtgttctgagacacttcctaaagctagaagctgggttttaaataatagcagaggtttggaaaatcatagaggttgagagaagggaatttccagcgaggaattgcatggacacaggcatgcagatagaagccaagaaatatatttaggggactttacccagttgtattggagtaatcataagaagaaagcagagtgagataaaataggaaggtattttacacacaggttattggccacatagaaaaccaggaccagaggtttggacctgatgcctcaggcactagggtatgagcatttgtgagctgaggaaggtcagttgctcccctctatctgcagattctgcattcgtgaagtcaactcaacatggatcgaaaagtattagaaaagaaaaaatgtcgggtgtggtggcctcctccgatagtctcagctacatgggaggcataggtgggagaattcactgagcccaggagttgaagaccagcctgggcaacatagtgagaccctgcctccagaaaaagaaagaaaaacaataaaaaataacaatacaacaacaaaaaaaatacaaccccgacccgcagtataacaaccatttacattgcattgatactgtattaggtactataatctagagatgacttaaagtgtttgggaggatgtgtataggttatatgcaaatactacaccattttatatcaaggacttgagcaccagtggattttggtgtctatgaataccaaatgaagaaactatttgcaaaggaaaaattagaatccacaaagcttagatatggctccgtgtagaaagcattggtgtcagaggcagcctgattttttggtcttgctttgactgggtagtaccattaaagaaaagttatgaggtataaacgtgtaagggatgggagaggctaatgaatgtggttttcaatatgctggtttttaaagggaaatagcatattcaggcagaattactcagccagtatttggagattttagtcagatttagagactggaaaaccctaagtgtagatatggtacttgccgttggagatgttgatagaagtgacatgagggattgagataaatatgaacaaataaggagaggcagcaaggctgaacatccacccacccacttatccagtatccatccacccatttatccagtcatccatccattcatccatccagttatccatccacccatgcatctccccactcacctacccacccattcacctacccatccatccacccaccgacctgtccacccacccatctatctagttatctattcactcacccatctacatatctattcacttacccatccatccaccccacccatccatctatccagtcatgtgtccatccacccaccacccatctatccagttatccatccatccacctacccacctgtccatctagttatccatctacccatctatgtggttacccatttgctcacccacccacccacccatgtatccacttacccattcattcacccatctatacagttattcattcacccatttatccatccacctatctatccagttacccatccacccatctatgcatccatccattcatccatccattcactcatctgtgcagacttatgtccaggaagggaagctgagcaggaactacctcagagaggtaggcctgtgaaggagaaccaaaagaagccttcgatttgggaatgagaaggacactggtgatgattaagaatgaaatttcagaagaataatgggtgtatctaagattaaaggaatttggggggagatagtgatgagatacctcattaaattctttaatttctcctttgatgtgtgaatgttgagaagggactttctaaggatttgggaagcatgcttaggattgcccccactgtggggcagaataaatttagtatctttgactaaagaacccagatcctttaatacacttctgtccattgtgagataccaaaggagctagcctgtggtatttcccaagctaatgcgccagcagaattctttttcattggagcgtcttgcaggactaacgttctgtagaacatactctgggatctagccagctttctggcttctcccacaactcattctagaagacagagctttatagggaactgccctgaaaaagaagctatctcacttgactttctttcttttgtgggaggtcctgacaagctaatcctgagggatttagacagctgagtataatccgagtgtgaaattgagggggagggtgatctggagtgtcctgaggcattggaaatgagtcagcgctaaggtgcgggaggcagctcatgtctgtctacttccactgagagcaaacttggggccctaaggaaggctgctcccatcttaccgaagcaacagcatttgagatatgttaacttcttgtcctaatcatttgtattacttttatgtcttagtttctgttaatagaactgcaagataaaagacttttacaaaacaagtacatatttaagaggcatcagagaatctaatcattattggtaattcttgctgttgggaaatgtgtattacttgcatgcactcgtttgtaggagattagggcatcgctcacaggcaggatgcaacagatagtggtagctcactgggacctgcccgtcaggactctcaggaggacactcctcattggagctctgtcggccttgtgaaaatgaacgtgcattctgtcttttgcctgaaaagatttcaggttacacgctcacggttcaagcttctgataatggcagtccacccagagtcaacacgacgaccgtgaacatcgatgtgtccgatgtcaatgacaacgcgcccgtcttctccaggggaaactacagtgtcattatccaggtgggcttcgagtgccaaggtggtcctggggttctttgaaatgagcttttccattggcctctgttgcttttctttcttatccaaaggtttctttctagaaaaatgggaaacgtagaattcaagaagtcgctatcagtattaaaagatgagcattttaaagataaagccaagcttttatctctttcctttccgtgctttgatttttgagatattctcactgttctaggattcctaagagcttattgcttaatcagacctgctgccactggtgcttttcctctgccctctcctccccccttgaccttcctcttccctcttgctcttcttctagcccccaatccttccttctcgctccatcttctactccactttcttcaccccactcttcttcctccctactcctcctccctccacctcttctccattccattcctcctccttctcttcctcctccccactcttccctcctccccactcctccctcccattcctcccccctccctccacctcttctccattcctcctccttctcttcctcctccccactcccctccccactcctcctcttccctccacctcttctccactcctcctcttcctcctccccactctgcctcttccccactccacctcctcccccaacctcctccctactccacttcctccccatttcactgtctccactctttctcctcctccctccacctcgtcgccactcctcctcatcacccctcctcctcactacccctcctcctcccccttctcacttttcctcctcttgtcaacctcttctctttctcctccccactcctcctcttccttcctctcttcctctttttttttttttttttttctgagatggagttacactcattgcccaggctagagtgcaatggcacgatctcagctcactgcaccctccacctcctggcttcaaacagttctcctgcctcagccccaagtgctgagattacaggcacccaccaccacgcccggctaatttttgtatttttagtagagacggggtttcaccacgttggccaggctggtctcgaactcctgacctcaggtgatccgcccgcctcagcctcccaaagtgctgggatgacaggcgtgagccaccgcgcctggcctcgtctcttccttttctcctccagtcctcttcttctcctacactcctcctcctctctgctcttcctcctgtcccccactcctcctcgtgtctgcttcccctcctccctcttcctcctcacactccctttcttaccatgtttccttcatcctcctttttcttcccttttcgctacaccccttctcctttccctcctcctcaccaccacactcccgtctccacctttcttctcttcctttcagatagagcagaaaggctttgttttgtatgtattttatattatagaaaactcatgtgtctgtcccatatagatttggctgtgttccaggatttttgtttaattttgtttctaatcttgtgcaggaaaataagccagtgggcttcagcgtgctgcagctggtagtaacagatgaggattcttcccataacggtccacccttcttctttactattgtaactggaaatgatgagaaggcttttgaagttaacccgcaaggagtcctcctgacatcatctgccatcaagaggaaggagaaagatcattacttactgcaggtgaaggtatgaatggctttctttgtggtttggttgtggattcatagagaagagctgcttggcgtggttcccctcttctatttgaacagcttacatttcatacaaaaacaaactttcatagaattgctgtctttgttttttgccataccttccatttttatcccgctgtgtttgaaggagctcattgttgattctgtcatagtcattgacagagctgggtttcagtgtgaattagtgaagtgctataaagaaatttgtccttttctttgatacgtttcttgtgtggagaaatgagtatctaccagttagtaattttttgggaacttgagtaatacaaatgtttttattgaagaagttataaacagtgccatagacaaaggtgattttatttttcttttcttgtaggtggcagataatggaaagcctcagttgtcatctttgacatacattgacattagggtaattgaggagagcatctatccgcctgcgattttgcccctggagattttcatcacckcttctggagaagaatactcaggtggcgtcattgggaagatccatgccacagaccaggacgtgtatgatactctaacctacagtctcgaccctcagatggacaacctgttctctgtttccagcacagggggcaagctgatagcacacaaaaagctagacatagggcaataccttctcaatgtcagcgtaacagatgggaagttcacgacggtggccgacatcacagtgcatatcagacaagtcacacaggagatgttgaaccacaccatcgcgatccgctttgccaacctcactccggaagaattcgttggtgactactggcgcaacttccagcgagctttacggaacatcctgggtgtgaggaggaacgacatacagattgttagtttgcagtcctctgaacctcacccacatctggacgtcttactttttgtagagaaaccaggtagtgctcagatctcaacaaaacaacttctgcacaagattaactcttccgtgactgacattgaggaaatcattggagttaggatactgaatgtattccagaaactctgcgcgggactggactgcccctggaagttctgcgatgaaaaggtgtctgtggatgaaagtgtgatgtcaacacacagcacagccagactgagttttgtgactccccgccaccacagggcagcggtgtgtctctgcaaaggtactgcacatgagtgtatgaaagtcggtgtcacagatggtttcagacggcctttgaagcctttgatggctatacatgaaaggtgatgttcttgaaatgtctgttaattatcttttccctttctcccctctttgaatgaagagggaaggtgcccacctgtccaccatggctgtgaagatgatccgtgccctgagggatccgaatgtgtgtctgatccctgggaggagaaacacacctgtgtctgtcccagcggcaggtttggtcagtgcccaggtgagagttgaatggttgggtatacttttatttttaaaaatcggtttcattgtttcttaccattttcttctttattggagtctaaaaatattgcaaagtatgtttattgtaagaagacagatgtggagatgaataaagaatgacagtagtaagttttgcttcctcttgttacagcacaccccttggttcatagttggtgtccctcctcctgtgtcttcaatgattatatagacatacagctaacaacgcagtgccattatgtagcagtttaattttcctggaaattcaagagtgatcatatctcacacagttctctccctgttgccttttccctccacttagcagtatggtaaagagcacctttcgtatggttccgagccagtacctttaaacctgattctttttgatagctggataatattttctagcatggctgtacttattcaacattgttcttatgtgtaaagattcgagttgttctctttctttcagataaagaaatctttatacatgtatctttacatactgatgcgttcatttctatacggtgtatagattataaaactagaatttgctatctcaaacatcaatgacattttaaatttttttgtatcactatattagttttccaaaaagtcgtattaatttttatttctcccagttatgtatgaaggtttcactctccattcttttgacatcattgaacattttcgattataaaacaaattccccaccatctattttatgggtgtgaagtgatatctaattattattttaatttgcatttctctggctactttaaaagttttcatctcatttgttcatttcctatcagtattgcctctgctgtggcctctacctgttcatgttgtttgatggtctgggttatttgtttcttagtatcaatgtgtagagttacttgtgtattaggagtattaacattcacagtgttgcagttacctctgtctttatatcatttatattgatgttatcttttgatttgttaagtatgcctcttgtcattttgaaattcaattgttatttagtcagttctacataattttatgtaacagttgccatctgtggagattgatgacatttgcatcttctttggggaatggggattgttttattttgcttgagaaggtttcttaaatcaccttgaggctatgtataactattttctgaaattttcttctaataattttatactgaacatttttatatttaatctttcattactttagaattcatttctctgtgatgcrgtcgtcaaatttattctttccaaacagagagccagtcatgctgggcttttcattaagcaaatagacgttttcccacagaattggatagtaaaatgttatcatcttaaataaaaccctaaatcagtctcaaggggtgcaggggagtcatactttaaaaaatatgcttaaacttggttctggtttattttttcctctgtgtgattcacagggagttcatctatgacactgactggaaacagctacgtgaaataccgtctgacrgaaaatgaaaacaaattagagatgaaactgaccatgaggctcagaacatattccacgcatgcggttgtcatgtatgctcgaggaactgactatagcatcttggaggtatgtttctccacagcgttgcatcttagtggaagactttcaaaccataaatttcattttatattcagattgttaatttgcacagataataaagtaagtgaaatggttgaattccacatttaaataattcattgatgcctgtacaagttttagactttgtgggatgaagagctctttttgccctgccatgacagagatttaggtccatgtgacagttaacattcaaactctaggtgatgccccattttaataatgttggctgggtgtggtggctcacgcctgtaatcccagcactttgggaagctgaggcgggtggatcacttgaggtcaggaatttgagaccagcctggccagcatggcaaaacctgctctctactaaaaatacaaaaaattagccagacttggtggcggacgcctgtaattccagctactcaggaggctgaggcacgagaatcacttgaacaggggaggcagaggttgcagtgagttgagatcacgccactgcactccagcctgggcgacaagagtgaaactgtctcaaaaaacaaaaactgttacagtttatagagacatcctacatgtgtttcaagtgttatgtatcttgcccaccttttttaaaaacattattcagttttgacttttgatattacctaatgcttatgtgttcagttaatagttaattattcgcttccatgatgctttgtggatgtgcattttaacttgatattcctaaaagacgcattttaatgatttatacagtggacaaatgaatctgtgctctgtagtacttaaatgtaaggcctaacttctaccctcactccctaagccctctcctgtagatttgtgatcaggtgctctcattgaaggttctatgagagtaatgtgaacagtcattgcttttatcctagattcatcatggaaggctgcagtacaagtttgactgtggaagtggccctggaattgtctctgttcagagcattcaggtcaatgatgggcagtggcacgcagtggccctggaagtgaatggaaactatgctcgcttggttctagaccaagttcatactgcatcgggcacagccccagggactctgaaaaccctgaacctggataactatgtgttttttggtggccacatccgtcagcagggaacaaggcatggaagaagtcctcaagttggtaatggtttcaggggttgtatggactccatttatttgaatgggcaggagctccctttaaacagcaaacccagaagctatgcacacatcgaagagtcggtggatgtatctccaggctgcttcctgacggccacggaagactgcgccagcaacccttgccagaatggaggcgtttgcaatccgtcacctgctggaggtgggctccgtaatgttgccatctttaaaatgctgcacgtgtgtacatttaaggggaagctccccaggtatttttttatttttttctcttttgattcagataattttgtcagacactgcagtcagtgttggaagcgagcagcatccctcatacgggggcagagctgggtctgctgcccttctggtgcccaggctgcaggtaccacggctcccactgctgagcgcctgcagtgccctcccagtgcccgagcaggaggcacctcaagtgactcagaactcttctttaactccatagtcatcggcccaggggaatttagaattcgtaccgcttagcccttctttgttatttgtatcaccttttatctaatggctcagcgtagggggcctgctagattcttgctggcaataatagttgggaatatgattttccagaaggccctgaatccctcctgaagtgttgccatatctgtctgtcttgaagccctggcgtagtggggtactttgtgtcttttgcaccctggactcctgttaggcccacttaccaataatagggtacagttaggaaaatgacagttgtcataccaaatagagcttggtataatgaacagagcaaaaataatactgtcattaagtgggatttccaaagctttggttttctgctgttctctcagggaggtatatttgcattctgtgtgcgtagagtgggccaggccatccccacagaaacactctcatttatactgtgctcatattagttacacgtccaaaccaggttagcaaggaggccgtcctcattttgtcacttagggatgaggctgagagaggtgctttcctgacatgcactcccatggtcccgaagcaggagtaaaggagtatggcagctgtgccctggctcctaaaggccctggcccagggcaccacagttcttcatcctgagaaaaacccacagccacgccagctttggaggggcggggaagcactgtgctgcggtgtgccctcaaggcaggcagttagaagatctgccacaccacctgtcacttgacaaagtggagtcgacattcttcgtactgctttgaacagatgtgatgatctcctttcttaaatctttacatgtttgagaggctgaggcaggaggatcacttgaggctagaagtgaggcagcctgggcgttacagggaaaccccatctctacaaaaacttaaaaaatcagccaggcatgtggcatgcctctagttccagctactcaggaggctgaggtgggaggatccctgagcccgggagtttgaggatgcaatgagctgagattgcgccactgcactccagcctgggtggcagagcgagaccctgtctcaaaaaataaaaatattgaaaggtaatttatagtcttcaaagtgcttttcacattcattattttctcatttaatccttaaatgcttgtgaattgcaaataaattgagatgttaagtggtgctgtctacgtacatggtcactcaggtaaagaactgtattttcctcctctatatgtgtgacctagtgatcttaatgcagattatgatgaactaaagctaactgagttctctcttcaattctgggtgttttggttataccagcagtggaaggcttgttgatgtagccaacttttttcccagagagagtgagaattttacgagcttttacagacctcggagaataaagataaaaagcaatataattaagaccaggtttctgagtcattgagtgataggacttttttgttcctttttgccgatttaggttattactgcaaatgcagtgccttgtacatagggacccactgtgagataagcgtcaatccgtgttcctccaasccatgcctctatgggggcacgtgtgttgtcgacaacggaggctttgtttgccagtgtagaggattatatactggtcagaggtatgtgtatttttcttaactttcataatcaattgtagtataaaattcttaaacatataatggtacatgtaaaaagtctgcacgttaatttgctaatgtgttctgttttgctttctgttttgctgtatttatttgcaataggatcrtgargtttatgtctgtttctccttactcttcaggtgtcagcttagtccatactgcaaagatgaaccctgtaagaatggcggaacatgctttgacagtttggatggcgccgtttgtcagtgtgattcgggttttaggggagaaaggtaaatggtttctttcaaaatttaggttcataccttttcatagcgtcatattgactggcagatgcaacagaccttcagtgatttgaactacattatgtcagattttgataatcctcatattggccccttatataatttataatcttttcactctttaaaatgaattatcaagcagatgaatacagtatatataatacttgaaaaaaactaatgtttagccaatggggttgatgtaatagtctgttgatcctaggaaaatggccattaatattggcttgatatgtttccttcccaaatatacatcaaaagcatgcgatcacatgattggtttctccacagaaggtcttttctcgtggttgtcgacgttacattttttagttgtcttttatctgggtttgatcttcacatagctcatcaaggagggattagtcttcagtctctaaggattgagcctgacatacgctggcgtatgctacagaggtctctcctcattcctgacttccttggcaggtgtcagagtgatatcgacgagtgctctggaaacccttgcctgcacggggccctctgtgagaacacgcacggctcctatcactgcaactgcagccacgagtacaggggacgtcactgcgaggatgctgcgcccaaccagtatgtgtccacgccgtggaacattgggttggcggaaggaattggaatcgttgtgtttgttgcagggatatttttactggtggtggtgtttgttctctgccgtaagatgattagtcggaaaaagaagcatcaggctgaacctaaagacaagcacctgggacccgctacggctttcttgcaaagaccgtattttgattccaagctaaataagaacatttactcagacataccaccccaggtgcctgtccggcctatttcctacaccccgagtattccaagtgactcaagaaacaatctggaccgaaattccttcgaaggatctgctatcccagagcatcccgaattcagcacttttaaccccgagtctgtgcacgggcaccgaaaagcagtggcggtctgcagcgtggcgccaaacctgcctcccccacccccttcaaactccccttctgacagcgactccatccagaagcctagctgggactttgactatgacagtaagagccactttttcatctcactaaagtgtcacttgagataaacagtgaggttacaaattctgtccagtcaaaatgtgtgtactcttcattagagacaagtatgctcagactgaaatccgtatgatgatttgaaacttttttggatgtcccgattgctgggttagcattgtggctttgatgttattttaattaagaaattgaatctagtcatgtttagttaatattctggagagatcatttcattttataattgataaaggttctcatctataattttttgagtttattaatatgtcaaaaaaataatcgggagcattttcatgctaggatttttaaaaatatgacctggcgaagtggaaatatttatattagtgacctaacatatatttgatgcttttttcttttaaagctgtacatactattttaaaacaaaattttagcagcctttactgtctaatgatacccataggcatgcttatgggcgatttctactctatgaataatctctaagatgatgtggaggtctttctgtgcaggggttttttatttcttcgtttgagtttgtaaggtgtttctgtttttctttcagtcattcatttattgagtaaatctcaggccatcgttgggtaatgcatggtgtgtctcaggattcagcggtgaacaaaaccgtcttggcccatatacttactgtttgtcatccttccttttagcaaaagtggtggatcttgatccctgtctttccaagaagcctctagaggaaaagccttcccagccatacagtgcccgggaaagcctgtctgaagtgcagtctytgagctccttccagtccgactcgtgcgatgacaatggtgaggcagcagtgtgctgcaacactgcgctttgcttgcttgttgtgttactgwgttatctcacaatgtagacgcactatctacccttaaagcctcaccatattttcatttttcttggaccacttttaaacaataccatacatacttgcatgaatgtctgctggggaaaatagccaaactgttcttawtgaataggtgtttgaacttgtcagtaatagagaagaacatattcattgaaggatctgatcgactaatcttatattggctttttttcacaacagttaatatcaccccagttttgtgggtttgtcagaaatcttcctcgcaactttcttaaccacgcagcacgcacacaaacacacactctctctcacacacacacacacacacaccccctctaagaagagatgtgatagtctgaaatgttcgatttcagtagcaccttgggtattacaaggttttatttgtctcctcccccaaccccactgcggccccagctcccatttggatcagagcgtactgccagagcacagtttaagagattgctagtaagtttgattatatttctaatgaagaaagtttaacagtgggatctttgccaccagttatgttgatctggtttccctatagggtaaattgcattgataactagctttgccatattaacacataccagtagagaaaaacgtcaatattcagtaacacaatgtagtttttagtggaaatttttgaatctcagaattagtaagtttaatgataatttttattgtataaacgtaatacgtttattattaaacatattaatatgttatacatttcaaatattttattggtgggaaaaggtaaccccactaataattattcaatccagagtaatatagtccataatacatcatatagtccataaatccatatagtcattatctattgcagtcatttttcaaactaaagatagctataagtatgtgctatcatttctgatttcaagtatatatatatatatatatatatactttcaagtatatatatatatacacactctattcctaatcaacacaggtatattttgataccgtatatatggtatcaaaatatacctgtgttgattaggaatagggtgtatggacatagctttgcttgttaaaccaacaagatagaattatgtcttttatcaattttaattctaagaagttcctgtttccagtttatgtaataatttatatcttggcccctaaatgctctttataaatacatttcagaattttatcagcattttctttctagttcttgttgttggaacatcagtcttcttaataatagtcccaggtggccgggcgcagtggctcacgcctgtaatcccagcactttgggaggtgaaggcgggcggatcacaaggttgggagatcgagaccgtcctggcagtgaaaccccgtctctactaaaaatataaaaaaaatcagccgggcgtggtggtgggcacctgtactcccagctactggggaggctgaggcaggagaatggcctgagcccgggagttgtagcttgcagtgagctgaggtcacgccactgcactccagcctgggcgacagagcgagactctgtctcaaaaaaaaaaatagtcccaagtgctttcaataagttatggtcaaattagttatgaaaactagcttccagatagtacaatgcagagaaaaaacatatatacaggagtgtttttattttactcattaccataatgtaattatcagttgtaaactgaaagtctgctgagagtctgttcctgcgtcgtattcccggttttccagggccattacagattaccacaaactttgtggcctaaaacaatgtagacttgtgttgctgtcattctgtagcttagaagcccaacccctgtgtctcacagggcttaagtcatgcgatcagtagggctgcattgccttctgcagctcgagggaagaacctgttttccttcctttcctggtttttagaggctgcccacattctttggctcttggtttcttccatctcagagacagcaacacagtgtttcattgaccacccttctgtcagcagagctccccccagccgtaactgggaacgcctctgtgcagaaaagttacccttatgattacactgggcccacccaggtaatccagggaaatccctcatctcaatttccttatctgaaccgcgtcaacaaagtcccttttgccatgtatagtgacatattcacaggctcctgggattaggacgtggactatttgagggccagccttctgcctaccacatgcgtacatatttaccttggcatagttattttaaaacaatctgatacacaaatgaaaagggaatttacacaggaaacagacggacagagtctcctcctatactcgtacattagctcaccagcgattagtccatctgcaaacagacgagaaatttgcagggaaagcaatcagaacagggacaaaaacattcttgttttagattgcatatgtttctccttcaatttcttttttttgggggacaatgtgtgagtgattaagcagccggaagagtggcgaaccacagactgtagtcttggaatgtcgagtcttgcctgtaagatgttaaaataagaatcgttttggcctggcatgatgcataattgtaattgtgaaaaaatttaaagctaaaagtttttttgtttaaattccaataaaatatgtttacgttattcctttgattaataataacaaatatatagctcgattaaaaaagtctctgagacacacagctaacagtgaggagaccacgcatcattatgtggttgtattaaagcactcatcggttggaatctgaggtagaaatggtataatacaaaactttatttcatgcactgttttctaactaactgaagatgttcctaattgactgcttttgtacctttttgcaagcttccatagtgaccgtaatgcaccttgtcaatgctgtggttgacacggtgagcacagaaggtacagttgtaagtgctcgtgcttgtgtctgtgtcttgcagcagccgtgtcgtagagctcggccgcattgctattgcaatggtaggaaaaaacagtttgtggttttgagggggttgagtccttttattttatgcaggtgtgtatatacgtaatatgagtgctttgcaaaaaatatctcaatgtatgttatgtctttgttcctttttaccattcatagtgatttacatgagatctggagagcttatctgtaggtggtgcaatmaagaaaaagttcagaagtaattttacctaaaatttttttgaaacaaaaattcttcttcgtatgtacaggaataagacttgaataatgtaaagctttcataattaacctgtaactgactcttcacttttggtttttccctgagtatgatcaaagaaaagggtgtagagcatggtaaaagaatattttcatatttggtttagtgaaaattatttaatgtgaagctttatctgctcagagttgtaagaaaggacattgagcaccttccccaaattattttaaattaaaatagccctttattagtgcactgaaattcatcacatggggaagacacaggctagtacttttcaaactgtaatgtgaatcagatcatctggagggctgttaaaagcccaattgctggttctcagcaccagcatttctgatccaacaggtctgggttgggactttagaatttgcatttttcaccagttcccaggtagctctgatggtgccggtctagagatcccactcgagaactgcttacagaggccatcgagtacacagacgtcatcatagagttcgggggatcctttatgctaggacatgatgctgtcaaggttcaggaggagggaggatagctctgggccccatttgctatcaggcagaagaggcttagatactccagtgttcatggttatagttactgatgacgcctacacttcttttcctaagaagtaccatgttttcactgggcaaatcagaaacatacctgtattttgtctagtgaagttttagtgttaaactgatgaccttcagcattttaaatattattgaagcttatagaagcagaatttcaacagttccttcattgcagtctccttcattgacaaatattttttggcatatccaagaagtgcacatggaatttaaagttgaaagctattactagtgcctacgtaacaaaagttgttgatctttttttgtttaaaggaattgctgcttttgttactagacttaaaaatgattctgtagatacatgttacctaaatgccggcgtattcatgttttagagacaccacagtcttttaaaattactgaaatttctagaagtgaactatcaatcaaatatttgtcccaaattataacactgttgaatgcatagaaagccttgtttaaaaaatcacggatgtgaccgtttttctgctgtgatgtctactttcacagttattgatagtttctaacagtgcttaacctgcatgatcctgtttaatcctcataacaactctaacggattttcaccccatttcaaagagagaacctgaaggttagccagggcctgggcaggcgtctcatggccctaactgatagtgggatttacatgcgtttggttacacttcctgctgggcttctgtgtatagatgcggggccctggcagggcaagtgtaattacagacatggggtaagtgtagccgtggtctctttagggaatggctcacagtgtcaatattaaagcatattcaaaatacatacagtttggagagatcttcagcttgtacatctgtctttaagtatctaagtttgcctatgaattcctctgaatttaatgtcctttgaagttgcagtgtgctataaagtgtgtgcattatgtagctgtagatgtatttggattatcacctatgtaatctgatcagtttatattttattagagcctgtttgttttgttttcattattcctaaggaaattwtgtgttttcaggctgtgattgtcagaaacaaggaaaagaaagtaatatttgagttttctggccaaatttctggttcctcattccgcagtcactttcctccacttccatattccattttatttgtggtacatacatttctcagccatttggaattgtcatagatactggcgtaaggacatgctgtatggctgattgggcctctgtaagcagttttcattgttctgtgtacttacctttattgccttttcagccgactgttttaattacaaatgttttcttttttttctactctgccaccaaaagaatctttggctgctcctgacctcagcaaaccaagaggtgacttatgcatgccagttattcattgatactcactaaaaacaatgggtcaagccttgccccagtgctgactgactacgctgggtcgcccagtgtttgcagcagtaactaagtacacggctgcatgtgtgtgcgcattgcatttgaagcacggattgcctaaccttctcccgcctctccaaggattttttaaatcctaaggaggaaaaaaaaaaaactattccaaaattggaagtttttttttaaaatagtggtgctttatatagtctaaaaggatcacagtccttttggaagtaacacagattgattgttacagtgtatcagaggctgtgttataatcttgcaacgcttgttcaattgtattttgcttgaattggcatgctttgatacatacctatattaagttaatttgttgtctgagggttaatgaaaaatagtaattttaatattaaacttgctacattattatgaaggaagcttattttctaaactttagcatgttaaaggttacttttattactttaaaaagttgcattggctgggcctgatggctcacacctgtaatcccagcacttcgggaagctgaattggcaagattgtttgtggccaggagtttaagaccagcctgggcaacatagtgagaccctgtctctataaaaaatagaaatgcaaaatttttttaagttgcattaatcagtatttttttaatgagaagtagtgcagatcaagtgacactggatatttcactaatgtcctgagactggatcagtactcttcttcctacagagcagtgggttctacagaagaaaagccagaaggatggttagaatcatttgtagaaaaatgtatataatttagatgctagcatcccatttctcttaggactccttttgtttccttgaatgttagttgcgtacataaaactgtaagtccacattttagaactagagcctagttcataggggtctttaaactggagtgggttgggggatgtcttcaaagcaaagacgaggaatttccagtggtgaactttgaatgaaggcataaatgcggttgcatctttgtgtctttgatacactgagctctctttccatggcacaaaagcttaggagaaaacacactgacatctgttttcttttccaccaagggtatcactgggatacatcagattggatgccaagcgttcctctgccggacatacaagagttccccaactatgaggtgattgatgagcagacacccctgtactcagcagatccaaacgccatcgatacggactattaccctggaggctacgacatcgaaagtgattttcctccacccccagaagacttccccgcagctgatgagctaccaccgttaccgcccgaattcagcaatcagtttgaatccatccaccctcctagagacatgcctgccgcgggtagcttgggttcttcatcaagaaaccggcagaggttcaacttgaatcagtatttgcccaatttttatcccctcgatatgtctgaacctcaaacaaaaggcactggtgagaatagtacttgtagagaaccccatgccccttacccgccagggtatcaaagacacttcgaggcgcccgctgtcgagagcatgcccatgtctgtgtacgcctccaccgcctcctgctctgacgtgtcagcctgctgcgaagtggagtccgaggtcatgatgagtgactatgagagcggggacgacggccacttcgaagaggtgacgatcccgcccctggattcccagcagcacacggaagtctgactctcaactccccccaaagtgcctgactttagtgaacctagaggtgatgtgagtaatccgcgctgttctttgcagcagtgcttccaagctttttttggtgagccgaatgggcatggctgcgctggatcctgcgcctctggacgtgctagccatttccagtgtcccaactactgtcatcgtgaggttttcatcggctgtgccatttcccaacgtcttttgggatttacatctgtctgtgttaaaataatcaaacgaaaaatcagtcctgtgttgtcagcatgattcatgtatttatatagatttgattattttaattttcctgtctcttttttttgtaaattttatgtacagatttgatttttcatagttttaactagatttccaagatattttgtgcatttgtttcaactgaattttggtggtgtcagtgccattatctagcaccctgatttttttttttttactataaccagggtttcattctgtctttttccactgaagtgtgacattttgttagtacatttcagtgtagtcattcatttctagctgtacataggatgaaggagagatcagatacatgaacatgtcttacatgggttgctgtatttagaattataaacatttttcattattggaaagtgtaacggggaccttctgcatacctgtttagaaccaaaaccaccatgacacagtttttatagtgtctgtatatttgtgatgcaatggtcttgtaaaggtttttaatgaaaactaccattagccagtctttcttactgacaataaattattaataaaatacttgagctttatgcctctttttccttcccattgatactcctgttcaggattcagtggctgagatagaagaatggatgaatggaagggtggatattgatgtatttgcttttagtggacaaggcgtaaatgtgaaaatcctctctaccctgaccaaaagccctgctgtccaagcggagccagccaccaggtggcagtatatagctacttgatacggctcaaaactatagccacaaaagtaaaaagcataactttttaatatttttaaacaaaaccaagaaaattttatacatttttaattagtatgaatttattggttgtaattttaatgcttacatgtttttcacctttgtcaatgttttatatcaaaaacagtctctgtgaagcattatacctttctgttgtcaacttacacagtactgtatgatttaatgtgagatccagaagctattttttatatatttttatacagtcaaaatccatccctacttccaacacatttattgagtacttactatatgccactatgatatgaatgacaaacaattaagctttcttagaactttggagaaacatgttttccctttaagtaaatgcctttttatcaaataatataaatacatagtttaaaaacatcaaagagagctaaaatatatataataaaacagcagtcccatgcaacctgctgtacccagttacaactccttaagctgtttttctcattcttcctttcatattttcaaatgtatatattattctttcttgagtcactaaatttagaaattgcctgttgactccctatgatgttggaggaagaccatgtaacctaatagacctccactgatcttgttttcctaccattagttataatttttggattaatccatatacagcgtttccattattttgattatgtaagtattcactgctgtgccaagcagtatatgatgtttattttcatttacttagcaactttctttttttaacaattgtctttttattttactcattttttaaacttgtgactattcatgtattcagaaaacattttccctcaataatactatattattttgcctgaacagacagacaaaagtcctccgtcatcttggaatttacatttggtggggacaggtggtaggaggagatgatcaggagatacatggtagtgtttatagtagtgagtgctgaggagagagactgtgattgaggaaggaatgtgtgagattgtgatgggaagggacggggaaggggttgtgatgggaagggacattctgagaagacaggaggcctcagtggcaagcggcatttgagtccaaccttgaagttgatgaggaagaataccacgtggtttctctgagaactgttccaggcagacagattagctgctactgtaaagggaggagtgtgagaacgcccaagaaaaacacaagtgcagcctgtcatgtacatggtcacagcaggaagtctgtccgctcctttttttcccttggacattctcctcagggaacccctcggcttggtggcccagactccatttttttgttctcttcggcatctatagttgttgttccggaatattctccttaccatggactctgtaattttctcttggcctaactcttgttttgatagagcatatcctgttattgtctgagaaagagttgcatgggaagtaattttttgtgactgtgtttctgaaagtgtctttattctactcttgatagattgggtgtaggtttctggtttggaggtaaattacattcagaatcttgaagtcatggaacccactgtcttccagcttcaaatgttaacattgaaaagtccagtgttgttcctagtcccagttcattattcgtggcctaatatgactctcacaaatctttggggatattcttctggggacatgctacttctctacaacattgtgagatttcctgctgctgtgtcttggcgtgggccttttccagggattgctactccgtttttaatgaaaatccacgttgtggggactgttcttgtgttatttctttgatcatttcttcccttctgctttctcagttcttcgtttaattctcaaaggaggtgatctcagttctcttgtcaagactcataactataggttgcttgacttgctaaattctctcattttttgattgaactcctattgtccatctttgtcttattccccttctgggattcattccacttttatcttctgttccttctgttaccagttttatttctgctgtcatttttaatgtttaagagactggtctggtcctgactgtcccttttctatattacacaggcatttgttcctgtttcgaagctccctagtatctcatttttgctaaatgtattttagaataagtccttccattttgcctggttttcccctggacggcttttaaatattcctttcaggagggcttgctcccctttcaccttgcaggctttcctcttgcttgacttgagcctaatgaggcacccaagctgacagaaagccatgactgtgctccttagctcctggacttctctgtgacattgtgggccagcttgctttgtccgtttctttgtcttatctttgaagagtttcagtttctcccgaggaaggaccttctgtttccagcctgggaaatatcattgctcttattttgaaattggccagataaggggactggagatttgtcattcccttagtcctctagttctgggttacgagaacattcctgcttctttctctttgtctatttcatcatcgaggcggctcaggatagagctgagaatctgcattctaacagcttgaacaccgttcagacagcatccctgccactcaccccattcttcattaccttccatgtgtgacaaaggggcctgttcccatggcctccaggctctgctgcccgatccttgcatctgctgtagcgtggtttacagttaacaacgcctcttaaaagcattgaaaatggaattgctttttaactttttcccccatttcctgtttcagattcattttccttttttaaaataatgttttatttacttaaacagttattgtttgattgattttcaggagaagaaggggcagaatgtagccctccgccatctgaacctggaagtccggaatgtgctcacaccaccggtttccagtgtttggaagaacattggggtatcagtgttctgttgctccacagcacatggccgtacatttagtggcttaaaattacccccatttattacttctcattgcatagatcaaaacccccgggcatggcatggccaggttttctgctcagcttctcacaggctggaatcaagctgtcagctaggctgtggtttcatctgaggctggggagctcacgaggctcttggtagaattcagtcccttgcacttgtagaactggtctcattttcttgctaactgtacttgctaactgtaggacagaggttccctgtttcttgctggctgtccccactttttttttttttttttttttttgacatggagtctggctctgtcgcccaggctggagtgcaatggcacaatcttggctcactgcaacctctgcctcccaggttcaagccgttctcctgcctcagcctcccaagtacctgggattacaggcacgcgccaccgtacccagctaatttttgtatttttagtagagatgaggtttcaccatgttgctcaggctggtattgacctcctgacctcagataatccacctgcctcagcctcccaaagtgctgggattacaggcgtgagccaccgcgcccagcccgtccccactttcttgacagcttctcttgagtcaggtcctagctccttagccctctcacatgtagctacatcttcacctgcttcctgatctcaggaacggcctgatccctttttaaaggctcatgtgatttggtcaggtgctcactcaggacatcaagtctcactttgagtaactaaaagtcagctggttagtaacctagccatgggaattcccatcacattcacaagtcccattcaccttcgggggggaattacaccatgtacccaaggacagggatgggttctttgaggccatcttaaacttctacctaccaaaactggcactattcgttagttcttggttttatttatttattttttttttttgagacggagttttgctcttgttgaccaggctagagtgcaatggcatgaccttggctcaccacaacctctgcctccaggattcaagcgattctcctgcctcagcgtcccgagtagctgggattacaggcatgtactgccacgcccggctaattttgtatttttagtagagacagggtttctccatattggtcaggctggtcttgaactcctgacctcaggtgatccacctgcttcgccctccaaaattgctgggattacaggcatgagccaccatgcctggccagttcttgttctttttctaagggataattcccttatttagcagaatcatttttaagaagtttaaggttttaaaatgcctaaacataaatattataaaagatttaaatatctttaatcccttgggggaaatggggaaatgttggttaaaggttacaaactttcaattacaagaggaacgagttctgatgtccctgggtttcacctggattctatgagacacgcacacactagatttcaaaaccacttactatttgtcacaatctttatttggtacccttggaaattatggttcagcttaaaggaagagatatacagaaaaaagtctagtaagtatttaaacttttaaatttctatttttcttaattctacctctaaaatttagggggttttttgtttttgtttttgtctgtctgatgcgaagaatcaattgaagtgattgtgcttatcacgttcatttttattctagagtgtgtggagccatcctactcaacctgctgaaaagtgaggtagactttcccagtgcccagaaacagcaaatgtttttccagaagctgtattagcctctgggggccactttttattgggggagcccacccccaatatttcaacataggttctttctattttccgtaagtgtcggctggctgagaaatagagacagtacaaagagaggaatgttacagctgggccaccggggtgacatcacatttcggtaggaccgtgatgcccgcctgagtctcagaccagcaagtttttattatgggtttcaaaaggggagggggcgtaaaacagggagtaggtacaaagatcacatgcttcaaagggcaaaaagcagaactactaataagggtctaacaaagatcacatgctcctgagggaacaggacaaagggcaaaagcagaaccactgatgagggtccatgttcagcagtgcctgtatgtcttgataaacatcttaaacaacagaaaacagggttcaagagcagagaactggtctgaccacaaatttaccagagtgtagttttcccaaccctagtaagcctgagggtacgtcaggagaccagggcgcatctcagtccttatctcaaccgcataagacagacattcccagagcggccatttatagacctccctccaggaatgcattcctttcccagggtactaatattaatattccttgctagaaaaagaatttagcgatatcttccctacttgcacgtccatttataggctctctgcaagaagaaaaatagggctttttttgcctgaccccacaggcagtcagaccttatggttatcttcccttgttccctaaaaatcgctgttactctgttctttttcaaggtgcactgatttcatattgttcaaacacacgttttacaatcaatttatacagttaacacaattatcacagtggtcctgaggtgatgtatatcctcagcttatgaagataacaggattaagagattaaagtaaaggcaggcataagaaattataaaactagtatttgggaactgataaacgtccatgaaatcttcacaatttatgtttctctgctgtggctccagctggtccctccgttcggggtccctgacttcccgcaacaacttttaatgcacagaagcattaaaagtggatgctacctcagtctgaatgcacttttgcaaagaaactaagcattagcctaggaaataagggactcattctaaaaagatagcgattgaagaaggctgcacccacgtacacattgctaatgtgggtgacatgaactcaaaagtttcagtttacctcttttaatgaaagcactggaataagctcttggaaagcagggctttgagttgccaaatgaactgtcatagaacttcacctactttaatgctggtattaattgataactcaaaatcggggctgggcatggtggcttatgcctgtaatcccagcactttgggaggccaaggtgaaacgatcacttgtggctgggagttcaagacagcctgggcaatgcagtgagaccccatctctagtagaactttaaaaattaaattttaaaaaatagctgtaatcctcagacatacaggaatcagggctagctattattttaaaaaaaatagggtatcaacagggattaggagaaactgaacatctggattctttgatacctaagagtgtagggaacttggcaaataaaccaagctatgaaatgccacctcccaggagctgccacaagcttacaggttagctttaggatgtttcaagtgcaacaccaagatgcaatctgaagtccttagtttcgagatggggtggggaaaaattagtctcaccatcccctgctaaaaagagaaggaaggatattgttggaactgaggtgacaggagtggaggagggttgggggaggacagttggggacatgatggcagggctgcaggaagggaggtggcatccactctgcacagggctcagccaatgtgagcaaatgaaaggccattaaagctgcatcacaggaatgcatggctgcttgctaaagtcagtggcaagttaagccaagaatgttgcagacagagaatgcattctcatgtcatctacttgggtgcattcagtgggaaggcttgttggcatgttcattcctgcatgcatctagcaggtgttcgctgaaccttcaccctgggccacactcccttccctgacctctctcttcctggtgtcttcaaataatctggggataaatctcttattaagcagaattgttttttttttaagtttagagttctaaaatgtctaaatataagcattataagagatttaaatctctaatctgttgtggtaatggggcaatgttggttaaaggataagatgagcaggttctaatgtacagcataacgattatagttagtaataatgcatacttgacatttaccgagagagtaaatcataagtgtgttccccacggaaaaaaggtaattatgggaggggatggatatgctaattagacagattatgtaatcatttcacaatgtatatgcatatcaaatcatcacatggtacaccttaaatacatacaattttcatttatccatcataccttaataaagttggggaaaaataagctccccagagttaaactccccaatgacagggaaaaccccagcttccttccactgctttgtagcatttctgcttccaggagatgatacgaaactacctcaaaaaggcacatggatttcagacagtccccttcatatctcaggatttcattcctggttcttcacagtttctttctgttcaaatcactagacaccactggaacaggtgcatcttctcttcattttgtcagtcatctgcactagaaggaagtccttcacggtgttgtatatattttaattttttaaaaaaggacatccgtactgtattcccaaccatgtaagttacaggtatagaaagtagaaatgaaatgattagacacattttcttaagatttctgggatacaagcaatcctacattttaaaactgccatcgaatacattatcacgtttttatttttatgttttattttatatttaagttgaaaaaagtaaactattttgaaaaaatagttataatcatatcacccaggaacactattttgcatgcaatgatctgattttattttctgagtgcggtttacaaaatccgtatcttgtatcaaattttgtgccctgcttttctcactttaaagtatgtgtccttttcagaaaaattccattttaaagagctgtatactatttcatagcatgaatttactataacttatttacctattttattttgctactttgaagatttttttaattttttcacaacaatgacatctttgtatattcatttttgttcctcttccagacggatttatagatatgtggattatttgttttcaaagtattacctataagataacaaaattctagaacggcagcactggaactctgacaggattgtgttcagatttccttaacactgatgtagtcctctgctgttacttcatgccaatgttttccccacctcttccatgtcatggcatacatagaaaacttttttatggcatcgtggatgacataaaggccctaggctgctctaaaccagagatttcagagtggtatgcttacagtgagctcctcccaaccgcccagcccggagggctggagcaccaggctctgtgcacagcagctctgctatagacagctctggataatcctgcctccctgtctcttcctgtgaatttctttttctttctttctgagacggcatctcactctgtcccccatgctggagtgcaatggcgctatctcagctcactgcaacttccacctcttgggttcaagtgattctcttgcctcagactcccaagtagctgggattacaggcatgcaccactacacccagctaattttgtatttttagtagagacagggtttcaccatgttggccaggctggtctcgaactcctgaccttaggtgatccacccgcctcggcctcccaaagtgctgtgattacaggcgtgagccactgtgcctggcctttttttttttttttttttttttttgaggcagagtcttgctctgtcacccaggctggagtgcagtggtgcaatctcagctcactgcgacctccgcctccggggttcaagcgattctcgtgcctcagtctccgagtagctgggattacagatgcctgccaccacgcctggctaatttttatatttttagtagaaaccgggtttcactatgttgaccaggtgggtcttgaactcctgaccttgggtgattcgcctgcctcggcctcacaaagtgttgagattacaggcgtgagccactgcgcccaggcctgtgaattttttccatgaaaaacaaagtctaaaccagaatggaatcgtctttgtgaaatttgctctaatatggattcaattttcagtgacaggggtaaatgtaaggcttaggtttgctgtccatcaggcccactggaaaaccctcccgccccccacgcgccacctcccgtcctcttctggttgatgggctcccctgtcgccgcaatccccactttgactcaagtctactgcattgggtcagtagctttctgaaggtcaacgcggctgctctttctctttggccgactggcgttccctatacgttctgacagctcttcgtgaaaggcgttaggctccatttcaattagtgcaccggcggggggagagctgtttgtcagaatctaatttcctcgctagcagtttactgcctttaccgcaaacagaacgagccacttgaatagaatggcaagtatttttttttccccaagcaaatctctgctcatgtgaacacttaggcaattactgcacttgtcactgtgccgttaaagtgtgggtgtgtaggagctgcattttccagtgatgccagacttgaagagaaagagcgtgtgtgacacatttgaatgaaaatgtcatcacaccaagtatatcctgtagggagctaacagtgcaggtctctatagaagagatgttaaatatgagctcaggcctggctcagtggctcatgcctgtaatcccagcactttgggaggccacggcgggcggatcacctgaggtcgggagttcaagaccagcctggccaacatggggaaaccccgtgtctaccaaaaatacaaaaattagctgggcgtgatgacgcatgcctgaaatcccaactacttgggaggctgaggcaggagaatcacttgaacctgggaggcagaggttgtagtgagccaagatagtgcctctaccctccagcctgggttacagagtaagactctgtctcaaaacaaacaaaaaagataaatatgagctcagattcctaaattccaagcattatccaattatctcaagggataaaataaaattctcaaaatgtctaatgactatcactttgcttcatttttttgtttgtttgtttctttgttttgagacagagtctcactctgtcacccaggctggagtgcagtggcatgatctcggctcagtgcagcctctgcctcccgggttcaagcaattctcctgtctcctccctagtagctgggactacaggcacgcaccgccatgcccggctaattttttgtatttttttttttttagtagagacagggtttcaccatcttggacaggatggtcttgatctcctgacctcgtgatccacctgcctcagcctcccaaagtgctgggattacaggcatgagccaccatgcccggccactttgctttgttttaatgttcatgagctctacctaagtttcagcataaggggagaatatggagagaggaacaagggctggccccggaggctgtgtgggctcttcctgcctgtcagccttggaagcacagttccacgtctttgtcaagttcgaaatgttagcccacctgccaagtaggattacaaacgcatttttaaatgatgctcttgttttgtgattggtgttcttaaaattttaatttgaaatgagtatttttctcttcttttgaaagaagagaaagtatactttttaaaattatacctatttaaggtatatgtgatgtttatttacatatacacacaaacacgtagaagtttacttcagtcaagcaaatgacatgtgcaccgtttcacatagttaccctttttggtgtgtgtggtgaaagtacctaaattctactctcttactaaatttccagcatacatgatgatttggatgtgtgtccccaccaaacctcacattaaattgaacacttcattgttggaggtggggcccggtaggtcattggaccgtgggggtggagttctcgtgactgctttagtaccgtccgccccacaccccacccccgggtactgtacagggagttctctcaacatctggctgtttaagtgtgtgtagcacttcccccctttctgtattcctcctgctccagccgtgtgagaagcctgctcccccttccccttccaccatgactggaagcttcctgaggcctcccaagaagcagaagctgccatcttcctgtacagccggcagaaccataaacctgttaaacctcttttctttatcaatcacccagtgccaggttcctttagagcaatgcaaaaactgactgatacacatgacaatattattcactgcagtcttagcattcttcacatctccaggcttattcatcctacatagtggacactttctacacttcaatctccatctcctcaattcccgccaccacccccaccccccttattttgcttattttgcaaaatgtcctcaggttaattataaattatattataaatcacagaatctccttttgtatagctgaaaaaaatattctaatgtgtacacataccacagtttctttgtccattcatccatcaatggccccacgggttgtttacatatcttggctcttaggaatcctgctgtgataaacaggggtaccgatattttcatgaggtggtgatgtcattttgcaaagagggattgctgggtcggggccacatactgttttccttaatggccgcaccaatctacagtctcaccagcagcgaaccagggttcccctttctacacaccctcaccgacatttgtgatctcctcttttttatcatagccattctaacaagggctcatgtggttgtaatttgcatttccttcatgattagaggtgttgagtacctttatcatatacccgttggctgtttttatttcttctttggagaaatgtctatttaggttctttgccttttttttttttttttgagatggagtctcgctgttgtcggcaggggctggagtgcagtggtgcgatctcggctcactgcaacccccacctcccgggttccagcaattctcccacctcagcctcctgagtagctgagattacaggcacccaccacctcacctggctaatttttgtatttttagtagacacagggtttcaccattttggccaggctgggctcgaactcctgacctcatgtgatccacccccaccaccttggcctcccatagtgctgggattacaggtgtgaggcaccgcacccggccctttgcccatttttaattgggttatttggtattttgctactgaattgtatgagttccttatattttccagatattaaccccttatccaatatgtggtttgcaaatatttcctcccaatctgtttgctgggttttcattttgttggttattttctttgctgtgcagaagcttttcagtttgatgtagccctacttacttaaatattttctcttttattgcctgtgcttttggtgagatatccaaaaaatccttgccaagttcaatctcaagaagctttctagctatgttttcttccaggagtttcatgatttcaggtcttacatttaggtccttaatccattttgagttgatttcgtatatggtgtaagataaggatgcattttcattcttttgcatgtggatatccagttttgccaataccatttactgaagagactatcttttttccattgtgtcttcttgattgcttaagtatactttaaaatagatactttaaattagtctgcacaaaagaaaatgttattcataagccttattaaaaacaaattgttctaattaaataaacaatgatcaagatgcagaaacttctccaactaaaagctcttattggatagaaatggtttttaataataatttcttaaaaatgctgagttttaacttctagtggggaatttcacaaaatttaaaaatacacagaggtccttgaaaacaggtcatacataccatgtgcttggagctgttaccgctcacactgaacgcagggcgggctctgtgctttttaccaaaatatcagcgaactctgctgagtctgtgcacaggctctgttcagtaagctttagctgcagttcatccttgggctctggtggctgagcccacgccaagtccctctgctgcacctggtggggttccagtttcaccgctgagtgactgtgaccacacctatggctgggtagctgcacagtctctgggtttgatttatttaatggatcaaagttcagtccattctctctgttggtcagagttttgcattttaaaaccatgtacccttagggtgcactttcaaactcccaggcctttgggcaccaggggcaggcagggacggccggcttcagagcacacacctaagttcaaaattgttgaactctgcagtcttttttttttttttttttgagacagagtctcgctctgtcttcaggctggagtgcagtggcgcgatctcggctcagtgtgacctccacctcccgggttcaagcaattctcctgcctcagcctcctgagtagctgggactacagacatgcaccaccacgcccggctaatttttttttttttttttttttttttttagtagagacggggtttcaccatgttgatcaggatggtctcgatcatctggacctcatgatccacccgccttggccacccaaagtgctgggattccaggcgtgagccactgcacccggcccaca

SEQ ID NO: 2 - DNA, 14756 bp, Homo sapiens
  CDS (187)..(13959)
ctgggcggccgggcgcggggagagggcgcgggagcggctcgtgcggcaggtaccatgcggacgcgcgagcccggcgaggccccggcaggcccgtccctgctcgggggcgcgctgagacggcgggtgagctccacgagagcgccgtcgccacttcgggccaactttgcgattcccgacagttaagcaatggggagacatttggctttgctcctgcttctgctccttctcttccaacattttggagacagtgatggcagccaacgacttgaacagactcctctgcagtttacacacctcgagtacaacgtcaccgtgcaggagaactctgcagctaagacttatgtggggcatcctgtcaagatgggtgtttacattacacatccagcgtgggaagtaaggtacaaaattgtttccggagacagtgaaaacctgttcaaagctgaagagtacattctcggagacttttgctttctaagaataaggaccaaaggaggaaatacagctattcttaatagagaagtgaaggatcactacacattgatagtgaaagcacttgaaaaaaatactaatgtggaggcgcgaacaaaggtcagggtgcaggtgctggatacaaatgacttgagaccgttattctcacccacctcatacagcgtttctttacctgaaaacacagctataaggaccagtatcgcaagagtcagcgccacggatgcagacataggaaccaacggggaattttactacagttttaaagatcgaacagatatgtttgctattcacccaaccagtggtgtgatagtgttaactggtagacttgattacctagagaccaagctctatgagatggaaatcctcgctgcggaccgtggcatgaagttgtatgggagcagtggcatcagcagcatggccaagctaacggtgcacatcgaacaggccaatgaatgtgctccggtgataacagcagtgacattgtcaccatcagaactggacagggacccagcatatgcaattgtgacagtggatgactgcgatcagggtgccaatggtgacatagcatctttaagcatcgtggcaggtgaccttctccagcagtttagaacagtgaggtcctttccagggagtaaggagtataaagtcaaagccatcggtgacattgattgggacagtcatcctttcggctacaatctcacactacaggctaaagataaaggaactccgccccagttctcttctgttaaagtcattcacgtgacttctccacagttcaaagccgggccagtcaagtttgaaaaggatgtttacagagcagaaataagtgaatttgctcctcccaacacacctgtggtcatggtaaaggccattcctgcttattcccatttgaggtatgtttttaaaaggacacctggaaaagctaaattcagtttaaattacaacactggtctcatttctattttagaaccagttaaaagacagcaggcagcccattttgaacttgaagtaacaacaagtgacagaaaagcgtccaccaaggtcttggtgaaagtcttaggtgcaaatagcaatccccctgaatttacccagacagcgtacaaagctgcttttgatgagaacgtgcccattggtactactatcatgagcctgagtgccgtagaccctgatgagggtgagaatgggtacgtgacatacagtatcgcaaatttaaatcatgtgccgtttgcgattgaccatttcactggtgccgtgagtacgtcagaaaacctggactacgaactgatgcctcgggtttatactctgaggattcgtgcatcagactggggcttgccgtaccgccgggaagtcgaagtccttgctacaattactctcaataacttgaatgacaacacacctttgtttgagaaaataaattgtgaagggacaattcccagagatctaggcgtgggagagcaaataaccactgtttctgctattgatgcagatgaacttcagttggtacagtatcagattgaagctggaaatgaactggatttgtttagtttaaaccccaactcgggggtattgtcattaaagcgatcgctaatggatggcttaggtgcaaaggtgtctttccacagtctgagaatcacagctacagatggagaaaattttgccacaccattatatatcaacataacagtggctgccagtcacaagctggtaaacttgcagtgtgaagagactggtgttgccaaaatgctggcagagaagctcctgcaggcaaataaattacacaaccagggagaggtggaggatattttcttcgattctcactctgtcaatgctcacataccgcagtttagaagcactcttccgactggtattcaggtaaaggaaaaccagcctgtgggttccagtgtaattttcatgaactccactgaccttgacactggcttcaatggaaaactggtctatgctgtttctggaggaaatgaggatagttgcttcatgattgatatggaaacaggaatgctgaaaattttatctcctcttgaccgtgaaacaacagacaaatacaccctgaatattaccgtctatgaccttgggataccccagaaggctgcgtggcgtcttctacatgtcgtggttgtcgatgccaatgataatccacccgagtttttacaggagagctattttgtggaagtgagtgaagacaaggaggtacatagtgaaatcatccaggttgaagccacagataaagacctggggcccaacggacacgtgacgtactcaattcttacagacacagacacattttcaattgacagcgtgacgggtgttgttaacatcgcacgccctctggatcgagagctgcagcatgagcactccttaaagattgaggccagggaccaagccagagaagagcctcagctgttctccactgtcgttgtgaaagtatcactagaagatgttaatgacaacccacctacatttattccacctaattatcgtgtgaaagtccgagaggatcttccagaaggaaccgtcatcatgtggttagaagcccacgatcctgatttaggtcagtctggtcaggtgagatacagccttctggaccacggagaaggaaacttcgatgtggataaactcagtggagcagttaggatcgtccagcagttggactttgagaagaagcaagtgtataatctcactgtgagggccaaagacaagggaaagccagtttctctgtcttctacttgctatgttgaagttgaggtggttgatgtgaatgagaacctgcacccacccgtgttttccagctttgtggaaaaggggacagtgaaagaagatgcacctgttggttcattggtaatgacggtgtcggctcatgatgaggacgccggaagagatggggagatccgatactccattagagatggctctggcgttggtgttttcaaaataggtgaagagacaggtgtcatagagacgtcagatcgactggaccgtgaatcgacctcccattattggctaacagtctttgcaaccgatcagggtgtcgtgcctctttcatcgttcatagagatctacatagaggttgaggatgtcaatgacaatgcaccacagacatcagagcctgtttattacccagaaatcatggaaaattctcctaaagatgtatctgtggtccagatcgaggcatttgatccagattcgagctctaatgacaagctcatgtacaaaattacaagtggaaatccacaaggattcttttcaatacatcctaaaacaggtctcatcacaactacgtcaaggaagctagaccgagaacagcaagatgaacacatattagaggttactgtgacagacaatggtagtccccccaaatcaaccattgcaagagtcattgtgaaaatccttgatgaaaatgacaacaaacctcagtttctgcaaaagttctacaaaatcagactccctgagcgggaaaagccagaccgagaaagaaatgccagacgggagccgctctatcgcgtcatagccaccgacaaggatgagggccccaatgcagaaatctcctacagcatcgaagacgggaatgagcatggcaaatttttcatcgaaccgaaaactggagtggtttcgtccaagaggttttcagcagctggagaatatgatattctttcaattaaggcagttgacaatggtcgccctcaaaagtcatcaaccaccagactccatattgaatggatctccaagcccaaacagtccctggagcccatttcatttgaagaatcattttttacctttactgtgatggaaagtgaccccgttgctcacatgattggagtaatatctgtggagcctcctggcatacccctttggtttgacatcactggtggcaactacgacagtcacttcgatgtggacaagggaactggaaccatcattgttgccaaacctcttgatgcagaacagaagtcaaactacaacctcacagtcgaggctacagatggaaccaccactatcctcactcaggtattcatcaaagtaatagacacaaatgaccatcgtcctcagttttctacatcaaagtatgaagttgttattcctgaagatacagcgccagaaacagaaattttgcaaatcagtgctgtggatcaggatgagaaaaacaaactaatctacactctgcagagcagtagagatccactgagtctcaagaaatttcgtcttgatcctgcaaccggctctctctatacttctgagaaactggatcatgaagctgtttcaccagcacacctcacggtcatggtacgagatcaagatgtgcctgtaaaacgcaactttgcaaggattgtggtcaatgtcagcgacacgaatgaccacgccccgtggttcaccgcttcctcctacaaagggcgggtttatgaatcggcagccgttggctcagttgtgttgcaggtgacggctctggacaaggacaaagggaaaaatgctgaagtgctgtactcgatcgagtcaggaaatattggaaatattggaaattcttttatgattgatcctgtcttgggctctattaaaactgccaaagaattagatcgaagtaaccaagcggagtatgatttaatggtaaaagctacagataagggcagtccaccaatgagtgaaataacttctgtgcgtatctttgtcacaattgctgacaacgcctctccgaagtttacatcaaaagaatattctgttgaacttagtgaaactgtcagcattgggagtttcgttgggatggttacagcccatagtcaatcatcagtggtgtatgaaataaaagatggaaatacaggtgatgcttttgatattaatccacattctggaactatcatcactcagaaagccctggactttgaaactttgcccatttacacattgataatacaaggaactaacatggctggtttgtccactaatacaacggttctagttcacttgcaggatgagaatgacaacgcgccagtttttatgcaggcagaatatacaggactcattagtgaatcagcctcaattaacagcgtggtcctaacagacaggaatgtcccactggtgattcgagcagctgatgctgataaagactcaaatgctttgcttgtatatcacattgttgaaccatctgtacacacatattttgctattgattctagcactggtgctattcatacagtactaagtctggactatgaagaaacaagtatttttcactttaccgtccaagtgcatgacatgggaaccccacgtttatttgctgagtatgcagcgaatgtaacagtacatgtaattgacattaatgactgcccccctgtgtttgccaagccattatatgaagcatctcttttgttaccaacatacaaaggagtaaaagtcatcacagtaaatgctacagatgctgattcaagtgcattctcacagttgatttactccatcaccgaaggcaacatcggggagaagttttctatggactacaagactggtgctctcactgtccaaaacacaactcagttaagaagccgctacgagctaaccgttagagcttccgatggcagatttgccggccttacctctgtcaaaattaatgtgaaagaaagcaaagaaagtcacctaaagtttacccaggatgtctactctgcggtagtgaaagagaattccaccgaggccgaaacattagctgtcattactgctattgggagtccaatcaatgagcctttgttttatcacatcctcaacccagatcgcagatttaaaataagccgcacttcaggggttctgtcaaccactggcacgcccttcgatcgtgagcagcaggaggcgtttgatgtggttgtagaagtgatagaggaacataagccttctgcagtggcccacgttgtcgtgaaggtcattgtagaagaccaaaatgataatgcgccggtgtttgtcaaccttccctactacgccgttgttaaagtggacactgaggtgggccatgtcattcgctatgtcactgctgtagacagagacagtggcagaaacggggaagtgcattactacctcaaggaacatcatgaacactttcaaattggacccttgggtgaaatttcactgaaaaagcaatttgagcttgacaccttaaataaagaatatcttgttacagtggttgcaaaagatggagggaacccggccttttcagcggaagttatcgttccgatcactgtcatgaataaagccatgcctgtgtttgaaaaacctttctacagtgcagagattgcagagagcatccaggtgcacagccctgtggtccacgtgcaggctaacagcccggaaggcctgaaagtgttctacagcatcacagacggagaccctttcagccagttcactattaacttcaatactggagttatcaatgtcatagctcctctggactttgaggcccacccggcatataagctgagcatacgcgcaactgactccttgacgggcgctcatgctgaagtatttgtggacatcatagtagacgacatcaatgataaccctcctgtgtttgctcagcagtcttatgcggtgaccctgtctgaggcatctgtaattggaacgtctgttgttcaagttagagccaccgattctgattcagaaccaaatagaggaatctcataccagatgtttgggaatcacagcaagagtcatgatcattttcatgtagacagcagcactggcctcatctcactactcagaaccctggattacgagcagtcccggcagcacacgatttttgtgagggcagttgatggtggtatgcccacgctgagcagtgatgtgattgtcacggtggacgttaccgacctcaatggtaatccaccactctttgaacaacagatttatgaagccagaattagcgagcacgcccctcatgggcatttcgtgacctgtgtaaaagcctatgatgcagacagttcagacatagacaagttgcagtattccattctgtctggcaatgatcataaacattttgtcattgacagtgcaacagggattatcaccctctcaaacctgcaccggcacgccctgaagccattttacagtcttaacctgtcagtgtctgatggagtttttagaagttccacccaggttcatgtaactgtaattggaggcaatttgcacagtcctgctttccttcagaacgaatatgaagtggaactagctgaaaacgctcccctacataccctggtgatggaggtgaaaactacggatggggattctggtatttatggtcacgttacttaccatattgtaaatgactttgccaaagacagattttacataaatgagagaggacagatatttactttggaaaaacttgatcgagaaaccccggcggagaaagtgatctcagtccgtttaatggctaaggatgctggaggaaaagttgctttctgcaccgtgaatgtcatccttacagatgacaatgacaatgcaccacaatttcgagcaaccaaatacgaagtgaatatcgggtccagtgctgctaaagggacttcagtcgtaaagtctgcaagtgatgccgatgagggctccaatgccgacatcacctatgccattgaagcagactctgaaagtgtaaaagagaatttggaaattaacaaactgtccggcgtaatcactacaaaggagagcctcattggcttggaaaatgaattcttcactttctttgttagagctgtggataatgggtctccatcaaaagaatctgttgttcttgtctatgttaaaatccttccaccggaaatgcagcttccaaaattttcagaacctttctatacctttacagtgtcagaggacgtgcctgttggaacagagatagatctcatccgagcagaacatagtgggactgttctttacagcctggtcaaagggaatactccagaaagcaatagggatgagtcctttgtgattgacagacagagcgggagactgaagttggagaagagtcttgatcatgagacaactaagtggtatcagttttccatactggccaggtgcactcaagatgaccatgagatggtggcttctgtagatgttagtatccaagtgaaagatgcaaatgacaacagcccggtctttgaatctagtccatatgaggcattcattgttgaaaacctgccagggggaagtagagtaattcagatcagggcatctgatgctgactcaggaaccaacggccaagttatgtatagcctggatcagtcacaaagtgtggaagtcattgaatcctttgccattaacatggaaacaggctggattacaactttaaaggaacttgaccatgaaaagagagacaattaccagattaaagtggttgcatcagatcatggtgaaaagatccagctatcctccacagccattgtggatgttaccgtcaccgatgtcaacgatagtccaccacgattcacggccgagatctataaagggactgtgagtgaggatgacccccaaggtggggtgattgccatcttaagtaccacggatgctgattctgaagagatcaacagacaagttacatatttcataacaggaggggatcctttaggacagtttgccgttgaaactatacagaatgaatggaaggtatatgtgaagaaacctctagacagggaaaaaagggacaattaccttcttactatcacggcaactgatggcaccttctcatcaaaagcgatagttgaagtgaaagttctggatgcaaatgacaacagtccagtttgtgaaaagactttatattcagacactattcctgaagacgtccttcctggaaaattgatcatgcagatctctgctacagacgcagacatccgctctaacgctgaaattacttacacgttattgggttcaggtgcagaaaaattcaaactaaatccagacacaggtgaactgaaaacgtcaaccccccttgatcgtgaggagcaagctgtttatcatcttctcgtcagggccacagatggaggaggaagattctgccaagccagtattgtcgtcacgctagaagatgtgaacgataacgcccccgaattctctgccgatccttatgccatcaccgtgtttgaaaacacagagccgggaacgctgctgacaagagtgcaggccacagatgccgacgcaggattaaatcggaagattttatactcactgattgactctgctgatgggcagttctccattaacgaattatctggaattattcagttagaaaaacctttggacagagaactccaggcagtatacaccctctctttgaaagctgtggatcaaggcttgccaaggaggctgactgccactggcactgtgattgtatcagttcttgacataaatgacaacccccctgtgtttgagtaccgtgaatatggtgccaccgtgtctgaggacattcttgttggaactgaagttcttcaagtgtatgcagcaagtcgggatattgaagcaaatgcagaaatcacctactcaataataagtggaaatgaacatgggaaattcagcatagattctaaaacaggggccgtatttatcattgagaatctggattatgagagctctcatgagtattacctaacagtagaggccactgatggaggcacgccttcactgagcgacgttgccactgtgaacgttaatgtaacagatatcaacgataatacccctgtgttcagccaagacacctacacgacagtcatcagtgaagatgccgttcttgagcagtctgtcatcacggttatggccgatgatgccgatggaccttccaacagccacatccactactcaattatagatggcaaccaaggaagctcgttcacaattgaccccgtcaggggagaagtcaaagtgaccaaacttctcgaccgagaaacgatttcaggttacacgctcacggttcaagcttctgataatggcagtccacccagagtcaacacgacgaccgtgaacatcgatgtgtccgatgtcaatgacaacgcgcccgtcttctccaggggaaactacagtgtcattatccaggaaaataagccagtgggcttcagcgtgctgcagctggtagtaacagatgaggattcttcccataacggtccacccttcttctttactattgtaactggaaatgatgagaaggcttttgaagttaacccgcaaggagtcctcctgacatcatctgccatcaagaggaaggagaaagatcattacttactgcaggtgaaggtggcagataatggaaagcctcagttgtcatctttgacatacattgacattagggtaattgaggagagcatctatccgcctgcgattttgcccctggagattttcatcacctcttctggagaagaatactcaggtggcgtcattgggaagatccatgccacagaccaggacgtgtatgatactctaacctacagtctcgaccctcagatggacaacctgttctctgtttccagcacagggggcaagctgatagcacacaaaaagctagacatagggcaataccttctcaatgtcagcgtaacagatgggaagttcacgacggtggccgacatcacagtgcatatcagacaagtcacacaggagatgttgaaccacaccatcgcgatccgctttgccaacctcactccggaagaattcgttggtgactactggcgcaacttccagcgagctttacggaacatcctgggtgtgaggaggaacgacatacagattgttagtttgcagtcctctgaacctcacccacatctggacgtcttactttttgtagagaaaccaggtagtgctcagatctcaacaaaacaacttctgcacaagattaactcttccgtgactgacattgaggaaatcattggagttaggatactgaatgtattccagaaactctgcgcgggactggactgcccctggaagttctgcgatgaaaaggtgtctgtggatgaaagtgtgatgtcaacacacagcacagccagactgagttttgtgactccccgccaccacagggcagcggtgtgtctctgcaaagagggaaggtgcccacctgtccaccatggctgtgaagatgatccgtgccctgagggatccgaatgtgtgtctgatccctgggaggagaaacacacctgtgtctgtcccagcggcaggtttggtcagtgcccagggagttcatctatgacactgactggaaacagctacgtgaaataccgtctgacggaaaatgaaaacaaattagagatgaaactgaccatgaggctcagaacatattccacgcatgcggttgtcatgtatgctcgaggaactgactatagcatcttggagattcatcatggaaggctgcagtacaagtttgactgtggaagtggccctggaattgtctctgttcagagcattcaggtcaatgatgggcagtggcacgcagtggccctggaagtgaatggaaactatgctcgcttggttctagaccaagttcatactgcatcgggcacagccccagggactctgaaaaccctgaacctggataactatgtgttttttggtggccacatccgtcagcagggaacaaggcatggaagaagtcctcaagttggtaatggtttcaggggttgtatggactccatttatttgaatgggcaggagctccctttaaacagcaaacccagaagctatgcacacatcgaagagtcggtggatgtatctccaggctgcttcctgacggccacggaagactgcgccagcaacccttgccagaatggaggcgtttgcaatccgtcacctgctggaggttattactgcaaatgcagtgccttgtacatagggacccactgtgagataagcgtcaatccgtgttcctccaacccatgcctctatgggggcacgtgtgttgtcgacaacggaggctttgtttgccagtgtagaggattatatactggtcagaggtgtcagcttagtccatactgcaaagatgaaccctgtaagaatggcggaacatgctttgacagtttggatggcgccgtttgtcagtgtgattcgggttttaggggagaaaggtgtcagagtgatatcgacgagtgctctggaaacccttgcctgcacggggccctctgtgagaacacgcacggctcctatcactgcaactgcagccacgagtacaggggacgtcactgcgaggatgctgcgcccaaccagtatgtgtccacgccgtggaacattgggttggcggaaggaattggaatcgttgtgtttgttgcagggatatttttactggtggtggtgtttgttctctgccgtaagatgattagtcggaaaaagaagcatcaggctgaacctaaagacaagcacctgggacccgctacggctttcttgcaaagaccgtattttgattccaagctaaataagaacatttactcagacataccaccccaggtgcctgtccggcctatttcctacaccccgagtattccaagtgactcaagaaacaatctggaccgaaattccttcgaaggatctgctatcccagagcatcccgaattcagcacttttaaccccgagtctgtgcacgggcaccgaaaagcagtggcggtctgcagcgtggcgccaaacctgcctcccccacccccttcaaactccccttctgacagcgactccatccagaagcctagctgggactttgactatgacacaaaagtggtggatcttgatccctgtctttccaagaagcctctagaggaaaagccttcccagccatacagtgcccgggaaagcctgtctgaagtgcagtccctgagctccttccagtccgaatcgtgcgatgacaatgggtatcactgggatacatcagattggatgccaagcgttcctctgccggacatacaagagttccccaactatgaggtgattgatgagcagacacccctgtactcagcagatccaaacgccatcgatacggactattaccctggaggctacgacatcgaaagtgattttcctccacccccagaagacttccccgcagctgatgagctaccaccgttaccgcccgaattcagcaatcagtttgaatccatccaccctcctagagacatgcctgccgcgggtagcttgggttcttcatcaagaaaccggcagaggttcaacttgaatcagtatttgcccaatttttatcccctcgatatgtctgaacctcaaacaaaaggcactggtgagaatagtacttgtagagaaccccatgccccttacccgccagggtatcaaagacacttcgaggcgcccgctgtcgagagcatgcccatgtctgtgtacgcctccaccgcctcctgctctgacgtgtcagcctgctgcgaagtggagtccgaggtcatgatgagtgactatgagagcggggacgacggccacttcgaagaggtgacgatcccgcccctggattcccagcagcacacggaagtctgactctcaactccccccaaagtgcctgactttagtgaacctagaggtgatgtgagtaatccgcgctgttctttgcagcagtgcttccaagctttttttggtgagccgaatgggcatggctgcgctggatcctgcgcctctggacgtgctagccatttccagtgtcccaactactgtcatcgtgaggttttcatcggctgtgccatttcccaacgtcttttgggatttacatctgtctgtgttaaaataatcaaacgaaaaatcagtcctgtgttgtcagcatgattcatgtatttatatagatttgattattttaattttcctgtctcttttttttgtaaattttatgtacagatttgatttttcatagttttaactagatttccaagatattttgtgcatttgtttcaactgaattttggtggtgtcagtgccattatctagcaccctgatttttttttttttactataaccagggtttcattctgtctttttccactgaagtgtgacattttgttagtacatttcagtgtagtcattcatttctagctgtacataggatgaaggagagatcagatacatgaacatgtcttacatgggttgctgtatttagaattataaacatttttcattattggaaagtgtaacggggaccttctgcatacctgtttagaaccaaaaccaccatgacacagtttttatagtgtctgtatatttgtgatgcaatggtcttgtaaaggtttttaatgaaaactaccattagccagtctttcttactgacaataaattattaataaaat

SEQ ID NO: 3 - protein, 4590 aa, Homo sapiens
MGRHLALLLLLLLLFQHFGDSDGSQRLEQTPLQFTHLEYNVTVQENSAAKTYVGHPVKMGVYITHPAWEVRYKIVSGDSENLFKAEEYILGDFCFLRIRTKGGNTAILNREVKDHYTLIVKALEKNTNVEARTKVRVQVLDTNDLRPLFSPTSYSVSLPENTAIRTSIARVSATDADIGTNGEFYYSFKDRTDMFAIHPTSGVIVLTGRLDYLETKLYEMEILAADRGMKLYGSSGISSMAKLTVHIEQANECAPVITAVTLSPSELDRDPAYAIVTVDDCDQGANGDIASLSIVAGDLLQQFRTVRSFPGSKEYKVKAIGDIDWDSHPFGYNLTLQAKDKGTPPQFSSVKVIHVTSPQFKAGPVKFEKDVYRAEISEFAPPNTPVVMVKAIPAYSHLRYVFKRTPGKAKFSLNYNTGLISILEPVKRQQAAHFELEVTTSDRKASTKVLVKVLGANSNPPEFTQTAYKAAFDENVPIGTTIMSLSAVDPDEGENGYVTYSIANLNHVPFAIDHFTGAVSTSENLDYELMPRVYTLRIRASDWGLPYRREVEVLATITLNNLNDNTPLFEKINCEGTIPRDLGVGEQITTVSAIDADELQLVQYQIEAGNELDLFSLNPNSGVLSLKRSLMDGLGAKVSFHSLRITATDGENFATPLYINITVAASHKLVNLQCEETGVAKMLAEKLLQANKLHNQGEVEDIFFDSHSVNAHIPQFRSTLPTGIQVKENQPVGSSVIFMNSTDLDTGFNGKLVYAVSGGNEDSCFMIDMETGMLKILSPLDRETTDKYTLNITVYDLGIPQKAAWRLLHVVVVDANDNPPEFLQESYFVEVSEDKEVHSEIIQVEATDKDLGPNGHVTYSILTDTDTFSIDSVTGVVNIARPLDRELQHEHSLKIEARDQAREEPQLFSTVVVKVSLEDVNDNPPTFIPPNYRVKVREDLPEGTVIMWLEAHDPDLGQSGQVRYSLLDHGEGNFDVDKLSGAVRIVQQLDFEKKQVYNLTVRAKDKGKPVSLSSTCYVEVEVVDVNENLHPPVFSSFVEKGTVKEDAPVGSLVMTVSAHDEDAGRDGEIRYSIRDGSGVGVFKIGEETGVIETSDRLDRESTSHYWLTVFATDQGVVPLSSFIEIYIEVEDVNDNAPQTSEPVYYPEIMENSPKDVSVVQIEAFDPDSSSNDKLMYKITSGNPQGFFSIHPKTGLITTTSRKLDREQQDEHILEVTVTDNGSPPKSTIARVIVKILDENDNKPQFLQKFYKIRLPEREKPDRERNARREPLYRVIATDKDEGPNAEISYSIEDGNEHGKFFIEPKTGVVSSKRFSAAGEYDILSIKAVDNGRPQKSSTTRLHIEWISKPKQSLEPISFEESFFTFTVMESDPVAHMIGVISVEPPGIPLWFDITGGNYDSHFDVDKGTGTIIVAKPLDAEQKSNYNLTVEATDGTTTILTQVFIKVIDTNDHRPQFSTSKYEVVIPEDTAPETEILQISAVDQDEKNKLIYTLQSSRDPLSLKKFRLDPATGSLYTSEKLDHEAVSPAHLTVMVRDQDVPVKRNFARIVVNVSDTNDHAPWFTASSYKGRVYESAAVGSVVLQVTALDKDKGKNAEVLYSIESGNIGNIGNSFMIDPVLGSIKTAKELDRSNQAEYDLMVKATDKGSPPMSEITSVRIFVTIADNASPKFTSKEYSVELSETVSIGSFVGMVTAHSQSSVVYEIKDGNTGDAFDINPHSGTIITQKALDFETLPIYTLIIQGTNMAGLSTNTTVLVHLQDENDNAPVFMQAEYTGLISESASINSVVLTDRNVPLVIRAADADKDSNALLVYHIVEPSVHTYFAIDSSTGAIHTVLSLDYEETSIFHFTVQVHDMGTPRLFAEYAANVTVHVIDINDCPPVFAKPLYEASLLLPTYKGVKVITVNATDADSSAFSQLIYSITEGNIGEKFSMDYKTGALTVQNTTQLRSRYELTVRASDGRFAGLTSVKINVKESKESHLKFTQDVYSAVVKENSTEAETLAVITAIGSPINEPLFYHILNPDRRFKISRTSGVLSTTGTPFDREQQEAFDVVVEVIEEHKPSAVAHVVVKVIVEDQNDNAPVFVNLPYYAVVKVDTEVGHVIRYVTAVDRDSGRNGEVHYYLKEHHEHFQIGPLGEISLKKQFELDTLNKEYLVTVVAKDGGNPAFSAEVIVPITVMNKAMPVFEKPFYSAEIAESIQVHSPVVHVQANSPEGLKVFYSITDGDPFSQFTINFNTGVINVIAPLDFEAHPAYKLSIRATDSLTGAHAEVFVDIIVDDINDNPPVFAQQSYAVTLSEASVIGTSVVQVRATDSDSEPNRGISYQMFGNHSKSHDHFHVDSSTGLISLLRTLDYEQSRQHTIFVRAVDGGMPTLSSDVIVTVDVTDLNGNPPLFEQQIYEARISEHAPHGHFVTCVKAYDADSSDIDKLQYSILSGNDHKHFVIDSATGIITLSNLHRHALKPFYSLNLSVSDGVFRSSTQVHVTVIGGNLHSPAFLQNEYEVELAENAPLHTLVMEVKTTDGDSGIYGHVTYHIVNDFAKDRFYINERGQIFTLEKLDRETPAEKVISVRLMAKDAGGKVAFCTVNVILTDDNDNAPQFRATKYEVNIGSSAAKGTSVVKSASDADEGSNADITYAIEADSESVKENLEINKLSGVITTKESLIGLENEFFTFFVRAVDNGSPSKESVVLVYVKILPPEMQLPKFSEPFYTFTVSEDVPVGTEIDLIRAEHSGTVLYSLVKGNTPESNRDESFVIDRQSGRLKLEKSLDHETTKWYQFSILARCTQDDHEMVASVDVSIQVKDANDNSPVFESSPYEAFIVENLPGGSRVIQIRASDADSGTNGQVMYSLDQSQSVEVIESFAINMETGWITTLKELDHEKRDNYQIKVVASDHGEKIQLSSTAIVDVTVTDVNDSPPRFTAEIYKGTVSEDDPQGGVIAILSTTDADSEEINRQVTYFITGGDPLGQFAVETIQNEWKVYVKKPLDREKRDNYLLTITATDGTFSSKAIVEVKVLDANDNSPVCEKTLYSDTIPEDVLPGKLIMQISATDADIRSNAEITYTLLGSGAEKFKLNPDTGELKTSTPLDREEQAVYHLLVRATDGGGRFCQASIVVTLEDVNDNAPEFSADPYAITVFENTEPGTLLTRVQATDADAGLNRKILYSLIDSADGQFSINELSGIIQLEKPLDRELQAVYTLSLKAVDQGLPRRLTATGTVIVSVLDINDNPPVFEYREYGATVSEDILVGTEVLQVYAASRDIEANAEITYSIISGNEHGKFSIDSKTGAVFIIENLDYESSHEYYLTVEATDGGTPSLSDVATVNVNVTDINDNTPVFSQDTYTTVISEDAVLEQSVITVMADDADGPSNSHIHYSIIDGNQGSSFTIDPVRGEVKVTKLLDRETISGYTLTVQASDNGSPPRVNTTTVNIDVSDVNDNAPVFSRGNYSVIIQENKPVGFSVLQLVVTDEDSSHNGPPFFFTIVTGNDEKAFEVNPQGVLLTSSAIKRKEKDHYLLQVKVADNGKPQLSSLTYIDIRVIEESIYPPAILPLEIFITSSGEEYSGGVIGKIHATDQDVYDTLTYSLDPQMDNLFSVSSTGGKLIAHKKLDIGQYLLNVSVTDGKFTTVADITVHIRQVTQEMLNHTIAIRFANLTPEEFVGDYWRNFQRALRNILGVRRNDIQIVSLQSSEPHPHLDVLLFVEKPGSAQISTKQLLHKINSSVTDIEEIIGVRILNVFQKLCAGLDCPWKFCDEKVSVDESVMSTHSTARLSFVTPRHHRAAVCLCKEGRCPPVHHGCEDDPCPEGSECVSDPWEEKHTCVCPSGRFGQCPGSSSMTLTGNSYVKYRLTENENKLEMKLTMRLRTYSTHAVVMYARGTDYSILEIHHGRLQYKFDCGSGPGIVSVQSIQVNDGQWHAVALEVNGNYARLVLDQVHTASGTAPGTLKTLNLDNYVFFGGHIRQQGTRHGRSPQVGNGFRGCMDSIYLNGQELPLNSKPRSYAHIEESVDVSPGCFLTATEDCASNPCQNGGVCNPSPAGGYYCKCSALYIGTHCEISVNPCSSNPCLYGGTCVVDNGGFVCQCRGLYTGQRCQLSPYCKDEPCKNGGTCFDSLDGAVCQCDSGFRGERCQSDIDECSGNPCLHGALCENTHGSYHCNCSHEYRGRHCEDAAPNQYVSTPWNIGLAEGIGIVVFVAGIFLLVVVFVLCRKMISRKKKHQAEPKDKHLGPATAFLQRPYFDSKLNKNIYSDIPPQVPVRPISYTPSIPSDSRNNLDRNSFEGSAIPEHPEFSTFNPESVHGHRKAVAVCSVAPNLPPPPPSNSPSDSDSIQKPSWDFDYDTKVVDLDPCLSKKPLEEKPSQPYSARESLSEVQSLSSFQSESCDDNGYHWDTSDWMPSVPLPDIQEFPNYEVIDEQTPLYSADPNAIDTDYYPGGYDIESDFPPPPEDFPAADELPPLPPEFSNQFESIHPPRDMPAAGSLGSSSRNRQRFNLNQYLPNFYPLDMSEPQTKGTGENSTCREPHAPYPPGYQRHFEAPAVESMPMSVYASTASCSDVSACCEVESEVMMSDYESGDDGHFEEVTIPPLDSQQHTEV

SEQ ID NO: 4 - DNA, 14792 bp, Homo sapiens
  CDS (187)..(13995)
ctgggcggccgggcgcggggagagggcgcgggagcggctcgtgcggcaggtaccatgcggacgcgcgagcccggcgaggccccggcaggcccgtccctgctcgggggcgcgctgagacggcgggtgagctccacgagagcgccgtcgccacttcgggccaactttgcgattcccgacagttaagcaatggggagacatttggctttgctcctgcttctgctccttctcttccaacattttggagacagtgatggcagccaacgacttgaacagactcctctgcagtttacacacctcgagtacaacgtcaccgtgcaggagaactctgcagctaagacttatgtggggcatcctgtcaagatgggtgtttacattacacatccagcgtgggaagtaaggtacaaaattgtttccggagacagtgaaaacctgttcaaagctgaagagtacattctcggagacttttgctttctaagaataaggaccaaaggaggaaatacagctattcttaatagagaagtgaaggatcactacacattgatagtgaaagcacttgaaaaaaatactaatgtggaggcgcgaacaaaggtcagggtgcaggtgctggatacaaatgacttgagaccgttattctcacccacctcatacagcgtttctttacctgaaaacacagctataaggaccagtatcgcaagagtcagcgccacggatgcagacataggaaccaacggggaattttactacagttttaaagatcgaacagatatgtttgctattcacccaaccagtggtgtgatagtgttaactggtagacttgattacctagagaccaagctctatgagatggaaatcctcgctgcggaccgtggcatgaagttgtatgggagcagtggcatcagcagcatggccaagctaacggtgcacatcgaacaggccaatgaatgtgctccggtgataacagcagtgacattgtcaccatcagaactggacagggacccagcatatgcaattgtgacagtggatgactgcgatcagggtgccaatggtgacatagcatctttaagcatcgtggcaggtgaccttctccagcagtttagaacagtgaggtcctttccagggagtaaggagtataaagtcaaagccatcggtgacattgattgggacagtcatcctttcggctacaatctcacactacaggctaaagataaaggaactccgccccagttctcttctgttaaagtcattcacgtgacttctccacagttcaaagccgggccagtcaagtttgaaaaggatgtttacagagcagaaataagtgaatttgctcctcccaacacacctgtggtcatggtaaaggccattcctgcttattcccatttgaggtatgtttttaaaaggacacctggaaaagctaaattcagtttaaattacaacactggtctcatttctattttagaaccagttaaaagacagcaggcagcccattttgaacttgaagtaacaacaagtgacagaaaagcgtccaccaaggtcttggtgaaagtcttaggtgcaaatagcaatccccctgaatttacccagacagcgtacaaagctgcttttgatgagaacgtgcccattggtactactatcatgagcctgagtgccgtagaccctgatgagggtgagaatgggtacgtgacatacagtatcgcaaatttaaatcatgtgccgtttgcgattgaccatttcactggtgccgtgagtacgtcagaaaacctggactacgaactgatgcctcgggtttatactctgaggattcgtgcatcagactggggcttgccgtaccgccgggaagtcgaagtccttgctacaattactctcaataacttgaatgacaacacacctttgtttgagaaaataaattgtgaagggacaattcccagagatctaggcgtgggagagcaaataaccactgtttctgctattgatgcagatgaacttcagttggtacagtatcagattgaagctggaaatgaactggatttgtttagtttaaaccccaactcgggggtattgtcattaaagcgatcgctaatggatggcttaggtgcaaaggtgtctttccacagtctgagaatcacagctacagatggagaaaattttgccacaccattatatatcaacataacagtggctgccagtcacaagctggtaaacttgcagtgtgaagagactggtgttgccaaaatgctggcagagaagctcctgcaggcaaataaattacacaaccagggagaggtggaggatattttcttcgattctcactctgtcaatgctcacataccgcagtttagaagcactcttccgactggtattcaggtaaaggaaaaccagcctgtgggttccagtgtaattttcatgaactccactgaccttgacactggcttcaatggaaaactggtctatgctgtttctggaggaaatgaggatagttgcttcatgattgatatggaaacaggaatgctgaaaattttatctcctcttgaccgtgaaacaacagacaaatacaccctgaatattaccgtctatgaccttgggataccccagaaggctgcgtggcgtcttctacatgtcgtggttgtcgatgccaatgataatccacccgagtttttacaggagagctattttgtggaagtgagtgaagacaaggaggtacatagtgaaatcatccaggttgaagccacagataaagacctggggcccaacggacacgtgacgtactcaattcttacagacacagacacattttcaattgacagcgtgacgggtgttgttaacatcgcacgccctctggatcgagagctgcagcatgagcactccttaaagattgaggccagggaccaagccagagaagagcctcagctgttctccactgtcgttgtgaaagtatcactagaagatgttaatgacaacccacctacatttattccacctaattatcgtgtgaaagtccgagaggatcttccagaaggaaccgtcatcatgtggttagaagcccacgatcctgatttaggtcagtctggtcaggtgagatacagccttctggaccacggagaaggaaacttcgatgtggataaactcagtggagcagttaggatcgtccagcagttggactttgagaagaagcaagtgtataatctcactgtgagggccaaagacaagggaaagccagtttctctgtcttctacttgctatgttgaagttgaggtggttgatgtgaatgagaacctgcacccacccgtgttttccagctttgtggaaaaggggacagtgaaagaagatgcacctgttggttcattggtaatgacggtgtcggctcatgatgaggacgccggaagagatggggagatccgatactccattagagatggctctggcgttggtgttttcaaaataggtgaagagacaggtgtcatagagacgtcagatcgactggaccgtgaatcgacctcccattattggctaacagtctttgcaaccgatcagggtgtcgtgcctctttcatcgttcatagagatctacatagaggttgaggatgtcaatgacaatgcaccacagacatcagagcctgtttattacccagaaatcatggaaaattctcctaaagatgtatctgtggtccagatcgaggcatttgatccagattcgagctctaatgacaagctcatgtacaaaattacaagtggaaatccacaaggattcttttcaatacatcctaaaacaggtctcatcacaactacgtcaaggaagctagaccgagaacagcaagatgaacacatattagaggttactgtgacagacaatggtagtccccccaaatcaaccattgcaagagtcattgtgaaaatccttgatgaaaatgacaacaaacctcagtttctgcaaaagttctacaaaatcagactccctgagcgggaaaagccagaccgagaaagaaatgccagacgggagccgctctatcgcgtcatagccaccgacaaggatgagggccccaatgcagaaatctcctacagcatcgaagacgggaatgagcatggcaaatttttcatcgaaccgaaaactggagtggtttcgtccaagaggttttcagcagctggagaatatgatattctttcaattaaggcagttgacaatggtcgccctcaaaagtcatcaaccaccagactccatattgaatggatctccaagcccaaacagtccctggagcccatttcatttgaagaatcattttttacctttactgtgatggaaagtgaccccgttgctcacatgattggagtaatatctgtggagcctcctggcatacccctttggtttgacatcactggtggcaactacgacagtcacttcgatgtggacaagggaactggaaccatcattgttgccaaacctcttgatgcagaacagaagtcaaactacaacctcacagtcgaggctacagatggaaccaccactatcctcactcaggtattcatcaaagtaatagacacaaatgaccatcgtcctcagttttctacatcaaagtatgaagttgttattcctgaagatacagcgccagaaacagaaattttgcaaatcagtgctgtggatcaggatgagaaaaacaaactaatctacactctgcagagcagtagagatccactgagtctcaagaaatttcgtcttgatcctgcaaccggctctctctatacttctgagaaactggatcatgaagctgtttcaccagcacacctcacggtcatggtacgagatcaagatgtgcctgtaaaacgcaactttgcaaggattgtggtcaatgtcagcgacacgaatgaccacgccccgtggttcaccgcttcctcctacaaagggcgggtttatgaatcggcagccgttggctcagttgtgttgcaggtgacggctctggacaaggacaaagggaaaaatgctgaagtgctgtactcgatcgagtcaggaaatattggaaatattggaaattcttttatgattgatcctgtcttgggctctattaaaactgccaaagaattagatcgaagtaaccaagcggagtatgatttaatggtaaaagctacagataagggcagtccaccaatgagtgaaataacttctgtgcgtatctttgtcacaattgctgacaacgcctctccgaagtttacatcaaaagaatattctgttgaacttagtgaaactgtcagcattgggagtttcgttgggatggttacagcccatagtcaatcatcagtggtgtatgaaataaaagatggaaatacaggtgatgcttttgatattaatccacattctggaactatcatcactcagaaagccctggactttgaaactttgcccatttacacattgataatacaaggaactaacatggctggtttgtccactaatacaacggttctagttcacttgcaggatgagaatgacaacgcgccagtttttatgcaggcagaatatacaggactcattagtgaatcagcctcaattaacagcgtggtcctaacagacaggaatgtcccactggtgattcgagcagctgatgctgataaagactcaaatgctttgcttgtatatcacattgttgaaccatctgtacacacatattttgctattgattctagcactggtgctattcatacagtactaagtctggactatgaagaaacaagtatttttcactttaccgtccaagtgcatgacatgggaaccccacgtttatttgctgagtatgcagcgaatgtaacagtacatgtaattgacattaatgactgcccccctgtgtttgccaagccattatatgaagcatctcttttgttaccaacatacaaaggagtaaaagtcatcacagtaaatgctacagatgctgattcaagtgcattctcacagttgatttactccatcaccgaaggcaacatcggggagaagttttctatggactacaagactggtgctctcactgtccaaaacacaactcagttaagaagccgctacgagctaaccgttagagcttccgatggcagatttgccggccttacctctgtcaaaattaatgtgaaagaaagcaaagaaagtcacctaaagtttacccaggatgtctactctgcggtagtgaaagagaattccaccgaggccgaaacattagctgtcattactgctattgggagtccaatcaatgagcctttgttttatcacatcctcaacccagatcgcagatttaaaataagccgcacttcaggggttctgtcaaccactggcacgcccttcgatcgtgagcagcaggaggcgtttgatgtggttgtagaagtgatagaggaacataagccttctgcagtggcccacgttgtcgtgaaggtcattgtagaagaccaaaatgataatgcgccggtgtttgtcaaccttccctactacgccgttgttaaagtggacactgaggtgggccatgtcattcgctatgtcactgctgtagacagagacagtggcagaaacggggaagtgcattactacctcaaggaacatcatgaacactttcaaattggacccttgggtgaaatttcactgaaaaagcaatttgagcttgacaccttaaataaagaatatcttgttacagtggttgcaaaagatggagggaacccggccttttcagcggaagttatcgttccgatcactgtcatgaataaagccatgcctgtgtttgaaaaacctttctacagtgcagagattgcagagagcatccaggtgcacagccctgtggtccacgtgcaggctaacagcccggaaggcctgaaagtgttctacagcatcacagacggagaccctttcagccagttcactattaacttcaatactggagttatcaatgtcatagctcctctggactttgaggcccacccggcatataagctgagcatacgcgcaactgactccttgacgggcgctcatgctgaagtatttgtggacatcatagtagacgacatcaatgataaccctcctgtgtttgctcagcagtcttatgcggtgaccctgtctgaggcatctgtaattggaacgtctgttgttcaagttagagccaccgattctgattcagaaccaaatagaggaatctcataccagatgtttgggaatcacagcaagagtcatgatcattttcatgtagacagcagcactggcctcatctcactactcagaaccctggattacgagcagtcccggcagcacacgatttttgtgagggcagttgatggtggtatgcccacgctgagcagtgatgtgattgtcacggtggacgttaccgacctcaatggtaatccaccactctttgaacaacagatttatgaagccagaattagcgagcacgcccctcatgggcatttcgtgacctgtgtaaaagcctatgatgcagacagttcagacatagacaagttgcagtattccattctgtctggcaatgatcataaacattttgtcattgacagtgcaacagggattatcaccctctcaaacctgcaccggcacgccctgaagccattttacagtcttaacctgtcagtgtctgatggagtttttagaagttccacccaggttcatgtaactgtaattggaggcaatttgcacagtcctgctttccttcagaacgaatatgaagtggaactagctgaaaacgctcccctacataccctggtgatggaggtgaaaactacggatggggattctggtatttatggtcacgttacttaccatattgtaaatgactttgccaaagacagattttacataaatgagagaggacagatatttactttggaaaaacttgatcgagaaaccccggcggagaaagtgatctcagtccgtttaatggctaaggatgctggaggaaaagttgctttctgcaccgtgaatgtcatccttacagatgacaatgacaatgcaccacaatttcgagcaaccaaatacgaagtgaatatcgggtccagtgctgctaaagggacttcagtcgtaaagtctgcaagtgatgccgatgagggctccaatgccgacatcacctatgccattgaagcagactctgaaagtgtaaaagagaatttggaaattaacaaactgtccggcgtaatcactacaaaggagagcctcattggcttggaaaatgaattcttcactttctttgttagagctgtggataatgggtctccatcaaaagaatctgttgttcttgtctatgttaaaatccttccaccggaaatgcagcttccaaaattttcagaacctttctatacctttacagtgtcagaggacgtgcctgttggaacagagatagatctcatccgagcagaacatagtgggactgttctttacagcctggtcaaagggaatactccagaaagcaatagggatgagtcctttgtgattgacagacagagcgggagactgaagttggagaagagtcttgatcatgagacaactaagtggtatcagttttccatactggccaggtgcactcaagatgaccatgagatggtggcttctgtagatgttagtatccaagtgaaagatgcaaatgacaacagcccggtctttgaatctagtccatatgaggcattcattgttgaaaacctgccagggggaagtagagtaattcagatcagggcatctgatgctgactcaggaaccaacggccaagttatgtatagcctggatcagtcacaaagtgtggaagtcattgaatcctttgccattaacatggaaacaggctggattacaactttaaaggaacttgaccatgaaaagagagacaattaccagattaaagtggttgcatcagatcatggtgaaaagatccagctatcctccacagccattgtggatgttaccgtcaccgatgtcaacgatagtccaccacgattcacggccgagatctataaagggactgtgagtgaggatgacccccaaggtggggtgattgccatcttaagtaccacggatgctgattctgaagagatcaacagacaagttacatatttcataacaggaggggatcctttaggacagtttgccgttgaaactatacagaatgaatggaaggtatatgtgaagaaacctctagacagggaaaaaagggacaattaccttcttactatcacggcaactgatggcaccttctcatcaaaagcgatagttgaagtgaaagttctggatgcaaatgacaacagtccagtttgtgaaaagactttatattcagacactattcctgaagacgtccttcctggaaaattgatcatgcagatctctgctacagacgcagacatccgctctaacgctgaaattacttacacgttattgggttcaggtgcagaaaaattcaaactaaatccagacacaggtgaactgaaaacgtcaaccccccttgatcgtgaggagcaagctgtttatcatcttctcgtcagggccacagatggaggaggaagattctgccaagccagtattgtcgtcacgctagaagatgtgaacgataacgcccccgaattctctgccgatccttatgccatcaccgtgtttgaaaacacagagccgggaacgctgctgacaagagtgcaggccacagatgccgacgcaggattaaatcggaagattttatactcactgattgactctgctgatgggcagttctccattaacgaattatctggaattattcagttagaaaaacctttggacagagaactccaggcagtatacaccctctctttgaaagctgtggatcaaggcttgccaaggaggctgactgccactggcactgtgattgtatcagttcttgacataaatgacaacccccctgtgtttgagtaccgtgaatatggtgccaccgtgtctgaggacattcttgttggaactgaagttcttcaagtgtatgcagcaagtcgggatattgaagcaaatgcagaaatcacctactcaataataagtggaaatgaacatgggaaattcagcatagattctaaaacaggggccgtatttatcattgagaatctggattatgagagctctcatgagtattacctaacagtagaggccactgatggaggcacgccttcactgagcgacgttgccactgtgaacgttaatgtaacagatatcaacgataatacccctgtgttcagccaagacacctacacgacagtcatcagtgaagatgccgttcttgagcagtctgtcatcacggttatggccgatgatgccgatggaccttccaacagccacatccactactcaattatagatggcaaccaaggaagctcgttcacaattgaccccgtcaggggagaagtcaaagtgaccaaacttctcgaccgagaaacgatttcaggttacacgctcacggttcaagcttctgataatggcagtccacccagagtcaacacgacgaccgtgaacatcgatgtgtccgatgtcaatgacaacgcgcccgtcttctccaggggaaactacagtgtcattatccaggaaaataagccagtgggcttcagcgtgctgcagctggtagtaacagatgaggattcttcccataacggtccacccttcttctttactattgtaactggaaatgatgagaaggcttttgaagttaacccgcaaggagtcctcctgacatcatctgccatcaagaggaaggagaaagatcattacttactgcaggtgaaggtggcagataatggaaagcctcagttgtcatctttgacatacattgacattagggtaattgaggagagcatctatccgcctgcgattttgcccctggagattttcatcacctcttctggagaagaatactcaggtggcgtcattgggaagatccatgccacagaccaggacgtgtatgatactctaacctacagtctcgaccctcagatggacaacctgttctctgtttccagcacagggggcaagctgatagcacacaaaaagctagacatagggcaataccttctcaatgtcagcgtaacagatgggaagttcacgacggtggccgacatcacagtgcatatcagacaagtcacacaggagatgttgaaccacaccatcgcgatccgctttgccaacctcactccggaagaattcgttggtgactactggcgcaacttccagcgagctttacggaacatcctgggtgtgaggaggaacgacatacagattgttagtttgcagtcctctgaacctcacccacatctggacgtcttactttttgtagagaaaccaggtagtgctcagatctcaacaaaacaacttctgcacaagattaactcttccgtgactgacattgaggaaatcattggagttaggatactgaatgtattccagaaactctgcgcgggactggactgcccctggaagttctgcgatgaaaaggtgtctgtggatgaaagtgtgatgtcaacacacagcacagccagactgagttttgtgactccccgccaccacagggcagcggtgtgtctctgcaaagagggaaggtgcccacctgtccaccatggctgtgaagatgatccgtgccctgagggatccgaatgtgtgtctgatccctgggaggagaaacacacctgtgtctgtcccagcggcaggtttggtcagtgcccagggagttcatctatgacactgactggaaacagctacgtgaaataccgtctgacggaaaatgaaaacaaattagagatgaaactgaccatgaggctcagaacatattccacgcatgcggttgtcatgtatgctcgaggaactgactatagcatcttggagattcatcatggaaggctgcagtacaagtttgactgtggaagtggccctggaattgtctctgttcagagcattcaggtcaatgatgggcagtggcacgcagtggccctggaagtgaatggaaactatgctcgcttggttctagaccaagttcatactgcatcgggcacagccccagggactctgaaaaccctgaacctggataactatgtgttttttggtggccacatccgtcagcagggaacaaggcatggaagaagtcctcaagttggtaatggtttcaggggttgtatggactccatttatttgaatgggcaggagctccctttaaacagcaaacccagaagctatgcacacatcgaagagtcggtggatgtatctccaggctgcttcctgacggccacggaagactgcgccagcaacccttgccagaatggaggcgtttgcaatccgtcacctgctggaggttattactgcaaatgcagtgccttgtacatagggacccactgtgagataagcgtcaatccgtgttcctccaacccatgcctctatgggggcacgtgtgttgtcgacaacggaggctttgtttgccagtgtagaggattatatactggtcagaggtgtcagcttagtccatactgcaaagatgaaccctgtaagaatggcggaacatgctttgacagtttggatggcgccgtttgtcagtgtgattcgggttttaggggagaaaggtgtcagagtgatatcgacgagtgctctggaaacccttgcctgcacggggccctctgtgagaacacgcacggctcctatcactgcaactgcagccacgagtacaggggacgtcactgcgaggatgctgcgcccaaccagtatgtgtccacgccgtggaacattgggttggcggaaggaattggaatcgttgtgtttgttgcagggatatttttactggtggtggtgtttgttctctgccgtaagatgattagtcggaaaaagaagcatcaggctgaacctaaagacaagcacctgggacccgctacggctttcttgcaaagaccgtattttgattccaagctaaataagaacatttactcagacataccaccccaggtgcctgtccggcctatttcctacaccccgagtattccaagtgactcaagaaacaatctggaccgaaattccttcgaaggatctgctatcccagagcatcccgaattcagcacttttaaccccgagtctgtgcacgggcaccgaaaagcagtggcggtctgcagcgtggcgccaaacctgcctcccccacccccttcaaactccccttctgacagcgactccatccagaagcctagctgggactttgactatgacacaaaagtggtggatcttgatccctgtctttccaagaagcctctagaggaaaagccttcccagccatacagtgcccgggaaagcctgtctgaagtgcagtccctgagctccttccagtccgaatcgtgcgatgacaatgaatctttggctgctcctgacctcagcaaaccaagagggtatcactgggatacatcagattggatgccaagcgttcctctgccggacatacaagagttccccaactatgaggtgattgatgagcagacacccctgtactcagcagatccaaacgccatcgatacggactattaccctggaggctacgacatcgaaagtgattttcctccacccccagaagacttccccgcagctgatgagctaccaccgttaccgcccgaattcagcaatcagtttgaatccatccaccctcctagagacatgcctgccgcgggtagcttgggttcttcatcaagaaaccggcagaggttcaacttgaatcagtatttgcccaatttttatcccctcgatatgtctgaacctcaaacaaaaggcactggtgagaatagtacttgtagagaaccccatgccccttacccgccagggtatcaaagacacttcgaggcgcccgctgtcgagagcatgcccatgtctgtgtacgcctccaccgcctcctgctctgacgtgtcagcctgctgcgaagtggagtccgaggtcatgatgagtgactatgagagcggggacgacggccacttcgaagaggtgacgatcccgcccctggattcccagcagcacacggaagtctgactctcaactccccccaaagtgcctgactttagtgaacctagaggtgatgtgagtaatccgcgctgttctttgcagcagtgcttccaagctttttttggtgagccgaatgggcatggctgcgctggatcctgcgcctctggacgtgctagccatttccagtgtcccaactactgtcatcgtgaggttttcatcggctgtgccatttcccaacgtcttttgggatttacatctgtctgtgttaaaataatcaaacgaaaaatcagtcctgtgttgtcagcatgattcatgtatttatatagatttgattattttaattttcctgtctcttttttttgtaaattttatgtacagatttgatttttcatagttttaactagatttccaagatattttgtgcatttgtttcaactgaattttggtggtgtcagtgccattatctagcaccctgatttttttttttttactataaccagggtttcattctgtctttttccactgaagtgtgacattttgttagtacatttcagtgtagtcattcatttctagctgtacataggatgaaggagagatcagatacatgaacatgtcttacatgggttgctgtatttagaattataaacatttttcattattggaaagtgtaacggggaccttctgcatacctgtttagaaccaaaaccaccatgacacagtttttatagtgtctgtatatttgtgatgcaatggtcttgtaaaggtttttaatgaaaactaccattagccagtctttcttactgacaataaattattaataaaat

SEQ ID NO: 5 - protein, 4602 aa, Homo sapiens
MGRHLALLLLLLLLFQHFGDSDGSQRLEQTPLQFTHLEYNVTVQENSAAKTYVGHPVKMGVYITHPAWEVRYKIVSGDSENLFKAEEYILGDFCFLRIRTKGGNTAILNREVKDHYTLIVKALEKNTNVEARTKVRVQVLDTNDLRPLFSPTSYSVSLPENTAIRTSIARVSATDADIGTNGEFYYSFKDRTDMFAIHPTSGVIVLTGRLDYLETKLYEMEILAADRGMKLYGSSGISSMAKLTVHIEQANECAPVITAVTLSPSELDRDPAYAIVTVDDCDQGANGDIASLSIVAGDLLQQFRTVRSFPGSKEYKVKAIGDIDWDSHPFGYNLTLQAKDKGTPPQFSSVKVIHVTSPQFKAGPVKFEKDVYRAEISEFAPPNTPVVMVKAIPAYSHLRYVFKRTPGKAKFSLNYNTGLISILEPVKRQQAAHFELEVTTSDRKASTKVLVKVLGANSNPPEFTQTAYKAAFDENVPIGTTIMSLSAVDPDEGENGYVTYSIANLNHVPFAIDHFTGAVSTSENLDYELMPRVYTLRIRASDWGLPYRREVEVLATITLNNLNDNTPLFEKINCEGTIPRDLGVGEQITTVSAIDADELQLVQYQIEAGNELDLFSLNPNSGVLSLKRSLMDGLGAKVSFHSLRITATDGENFATPLYINITVAASHKLVNLQCEETGVAKMLAEKLLQANKLHNQGEVEDIFFDSHSVNAHIPQFRSTLPTGIQVKENQPVGSSVIFMNSTDLDTGFNGKLVYAVSGGNEDSCFMIDMETGMLKILSPLDRETTDKYTLNITVYDLGIPQKAAWRLLHVVVVDANDNPPEFLQESYFVEVSEDKEVHSEIIQVEATDKDLGPNGHVTYSILTDTDTFSIDSVTGVVNIARPLDRELQHEHSLKIEARDQAREEPQLFSTVVVKVSLEDVNDNPPTFIPPNYRVKVREDLPEGTVIMWLEAHDPDLGQSGQVRYSLLDHGEGNFDVDKLSGAVRIVQQLDFEKKQVYNLTVRAKDKGKPVSLSSTCYVEVEVVDVNENLHPPVFSSFVEKGTVKEDAPVGSLVMTVSAHDEDAGRDGEIRYSIRDGSGVGVFKIGEETGVIETSDRLDRESTSHYWLTVFATDQGVVPLSSFIEIYIEVEDVNDNAPQTSEPVYYPEIMENSPKDVSVVQIEAFDPDSSSNDKLMYKITSGNPQGFFSIHPKTGLITTTSRKLDREQQDEHILEVTVTDNGSPPKSTIARVIVKILDENDNKPQFLQKFYKIRLPEREKPDRERNARREPLYRVIATDKDEGPNAEISYSIEDGNEHGKFFIEPKTGVVSSKRFSAAGEYDILSIKAVDNGRPQKSSTTRLHIEWISKPKQSLEPISFEESFFTFTVMESDPVAHMIGVISVEPPGIPLWFDITGGNYDSHFDVDKGTGTIIVAKPLDAEQKSNYNLTVEATDGTTTILTQVFIKVIDTNDHRPQFSTSKYEVVIPEDTAPETEILQISAVDQDEKNKLIYTLQSSRDPLSLKKFRLDPATGSLYTSEKLDHEAVSPAHLTVMVRDQDVPVKRNFARIVVNVSDTNDHAPWFTASSYKGRVYESAAVGSVVLQVTALDKDKGKNAEVLYSIESGNIGNIGNSFMIDPVLGSIKTAKELDRSNQAEYDLMVKATDKGSPPMSEITSVRIFVTIADNASPKFTSKEYSVELSETVSIGSFVGMVTAHSQSSVVYEIKDGNTGDAFDINPHSGTIITQKALDFETLPIYTLIIQGTNMAGLSTNTTVLVHLQDENDNAPVFMQAEYTGLISESASINSVVLTDRNVPLVIRAADADKDSNALLVYHIVEPSVHTYFAIDSSTGAIHTVLSLDYEETSIFHFTVQVHDMGTPRLFAEYAANVTVHVIDINDCPPVFAKPLYEASLLLPTYKGVKVITVNATDADSSAFSQLIYSITEGNIGEKFSMDYKTGALTVQNTTQLRSRYELTVRASDGRFAGLTSVKINVKESKESHLKFTQDVYSAVVKENSTEAETLAVITAIGSPINEPLFYHILNPDRRFKISRTSGVLSTTGTPFDREQQEAFDVVVEVIEEHKPSAVAHVVVKVIVEDQNDNAPVFVNLPYYAVVKVDTEVGHVIRYVTAVDRDSGRNGEVHYYLKEHHEHFQIGPLGEISLKKQFELDTLNKEYLVTVVAKDGGNPAFSAEVIVPITVMNKAMPVFEKPFYSAEIAESIQVHSPVVHVQANSPEGLKVFYSITDGDPFSQFTINFNTGVINVIAPLDFEAHPAYKLSIRATDSLTGAHAEVFVDIIVDDINDNPPVFAQQSYAVTLSEASVIGTSVVQVRATDSDSEPNRGISYQMFGNHSKSHDHFHVDSSTGLISLLRTLDYEQSRQHTIFVRAVDGGMPTLSSDVIVTVDVTDLNGNPPLFEQQIYEARISEHAPHGHFVTCVKAYDADSSDIDKLQYSILSGNDHKHFVIDSATGIITLSNLHRHALKPFYSLNLSVSDGVFRSSTQVHVTVIGGNLHSPAFLQNEYEVELAENAPLHTLVMEVKTTDGDSGIYGHVTYHIVNDFAKDRFYINERGQIFTLEKLDRETPAEKVISVRLMAKDAGGKVAFCTVNVILTDDNDNAPQFRATKYEVNIGSSAAKGTSVVKSASDADEGSNADITYAIEADSESVKENLEINKLSGVITTKESLIGLENEFFTFFVRAVDNGSPSKESVVLVYVKILPPEMQLPKFSEPFYTFTVSEDVPVGTEIDLIRAEHSGTVLYSLVKGNTPESNRDESFVIDRQSGRLKLEKSLDHETTKWYQFSILARCTQDDHEMVASVDVSIQVKDANDNSPVFESSPYEAFIVENLPGGSRVIQIRASDADSGTNGQVMYSLDQSQSVEVIESFAINMETGWITTLKELDHEKRDNYQIKVVASDHGEKIQLSSTAIVDVTVTDVNDSPPRFTAEIYKGTVSEDDPQGGVIAILSTTDADSEEINRQVTYFITGGDPLGQFAVETIQNEWKVYVKKPLDREKRDNYLLTITATDGTFSSKAIVEVKVLDANDNSPVCEKTLYSDTIPEDVLPGKLIMQISATDADIRSNAEITYTLLGSGAEKFKLNPDTGELKTSTPLDREEQAVYHLLVRATDGGGRFCQASIVVTLEDVNDNAPEFSADPYAITVFENTEPGTLLTRVQATDADAGLNRKILYSLIDSADGQFSINELSGIIQLEKPLDRELQAVYTLSLKAVDQGLPRRLTATGTVIVSVLDINDNPPVFEYREYGATVSEDILVGTEVLQVYAASRDIEANAEITYSIISGNEHGKFSIDSKTGAVFIIENLDYESSHEYYLTVEATDGGTPSLSDVATVNVNVTDINDNTPVFSQDTYTTVISEDAVLEQSVITVMADDADGPSNSHIHYSIIDGNQGSSFTIDPVRGEVKVTKLLDRETISGYTLTVQASDNGSPPRVNTTTVNIDVSDVNDNAPVFSRGNYSVIIQENKPVGFSVLQLVVTDEDSSHNGPPFFFTIVTGNDEKAFEVNPQGVLLTSSAIKRKEKDHYLLQVKVADNGKPQLSSLTYIDIRVIEESIYPPAILPLEIFITSSGEEYSGGVIGKIHATDQDVYDTLTYSLDPQMDNLFSVSSTGGKLIAHKKLDIGQYLLNVSVTDGKFTTVADITVHIRQVTQEMLNHTIAIRFANLTPEEFVGDYWRNFQRALRNILGVRRNDIQIVSLQSSEPHPHLDVLLFVEKPGSAQISTKQLLHKINSSVTDIEEIIGVRILNVFQKLCAGLDCPWKFCDEKVSVDESVMSTHSTARLSFVTPRHHRAAVCLCKEGRCPPVHHGCEDDPCPEGSECVSDPWEEKHTCVCPSGRFGQCPGSSSMTLTGNSYVKYRLTENENKLEMKLTMRLRTYSTHAVVMYARGTDYSILEIHHGRLQYKFDCGSGPGIVSVQSIQVNDGQWHAVALEVNGNYARLVLDQVHTASGTAPGTLKTLNLDNYVFFGGHIRQQGTRHGRSPQVGNGFRGCMDSIYLNGQELPLNSKPRSYAHIEESVDVSPGCFLTATEDCASNPCQNGGVCNPSPAGGYYCKCSALYIGTHCEISVNPCSSNPCLYGGTCVVDNGGFVCQCRGLYTGQRCQLSPYCKDEPCKNGGTCFDSLDGAVCQCDSGFRGERCQSDIDECSGNPCLHGALCENTHGSYHCNCSHEYRGRHCEDAAPNQYVSTPWNIGLAEGIGIVVFVAGIFLLVVVFVLCRKMISRKKKHQAEPKDKHLGPATAFLQRPYFDSKLNKNIYSDIPPQVPVRPISYTPSIPSDSRNNLDRNSFEGSAIPEHPEFSTFNPESVHGHRKAVAVCSVAPNLPPPPPSNSPSDSDSIQKPSWDFDYDTKVVDLDPCLSKKPLEEKPSQPYSARESLSEVQSLSSFQSESCDDNESLAAPDLSKPRGYHWDTSDWMPSVPLPDIQEFPNYEVIDEQTPLYSADPNAIDTDYYPGGYDIESDFPPPPEDFPAADELPPLPPEFSNQFESIHPPRDMPAAGSLGSSSRNRQRFNLNQYLPNFYPLDMSEPQTKGTGENSTCREPHAPYPPGYQRHFEAPAVESMPMSVYASTASCSDVSACCEVESEVMMSDYESGDDGHFEEVTIPPLDSQQHTEV

SEQ ID NO: 6 - DNA, 16781 bp, Homo sapiens
gaaaataagccagtgggcttcagcgtgctgcagctggtagtaacagatgaggattcttcccataacggtccacccttcttctttactattgtaactggaaatgatgagaaggcttttgaagttaacccgcaaggagtcctcctgacatcatctgccatcaagaggaaggagaaagatcattacttactgcaggtgaaggtatgaatggctttctttgtggtttggttgtggattcatagagaagagctgcttggcgtggttcccctcttctatttgaacagcttacatttcatacaaaaacaaactttcatagaattgctgtctttgttttttgccataccttccatttttatcccgctgtgtttgaaggagctcattgttgattctgtcatagtcattgacagagctgggtttcagtgtgaattagtgaagtgctataaagaaatttgtccttttctttgatacgtttcttgtgtggagaaatgagtatctaccagttagtaattttttgggaacttgagtaatacaaatgtttttattgaagaagttataaacagtgccatagacaaaggtgattttatttttcttttcttgtaggtggcagataatggaaagcctcagttgtcatctttgacatacattgacattagggtaattgaggagagcatctatccgcctgcgattttgcccctggagattttcatcacctcttctggagaagaatactcaggtggcgtcattgggaagatccatgccacagaccaggacgtgtatgatactctaacctacagtctcgaccctcagatggacaacctgttctctgtttccagcacagggggcaagctgatagcacacaaaaagctagacatagggcaataccttctcaatgtcagcgtaacagatgggaagttcacgacggtggccgacatcacagtgcatatcagacaagtcacacaggagatgttgaaccacaccatcgcgatccgctttgccaacctcactccggaagaattcgttggtgactactggcgcaacttccagcgagctttacggaacatcctgggtgtgaggaggaacgacatacagattgttagtttgcagtcctctgaacctcacccacatctggacgtcttactttttgtagagaaaccaggtagtgctcagatctcaacaaaacaacttctgcacaagattaactcttccgtgactgacattgaggaaatcattggagttaggatactgaatgtattccagaaactctgcgcgggactggactgcccctggaagttctgcgatgaaaaggtgtctgtggatgaaagtgtgatgtcaacacacagcacagccagactgagttttgtgactccccgccaccacagggcagcggtgtgtctctgcaaaggtactgcacatgagtgtatgaaagtcggtgtcacagatggtttcagacggcctttgaagcctttgatggctatacatgaaaggtgatgttcttgaaatgtctgttaattatcttttccctttctcccctctttgaatgaagagggaaggtgcccacctgtccaccatggctgtgaagatgatccgtgccctgagggatccgaatgtgtgtctgatccctgggaggagaaacacacctgtgtctgtcccagcggcaggtttggtcagtgcccaggtgagagttgaatggttgggtatacttttatttttaaaaatcggtttcattgtttcttaccattttcttctttattggagtctaaaaatattgcaaagtatgtttattgtaagaagacagatgtggagatgaataaagaatgacagtagtaagttttgcttcctcttgttacagcacaccccttggttcatagttggtgtccctcctcctgtgtcttcaatgattatatagacatacagctaacaacgcagtgccattatgtagcagtttaattttcctggaaattcaagagtgatcatatctcacacagttctctccctgttgccttttccctccacttagcagtatggtaaagagcacctttcgtatggttccgagccagtacctttaaacctgattctttttgatagctggataatattttctagcatggctgtacttattcaacattgttcttatgtgtaaagattcgagttgttctctttctttcagataaagaaatctttatacatgtatctttacatactgatgcgttcatttctatacggtgtatagattataaaactagaatttgctatctcaaacatcaatgacattttaaatttttttgtatcactatattagttttccaaaaagtcgtattaatttttatttctcccagttatgtatgaaggtttcactctccattcttttgacatcattgaacattttcgattataaaacaaattccccaccatctattttatgggtgtgaagtgatatctaattattattttaatttgcatttctctggctactttaaaagttttcatctcatttgttcatttcctatcagtattgcctctgctgtggcctctacctgttcatgttgtttgatggtctgggttatttgtttcttagtatcaatgtgtagagttacttgtgtattaggagtattaacattcacagtgttgcagttacctctgtctttatatcatttatattgatgttatcttttgatttgttaagtatgcctcttgtcattttgaaattcaattgttatttagtcagttctacataattttatgtaacagttgccatctgtggagattgatgacatttgcatcttctttggggaatggggattgttttattttgcttgagaaggtttcttaaatcaccttgaggctatgtataactattttctgaaattttcttctaataattttatactgaacatttttatatttaatctttcattactttagaattcatttctctgtgatgcagtcgtcaaatttattctttccaaacagagagccagtcatgctgggcttttcattaagcaaatagacgttttcccacagaattggatagtaaaatgttatcatcttaaataaaaccctaaatcagtctcaaggggtgcaggggagtcatactttaaaaaatatgcttaaacttggttctggtttattttttcctctgtgtgattcacagggagttcatctatgacactgactggaaacagctacgtgaaataccgtctgacggaaaatgaaaacaaattagagatgaaactgaccatgaggctcagaacatattccacgcatgcggttgtcatgtatgctcgaggaactgactatagcatcttggaggtatgtttctccacagcgttgcatcttagtggaagactttcaaaccataaatttcattttatattcagattgttaatttgcacagataataaagtaagtgaaatggttgaattccacatttaaataattcattgatgcctgtacaagttttagactttgtgggatgaagagctctttttgccctgccatgacagagatttaggtccatgtgacagttaacattcaaactctaggtgatgccccattttaataatgttggctgggtgtggtggctcacgcctgtaatcccagcactttgggaagctgaggcgggtggatcacttgaggtcaggaatttgagaccagcctggccagcatggcaaaacctgctctctactaaaaatacaaaaaattagccagacttggtggcggacgcctgtaattccagctactcaggaggctgaggcacgagaatcacttgaacaggggaggcagaggttgcagtgagttgagatcacgccactgcactccagcctgggcgacaagagtgaaactgtctcaaaaaacaaaaactgttacagtttatagagacatcctacatgtgtttcaagtgttatgtatcttgcccaccttttttaaaaacattattcagttttgacttttgatattacctaatgcttatgtgttcagttaatagttaattattcgcttccatgatgctttgtggatgtgcattttaacttgatattcctaaaagacgcattttaatgatttatacagtggacaaatgaatctgtgctctgtagtacttaaatgtaaggcctaacttctaccctcactccctaagccctctcctgtagatttgtgatcaggtgctctcattgaaggttctatgagagtaatgtgaacagtcattgcttttatcctagattcatcatggaaggctgcagtacaagtttgactgtggaagtggccctggaattgtctctgttcagagcattcaggtcaatgatgggcagtggcacgcagtggccctggaagtgaatggaaactatgctcgcttggttctagaccaagttcatactgcatcgggcacagccccagggactctgaaaaccctgaacctggataactatgtgttttttggtggccacatccgtcagcagggaacaaggcatggaagaagtcctcaagttggtaatggtttcaggggttgtatggactccatttatttgaatgggcaggagctccctttaaacagcaaacccagaagctatgcacacatcgaagagtcggtggatgtatctccaggctgcttcctgacggccacggaagactgcgccagcaacccttgccagaatggaggcgtttgcaatccgtcacctgctggaggtgggctccgtaatgttgccatctttaaaatgctgcacgtgtgtacatttaaggggaagctccccaggtatttttttatttttttctcttttgattcagataattttgtcagacactgcagtcagtgttggaagcgagcagcatccctcatacgggggcagagctgggtctgctgcccttctggtgcccaggctgcaggtaccacggctcccactgctgagcgcctgcagtgccctcccagtgcccgagcaggaggcacctcaagtgactcagaactcttctttaactccatagtcatcggcccaggggaatttagaattcgtaccgcttagcccttctttgttatttgtatcaccttttatctaatggctcagcgtagggggcctgctagattcttgctggcaataatagttgggaatatgattttccagaaggccctgaatccctcctgaagtgttgccatatctgtctgtcttgaagccctggcgtagtggggtactttgtgtcttttgcaccctggactcctgttaggcccacttaccaataatagggtacagttaggaaaatgacagttgtcataccaaatagagcttggtataatgaacagagcaaaaataatactgtcattaagtgggatttccaaagctttggttttctgctgttctctcagggaggtatatttgcattctgtgtgcgtagagtgggccaggccatccccacagaaacactctcatttatactgtgctcatattagttacacgtccaaaccaggttagcaaggaggccgtcctcattttgtcacttagggatgaggctgagagaggtgctttcctgacatgcactcccatggtcccgaagcaggagtaaaggagtatggcagctgtgccctggctcctaaaggccctggcccagggcaccacagttcttcatcctgagaaaaacccacagccacgccagctttggaggggcggggaagcactgtgctgcggtgtgccctcaaggcaggcagttagaagatctgccacaccacctgtcacttgacaaagtggagtcgacattcttcgtactgctttgaacagatgtgatgatctcctttcttaaatctttacatgtttgagaggctgaggcaggaggatcacttgaggctagaagtgaggcagcctgggcgttacagggaaaccccatctctacaaaaacttaaaaaatcagccaggcatgtggcatgcctctagttccagctactcaggaggctgaggtgggaggatccctgagcccgggagtttgaggatgcaatgagctgagattgcgccactgcactccagcctgggtggcagagcgagaccctgtctcaaaaaataaaaatattgaaaggtaatttatagtcttcaaagtgcttttcacattcattattttctcatttaatccttaaatgcttgtgaattgcaaataaattgagatgttaagtggtgctgtctacgtacatggtcactcaggtaaagaactgtattttcctcctctatatgtgtgacctagtgatcttaatgcagattatgatgaactaaagctaactgagttctctcttcaattctgggtgttttggttataccagcagtggaaggcttgttgatgtagccaacttttttcccagagagagtgagaattttacgagcttttacagacctcggagaataaagataaaaagcaatataattaagaccaggtttctgagtcattgagtgataggacttttttgttcctttttgccgatttaggttattactgcaaatgcagtgccttgtacatagggacccactgtgagataagcgtcaatccgtgttcctccaagccatgcctctatgggggcacgtgtgttgtcgacaacggaggctttgtttgccagtgtagaggattatatactggtcagaggtatgtgtatttttcttaactttcataatcaattgtagtataaaattcttaaacatataatggtacatgtaaaaagtctgcacgttaatttgctaatgtgttctgttttgctttctgttttgctgtatttatttgcaataggatcatgaggtttatgtctgtttctccttactcttcaggtgtcagcttagtccatactgcaaagatgaaccctgtaagaatggcggaacatgctttgacagtttggatggcgccgtttgtcagtgtgattcgggttttaggggagaaaggtaaatggtttctttcaaaatttaggttcataccttttcatagcgtcatattgactggcagatgcaacagaccttcagtgatttgaactacattatgtcagattttgataatcctcatattggccccttatataatttataatcttttcactctttaaaatgaattatcaagcagatgaatacagtatatataatacttgaaaaaaactaatgtttagccaatggggttgatgtaatagtctgttgatcctaggaaaatggccattaatattggcttgatatgtttccttcccaaatatacatcaaaagcatgcgatcacatgattggtttctccacagaaggtcttttctcgtggttgtcgacgttacattttttagttgtcttttatctgggtttgatcttcacatagctcatcaaggagggattagtcttcagtctctaaggattgagcctgacatacgctggcgtatgctacagaggtctctcctcattcctgacttccttggcaggtgtcagagtgatatcgacgagtgctctggaaacccttgcctgcacggggccctctgtgagaacacgcacggctcctatcactgcaactgcagccacgagtacaggggacgtcactgcgaggatgctgcgcccaaccagtatgtgtccacgccgtggaacattgggttggcggaaggaattggaatcgttgtgtttgttgcagggatatttttactggtggtggtgtttgttctctgccgtaagatgattagtcggaaaaagaagcatcaggctgaacctaaagacaagcacctgggacccgctacggctttcttgcaaagaccgtattttgattccaagctaaataagaacatttactcagacataccaccccaggtgcctgtccggcctatttcctacaccccgagtattccaagtgactcaagaaacaatctggaccgaaattccttcgaaggatctgctatcccagagcatcccgaattcagcacttttaaccccgagtctgtgcacgggcaccgaaaagcagtggcggtctgcagcgtggcgccaaacctgcctcccccacccccttcaaactccccttctgacagcgactccatccagaagcctagctgggactttgactatgacagtaagagccactttttcatctcactaaagtgtcacttgagataaacagtgaggttacaaattctgtccagtcaaaatgtgtgtactcttcattagagacaagtatgctcagactgaaatccgtatgatgatttgaaacttttttggatgtcccgattgctgggttagcattgtggctttgatgttattttaattaagaaattgaatctagtcatgtttagttaatattctggagagatcatttcattttataattgataaaggttctcatctataattttttgagtttattaatatgtcaaaaaaataatcgggagcattttcatgctaggatttttaaaaatatgacctggcgaagtggaaatatttatattagtgacctaacatatatttgatgcttttttcttttaaagctgtacatactattttaaaacaaaattttagcagcctttactgtctaatgatacccataggcatgcttatgggcgatttctactctatgaataatctctaagatgatgtggaggtctttctgtgcaggggttttttatttcttcgtttgagtttgtaaggtgtttctgtttttctttcagtcattcatttattgagtaaatctcaggccatcgttgggtaatgcatggtgtgtctcaggattcagcggtgaacaaaaccgtcttggcccatatacttactgtttgtcatccttccttttagcaaaagtggtggatcttgatccctgtctttccaagaagcctctagaggaaaagccttcccagccatacagtgcccgggaaagcctgtctgaagtgcagtctctgagctccttccagtccgaatcgtgcgatgacaatggtgaggcagcagtgtgctgcaacactgcgctttgcttgcttgttgtgttactgtgttatctcacaatgtagacgcactatctacccttaaagcctcaccatattttcatttttcttggaccacttttaaacaataccatacatacttgcatgaatgtctgctggggaaaatagccaaactgttcttaatgaataggtgtttgaacttgtcagtaatagagaagaacatattcattgaaggatctgatcgactaatcttatattggctttttttcacaacagttaatatcaccccagttttgtgggtttgtcagaaatcttcctcgcaactttcttaaccacgcagcacgcacacaaacacacactctctctcacacacacacacacacacaccccctctaagaagagatgtgatagtctgaaatgttcgatttcagtagcaccttgggtattacaaggttttatttgtctcctcccccaaccccactgcggccccagctcccatttggatcagagcgtactgccagagcacagtttaagagattgctagtaagtttgattatatttctaatgaagaaagtttaacagtgggatctttgccaccagttatgttgatctggtttccctatagggtaaattgcattgataactagctttgccatattaacacataccagtagagaaaaacgtcaatattcagtaacacaatgtagtttttagtggaaatttttgaatctcagaattagtaagtttaatgataatttttattgtataaacgtaatacgtttattattaaacatattaatatgttatacatttcaaatattttattggtgggaaaaggtaaccccactaataattattcaatccagagtaatatagtccataatacatcatatagtccataaatccatatagtcattatctattgcagtcatttttcaaactaaagatagctataagtatgtgctatcatttctgatttcaagtatatatatatatatatatatatactttcaagtatatatatatatacacactctattcctaatcaacacaggtatattttgataccgtatatatggtatcaaaatatacctgtgttgattaggaatagggtgtatggacatagctttgcttgttaaaccaacaagatagaattatgtcttttatcaattttaattctaagaagttcctgtttccagtttatgtaataatttatatcttggcccctaaatgctctttataaatacatttcagaattttatcagcattttctttctagttcttgttgttggaacatcagtcttcttaataatagtcccaggtggccgggcgcagtggctcacgcctgtaatcccagcactttgggaggtgaaggcgggcggatcacaaggttgggagatcgagaccgtcctggcagtgaaaccccgtctctactaaaaatataaaaaaaatcagccgggcgtggtggtgggcacctgtactcccagctactggggaggctgaggcaggagaatggcctgagcccgggagttgtagcttgcagtgagctgaggtcacgccactgcactccagcctgggcgacagagcgagactctgtctcaaaaaaaaaaatagtcccaagtgctttcaataagttatggtcaaattagttatgaaaactagcttccagatagtacaatgcagagaaaaaacatatatacaggagtgtttttattttactcattaccataatgtaattatcagttgtaaactgaaagtctgctgagagtctgttcctgcgtcgtattcccggttttccagggccattacagattaccacaaactttgtggcctaaaacaatgtagacttgtgttgctgtcattctgtagcttagaagcccaacccctgtgtctcacagggcttaagtcatgcgatcagtagggctgcattgccttctgcagctcgagggaagaacctgttttccttcctttcctggtttttagaggctgcccacattctttggctcttggtttcttccatctcagagacagcaacacagtgtttcattgaccacccttctgtcagcagagctccccccagccgtaactgggaacgcctctgtgcagaaaagttacccttatgattacactgggcccacccaggtaatccagggaaatccctcatctcaatttccttatctgaaccgcgtcaacaaagtcccttttgccatgtatagtgacatattcacaggctcctgggattaggacgtggactatttgagggccagccttctgcctaccacatgcgtacatatttaccttggcatagttattttaaaacaatctgatacacaaatgaaaagggaatttacacaggaaacagacggacagagtctcctcctatactcgtacattagctcaccagcgattagtccatctgcaaacagacgagaaatttgcagggaaagcaatcagaacagggacaaaaacattcttgttttagattgcatatgtttctccttcaatttcttttttttgggggacaatgtgtgagtgattaagcagccggaagagtggcgaaccacagactgtagtcttggaatgtcgagtcttgcctgtaagatgttaaaataagaatcgttttggcctggcatgatgcataattgtaattgtgaaaaaatttaaagctaaaagtttttttgtttaaattccaataaaatatgtttacgttattcctttgattaataataacaaatatatagctcgattaaaaaagtctctgagacacacagctaacagtgaggagaccacgcatcattatgtggttgtattaaagcactcatcggttggaatctgaggtagaaatggtataatacaaaactttatttcatgcactgttttctaactaactgaagatgttcctaattgactgcttttgtacctttttgcaagcttccatagtgaccgtaatgcaccttgtcaatgctgtggttgacacggtgagcacagaaggtacagttgtaagtgctcgtgcttgtgtctgtgtcttgcagcagccgtgtcgtagagctcggccgcattgctattgcaatggtaggaaaaaacagtttgtggttttgagggggttgagtccttttattttatgcaggtgtgtatatacgtaatatgagtgctttgcaaaaaatatctcaatgtatgttatgtctttgttcctttttaccattcatagtgatttacatgagatctggagagcttatctgtaggtggtgcaatcaagaaaaagttcagaagtaattttacctaaaatttttttgaaacaaaaattcttcttcgtatgtacaggaataagacttgaataatgtaaagctttcataattaacctgtaactgactcttcacttttggtttttccctgagtatgatcaaagaaaagggtgtagagcatggtaaaagaatattttcatatttggtttagtgaaaattatttaatgtgaagctttatctgctcagagttgtaagaaaggacattgagcaccttccccaaattattttaaattaaaatagccctttattagtgcactgaaattcatcacatggggaagacacaggctagtacttttcaaactgtaatgtgaatcagatcatctggagggctgttaaaagcccaattgctggttctcagcaccagcatttctgatccaacaggtctgggttgggactttagaatttgcatttttcaccagttcccaggtagctctgatggtgccggtctagagatcccactcgagaactgcttacagaggccatcgagtacacagacgtcatcatagagttcgggggatcctttatgctaggacatgatgctgtcaaggttcaggaggagggaggatagctctgggccccatttgctatcaggcagaagaggcttagatactccagtgttcatggttatagttactgatgacgcctacacttcttttcctaagaagtaccatgttttcactgggcaaatcagaaacatacctgtattttgtctagtgaagttttagtgttaaactgatgaccttcagcattttaaatattattgaagcttatagaagcagaatttcaacagttccttcattgcagtctccttcattgacaaatattttttggcatatccaagaagtgcacatggaatttaaagttgaaagctattactagtgcctacgtaacaaaagttgttgatctttttttgtttaaaggaattgctgcttttgttactagacttaaaaatgattctgtagatacatgttacctaaatgccggcgtattcatgttttagagacaccacagtcttttaaaattactgaaatttctagaagtgaactatcaatcaaatatttgtcccaaattataacactgttgaatgcatagaaagccttgtttaaaaaatcacggatgtgaccgtttttctgctgtgatgtctactttcacagttattgatagtttctaacagtgcttaacctgcatgatcctgtttaatcctcataacaactctaacggattttcaccccatttcaaagagagaacctgaaggttagccagggcctgggcaggcgtctcatggccctaactgatagtgggatttacatgcgtttggttacacttcctgctgggcttctgtgtatagatgcggggccctggcagggcaagtgtaattacagacatggggtaagtgtagccgtggtctctttagggaatggctcacagtgtcaatattaaagcatattcaaaatacatacagtttggagagatcttcagcttgtacatctgtctttaagtatctaagtttgcctatgaattcctctgaatttaatgtcctttgaagttgcagtgtgctataaagtgtgtgcattatgtagctgtagatgtatttggattatcacctatgtaatctgatcagtttatattttattagagcctgtttgttttgttttcattattcctaaggaaattctgtgttttcaggctgtgattgtcagaaacaaggaaaagaaagtaatatttgagttttctggccaaatttctggttcctcattccgcagtcactttcctccacttccatattccattttatttgtggtacatacatttctcagccatttggaattgtcatagatactggcgtaaggacatgctgtatggctgattgggcctctgtaagcagttttcattgttctgtgtacttacctttattgccttttcagccgactgttttaattacaaatgttttcttttttttctactctgccaccaaaagaatctttggctgctcctgacctcagcaaaccaagaggtgacttatgcatgccagttattcattgatactcactaaaaacaatgggtcaagccttgccccagtgctgactgactacgctgggtcgcccagtgtttgcagcagtaactaagtacacggctgcatgtgtgtgcgcattgcatttgaagcacggattgcctaaccttctcccgcctctccaaggattttttaaatcctaaggaggaaaaaaaaaaaactattccaaaattggaagtttttttttaaaatagtggtgctttatatagtctaaaaggatcacagtccttttggaagtaacacagattgattgttacagtgtatcagaggctgtgttataatcttgcaacgcttgttcaattgtattttgcttgaattggcatgctttgatacatacctatattaagttaatttgttgtctgagggttaatgaaaaatagtaattttaatattaaacttgctacattattatgaaggaagcttattttctaaactttagcatgttaaaggttacttttattactttaaaaagttgcattggctgggcctgatggctcacacctgtaatcccagcacttcgggaagctgaattggcaagattgtttgtggccaggagtttaagaccagcctgggcaacatagtgagaccctgtctctataaaaaatagaaatgcaaaatttttttaagttgcattaatcagtatttttttaatgagaagtagtgcagatcaagtgacactggatatttcactaatgtcctgagactggatcagtactcttcttcctacagagcagtgggttctacagaagaaaagccagaaggatggttagaatcatttgtagaaaaatgtatataatttagatgctagcatcccatttctcttaggactccttttgtttccttgaatgttagttgcgtacataaaactgtaagtccacattttagaactagagcctagttcataggggtctttaaactggagtgggttgggggatgtcttcaaagcaaagacgaggaatttccagtggtgaactttgaatgaaggcataaatgcggttgcatctttgtgtctttgatacactgagctctctttccatggcacaaaagcttaggagaaaacacactgacatctgttttcttttccaccaagggtatcactgggatacatcagattggatgccaagcgttcctctgccggacatacaagagttccccaactatgaggtgattgatgagcagacacccctgtactcagcagatccaaacgccatcgatacggactattaccctggaggctacgacatcgaaagtgattttcctccacccccagaagacttccccgcagctgatgagctaccaccgttaccgcccgaattcagcaatcagtttgaatccatccaccctcctagagacatgcctgccgcgggtagcttgggttcttcatcaagaaaccggcagaggttcaacttgaatcagtatttgcccaatttttatcccctcgatatgtctgaacctcaaacaaaaggcactggtgagaatagtacttgtagagaaccccatgccccttacccgccagggtatcaaagacacttcgaggcgcccgctgtcgagagcatgcccatgtctgtgtacgcctccaccgcctcctgctctgacgtgtcagcctgctgcgaagtggagtccgaggtcatgatgagtgactatgagagcggggacgacggccacttcgaagaggtgacgatcccgcccctggattcccagcagcacacggaagtctgactctcaactccccccaaagtgcctgactttagtgaacctagaggtgatgtgagtaatccgcgctgttctttgcagcagtgcttccaagctttttttggtgagccgaatgggcatggctgcgctggatcctgcgcctctggacgtgctagccatttccagtgtcccaactactgtcatcgtgaggttttcatcggctgtgccatttcccaacgtcttttgggatttacatctgtctgtgttaaaataatcaaacgaaaaatcagtcctgtgttgtcagcatgattcatgtatttatatagatttgattattttaattttcctgtctcttttttttgtaaattttatgtacagatttgatttttcatagttttaactagatttccaagatattttgtgcatttgtttcaactgaattttggtggtgtcagtgccattatctagcaccctgatttttttttttttactataaccagggtttcattctgtctttttccactgaagtgtgacattttgttagtacatttcagtgtagtcattcatttctagctgtacataggatgaaggagagatcagatacatgaacatgtcttacatgggttgctgtatttagaattataaacatttttcattattggaaagtgtaacggggaccttctgcatacctgtttagaaccaaaaccaccatgacacagtttttatagtgtctgtatatttgtgatgcaatggtcttgtaaaggtttttaatgaaaactaccattagccagtctttcttactgacaataaattattaataaaata

SEQ ID NO: 7 - DNA, 19 bp, artificial sequence
  synthetic oligonucleotide
tgagctccttccagtccga

SEQ ID NO: 8 - DNA, 20 bp, artificial sequence
  synthetic oligonucleotide
ggaaagcctgtctgaagtgc

SEQ ID NO: 9 - DNA, 20 bp, artificial sequence
  synthetic oligonucleotide
tttccccagcagacattcat

SEQ ID NO: 10 - DNA, 27 bp, artificial sequence
  synthetic oligonucleotide
ttactgacaagttcaaacacctattca

SEQ ID NO: 11 - DNA, 20 bp, artificial sequence
  synthetic oligonucleotide
tgagtcagcaaagacggaaa

SEQ ID NO: 12 - DNA, 20 bp, artificial sequence
  synthetic oligonucleotide
atggaaggctacatcaagca

SEQ ID NO: 13 - DNA, 18 bp, artificial sequence
  synthetic oligonucleotide
tgaccggcagcaaaattg

SEQ ID NO: 14 - DNA, 18 bp, artificial sequence
  synthetic oligonucleotide
tgtaaaacgacggccagt

SEQ ID NO: 15 - DNA, 38 bp, artificial sequence
  synthetic oligonucleotide
tgaccggcagcaaaattgggaaagcctgtctgaagtgc

SEQ ID NO: 16 - DNA, 38 bp, artificial sequence
  synthetic oligonucleotide
tgtaaaacgacggccagttttccccagcagacattcat

SEQ ID NO: 17 - DNA, 38 bp, artificial sequence
  synthetic oligonucleotide
tgaccggcagcaaaattgtgagtcagcaaagacggaaa

SEQ ID NO: 18 - DNA, 38 bp, artificial sequence
  synthetic oligonucleotide
tgtaaaacgacggccagtatggaaggctacatcaagca

SEQ ID NO: 19 - DNA, 18 bp, artificial sequence
  synthetic oligonucleotide
  modified_base (1)..(1): Cy5-conjugated base
tgaccggcagcaaaattg

SEQ ID NO: 20 - DNA, 18 bp, artificial sequence
  synthetic oligonucleotide
  modified_base (1)..(2): Cy-5 conjugated base
tgtaaaacgacggccagt

SEQ ID NO: 21 - DNA, 217 bp, artificial sequence
  microsatellite marker D4S1164
gatctgaaaagcaatgaagaatggctactgacaggttgtagaggcatgagagcgacaggatatatttgactttgagcaaattgcatttattttacagtaaggactgtgaactggtgggtcccaagatgggagtccaggctcgccatgatggcatggctgccaagggaatttctcctcctaaacacaaggtccacttagatccctctggtggctgaga

SEQ ID NO: 22 - DNA, 368 bp, artificial sequence
  microsatellite marker D4S910
  misc_feature (368)..(368): n is a, c, g, or t
gatcacgctccactcagtggtctcacctctgcctgctgaggccagatgctttctggtgccctgctcagaccccttctaacagcacagtgtgtgtttctcttgctccttcacagctgtatgcactcaggagcatggggacaccctcctttcatcttggtgtcctcttcaacatcctagcacatgggcctgaacgtaggagtggctccatatgtgtggaattccaatgcctaggttttctgccttgctagaaaacaggctttttaagggtaggacaaaactataagaaacatttgtattgttttatagtattgaacatgttgcattatacagagctgatagtaaatattgaattgaatgaatgaatgaan

SEQ ID NO: 23 - DNA, 166 bp, artificial sequence
  microsatellite marker D4S3173
atgggttcccaggggccattggagtccctgaaggacccaggatttgctgggagagggtgttgagttcactgtgccagcatgcttcctccacagtaacacgctgaaggggcttggtgtttgtaagaaaatgctagaagaaaacaaactgtcacaagttgttatgtcc

SEQ ID NO: 24 - DNA, 260 bp, artificial sequence
  microsatellite marker D4S3236
  misc_feature (99)..(99): n is a, c, g, or t
  misc_feature (253)..(253): n is a, c, g, or t
ctggagcaggcctgaagtctaggttttgtgtggccatcttggagcctgaagccccaggggctgacctggtctggggtgagcatggggctgaggccacanaggctggtctggcctgtggaggcctgaatcctggtgctggggtttactggagtgggcttggtgcttggaatctgtggtgaagttaggttctatcttaactgtccctcctccatgcaagagggcatctctctccatactgtgctgcccaggcttnaaggtga

SEQ ID NO: 25 - DNA, 184 bp, artificial sequence
  microsattellite marker D4S2827
  misc_feature (7)..(7): n is a, c, g, or t
  misc_feature (13)..(13): n is a, c, g, or t
  misc_feature (21)..(21): n is a, c, g, or t
  misc_feature (27)..(27): n is a, c, g, or t
  misc_feature (72)..(72): n is a, c, g, or t
  misc_feature (143)..(143): n is a, c, g, or t
  misc_feature (156)..(156): n is a, c, g, or t
ctctatncacttncggcaatncaattnaaggaatcagtctcagaagaagagacgggagagaaaatgcatttncacggtgctgaatattatttaagttaatggaggtgaatttaagttgttagggtgcacggtttaaaagcgtnctcacttgctttnatactgagtagttttggtgcttattgag

SEQ ID NO: 26 - DNA, 492 bp, artificial sequence
  microsatellite marker D4S1192
  misc_feature (115)..(115): n is a, c, g, or t
  misc_feature (302)..(302): n is a, c, g, or t
  misc_feature (402)..(403): n is a, c, g, or t
  misc_feature (411)..(411): n is a, c, g, or t
  misc_feature (441)..(441): n is a, c, g, or t
  misc_feature (455)..(455): n is a, c, g, or t
  misc_feature (462)..(462): n is a, c, g, or t
  misc_feature (465)..(467): n is a, c, g, or t
  misc_feature (471)..(471): n is a, c, g, or t
  misc_feature (475)..(475): n is a, c, g, or t
  misc_feature (477)..(477): n is a, c, g, or t
  misc_feature (479)..(481): n is a, c, g, or t
gatccaatgtgctttttggctacagtccaactccttgggaggtcacagttttcaaatgtgagtttttagttttacatgtttacttatttttagagggaacatcactggggctttngggggcttaaactcttactatttcttaaacatatttcttaaactaaggatatttagatattgtcttccatgtacaactttaaattcggttcagaaaccccaggcaagggtattctgtgtgtcctaacccttgaagttaatgacttctaattaactcagcaagaagctaatgacttgtaagcacttgncaattgattcggagtattttctttgtaagatgagtctgggccggcggaactgggaagcctctggcagagagaaggcacaggagggtgggggatggaaacnncatccaangatgagctctggaagatatttagggcaaanggaaagtattgttnggagccnttnnncccncttntntnnnccttcatgact

SEQ ID NO: 27 - DNA, 373 bp, artificial sequence
  microsatellite marker D4S1374
  misc_feature (253)..(253): n is a, c, g, or t
  misc_feature (280)..(280): n is a, c, g, or t
  misc_feature (317)..(317): n is a, c, g, or t
gatccagtcagaagtcagaaaaaagaaaggcatttgaatggtatttctttgccaattcatattcaccgcctctaaaccctatactcttattttctgctggaagtatgaaaagggagggaataaatgtaggaagaaaggaaactgcgaaaatagggaggaaaagaacagagaagaggagagagtggaatggaaacaggaacaggacatttctgaagggtattatccactcttggctaaggtgaagtagcagctnattaggctatggggagacccagagcancctcttttcttttagcaaacccccacgagcttatggnttttccccagcttgggcaggtttcctattgaaatctttctcctggagggatcttgc

SEQ ID NO: 28 - DNA, 389 bp, artificial sequence
  microsatellite marker D4S1375
gatcattatatgtcaggccctgaagcaagtgttgtacaaacattacatcgtttaatcctctcagcaatccagtaaagcagttgttattatcaccattttacagagggagaaactgaggctcaaagtgagcaaattgacttgctcaaacccaaacagctagaaaatgataaggctgccatgcaatcctgggcttattttatccctaagcctgcattccatttcctacaaataaagagatagttttggggtgggaaagttctgataaaggagtggggagactgattgcccctgcacaagcttcccattccatttacatattttaggccctttagcctccagggtttgaatcagagctgggcaggtgacaaaggaggactaggaagccgagc

SEQ ID NO: 29 - DNA, 333 bp, artificial sequence
  microsatellite marker D4S3051
  misc_feature (3)..(3): n is a, c, g, or t
  misc_feature (32)..(32): n is a, c, g, or t
  misc_feature (253)..(253): n is a, c, g, or t
  misc_feature (322)..(322): n is a, c, g, or t
tgntctaatgttggcctttggtggttgcaaanattgtgttaagattttgtttggtctatcaaaagtcataatgatgactactgaaatgattttgaaatcagtagttattctttgtaaaaggaggattttgaagatccaatgtcttttatcaaacatctcaccagtaaattaagaagatctaaatcttgccacacacacacacacacacacacacacacacacccttatgctttaaaatctatctttccaatgnggttcccactgcacatcttgaggggatcttctctggatttccacataagcattgttcaggaaatgcttntcagcagagct

SEQ ID NO: 30 - DNA, 253 bp, artificial sequence
  microsatellite marker D4S2643
  misc_feature (168)..(168): n is a, c, g, or t
ctgaaatgatacatgtcagaattggaaaaataatcttaagtgttctggaagaactctttgatatattgtccaataactcttttcctaacaatacaatgaccatctttaatatgttaaggtctattttcacatccaattcaggactaatctctgctaaagtaccttctnttctcaccttacccaaatcaagtaaccctttacctagatgaaactctccatttggccaagattgatataggaatcagtaattagg

SEQ ID NO: 31 - protein, 6 aa, artificial sequence
  synthetic peptide
QQHTEV

SEQ ID NO: 32 - DNA, 24 bp, artificial sequence
  synthetic oligonucleotide
tgaacgatgaaagaggcacgacac

SEQ ID NO: 33 - DNA, 24 bp, artificial sequence
  synthetic oligonucleotide
gctgagtcacagaaaggctggtta

SEQ ID NO: 34 - DNA, 21 bp, artificial sequence
  synthetic oligonucleotide
ctgaatctggggtgtgttttg

SEQ ID NO: 35 - DNA, 22 bp, artificial sequence
  synthetic oligonucleotide
caagtgagtccatttgtttttg

SEQ ID NO: 36 - DNA, 24 bp, artificial sequence
  synthetic oligonucleotide
tgtgctggaaacagagaacaggtt

SEQ ID NO: 37 - DNA, 24 bp, artificial sequence
  synthetic oligonucleotide
acagtgccatagacaaaggtgatt

SEQ ID NO: 38 - DNA, 22 bp, artificial sequence
  synthetic oligonucleotide
gcaccccttgagactgatttag

SEQ ID NO: 39 - DNA, 24 bp, artificial sequence
  synthetic oligonucleotide
atctgtggagattgatgacatttg

SEQ ID NO: 40 - DNA, 22 bp, artificial sequence
  synthetic oligonucleotide
caacgctgtggagaaacatacc

SEQ ID NO: 41 - DNA, 24 bp, artificial sequence
  synthetic oligonucleotide
taaaaccctaaatcagtctcaagg

SEQ ID NO: 42 - DNA, 22 bp, artificial sequence
  synthetic oligonucleotide
cctctacactggcaaacaaagc

SEQ ID NO: 43 - DNA, 24 bp, artificial sequence
  synthetic oligonucleotide
ttacagacctcggagaataaagat

SEQ ID NO: 44 - DNA, 24 bp, artificial sequence
  synthetic oligonucleotide
atgacgctatgaaaaggtatgaac

SEQ ID NO: 45 - DNA, 24 bp, artificial sequence
  synthetic oligonucleotide
ttgtttgccagtgtagaggattat

SEQ ID NO: 46 - DNA, 24 bp, artificial sequence
  synthetic oligonucleotide
atgacgctatgaaaaggtatgaac

SEQ ID NO: 47 - DNA, 24 bp, artificial sequence
  synthetic oligonucleotide
ttgtttgccagtgtagaggattat

SEQ ID NO: 48 - DNA, 24 bp, artificial sequence
  synthetic oligonucleotide
ttcttcgtttgagtttgtaaggtg

SEQ ID NO: 49 - DNA, 24 bp, artificial sequence
  synthetic oligonucleotide
gttaagaaagttgcgaggaagatt

SEQ ID NO: 50 - DNA, 28 bp, artificial sequence
  synthetic oligonucleotide
gttggatgttcctgtacatacgaagaag

SEQ ID NO: 51 - DNA, 30 bp, artificial sequence
  synthetic oligonucleotide
acgttggatggagagcttatctgtaggtgg

SEQ ID NO: 52 - DNA, 24 bp, artificial sequence
  synthetic oligonucleotide
aaaattacttctgaactttttctt

SEQ ID NO: 53 - DNA, 30 bp, artificial sequence
  synthetic oligonucleotide
acgttggatgtgtttctgacaatcacagcc

SEQ ID NO: 54 - DNA, 31 bp, artificial sequence
  synthetic oligonucleotide
acgttggatggagcctgtttgttttgttttc

SEQ ID NO: 55 - DNA, 20 bp, artificial sequence
  synthetic oligonucleotide
aatcacagcctgaaaacaca

SEQ ID NO: 56 - DNA, 30 bp, artificial sequence
  synthetic oligonucleotide
acgttggatgtactgacaagttcaaacacc

SEQ ID NO: 57 - DNA, 30 bp, artificial sequence
  synthetic oligonucleotide
acgttggatgttgcatgaatgtctgctggg

SEQ ID NO: 58 - DNA, 20 bp, artificial sequence
  synthetic oligonucleotide
caagttcaaacacctattca

SEQ ID NO: 59 - DNA, 22 bp, artificial sequence
  synthetic oligonucleotide
ccgccgttgtgttgttcttttg

SEQ ID NO: 60 - DNA, 22 bp, artificial sequence
  synthetic oligonucleotide
aacgacgctaacaccaccacac

SEQ ID NO: 61 - DNA, 21 bp, artificial sequence
  synthetic oligonucleotide
aactttggcattgtggaaggg

SEQ ID NO: 62 - DNA, 24 bp, artificial sequence
  synthetic oligonucleotide
tcatcatacttggcaggtttctcc

SEQ ID NO: 63 - DNA, 22 bp, artificial sequence
  synthetic oligonucleotide
tgggaatgggtcagaaggactc

SEQ ID NO: 64 - DNA, 21 bp, artificial sequence
  synthetic oligonucleotide
ggtcatcttttcacggttggc

SEQ ID NO: 65 - DNA, 21 bp, artificial sequence
  synthetic oligonucleotide
gctggtgaaaaggacctctcg

SEQ ID NO: 66 - DNA, 22 bp, artificial sequence
  synthetic oligonucleotide
ctggcaacatcaacaggactcc

SEQ ID NO: 67 - DNA, 24 bp, artificial sequence
  synthetic oligonucleotide
ttcttcgtttgagtttgtaaggtg

SEQ ID NO: 68 - DNA, 24 bp, artificial sequence
  synthetic oligonucleotide
gttaagaaagttgcgaggaagatt

SEQ ID NO: 69 - DNA, 24 bp, artificial sequence
  synthetic oligonucleotide
ttcttcgtttgagtttgtaaggtg

SEQ ID NO: 70 - DNA, 22 bp, artificial sequence
  synthetic oligonucleotide
ctattttccccagcagacattc

SEQ ID NO: 71 - DNA, 60 bp, artificial sequence
  synthetic oligonucleotide
ggccagtgaattgtaatacgactcactatagggaggcggttttttttttttttttttttt

SEQ ID NO: 72 - protein, 19 aa, artificial sequence
  synthetic peptide derived from FAT
SKPKPSSEPISFEESVFSC

(19) United States
(12) Patent Application Publication
(10) Pub. No.: US 2007/0202502 A1
(43) Pub. Date: Aug. 30, 2007

(54) Assay For Bipolar Affective Disorder

(76) Inventors: Peter Schofield, New South Wales (AU); Ian Blair, New South Wales (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/586,772

(22) PCT Filed: Jan. 24, 2004
(86) PCT No.: PCT/AU05/00078

§ 371 Date: Feb. 15, 2007

(30) Foreign Application Priority Data
Jan. 23, 2004 (AU) 2004900344

(51) Int. Cl. C12Q 1/68; C12P 19/34; C07H 21/04

(52) U.S. Cl. 435/6; 435/91.2; 536/24.3

(57) ABSTRACT

The present invention provides a method of diagnosing bipolar affective disorder (BAD) and for determining a predisposition of a subject to bipolar affective disorder. In particular, the methods of the present invention comprise detecting a marker that comprises one or more polymorphisms at position 4q35.2 of the human genome and/or one or more allelic variants of the FAT gene associated with BAD or linked thereto. The present invention also relates to identifying new markers that are diagnostic of bipolar affective disorder. Furthermore, the present invention relates to methods of identifying and producing candidate compounds for the treatment of bipolar affective disorder.

### FIELD OF THE INVENTION

The present invention relates to a method of diagnosing bipolar affective disorder (BAD) and for determining a predisposition of a subject to bipolar affective disorder. Furthermore, the present invention relates to methods of identifying and producing candidate compounds for the treatment of bipolar affective disorder.

### BACKGROUND OF THE INVENTION

1. General

This specification contains nucleotide and amino acid sequence information prepared using PatentIn Version 3.1, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, histology and immunology. Such procedures are described, for example, in the following texts that are incorporated by reference:
(i) Sambrook, Fritsch & Maniatis, Molecular Cloning: A Laboratory Manual, Cold Spring Harbor Laboratories, New York, Second Edition (1989), whole of Vols I, II, and III;
(ii) DNA Cloning: A Practical Approach, Vols. I and II (D. N. Glover, ed., 1985), IRL Press, Oxford, whole of text;
(iii) Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
(iv) Nucleic Acid Hybridisation: A Practical Approach (B. D. Hames & S. J. Higgins, eds., 1985) IRL Press, Oxford, whole of text;
(v) Immobilized Cells and Enzymes: A Practical Approach (1986) IRL Press, Oxford, whole of text;
(vi) Perbal, B., A Practical Guide to Molecular Cloning (1984);
(vii) Methods In Enzymology (S. Colowick and N. Kaplan, eds., Academic Press, Inc.), whole of series;
(viii) J. F. Ramalho Ortigao, “The Chemistry of Peptide Synthesis” In: Knowledge database of Access to Virtual Laboratory website (Interactiva, Germany);
(ix) Sakakibara, D., Teichman, J., Lien, E. Land Fenichel, R. L. (1976). Biochem. Biophys. Res. Commun. 73 336-342
(x) Bodanszky, M. (1984) Principles of Peptide Synthesis, Springer-Verlag, Heidelberg.

2. Description of the Related Art

Bipolar affective disorder, also known as manic-depressive illness, is one of the most common mental disorders with a population lifetime prevalence of approximately 1%. Currently, there are approximately 2-3 million adults in the US that are diagnosed with bipolar affective disorder. This disease usually commences in late adolescence or early adulthood and is characterised by periods of elevated mood (mania) and/or periods of depression (Goodwin, et al., 1990, Manic Depressive Illness, Oxford University Press, New York).

The most severe and clinically distinct forms of bipolar affective disorder are bipolar I disorder (severe bipolar affective (mood) disorder) and schizoaffective disorder (manic type). These disorders are characterized by at least one full episode of mania, with or without episodes of major depression (defined by lowered mood, or depression, with associated disturbances in rhythmic behaviours such as sleeping, eating, and sexual activity). Other forms of bipolar affective disorder include bipolar II disorder (characterised by at least one major depressive episode and at least one hypomanic episode), and unipolar disorder (characterised by recurrent major depressive episodes).

Symptoms of a manic episode include, for example, increased energy, activity and restlessness, extreme irritability and provocative, aggressive or intrusive behaviour. A manic episode is deemed to have occurred if several of these symptoms are present all day, nearly every day, for a week. Symptoms of a depressive episode include, for example, anxiety, pessimism, decreased energy, fatigue, loss of appetite or unexplained weight loss or gain. A depressive episode is diagnosed if several of these symptoms last all day, nearly every day for a period of two weeks or longer. Severe episodes of mania or depression may also be accompanied by periods of psychosis or psychotic symptoms.

Currently, bipolar affective disorder is only diagnosed by clinical assessment. Diagnosis is based upon two main schemes, the International Classification of Diseases of the World Health Organisation (10th Edition) and the Diagnostic and Statistic Manual (4th Edition). However, these schemes only detect bipolar affective disorder following onset of the disease. Furthermore, delays in accurate diagnosis using these schemes may extend many years and be associated with instability of presentation. For example, in an adult cohort diagnosed with their first psychotic episode, only 75% of patients were diagnosed with bipolar affective disorder after six months (Fennig et al., Am. J. Psychiatry 1994).

Additionally, the subjective classification systems currently in use for the diagnosis of bipolar affective disorder often lead to incorrect diagnoses as schizophrenia, shizoaffective disorder or psychotic depression.

As a consequence of the current inadequacies in diagnostics for bipolar affective disorder, several groups have attempted to identify a marker useful in the early diagnosis, and determination of a predisposition to the disorder.

Segregation analyses and twin studies have suggested that there is a genetic component to bipolar affective disorder (Bertelson, et al., Br. J. Psychiat. 130: 330-351, 1977; Freimer and Reus, in The Molecular and Genetic Basis of Neurological Disease, Rosenberg et al., eds., Butterworths, New York, 1992 pp. 951-965; Pauls et al., Arch. Gen. Psychiat. 49: 703-708, 1992). However, efforts to identify the chromosomal location of genes that might be linked to bipolar affective disorder have been disappointing. For example, reports of linkage between bipolar affective disorder and markers on the X chromosome and chromosome 11 could not be independently replicated nor confirmed in the re-analyses of the original pedigrees (Baron et al., Nature 326: 289 292, 1987; Egeland et al. Nature 325: 783-787, 1987; Kelsoe, et al., Nature 342: 238-243, 1989; Baron et al., 1993, Nature Genet. 3: 49-55, 1993).

Recent investigations have suggested possible localization of gene that are linked to bipolar affective disorder on chromosomes 18p and 21q (Berrettini, et al. Proc. Natl. Acad. Sci. USA 91, 5918-5921, 1991; Murray et al., Science 265: 2049-2054, 1994; Pauls et al., Am. J. Hum. Genet. 57: 636-643, 1995; Maier et al., Psych. Res. 59: 7-15, 1995; Straub et al., Nature Genet. 8: 291-296, 1994). However, such linkage studies have failed to sufficiently define or validate a locus that is closely linked to the disorder or a predisposition thereto.

A further recent study has suggested that map position 4q35 of the human genome comprises a bipolar affective disorder susceptibility locus (U.S. Pat. No. 6,274,352). This study used linkage analysis with a limited number of microsatellite markers to map a bipolar affective disorder locus to map position 4q35 of the genome. However, the study provides no fine mapping results or association studies to suggest a candidate gene or a candidate allele of a gene that is associated with a bipolar affective disorder.

Efforts to identify candidate bipolar susceptibility genes based upon function alone have also met with limited success. This is partly because our understanding of bipolar disorder aetiology is poor.

Furthermore, there are currently no diagnostic approaches available for determining severity of a bipolar affective disorder. Such methods are useful from the perspective of improving treatment regimes. In particular, the availability of such methods permits more appropriate and targeted treatment to be commenced at an early stage of the disorder. This is in marked contrast to current empirical treatment regimes, which generally commence when severe symptoms have already developed.

Accordingly, there is a clear need to develop improved diagnostic methods for determining a predisposition towards bipolar affective disorder in a subject, and for the early diagnosis of the disorder. Diagnostic assays that rapidly and reliably diagnose bipolar affective disorder prior to onset of the disease are particularly desirable as are indicators of whether or not a subject will respond to a particular treatment.

### SUMMARY OF INVENTION

In work leading up to the present invention the inventors sought to identify markers that are sufficiently tightly linked or associated with a bipolar affective disorder or a predisposition thereto in a human subject, to permit their application in improved diagnostics and prognostics.

As exemplified herein, the present inventors have identified a bipolar affective disease susceptibility locus 4q35.2, by conducting linkage analysis of a large bipolar pedigree. Further analysis, using markers for single nucleotide polymorphisms (SNPs) in the 4q35.2 region confirms the significant association between this region of the human genome and a bipolar affective disorder. The inventors have also shown that several SNPs that are significantly associated with a bipolar affective disorder phenotype are within the human FAT gene, suggesting that the human FAT gene is a susceptibility gene for bipolar affective disorder in humans. Accordingly, polymorphisms within the human FAT gene appear to be statistically correlated with the development of a bipolar affective disorder. This conclusion is further supported by a highly significant association between SNPs located at the 3′ end of the FAT gene and the development of a bipolar affective disorder.

Accordingly, one aspect of the present invention provides a method for determining a bipolar affective disorder or a predisposition to a bipolar affective disorder, said method comprising detecting a marker within a FAT gene or an expression product thereof that is associated with a bipolar affective disorder in a sample derived from a subject, wherein the presence of said marker is indicative of a bipolar affective disorder or a predisposition to a bipolar affective disorder in the subject.

For the purposes of nomenclature, the nucleotide sequence of a human FAT gene is exemplified herein as SEQ ID NO: 1. Preferably, a FAT gene is at least about 80% identical to the sequence set forth in SEQ ID NO: 1.

As used herein, the term “marker” shall be taken to mean a nucleic acid that comprises a nucleotide sequence associated with and/or linked to an allele of a gene that is associated with a bipolar affective disorder and/or is associated with a polymorphism in a genome wherein said polymorphism is associated with a bipolar affective disorder. In those embodiments related to detection of a marker that is in a region of the genome that is transcribed the term “marker” shall also be taken to mean an expression product of a gene or an allele of a gene that is associated with a bipolar affective disorder, such as, for example, a pre-mRNA molecule, a 5′capped mRNA, a polyadenylated mRNA and/or a mature or processed mRNA. In those embodiments related to antigen-based or antibody-based assays, those skilled in the art will appreciate that the term “marker” also means a peptide, polypeptide or protein that comprises an amino acid sequence encoded by an allele of a gene that is associated with a bipolar affective disorder and/or is associated with a polymorphism in a genome wherein said polymorphism is associated with a bipolar affective disorder. A “marker” will generally comprise a sequence that comprises the allele or polymorphism and/or is linked thereto.

As used herein, the term “associated with a bipolar affective disorder” shall be taken to mean that the detection of a marker is significantly correlated with the development of a bipolar affective disorder in a subject. More preferably, the detection of the marker is significantly correlated with the development of a bipolar affective disorder in a plurality of subjects. Even more preferably, the detection of the marker is significantly correlated with the development of a bipolar affective disorder in a plurality of unrelated subjects.

In one embodiment, the marker that is associated with a bipolar affective disorder is within a nucleic acid that comprises a nucleotide sequence at least about 80% identical to a FAT cDNA or FAT mRNA. For example, the nucleotide sequences of splice variants of FAT mRNA are set forth in SEQ ID NO: 2 and SEQ ID NO: 4.

In another embodiment, a marker that is associated with a bipolar affective disorder is within a nucleic acid that is capable of encoding a FAT polypeptide. For example, the amino acid sequences of isoforms of a FAT polypeptide are set forth in SEQ ID NO: 3 and SEQ ID NO: 5.

In a preferred embodiment, a marker that is associated with a bipolar affective disorder is a polymorphism of a FAT gene. As used herein, the term “polymorphism” shall be taken to mean a difference in the nucleotide sequence of the genome that occurs in a normal population of individuals. Such a polymorphism may induce changes in a mRNA and/or polypeptide encoded by the genomic DNA in which it occurs, eg. by changing an encoded amino acid or by altering the splicing or the frequency of splicing of an mRNA. Accordingly, a “polymorphism that is associated with a bipolar affective disorder” means that a particular form of the polymorphism is correlated with the development of a bipolar affective disorder in a subject, ie. in a population of subjects, those that suffer from a bipolar affective disorder are more likely to comprise the specific polymorphism that those subjects that do not suffer from a bipolar affective disorder.

As will be apparent to the skilled artisan a polymorphism that is associated with a bipolar affective disorder that occurs in a region of a FAT gene that is transcribed into mRNA or controls the transcription of mRNA (eg. controls splicing of mRNA) is detectable in an expression product of a FAT gene. Clearly, the present invention encompasses a method of determining a bipolar affective disorder or a predisposition to a bipolar affective disorder comprising detecting a marker associated with a bipolar affective disorder in a FAT expression product, such as, for example, a pre-mRNA molecule, a 5′ capped mRNA, a polyadenylated mRNA, and/or a mature mRNA or cDNA derived therefrom.

Furthermore, a polymorphism that is associated with a bipolar affective disorder that occurs within a protein-coding region of a FAT gene may also cause a change in a polypeptide encoded by the FAT gene. Accordingly, the present invention also encompasses a method of determining a bipolar affective disorder or a predisposition to a bipolar affective disorder comprising detecting a marker associated with a bipolar affective disorder in a FAT polypeptide.

In a further particularly preferred embodiment, the marker comprises, consists of or is located within the 3′ region of a FAT genomic gene, or the corresponding region of an expression product thereof. Preferably, the polymorphism occurs within the 3′ region of the FAT genomic gene that encodes one or more intracellular EVHI1 domains of the FAT protein (i.e., comprising the sequence F/L PPPP). Preferably, the 3′ region of the FAT genomic gene comprises or consists of the region spanning from nucleotide position 139,260 to nucleotide position 170,001 of SEQ ID NO: 1 (ie a nucleic acid that comprises of consists of the sequence set forth in SEQ ID NO: 6).

In another preferred embodiment, the marker comprises, consists of or is located within the region of the FAT genomic gene corresponding to the region spanning from nucleotide position 146,012 to nucleotide position 170,001 of SEQ ID NO: 1, or the corresponding region of an expression product thereof.

In a further preferred embodiment, the marker comprises, consists of or is located within the region of the FAT genomic gene corresponding to the region spanning from nucleotide position 148,108 to nucleotide position 170,001 of SEQ ID NO: 1, or the corresponding region of an expression product thereof.

In a still further preferred embodiment, the marker comprises, consists of or is located within the region of the FAT genomic gene corresponding to the region spanning from nucleotide position 148,199 to nucleotide position 170,001 of SEQ ID NO: 1, or the corresponding region of an expression product thereof.

In another preferred embodiment, the marker comprises, consists of or is located within the region of the FAT genomic gene corresponding to the region spanning from nucleotide position 148,333 to nucleotide position 170,001 of SEQ ID NO: 1, or the corresponding region of an expression product thereof.

In a preferred embodiment, a marker that is associated with a bipolar affective disorder comprises or consists of a single nucleotide polymorphism (SNP). Methods of determining a SNP that is associated with a specific disorder are known in the art and/or described herein.

In a particularly preferred embodiment, a marker that is associated with a bipolar affective disorder comprises or consists of a SNP selected from the group consisting of a cytosine at position 80,217 of SEQ ID NO: 1 (designated rs172903), a thymine at position 130,625 of SEQ ID NO: 1 (designated rs2249916), a thymine at position 130,613 of SEQ ID NO: 1 (designated rs2249917), a guanine at position 139,968 of SEQ ID NO: 1 (designated rs2637777), a guanine at position 142,199 of SEQ ID NO: 1 (designated rs767168), an adenine at position 142,460 of SEQ ID NO: 1 (designated rs2289550), a guanine at position 145,782 of SEQ ID NO: 1 (designated rs1280097), a guanine at position 146,008 of SEQ ID NO: 1 (designated rs1280096), a guanine at position 146,012 of SEQ ID NO: 1 (designated rs2306990), a thymine at position 148,108 of SEQ ID NO: 1 (designated rs1298865), an adenine at position 148,199 of SEQ ID NO: 1 (designated rs2306988), an adenine at position 148,333 of SEQ ID NO: 1 (designated rs2306987), a cytosine at position 151,403 of SEQ ID NO: 1 (designated rs3775309) and a thymine at position 153,127 of SEQ ID NO: 1 (designated rs1973352).

In an even more particularly preferred embodiment, a marker associated with a bipolar affective disorder comprises or consists of a SNP selected from the group consisting of a guanine at position 139,968 of SEQ ID NO: 1 (designated rs2637777), a guanine at position 146,012 of SEQ ID NO: 1 (designated rs2306990), a thymine at position 148,108 of SEQ ID NO: 1 (designated rs1298865), a cytosine at position 148,108 of SEQ ID NO: 1 (designated rs1298865), an adenine at position 148,333 of SEQ ID NO: 1 (designated rs2306987) and a thymidine at position 148,333 of SEQ ID NO: 1 (designated rs2306987).

In one embodiment, a marker associated with a bipolar affective disorder in a subject without a family history of psychiatric illness comprises or consists of a SNP selected from the group consisting of a guanine at position 139,968 of SEQ ID NO: 1 (designated rs2637777), a guanine at position 146,012 of SEQ ID NO: 1 (designated rs2306990), a thymine at position 148,108 of SEQ ID NO: 1 (designated rs1298865 and an adenine at position 148,333 of SEQ ID NO: 1 (designated rs2306987).

In another embodiment, a marker associated with a bipolar affective disorder in a subject with a family history of psychiatric illness comprises or consists of a SNP selected from the group consisting of a thymidine at position 139,968 of SEQ ID NO: 1 (designated rs2637777), an adenine at position 146,012 of SEQ ID NO: 1 (designated rs2306990), a cytosine at position 148,108 of SEQ ID NO: 1 (designated rs1298865 and a cytosine at position 148,333 of SEQ ID NO: 1 (designated rs2306987).

In one embodiment, the marker is in its homozygous form. As used herein, the term “homozygous form” shall be taken to mean that the same form of the marker associated with a bipolar affective disorder occurs at the same locus on homologous chromosomes. For example, the same nucleotide is found at the site of a SNP located FAT gene on both copies of chromosome 4.

As will be apparent to the skilled artisan, a marker that is associated with a bipolar affective disorder is preferably detectable by standard procedures, for example by nucleic acid hybridisation or antibody binding. Accordingly, a nucleic acid marker is preferably at least about 8 nucleotides in length (for example, for detection using a locked nucleic acid (LNA) probe). To provide more specific hybridisation, a marker is preferably at least about 15 nucleotides in length or more preferably at least 20 to 30 nucleotides in length. Such markers are particularly amenable to detection by nucleic acid hybridisation-based detection means assays, such as, for example any known format of PCR or ligase chain reaction.

Preferably, a protein marker encoded by nucleic acid that is associated with a bipolar affective disorder is suitable for antigen-based detection. As will be apparent to the skilled artisan, even a single amino acid change in a polypeptide is detectable in an antigen-based assay. However, for an antibody or ligand to detect such a change, a marker is preferably at least about 6 amino acids in length, more preferably at least about 8 to 10 amino acids in length, even more preferably at least about 14 amino acids in length. A protein marker may also be an entire protein, e.g. wherein the protein that is associated with a bipolar affective disorder is, for example, a conformation different to the protein in a normal or healthy individual or is truncated relative to the protein in a normal or healthy individual.

Means for detecting a marker will be known to the skilled artisan and, in general, any nucleic acid-based or antigen-based detection means can be employed.

In one embodiment, the marker is detected by hybridising a nucleic acid probe comprising the sequence of the marker to a marker linked to nucleic acid in a biological sample derived from a subject under at least moderate, or preferably high stringency hybridisation conditions and detecting the hybridisation using a detection means, wherein hybridisation of the probe to the sample nucleic acid indicates that the subject being tested is predisposed to or suffers from a bipolar affective disorder. Preferably, the detection means is a nucleic acid hybridisation or amplification reaction.

In another embodiment, the marker is detected by hybridising a nucleic acid probe comprising the sequence of the marker to a nucleic acid that is linked to the marker in nucleic acid in a biological sample derived from a subject and detecting the hybridisation by a detection means, wherein hybridisation of the probe to the sample nucleic acid indicates that the subject being tested is predisposed to or suffers from a bipolar affective disorder. Preferably, the detection means is an amplification reaction, or a nucleic acid extension reaction.

Methods of designing and producing or synthesizing probes will be known to the skilled artisan and/or described herein.

The present invention also provides a method for determining a bipolar affective disorder or a predisposition to a bipolar affective disorder in a subject, said method comprising:
(i) amplifying nucleic acid from the subject using an amplification reaction, wherein the amplification reaction is performed using a pair of primers selected from the group consisting of:
   (a) a primer comprising the nucleotide sequence set forth in SEQ ID NO: 32 and a primer comprising the nucleotide sequence set forth in SEQ ID NO: 33;
   (b) a primer comprising the nucleotide sequence set forth in SEQ ID NO: 34 and a primer comprising the nucleotide sequence set forth in SEQ ID NO: 35;
   (c) a primer comprising the nucleotide sequence set forth in SEQ ID NO: 36 and a primer comprising the nucleotide sequence set forth in SEQ ID NO: 37;
   (d) a primer comprising the nucleotide sequence set forth in SEQ ID NO: 38 and a primer comprising the nucleotide sequence set forth in SEQ ID NO: 39;
   (e) a primer comprising the nucleotide sequence set forth in SEQ ID NO: 40 and a primer comprising the nucleotide sequence set forth in SEQ ID NO: 41;
   (f) a primer comprising the nucleotide sequence set forth in SEQ ID NO: 42 and a primer comprising the nucleotide sequence set forth in SEQ ID NO: 43;
   (g) a primer comprising the nucleotide sequence set forth in SEQ ID NO: 44 and a primer comprising the nucleotide sequence set forth in SEQ ID NO: 45;
   (h) a primer comprising the nucleotide sequence set forth in SEQ ID NO: 46 and a primer comprising the nucleotide sequence set forth in SEQ ID NO: 47;
   (i) a primer comprising the nucleotide sequence set forth in SEQ ID NO: 48 and a primer comprising the nucleotide sequence set forth in SEQ ID NO: 49;
   (j) a primer comprising the nucleotide sequence set forth in SEQ ID NO: 50 and a primer comprising the nucleotide sequence set forth in SEQ ID NO: 51;
   (k) a primer comprising the nucleotide sequence set forth in SEQ ID NO: 53 and a primer comprising the nucleotide sequence set forth in SEQ ID NO: 54; and
   (l) a primer comprising the nucleotide sequence set forth in SEQ ID NO: 56 and a primer comprising the nucleotide sequence set forth in SEQ ID NO: 57;
(ii) detecting a polymorphism in the amplified nucleic acid from (i), wherein said polymorphism is indicative of a bipolar affective disorder or a predisposition to a bipolar affective disorder in the subject.

Preferably, the polymorphism is detected by determining the nucleotide sequence of the amplified nucleic acid.

In another embodiment, the marker is detected by contacting a biological sample derived from a subject with an antibody or ligand capable of specifically binding to said marker for a time and under conditions sufficient for an antibody/ligand complex to form and then detecting the complex wherein detection of the complex indicates that the subject being tested is predisposed to or suffers from a bipolar affective disorder.

Clearly the present invention encompasses the use of a multiplex assay to determine the predisposition of a subject to a bipolar affective disorder or to diagnose a bipolar affective disorder. In this regard, such a multiplexed assay may detect two or more nucleic acid markers that are associated with bipolar affective disorder. Alternatively, or in addition, a multiplexed assay may detect two or more peptide, polypeptide or protein markers that are associated with bipolar affective disorder. Clearly, the combination of nucleic acid-based and antigen-based detection methods is contemplated by the invention.

The biological sample used in an assay to determine the presence of a bipolar affective disorder or a predisposition to a bipolar affective disorder will comprise a nucleated cell. Preferably, the biological sample is selected from the group consisting of whole blood, serum, plasma, peripheral blood mononuclear cells (PBMC), a buffy coat fraction, saliva, urine, a buccal cell and a skin cell.

Alternatively, the biological sample is a cell isolated using a method selected from the group consisting of aminocentesis, chorionic villus sampling, fetal blood sampling (e.g. cordocensesis or percutaneous umbilical blood sampling and other fetal tissue sampling (e.g. fetal skin biopsy).

As the FAT polypeptide is expressed in a variety of tissues in developing embryo, an antigen based assay is particularly useful in prenatal testing to determine predisposition to a bipolar affective disorder in a suitable biological sample for prenatal testing comprises a cell derived from a tissue selected from the group consisting of brain (a neural crest cell), spinal cord, skin, lung, kidney, pancreas.

As will be apparent to the skilled artisan, the size of a biological sample will depend upon the detection means used. For example, an assay, such as, for example, PCR or single nucleotide primer extension may be performed on a sample comprising a single cell, although greater numbers of cells are preferred. Alternative forms of nucleic acid detection may require significantly more cells than a single cell. Furthermore, protein-based assays require sufficient cells to provide sufficient protein for an antigen based assay.

Preferably, the biological sample has been derived previously from the subject.

In one embodiment, the method is performed using genomic DNA derived from a biological sample. In another embodiment, the method is performed using mRNA or cDNA derived from the biological sample. In a still further embodiment, the method of the present invention is performed using protein derived from the biological sample.

Another aspect of the present invention provides a method for determining a bipolar affective disorder or a predisposition to a bipolar affective disorder, said method comprising detecting a marker that is linked to map position 4q35.2 of the human genome in a sample derived from a subject, wherein the detection is indicative of a bipolar affective disorder or a predisposition to a bipolar affective disorder in the subject.

As used herein, the terms “linked” and “map to” shall be taken to refer to a sufficient proximity between a marker and nucleic acid comprising all or part of map position 4q35.2 of the human genome or an expression product thereof to permit said linked nucleic acid to be useful for diagnosing bipolar affective disorder in a subject. Those skilled in the art will be aware that for linked nucleic acid to be used in this manner, it must be sufficiently close to map position 4q35.2 so as to be in linkage disequilibrium or for there to be a low recombination frequency between the linked nucleic acid and map position 4q35.2. Preferably, the linked nucleic acid and the locus are less than about 25 cM apart, more preferably less than about 10 cM apart, even more preferably less than about 5 cM apart, still even more preferably less than about 3 cM apart and still even more preferably less than about 1 cM apart.

In one embodiment the method of determining the predisposition of a subject to bipolar affective disorder or diagnosing bipolar affective disorder comprises determining the presence or absence of the a marker linked to chromosome 4q35.2 in a test sample derived from a subject, wherein a presence or absence of the marker linked to chromosome 4q35.2 indicates that the subject being tested is predisposed to or suffers from bipolar affective disorder.

In another embodiment of the invention, the marker is a peptide, polypeptide or protein that is encoded by nucleic acid that is linked with map position 4q35.2 of the human genome.

In one embodiment, a marker that is linked to map position 4q35.2 of the human genome occurs in a region of the genome that does not encode a protein, such as, for example, an intron, a 5′ untranslated region, a 3′ untranslated region or a promoter region of a genomic gene. In another embodiment, a marker that is linked to map position 4q35.2 of the human genome occurs in a region of the genome that encodes a protein. Accordingly, the term “a marker that is linked to map position 4q35.2” clearly extends to a marker that comprises and/or is detected in an mRNA encoded by a gene that is linked to map position 4q35.2 of the human genome.

In one embodiment, the marker linked to map position 4q35.2 is located between or comprises the microsatellite markers designated D4S1164 (SEQ ID NO: 21) and D4S1192 (SEQ ID NO: 27). Preferably, the marker linked to map position 4q35.2 is located between or comprises the microsatellite markers designated D4S910 (SEQ ID NO: 22) and D4S1374 (SEQ ID NO: 28). More preferably, the marker linked to map position 4q35.2 is located between the microsatellite markers designated D4S3173 (SEQ ID NO: 23) and D4S1375 (SEQ ID NO: 29). Even more preferably, the marker linked to map position 4q35.2 is located between or comprises the microsatellite markers D4S3236 (SEQ ID NO: 24) and designated D4S3051 (SEQ ID NO: 30). Still more preferably, the marker linked to map position 4q35.2 is located between or comprises the microsatellite markers designated D4S2827 (SEQ ID NO: 25) and D4S2643 (SEQ ID NO: 31).).

In a preferred embodiment, the marker linked to map position 4q35.2 is located between or comprises the microsatellite markers designated D4S2827 (SEQ ID NO: 25) and D4S2643 (SEQ ID NO: 31).

In a preferred embodiment, the marker linked to map position 4q35.2 comprises or consists of a nucleic acid that comprises a nucleotide sequence at least about 80% identical to the genomic region that comprises a human FAT gene (i.e. the sequence set forth in SEQ ID NO: 1). More preferably, the marker comprises or consists of a genomic gene capable of encoding a nucleic acid (i.e. mRNA) that comprises a nucleotide sequence at least about 80% identical to any FAT cDNA or mRNA encoded by a FAT genomic gene. For example, the nucleotide sequence of two alternative splice forms of FAT are set forth in SEQ ID NO: 2 or SEQ ID NO: 4. In an alternative embodiment, the marker comprises or consists of a genomic gene that encodes an isoform of a FAT protein. For example, the amino acid sequence of two forms of a FAT protein encoded by different splice forms of a FAT gene are set forth in SEQ ID NO: 3 or SEQ ID NO: 5.

In a further particularly preferred embodiment, the marker comprises, consists of or is located within the 3′ region of a FAT genomic gene, or the corresponding region of an expression product thereof. Preferably, the polymorphism occurs within the 3′ region of the FAT genomic gene that encodes one or more intracellular EVH1 domains of the FAT protein (i.e., comprising the sequence F/L PPPP). Preferably, the 3′ region of the FAT genomic gene comprises or consists of the region spanning from nucleotide position 139,260 to nucleotide position 170,001 of SEQ ID NO: 1 (ie a nucleic acid that comprises of consists of the sequence set forth in SEQ ID NO: 6).

In another preferred embodiment, the marker comprises, consists of or is located within the region of the FAT genomic gene corresponding to the region spanning from nucleotide position 146,012 to nucleotide position 170,001 of SEQ ID NO: 1, or the corresponding region of an expression product thereof.

In a further preferred embodiment, the marker comprises, consists of or is located within the region of the FAT genomic gene corresponding to the region spanning from nucleotide position 148,108 to nucleotide position 170,001 of SEQ ID NO: 1, or the corresponding region of an expression product thereof.

In a still further preferred embodiment, the marker comprises, consists of or is located within the region of the FAT genomic gene corresponding to the region spanning from nucleotide position 148,199 to nucleotide position 170,001 of SEQ ID NO: 1, or the corresponding region of an expression product thereof.

In another preferred embodiment, the marker comprises, consists of or is located within the region of the FAT genomic gene corresponding to the region spanning from nucleotide position 148,333 to nucleotide position 170,001 of SEQ ID NO: 1, or the corresponding region of an expression product thereof.

Preferably, a marker comprises or is contained within a FAT gene or a transcription product thereof, such as, for example, a pre-mRNA molecule, a 5′capped mRNA, a polyadenylated mRNA and/or a mature or processed mRNA. More preferably, a marker comprises is contained within or consists of a nucleotide sequence at least about 80% identical to a sequence selected from the group consisting of:
(i) a sequence at least about 80% homologous to a sequence selected from the group consisting of SEQ ID NO: 1, SEQ ID NO: 2 and SEQ ID NO: 4;
(ii) a sequence capable of encoding an amino acid sequence at least 80% homologous to the sequence set forth in SEQ ID NO: 3 and SEQ ID NO: 5; and
(iii) a sequence complementary to a sequence set forth in (i) or (ii).

In one embodiment, the marker linked to map position 4q35.2 comprises or consists of a microsatellite marker selected from the group consisting of D4S1164 (SEQ ID NO: 21), D4S1192 (SEQ ID NO: 27), D4S910 (SEQ ID NO: 22), D4S1374 (SEQ ID NO: 28), D4S3173 (SEQ ID NO: 23), D4S1375 (SEQ ID NO: 29), D4S3236 (SEQ ID NO: 24), D4S3051 (SEQ ID NO: 30), D4S2827 (SEQ ID NO: 25) and D4S2643 (SEQ ID NO: 31).

In another embodiment, the marker linked to map position 4q35.2 comprises or consists of a single nucleotide polymorphism (SNP). Preferably, the SNP is selected from the group consisting of a cytosine at position 80,217 of SEQ ID NO: 1 (designated rs172903), a thymine at position 130,625 of SEQ ID NO: 1 (designated rs2249916), a thymine at position 130,613 of SEQ ID NO: 1 (designated rs2249917), a guanine at position 139,968 of SEQ ID NO: 1 (designated rs2637777), a guanine at position 142,199 of SEQ ID NO: 1 (designated rs767168), an adenine at position 142,460 of SEQ ID NO: 1 (designated rs2289550), a guanine at position 145,782 of SEQ ID NO: 1 (designated rs1280097), a guanine at position 146,008 of SEQ ID NO: 1 (designated rs1280096), a guanine at position 146,012 of SEQ ID NO: 1 (designated rs2306990), a thymine at position 148,108 of SEQ ID NO: 1 (designated rs1298865), an adenine at position 148,199 of SEQ ID NO: 1 (designated rs2306988), an adenine at position 148,333 of SEQ ID NO: 1 (designated rs2306987), a cytosine at position 151,403 of SEQ ID NO: 1 (designated rs3775309) and a thymine at position 153,127 of SEQ ID NO: 1 (designated rs1973352).

In a particularly preferred embodiment, a marker linked to map position 4q35.2 is a SNP selected from the group consisting of a guanine at position 139,968 of SEQ ID NO: 1 (designated rs2637777), a guanine at position 146,012 of SEQ ID NO: 1 (designated rs2306990), a thymine at position 148,108 of SEQ ID NO: 1 (designated rs1298865) and an adenine at position 148,333 of SEQ ID NO: 1 (designated rs2306987).

In one embodiment, a marker associated with a bipolar affective disorder in a subject without a family history of psychiatric illness comprises or consists of a SNP selected from the group consisting of a guanine at position 139,968 of SEQ ID NO: 1 (designated rs2637777), a guanine at position 146,012 of SEQ ID NO: 1 (designated rs2306990), a thymine at position 148,108 of SEQ ID NO: 1 (designated rs1298865 and an adenine at position 148,333 of SEQ ID NO: 1 (designated rs2306987).

In another embodiment, a marker associated with a bipolar affective disorder in a subject with a family history of psychiatric illness comprises or consists of a SNP selected from the group consisting of a thymidine at position 139,968 of SEQ ID NO: 1 (designated rs2637777), an adenine at position 146,012 of SEQ ID NO: 1 (designated rs2306990), a cytosine at position 148,108 of SEQ ID NO: 1 (designated rs1298865 and a cytosine at position 148,333 of SEQ ID NO: 1 (designated rs2306987).

Methods of detecting a marker associated with a bipolar affective disorder as described supra shall be taken to apply mutatis mutandis to the methods of detecting a marker linked to map position 4q35.2 of the human genome.

The present invention also encompasses methods of genetic screening to determine a subject that carries a gene or allele of a gene or a polymorphism that confers susceptibility to a bipolar affective disorder or is associated with a bipolar affective disorder or carries a marker for a bipolar affective disorder that is linked to map position 4q35.2 of the human genome, ie. determining a subject that is homozygous or heterozygous for a susceptibility gene or allele. Accordingly, a further aspect of the present invention provides a method for determining a subject that carries a gene or allele of a gene or a polymorphism that is associated with a bipolar affective disorder comprising detecting a marker within a FAT gene that is associated with a bipolar affective disorder in a sample derived from the subject, wherein detection of said marker indicates that the subject is a carrier of a gene or allele of a gene or a polymorphism is associated with a bipolar affective disorder.

In one embodiment, the subject is heterozygous for one gene or allele of a gene or one polymorphism that confers susceptibility to a bipolar affective disorder or is associated with a bipolar affective disorder. Accordingly, an assay to detect a marker associated with a bipolar affective disorder and/or linked to map position 4q35.2 of the human genome may indicate that the subject comprises a marker associated with a bipolar affective disorder and a marker that is not associated with a bipolar affective disorder (eg., two different nucleotides at a SNP position, one of which is associated with a bipolar affective disorder). Alternatively, or in addition, the subject may comprise a marker that is associated with a bipolar affective disorder in a subject with a family history of psychiatric illness and a marker that is associated with a bipolar affective disorder in a subject that does not have a family history of psychiatric illness. Clearly, this aspect of the present invention is useful for determining the likelihood, or susceptibility of a child to develop a bipolar affective disorder based upon screening of the parents of the child (eg., genetic counselling). Such an assay may be performed prior to pregnancy, during pregnancy or even after birth. Methods of determining the risk of a child developing a disorder or the predisposition of a child to the development of a bipolar affective disorder based upon the genotype of the parents of the child are known in the art and described, for example, in Young (In: Introduction to Risk Calculation in Genetic Counselling (Oxford Medical Publications), Oxford University Press; 1991.

As the methods of the present invention are particularly useful for determining whether or not a subject is likely to suffer from a bipolar affective disorder or is susceptible to a bipolar affective disorder, these methods are particularly useful in methods of treatment or prophylaxis of a bipolar affective disorder.

Accordingly, the present invention additionally provides a method of treatment or prophylaxis of a bipolar affective disorder comprising:
(i) performing a method described herein for determining a bipolar affective disorder or a predisposition to a bipolar affective disorder; and
(ii) administering or recommending a therapeutic for the treatment of bipolar affective disorder.

In one embodiment, the administration or recommendation of a therapeutic for the treatment of a bipolar affective disorder is based upon the diagnosis of a bipolar affective disorder or the diagnosis of a predisposition to a bipolar affective disorder.

This embodiment of the invention applies mutatis mutandis to a method of monitoring the efficacy of treatment. In such a monitoring procedure, it is possible to modify the therapeutic regimen based upon altered expression of the marker that is detected.

As will be apparent to the skilled artisan, this aspect of the present invention also encompasses methods for the prophylaxis or prophylactic therapy of a bipolar affective disorder. Accordingly, a preferred embodiment of the invention provides a method for the prophylaxis of a bipolar affective disorder comprising determining a subject at risk of developing a bipolar affective disorder using a method described herein; and administering or recommending a compound that delays or prevents onset of a bipolar affective disorder, such as, for example, lithium or valproate. Preferably, the administration or recommendation of the compound is based upon the determination of a subject at risk of developing (ie. predisposed to developing) a bipolar affective disorder.

A marker useful for determining a subject at risk of developing a bipolar affective disorder and means for detecting said marker are described herein.

Given the tight association of the human FAT gene to a bipolar affective disorder, and the provision of numerous markers, the present invention further provides methods for identifying new markers of a bipolar affective disorder.

Accordingly, the present invention additionally provides a method for identifying a marker that is associated with a bipolar affective disorder, said method comprising:
(i) identifying a polymorphism or allele within a FAT gene or an expression product thereof;
(ii) analyzing a panel of subjects to determine those that suffer from a bipolar affective disorder, wherein not all members of the panel comprise the polymorphism or allele; and
(iii) determining the variation in the development of a bipolar affective disorder wherein said variation indicates that the polymorphism or allele is associated with a subject's predisposition to a bipolar affective disorder.

Another aspect of the present invention provides a method of identifying a marker for bipolar affective disorder comprising identifying a marker that is linked to chromosome position 4q35.2 of the human genome, wherein said marker is present in an individual suffering from a bipolar affective disorder and said marker is not present in a suitable control subject.

In another embodiment, the present invention provides a method of identifying a marker that determines a subject s predisposition to a bipolar affective disorder and/or is diagnostic of a bipolar affective disorder comprising:
(i) identifying a locus that is associated with the genetic variation in a bipolar affective disorder;
(ii) identifying a marker that is linked to the locus (i), wherein said marker is associated with a subjects predisposition for a bipolar affective disorder and/or is diagnostic of a bipolar affective disorder;
(iii) analyzing a panel of subjects to determine those that suffer from a bipolar affective disorder, wherein not all members of the panel comprise or express the marker; and
(iv) determining the variation in the presence or absence of the marker between the members of the panel wherein said variation indicates that the marker is involved in determining or is associated with a subjects predisposition for bipolar affective disorder and/or is diagnostic of bipolar affective disorder.

In a related embodiment, the present invention provides a method of identifying a marker that determines a subject's predisposition for a bipolar affective disorder and/or is diagnostic of a bipolar affective disorder comprising:
(i) identifying a locus that is associated with genetic variation in a bipolar affective disorder in a subject;
(ii) identifying an allele of a gene that is linked to the locus at (i), wherein said allele is a candidate allele that determines or is associated with a subjects predisposition for a bipolar affective disorder and/or is diagnostic of a bipolar affective disorder;
(iii) analyzing a panel of subjects to determine those that suffer from bipolar affective disorder, wherein not all members of the panel comprise or express the allele; and
(iv) determining the variation in expression at (iii) wherein said variation in the presence or absence of the allele between the members of the panel indicates that the allele is a marker involved in determining or is associated with a subjects predisposition for a bipolar affective disorder and/or is diagnostic of a bipolar affective disorder.

In a further embodiment, the present invention provides a method of identifying a marker that determines a subject's predisposition for a bipolar affective disorder and/or is diagnostic of a bipolar affective disorder comprising:
(i) identifying a locus that is associated with genetic variation in a bipolar affective disorder in a subject;
(ii) identifying several alleles of a gene that are linked to the locus (i), wherein said gene is a candidate gene that determines or is associated with a subjects predisposition for bipolar affective disorder and/or is diagnostic of a bipolar affective disorder;
(iii) analyzing a panel of subjects to determine those that suffer from bipolar affective disorder, wherein each of the members of the panel comprise or express at least one of the alleles; and
(iv) determining the variation at (iii) wherein said variation in the presence or absence of the alleles between the members of the panel indicates that the gene is a marker involved in determining or is associated with a subjects predisposition for a bipolar affective disorder and/or is diagnostic of a bipolar affective disorder.

In a preferred embodiment, a marker, allele and/or locus is linked to map position 4q35.2 of the human genome and is associated with a bipolar affective disorder.

In a preferred embodiment, the identified marker consists of a FAT gene, comprises a FAT gene or is contained within a FAT gene.

In another preferred embodiment, the identified marker is an allele of a FAT gene and/or an expression product thereof and/or a polymorphism that occurs within a FAT gene and/or an expression product thereof.

Clearly, the present invention extends to the use of a marker that is associated with a bipolar affective disorder and is within a FAT gene or an expression product thereof and/or is linked to map position 4q35.2 of the human genome in the manufacture of a diagnostic reagent for determining the predisposition of a subject to a bipolar affective disorder or diagnosing a bipolar affective disorder.

The present invention also provides a marker that is associated with a bipolar affective disorder and is within a FAT gene or an expression product thereof and/or is linked to map position 4q35.2 of the human genome when used to determine the predisposition of a subject to a bipolar affective disorder or diagnose a bipolar affective disorder.

The present invention additionally provides a probe or primer comprises at least about 20 nucleotides and is capable of hybridizing to the sequence set forth in SEQ ID NO: 1 and detect a marker in a FAT gene that is associated with a bipolar affective disorder or a predisposition to a bipolar affective disorder.

Preferably, the probe or primer comprises a nucleotide sequence selected from the group consisting of SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58.

Furthermore, the invention provides for the use of a probe or primer comprising at least about 20 nucleotides that is capable of hybridizing to the sequence set forth in SEQ ID NO: 1 and detecting a marker in a FAT gene that is associated with a bipolar affective disorder or a predisposition to a bipolar affective disorder in the manufacture of a diagnostic reagent for determining the predisposition of a subject to a bipolar affective disorder or diagnosing a bipolar affective disorder. Preferably, the probe or primer comprises a nucleotide sequence selected from the group consisting of SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58.

As exemplified herein, the level of FAT gene expression is modulated by known therapeutics of a bipolar affective disorder, and, in particular, lithium and valproate. Accordingly, another aspect of the present invention provides a method of determining a candidate compound for the treatment of a bipolar affective disorder comprising:
(i) administering a candidate compound to an animal or cell comprising or expressing a marker that is associated with a bipolar affective disorder and/or is linked to map position 4q35.2 of the human genome and determining the level of FAT expression in said cell or animal;
(ii) administering a candidate compound to an animal or cell that does not comprise or express a marker that is associated with a bipolar affective disorder and/or is linked to map position 4q35.2 of the human genome and determining the level of FAT expression in said cell or animal; and
(iii) comparing the level of FAT expression at (i) and (ii),
   wherein a decreased level of FAT expression at (i) relative to (ii) indicates that the compound is a candidate compound for the treatment of a bipolar affective disorder.

Preferably, the level of FAT expression is determined by determining the level of a FAT polypeptide in a cell or animal.

In a particularly preferred embodiment, the level of FAT expression is determined by determining the level of a FAT mRNA in a cell.

### BRIEF DESCRIPTION OF THE DRAWINGS

FIG. 1 is a graphical representation showing selected microsatellite markers on human chromosome 4 that show a significant association with bipolar affective disorder. The markers that showed a significant association with bipolar affective disorder are located at map position 4q35.2 and span approximately 5 megabases (Mb).

FIG. 2 is a graphical representation showing a transcript map of known and predicted transcripts that map to chromosome 4q35.2. Expressed tag sequences that map to this region of the genome are also indicated at the base of the figure.

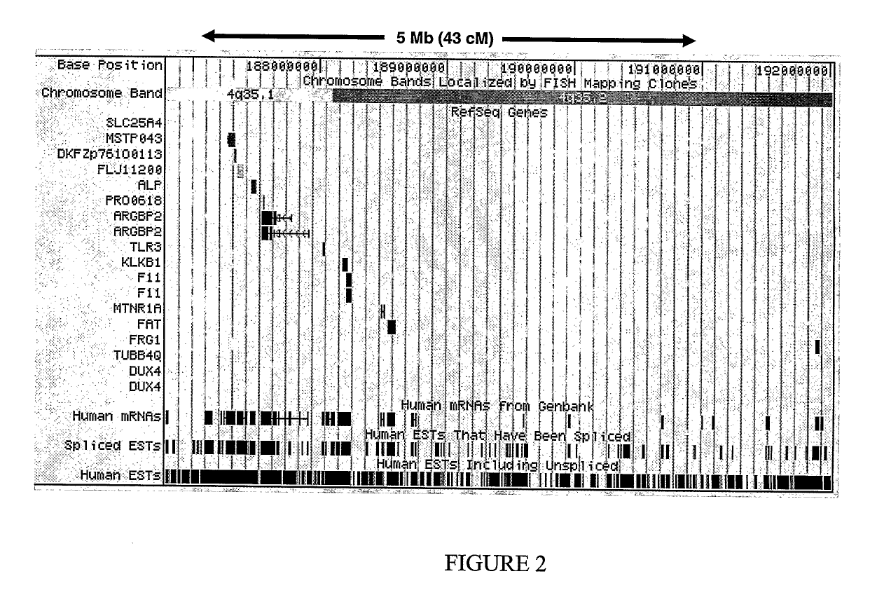

FIG. 3 is a schematic representation of the human FAT genomic gene showing the location of the single nucleotide polymorphisms (SNPs) that show an association with bipolar affective disorder. The SNPs that show a significant association with bipolar affective disorder are located in exon 26 and intron 26 of the FAT gene.

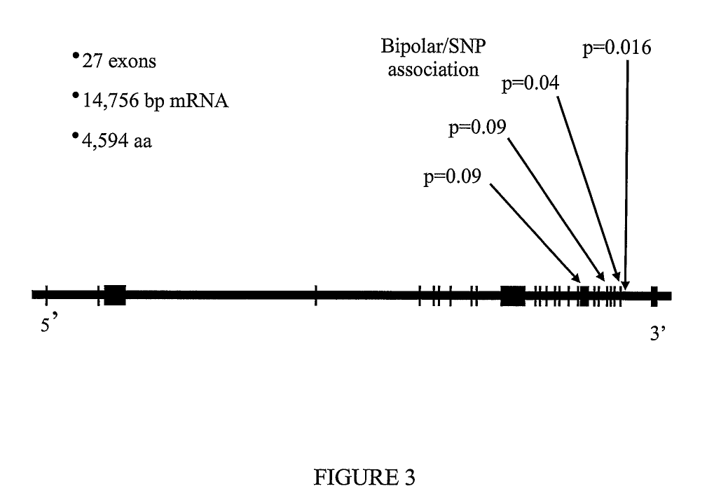

FIG. 4 is a tabular representation showing linkage disequilibrium in the SNPs detected in control subjects (ie., subjects that do not suffer from bipolar affective disorder). Note that the SNPs located between exon 19 and intron 26 are in linkage disequilibrium, representing a haplotype block in this region.

FIG. 5 is a tabular representation showing linkage disequilibrium in the SNPs detected in subjects that suffer from a bipolar affective disorder. Note that specific SNPs located between exon 19 and intron 26 are in linkage disequilibrium in bipolar affective disorder subjects, indicating that there is a haplotype block in this region of the FAT gene.

FIG. 6 is a tabular representation showing the linkage disequilibrium (LD) 26 SNPs (indicated by their RefSNP ID) analysed in the Australian case-control cohort. SNPs showing significant evidence of association with bipolar disorder are boxed. The output of LD analysis using Haploview is shown. Haplotype blocks, determined using the default confidence interval algorithm, are indicated on the LD output. The associated SNPs all fall within an 8.7 kb haplotype block (Block 3).

FIG. 7 is a graphical representation showing the effect of lithium and valproate on the level of FAT mRNA. Mice were administered with lithium or valproate and the level of FAT mRNA expression relative to GAPDH mRNA expression was determined using real time PCR. High doses of lithium were observed to significantly reduce the levels of FAT expression, while high doses of valproate also appear to reduce FAT mRNA expression. *, p=0.027

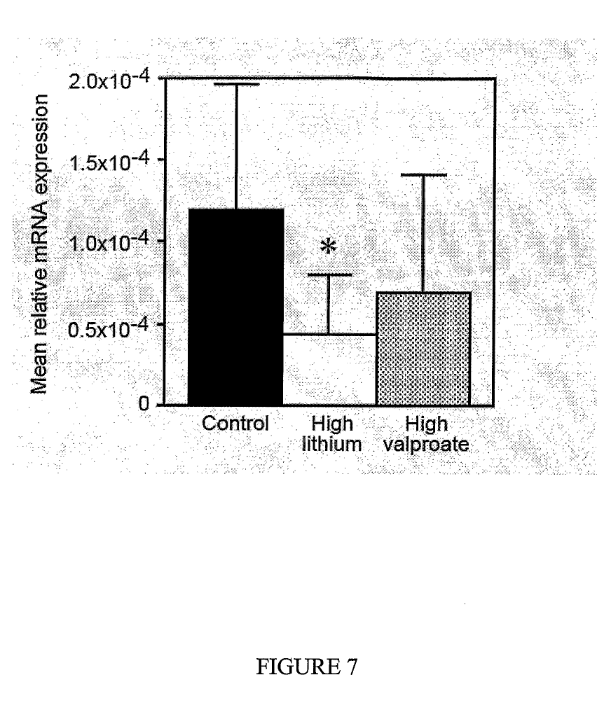

FIG. 8 is a graphical representation showing the region of the exon trap vectors used in the analysis of alternative splicing of the human FAT gene. Clones 1 to 3 all comprised exon 26 of the FAT gene in addition to regions of introns 25 and 26. Clone 1 comprises thymine at a position corresponding to nucleotide position 148,129 of SEQ ID NO: 1 (rs1298865) and an adenine at a position corresponding to nucleotide position 148,333 of SEQ ID NO: 1 (rs2306987); clone 2 comprises a cytosine at a position corresponding to nucleotide position 148,129 (rs1298865) of SEQ ID NO: 1 and a thymine at a position corresponding to nucleotide position 148,333 of SEQ ID NO: 1 (rs2306987); and clone 3 comprises a thymine at a position corresponding to nucleotide position 148,129 of SEQ ID NO: 1 (rs1298865).

FIG. 9 is a graphical representation of a photograph showing analysis by RT-PCR of spliced mRNA derived from exon-trap clones following transfection in HEK293 cells. Lane M, 100 bp DNA size marker. Lane C1, exon-trap assay positive control. Lane C2, exon-trap assay negative control. Lane 1, cDNA derived from clone 1. Lane 2, cDNA derived from clone 2. Lane 3, cDNA derived from clone 3. The identity of the arrowed bands was determined by DNA sequencing. The band labelled “ex26” represents correctly spliced FAT exon 26 mRNA. The band labelled “cryptic” represents FAT exon 26 mRNA that has been spliced to the final 55 bp of the cloned FAT genomic sequence (and additional flanking vector sequence) via activation of a cryptic splice acceptor site that lies 93 bp downstream of the SNP designated rs2306987.

### DETAILED DESCRIPTION OF THE PREFERRED EMBODIMENTS

Methods of Detecting a Marker within a FAT Gene that is Associated with a Bipolar Affective Disorder

One aspect of the present invention provides a method for determining a bipolar affective disorder or a predisposition to a bipolar affective disorder, said method comprising detecting a marker within a FAT gene or an expression product thereof that is associated with a bipolar affective disorder in a sample derived from a subject, wherein the presence of said marker is indicative of a bipolar affective disorder or a predisposition to a bipolar affective disorder in the subject.

As used herein, the term “predisposition to a bipolar affective disorder” shall be taken to mean that a subject is susceptible to a form of bipolar affective disorder or is more likely to develop a bipolar affective disorder than a normal individual or a normal population of individuals. In this regard, a marker that is indicative of a predisposition to a bipolar affective disorder may itself cause a bipolar affective disorder or, alternatively, be correlated with the development of a bipolar affective disorder.

The term “a bipolar affective disorder” shall be taken to include all forms of bipolar affective disorder, including bipolar I disorder (severe bipolar affective (mood) disorder), schizoaffective disorder, bipolar II disorder or unipolar disorder.

In one embodiment, a bipolar affective disorder is a type I bipolar affective disorder.

In another embodiment, a bipolar affective disorder is a type II bipolar affective disorder.

In a further embodiment, a bipolar affective disorder is a schizoaffective disorder.

In a still further embodiment, a bipolar affective disorder is unipolar disorder.

In one embodiment, the marker that is associated with a bipolar affective disorder comprises, consists of or is within a nucleic acid that comprises a nucleotide sequence at least about 80% identical to a FAT cDNA or FAT mRNA. For example, the nucleotide sequences of splice variants of FAT mRNA are set forth in SEQ ID NO: 2 and SEQ ID NO: 4.

As used herein, the term “FAT gene” shall be taken to include a genomic FAT gene, i.e. a gene that comprises a nucleotide sequence at least about 80% identical to the sequence set forth in SEQ ID NO: 1. Preferably, the degree of sequence identity is at least about 85% to 90% identity, more preferably, 90% to 95% identity and even more preferably 98% to 99% identity.

Preferably, the FAT gene is a human FAT gene.

In another embodiment, a FAT gene is a gene that encodes a nucleic acid (i.e. a FAT mRNA or FAT cDNA) at least about 80% identical to the sequence set forth in SEQ ID NO: 2 or 4. Preferably, the degree of sequence identity is at least about 85% to 90% identity, more preferably, 90% to 95% identity and even more preferably 98% to 99% identity.

In yet another embodiment, a FAT gene is capable of encoding a FAT polypeptide, e.g. a polypeptide comprising an amino acid sequence at least about 80% identical to the sequence set forth in SEQ ID NO: 3 or 5. Preferably, the degree of sequence identity is at least about 85% to 90% identity, more preferably, 90% to 95% identity and even more preferably 98% to 99% identity.

In determining whether or not two amino acid sequences fall within the defined percentage identity limits supra, those skilled in the art will be aware that it is possible to conduct a side-by-side comparison of the amino acid sequences. In such comparisons or alignments, differences will arise in the positioning of non-identical residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using software of the Computer Genetics Group, Inc., University Research Park, Maddison, Wis., United States of America, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximise the number of identical/similar residues and to minimise the number and/or length of sequence gaps in the alignment. Amino acid sequence alignments can also be performed using a variety of other commercially available sequence analysis programs, such as, for example, the BLAST program available at NCBI.

As used herein the term “NCBI” shall be taken to mean the database of the National Center for Biotechnology Information at the National Library of Medicine at the National Institutes of Health of the Government of the United States of America, Bethesda, Md., 20894.

In determining whether or not two nucleotide sequences fall within a particular percentage identity limitation recited herein, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more nucleotide sequences shall be taken to refer to the number of identical residues between said sequences as determined using any standard algorithm known to those skilled in the art. For example, nucleotide sequences may be aligned and their identity calculated using the BESTFIT program or other appropriate program of the Computer Genetics Group, Inc., University Research Park, Madison, Wis., United States of America (Devereaux et al., Nucl. Acids Res. 12, 387-395, 1984).

In a preferred embodiment, a marker associated with a bipolar affective disorder comprises, consists of or is located within a nucleic acid that comprises a nucleotide sequence at least about 80% identical to a sequence selected from the group consisting of:
(i) a sequence at least about 80% identical to a sequence selected from the group consisting of SEQ ID NO: 1, SEQ ID NO: 2 and SEQ ID NO: 4;
(ii) a sequence capable of encoding a polypeptide comprising an amino acid sequence at least 80% homologous to the sequence set forth in SEQ ID NO: 3 and SEQ ID NO: 5; and
(iii) a sequence complementary to a sequence set forth in (i) or (ii).

Preferably a marker associated with a bipolar affective disorder comprises a nucleic acid comprising a nucleotide sequence at least about 80% identical to at least about 20 contiguous nucleotides in a sequence selected from the group consisting of:
(i) a sequence selected from the group consisting of SEQ ID NO: 1, SEQ ID NO: 2 and SEQ ID NO: 4;
(ii) a sequence capable of encoding a polypeptide comprising an amino acid sequence at least 80% homologous to the sequence set forth in SEQ ID NO: 3 and SEQ ID NO: 5; and
(iii) a sequence complementary to a sequence set forth in (i) or (ii).

In a preferred embodiment, a marker that is associated with a bipolar affective disorder is a polymorphism of a FAT gene. As used herein, the term “polymorphism” shall be taken to mean a difference in the nucleotide sequence of the genome that occurs in a normal population of individuals. Such a polymorphism may induce changes in a mRNA and/or polypeptide encoded by the genomic DNA in which it occurs, eg. by changing an encoded amino acid or by altering the splicing or the frequency of splicing of an mRNA. Accordingly, a “polymorphism that is associated with a bipolar affective disorder” means that a particular form of the polymorphism is correlated with the development of a bipolar affective disorder in a subject, ie. in a population of subjects, those that suffer from a bipolar affective disorder are more likely to comprise the specific polymorphism that those subjects that do not suffer from a bipolar affective disorder.

As will be apparent to the skilled artisan a polymorphism that is associated with a bipolar affective disorder that occurs in a region of a FAT gene that is transcribed into mRNA or controls the transcription of mRNA (eg. controls splicing of mRNA) is detectable in an expression product of a FAT gene. Clearly the present invention encompasses a method of determining a bipolar affective disorder or a predisposition to a bipolar affective disorder comprising detecting a marker associated with a bipolar affective disorder in a FAT expression product, such as, for example, a pre-mRNA molecule, a 5′ capped mRNA, a polyadenylated mRNA, and/or a mature mRNA or cDNA derived therefrom. For example, the present inventors have demonstrated that a polymorphism in the FAT gene that is associated with a bipolar affective disorder is capable of inducing production of an altered splice form of the FAT gene. Accordingly, detection of the altered splice form in a sample from a subject is indicative of a bipolar affective disorder or a predisposition to a bipolar affective disorder.

Furthermore, a polymorphism that is associated with a bipolar affective disorder that occurs within a protein-coding region of a FAT gene may also cause a change in a polypeptide encoded by the FAT gene. Accordingly, the present invention also encompasses a method of determining a bipolar affective disorder or a predisposition to a bipolar affective disorder comprising detecting a marker associated with a bipolar affective disorder in a FAT polypeptide.

In a further particularly preferred embodiment, the marker comprises, consists of or is located within the 3′ region of a FAT genomic gene, or the corresponding region of an expression product thereof. Preferably, the 3′ region of the FAT genomic gene comprises or consists of the region spanning from nucleotide position 139,260 to nucleotide position 170,001 of SEQ ID NO: 1 (ie a nucleic acid that comprises of consists of the sequence set forth in SEQ ID NO: 6).

In another preferred embodiment, the marker comprises, consists of or is located within the region of the FAT genomic gene corresponding to the region spanning from nucleotide position 146,012 to nucleotide position 170,001 of SEQ ID NO: 1, or the corresponding region of an expression product thereof.

In a further preferred embodiment, the marker comprises, consists of or is located within the region of the FAT genomic gene corresponding to the region spanning from nucleotide position 148,108 to nucleotide position 170,001 of SEQ ID NO: 1, or the corresponding region of an expression product thereof.

In a still further preferred embodiment, the marker comprises, consists of or is located within the region of the FAT genomic gene corresponding to the region spanning from nucleotide position 148,199 to nucleotide position 170,001 of SEQ ID NO: 1, or the corresponding region of an expression product thereof.

In another preferred embodiment, the marker comprises, consists of or is located within the region of the FAT genomic gene corresponding to the region spanning from nucleotide position 148,333 to nucleotide position 170,001 of SEQ ID NO: 1, or the corresponding region of an expression product thereof.

In a preferred embodiment, the marker comprises, consists of or is located within the region of the FAT genomic gene or an expression product thereof that encodes one or more EVH1 domains of FAT. As used herein, the term EVH1 domain shall be taken to refer to the region of the FAT gene that comprises the amino acid sequence F/L PPPP. Accordingly, it is preferred that the marker comprises, consists of or is located within the region of the FAT genomic gene or an expression product thereof that encodes a polypeptide comprising an amino acid sequence from amino acid number 476 to amino acid number 608 of SEQ ID NO: 3.

In one embodiment, a marker associated with a bipolar affective disorder is a simple nucleotide polymorphism. As used herein, the term “simple nucleotide polymorphism” shall be taken to mean a polymorphism that comprises or consists of one or more small changes (i.e., an insertion, a deletion, a transition or a transversion) to the genome of a subject or an expression product thereof. For example, a simple nucleotide polymorphism comprises or consists of, a single nucleotide insertion or deletion, insertion or deletion of two, three or four or more nucleotides, transition of one or more nucleotides, or transversion of one or more nucleotides.

In a preferred embodiment, a marker that is associated with a bipolar affective disorder comprises or consists of a single nucleotide polymorphism (SNP). Methods of determining a SNP that is associated with a specific disorder are known in the art and/or described herein.

In a particularly preferred embodiment, a marker that is associated with a bipolar affective disorder comprises or consists of a SNP selected from the group consisting of a cytosine at a position corresponding to nucleotide 80,217 of SEQ ID NO: 1, a thymine at a position corresponding to nucleotide 130,625 of SEQ ID NO: 1, a thymine at a position corresponding to nucleotide 130,613 of SEQ ID NO: 1, a guanine at a position corresponding to nucleotide 139,968 of SEQ ID NO: 1, a thymine at a position corresponding to nucleotide 139,968 of SEQ ID NO: 1, a guanine at a position corresponding to nucleotide 142,199 of SEQ ID NO: 1, an adenine at a position corresponding to nucleotide 142,460 of SEQ ID NO: 1, a guanine at a position corresponding to nucleotide 145,782 of SEQ ID NO: 1, a guanine at a position corresponding to nucleotide 146,008 of SEQ ID NO: 1, a guanine at a position corresponding to nucleotide 146,012 of SEQ ID NO: 1, an adenine at a position corresponding to nucleotide 146,012 of SEQ ID NO: 1, a thymine at a position corresponding to nucleotide 148,108 of SEQ ID NO: 1, a cytosine at a position corresponding to nucleotide 148,108 of SEQ ID NO: 1, a thymine at a position corresponding to nucleotide 148,108 of SEQ ID NO: 1, an adenine at a position corresponding to nucleotide 148,199 of SEQ ID NO: 1, an adenine at a position corresponding to nucleotide 148,333 of SEQ ID NO: 1, a thymine at position 148,333 of SEQ ID NO: 1, a cytosine at a position corresponding to 148,333 of SEQ ID NO: 1, a cytosine at a position corresponding to nucleotide 151,403 of SEQ ID NO: 1 and a thymine at a position corresponding to nucleotide 153,127 of SEQ ID NO: 1.

In an even more particularly preferred embodiment, a marker associated with a bipolar affective disorder comprises or consists of a SNP selected from the group consisting of a guanine at a position corresponding to nucleotide 139,968 of SEQ ID NO: 1, a guanine at a position corresponding to nucleotide 146,012 of SEQ ID NO: 1, a thymine at a position corresponding to nucleotide 148,108 of SEQ ID NO: 1, a cytosine at a position corresponding to nucleotide 148,108 of SEQ ID NO: 1, an adenine at a position corresponding to nucleotide 148,333 of SEQ ID NO: 1 and a thymine at position 148,333 of SEQ ID NO: 1.

In an even more particularly preferred embodiment, a marker associated with a bipolar affective disorder comprises or consists of a SNP selected from the group consisting of a guanine at position 139,968 of SEQ ID NO: 1 (designated rs2637777), a guanine at position 146,012 of SEQ ID NO: 1 (designated rs2306990), a thymine at position 148,108 of SEQ ID NO: 1 (designated rs1298865), a cytosine at position 148,108 of SEQ ID NO: 1 (designated rs2306990), an adenine at position 148,333 of SEQ ID NO: 1 (designated rs2306987) and a thymidine at position 148,333 of SEQ ID NO: 1 (designated rs2306987).

In one embodiment, a marker associated with a bipolar affective disorder in a subject without a family history of psychiatric illness comprises or consists of a SNP selected from the group consisting of a guanine at a position corresponding to 139,968 of SEQ ID NO: 1, a guanine at a position corresponding to 146,012 of SEQ ID NO: 1, a thymine at a position corresponding to 148,108 of SEQ ID NO: 1 and a thymine at a position corresponding to 148,333 of SEQ ID NO: 1.

In another embodiment, a marker associated with a bipolar affective disorder in a subject with a family history of psychiatric illness comprises or consists of a SNP selected from the group consisting of a thymine at a position corresponding to 139,968 of SEQ ID NO: 1, an adenine at a position corresponding to 146,012 of SEQ ID NO: 1, a cytosine at a position corresponding to 148,108 of SEQ ID NO: 1 and a cytosine at a position corresponding to 148,333 of SEQ ID NO: 1.

In one embodiment, the marker is in its homozygous form.

A subject is considered to have a family history of psychiatric illness if a first, second, third or fourth, preferably a first or second degree relative suffers from a psychiatric illness. Preferably, the psychiatric illness is a bipolar affective disorder.

In one embodiment the method of determining the predisposition of a subject to bipolar affective disorder or diagnosing bipolar affective disorder comprises determining the presence or absence of the a marker within a FAT gene that is associated with a bipolar affective disorder in a test sample derived from a subject, wherein a presence or absence of the marker linked to chromosome 4q35.2 indicates that the subject being tested is predisposed to or suffers from a bipolar affective disorder.

Preferably, the marker that is present and indicates that the subject being tested is predisposed to or suffers from bipolar affective disorder comprises or consists of a nucleotide selected from the group consisting of a cytosine at position 80,217 of SEQ ID NO: 1 (designated rs172903), a cytosine at position 130,625 of SEQ ID NO: 1 (designated rs2249916), a cytosine at position 130,613 of SEQ ID NO: 1 (designated rs2249917), a guanine at position 139,968 of SEQ ID NO: 1 (designated rs2637777), a guanine at position 142,199 of SEQ ID NO: 1 (designated rs767168), an adenine at position 142,460 of SEQ ID NO: 1 (designated rs2289550), a guanine at position 145,782 of SEQ ID NO: 1 (designated rs1280097), a guanine at position 146,008 of SEQ ID NO: 1 (designated rs1280096), a guanine at position 146,012 of SEQ ID NO: 1 (designated rs2306990), a thymine at position 148,108 of SEQ ID NO: 1 (designated rs1298865), an adenine at position 148,199 of SEQ ID NO: 1 (designated rs2306988), an adenine at position 148,333 of SEQ ID NO: 1 (designated rs2306987), a cytosine at position 151,403 of SEQ ID NO: 1 (designated rs3775309) and a thymine at position 153,127 of SEQ ID NO: 1 (designated rs1973352).

In one embodiment, a marker associated with a bipolar affective disorder in a subject without a family history of psychiatric illness comprises or consists of a SNP selected from the group consisting of a guanine at position 139,968 of SEQ ID NO: 1 (designated rs2637777), a guanine at position 146,012 of SEQ ID NO: 1 (designated rs2306990), a thymine at position 148,108 of SEQ ID NO: 1 (designated rs1298865 and an adenine at position 148,333 of SEQ ID NO: 1 (designated rs2306987).

In another embodiment, a marker associated with a bipolar affective disorder in a subject with a family history of psychiatric illness comprises or consists of a SNP selected from the group consisting of a thymidine at position 139,968 of SEQ ID NO: 1 (designated rs2637777), an adenine at position 146,012 of SEQ ID NO: 1 (designated rs2306990), a cytosine at position 148,108 of SEQ ID NO: 1 (designated rs1298865 and a cytosine at position 148,333 of SEQ ID NO: 1 (designated rs2306987).

In another embodiment, the marker that is absent and indicates that the subject being tested is predisposed to or suffers from bipolar affective disorder comprises or consists of a nucleotide selected from the group consisting of a guanine at position 80,217 of SEQ ID NO: 1, a thymine at position 130,625 of SEQ ID NO: 1 (designated rs2249916), a thymine at position 130,613 of SEQ ID NO: 1 (designated rs2249917), a thymine at position 139,968 of SEQ ID NO: 1 (designated rs2637777), an adenine at position 142,199 of SEQ ID NO: 1 (designated rs767168), a guanine at position 142,460 of SEQ ID NO: 1 (designated rs2289550), a cytosine at position 145,782 of SEQ ID NO: 1 (designated rs1280097), an adenine at position 146,008 of SEQ ID NO: 1 (designated rs1280096), an adenine at position 146,012 of SEQ ID NO: 1 (designated rs2306990), a cytosine at position 148,108 of SEQ ID NO: 1 (designated rs1298865), a thymine at position 148,199 of SEQ ID NO: 1 (designated rs2306988), a thymine at position 148,333 of SEQ ID NO: 1 (designated rs2306987), an adenine at position 151,403 of SEQ ID NO: 1 (designated rs3775309) and an adenine at position 153,127 of SEQ ID NO: 1 (designated rs1973352).

Furthermore, the present invention is useful for determining a subject that will respond to treatment for a bipolar affective disorder. Such a method is useful as a subject that suffers from a psychiatric illness other than bipolar affective disorder that is mistakenly diagnosed with a bipolar affective disorder using the classification system currently in use may not respond to treatments designed for subjects that suffer from a bipolar affective disorder.

The present invention also encompasses a method of determining a subject that will respond to a particular treatment for bipolar affective disorder (i.e. pharmacogenomics). As will be apparent to the skilled artisan, a marker that is associated with a bipolar affective disorder is preferably detectable by standard procedures, for example by nucleic acid hybridisation or antibody binding. Accordingly, a nucleic acid marker is preferably at least about 8 nucleotides in length (for example, for detection using a locked nucleic acid (LNA) probe). To provide more specific hybridisation, a marker is preferably at least about 15 nucleotides in length or more preferably at least about 20 to 30 nucleotides in length. Such markers are particularly amenable to detection by nucleic acid hybridisation-based detection means assays, such as, for example any known format of PCR or ligase chain reaction.

Preferably, a protein marker encoded by nucleic acid that is associated with a bipolar affective disorder is suitable for antigen-based detection. As will be apparent to the skilled artisan, even a single amino acid change in a polypeptide is detectable in an antigen-based assay. However, for an antibody or ligand to detect such a change, a marker is preferably at least about 6 amino acids in length, more preferably at least about 8 to 10 amino acids in length, even more preferably at least about 14 amino acids in length. A protein marker may also be an entire protein, e.g. wherein the protein that is associated with a bipolar affective disorder is, for example, a conformation different to the protein in a normal or healthy individual.

Means for detecting the marker will be known to the skilled artisan and, in general, any nucleic acid-based or antigen-based detection means can be employed.

In one embodiment, the marker is detected by hybridising a nucleic acid probe or primer comprising the sequence of the marker to a marker linked to nucleic acid in a biological sample derived from a subject under at least moderate, or preferably high, stringency hybridisation conditions and detecting the hybridisation using a detection means, wherein hybridisation of the probe to the sample nucleic acid indicates that the subject being tested is predisposed to or suffers from a bipolar affective disorder. Preferably, the detection means is a nucleic acid hybridisation or amplification reaction.

In another embodiment, the marker is detected by hybridising a nucleic acid probe comprising the sequence of the marker to a nucleic acid that is linked to the marker in nucleic acid in a biological sample derived from a subject and detecting the hybridisation by a detection means, wherein hybridisation of the probe to the sample nucleic acid indicates that the subject being tested is predisposed to or suffers from a bipolar affective disorder. Preferably, the detection means is an amplification reaction, or a nucleic acid extension reaction.

For the purposes of defining the level of stringency to be used in these diagnostic assays, a low stringency is defined herein as being a hybridisation and/or a wash carried out in 6×SSC buffer, 0.1% (w/v) SDS at 28° C., or equivalent conditions. A moderate stringency is defined herein as being a hybridisation and/or washing carried out in 2×SSC buffer, 0.1% (w/v) SDS at a temperature in the range 45° C. to 65° C., or equivalent conditions. A high stringency is defined herein as being a hybridisation and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridisation solutions other than SSC known to those skilled in the art.

Generally, the stringency is increased by reducing the concentration of SSC buffer, and/or increasing the concentration of SDS and/or increasing the temperature of the hybridisation and/or wash. Those skilled in the art will be aware that the conditions for hybridisation and/or wash may vary depending upon the nature of the hybridisation matrix used to support the sample DNA, or the type of hybridisation probe used.

As will be apparent to the skilled artisan a probe or primer capable of specifically detecting a marker that is associated with a bipolar affective disorder is any probe or primer that is capable of specifically hybridising to the region of the genome that comprises said marker, or an expression product thereof. As used herein “specifically hybridises” means that the polynucleotide used as a probe is used under conditions where a target polynucleotide is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction are measured, for example, by radiolabelling the probe, e.g. with ³²P.

In one embodiment, a preferred probe or primer comprises, consists of or is within a nucleic acid comprising nucleotide sequence at least about 80% identical to a sequence selected from the group consisting of:
(i) a sequence at least about 80% identical to a sequence selected from the group consisting of SEQ ID NO: 1, SEQ ID NO: 2 and SEQ ID NO: 4;
(ii) a sequence capable of encoding an amino acid sequence at least 80% identical to the sequence set forth in SEQ ID NO: 3 or SEQ ID NO: 5; and
(iii) a sequence complementary to a sequence set forth in (i) or (ii).

Preferably, a probe or primer comprises a nucleic acid comprising a nucleotide sequence at least about 80% identical to a sequence of 20 contiguous nucleotides from a nucleotide sequence selected from the group consisting of:
(i) a sequence selected from the group consisting of SEQ ID NO: 1, SEQ ID NO: 2 and SEQ ID NO: 4;
(ii) a sequence capable of encoding an amino acid sequence at least 80% identical to the sequence set forth in SEQ ID NO: 3 or SEQ ID NO: 5; and
(iii) a sequence complementary to a sequence set forth in (i) or (ii).
   Probe/Primer Design

As will be apparent to the skilled artisan, the specific probe or primer used in an assay of the present invention will depend upon the assay format used. Clearly, a probe or primer that is capable of specifically hybridising to or detecting the marker of interest is preferred. Methods of designing probes and/or primers for, for example, PCR or hybridisation are known in the art and described, for example, in Dieffenbach and Dveksler (Eds) (In: PCR Primer: A Laboratory Manual, Cold Spring Harbour Laboratories, NY, 1995). Furthermore, several software packages are publicly available that design optimal probes and/or primers for a variety of assays, e.g. Primer 3 available from the Center for Genome Research, Cambridge, Mass., USA. Probes and/or primers useful for detection of a marker associated with bipolar affective disorder are assessed to determine those that do not form hairpins, self-prime or form primer dimers (e.g. with another probe or primer used in a detection assay). Furthermore, a probe or primer (or the sequence thereof) is assessed to determine the temperature at which it denatures from a target nucleic acid (i.e. the melting temperature of the probe or primer, or Tm). Methods of determining Tm are known in the art and described, for example, in Santa Lucia, Proc. Natl. Acad. Sci. USA, 95: 1460-1465, 1995 or Bresslauer et al., Proc. Natl. Acad. Sci. USA, 83: 3746-3750, 1986.

For example, a primer or probe useful for detecting a SNP in an allele specific PCR assay or a ligase chain reaction assay is designed such that the 3′ terminal nucleotide hybridises to the site of the SNP. The 3′ terminal nucleotide may be complementary to any of the nucleotides known to be present at the site of the SNP. When complementary nucleotides occur in both the probe or primer and at the site of the polymorphism the 3′ end of the probe or primer hybridises completely to the marker of interest and facilitates, for example, PCR amplification or ligation to another nucleic acid. Accordingly, a probe or primer that completely hybridises to the target nucleic acid produces a positive result in an assay.

A primer useful for a primer extension reaction is designed such that it specifically hybridises to a region adjacent to a specific nucleotide of interest, eg a SNP. While the specific hybridisation of a probe or primer may be estimated by determining the degree of homology of the probe or primer to any nucleic acid using software, such as, for example, BLAST, the specificity of a probe or primer can only be determined empirically using methods known in the art.

A locked nucleic acid (LNA) or protein-nucleic acid (PNA) probe or a molecular beacon useful for, for example, detection of a SNP or microsatellite by hybridisation is at least about 8 to 12 nucleotides in length. Preferably, the nucleic acid, or derivative thereof, that hybridises to the site of the SNP or microsatellite is positioned at approximately the centre of the probe, thereby facilitating selective hybridisation and accurate detection.

Methods of producing/synthesising probes and/or primers useful in the present invention are known in the art. For example, oligonucleotide synthesis is described, in Gait (Ed) (In: Oligonucleotide Synthesis: A Practical Approach, IRL Press, Oxford, 1984); LNA synthesis is described, for example, in Nielsen et al., J. Chem. Soc. Perkin Trans., 1: 3423, 1997; Singh and Wengel, Chem. Commun. 1247, 1998; and PNA synthesis is described, for example, in Egholm et al., Am. Chem. Soc., 114: 1895, 1992; Egholm et al., Nature, 365: 566, 1993; and Orum et al., Nucl. Acids Res., 21: 5332, 1993.

Methods of detecting a marker genetically linked to a region of the genome, such as, for example a microsatellite marker or a SNP are known in the art and/or described herein.

Nucleic Acid Detection Methods

Methods for detecting nucleic acids are known in the art and include for example, hybridisation based assays, amplification based assays and restriction endonuclease based assays. For example, a change in the sequence of a region of the genome or an expression product thereof, such as, for example, an insertion, a deletion, a transversion, a transition, alternative splicing or a change in the preference of or occurrence of a splice form of a gene is detected using a method, such as, polymerase chain reaction (PCR) strand displacement amplification, ligase chain reaction, cycling probe technology or a DNA microarray chip amongst others.

Methods of PCR are known in the art and described, for example, in Dieffenbach (ed) and Dveksler (ed) (In: PCR Primer: A Laboratory Manual, Cold Spring Harbour Laboratories, NY, 1995). Generally, for PCR two non-complementary nucleic acid primer molecules comprising at least about 20 nucleotides in length, and more preferably at least 30 nucleotides in length are hybridised to different strands of a nucleic acid template molecule, and specific nucleic acid molecule copies of the template are amplified enzymatically. PCR products may be detected using electrophoresis and detection with a detectable marker that binds nucleic acids. Alternatively, one or more of the oligonucleotides are labelled with a detectable marker (e.g. a fluorophore) and the amplification product detected using, for example, a lightcycler (Perkin Elmer, Wellesley, Mass., USA). Clearly, the present invention also encompasses quantitative forms of PCR, such as, for example, Taqman assays.

Strand displacement amplification (SDA) utilises oligonucleotides, a DNA polymerase and a restriction endonuclease to amplify a target sequence. The oligonucleotides are hybridised to a target nucleic acid and the polymerase used to produce a copy of this region. The duplexes of copied nucleic acid and target nucleic acid are then nicked with an endonuclease that specifically recognises a sequence at the beginning of the copied nucleic acid. The DNA polymerase recognises the nicked DNA and produces another copy of the target region at the same time displacing the previously generated nucleic acid. The advantage of SDA is that it occurs in an isothermal format, thereby facilitating high-throughput automated analysis.

Ligase chain reaction (described in EU 320,308 and U.S. Pat. No. 4,883,750) uses at least two oligonucleotides that bind to a target nucleic acid in such a way that they are adjacent. A ligase enzyme is then used to link the oligonucleotides. Using thermocycling the ligated oligonucleotides then become a target for further oligonucleotides. The ligated fragments are then detected, for example, using electrophoresis, or MALDI-TOF. Alternatively, or in addition, one or more of the probes is labelled with a detectable marker, thereby facilitating rapid detection.

Cycling Probe Technology uses chimeric synthetic probe that comprises DNA-RNA-DNA that is capable of hybridising to a target sequence. Upon hybridisation to a target sequence the RNA-DNA duplex formed is a target for RNase H thereby cleaving the probe. The cleaved probe is then detected using, for example, electrophoresis or MALDI-TOF.

In one embodiment, a marker that is associated with a bipolar affective disorder occurs within a protein coding region of a genomic gene (e.g. a FAT gene) and is detectable in mRNA encoded by that gene. For example, such a marker may be an alternate splice-form of a mRNA encoded by a genomic gene (eg. a splice form not observed in a subject that does not carry the polymorphism, or, alternatively, an increase or decrease in the level of a splice form in a subject that carries a polymorphism). Such a marker may be detected using, for example, reverse-transcriptase PCR (RT-PCR), transcription mediated amplification (TMA) or nucleic acid sequence based amplification (NASBA), although any mRNA or cDNA based hybridisation and/or amplification protocol is clearly amenable to the instant invention

Methods of RT-PCR are known in the art and described, for example, in Dieffenbach (ed) and Dveksler (ed) (In: PCR Primer: A Laboratory Manual, Cold Spring Harbour Laboratories, NY, 1995).

Methods of TMA or self-sustained sequence replication (3SR) use two or more oligonucleotides that flank a target sequence, a RNA polymerase, RNase H and a reverse transcriptase. One oligonucleotide (that also comprises a RNA polymerase binding site) hybridises to an RNA molecule that comprises the target sequence and the reverse transcriptase produces cDNA copy of this region. RNase H is used to digest the RNA in the RNA-DNA complex, and the second oligonucleotide used to produce a copy of the cDNA. The RNA polymerase is then used to produce a RNA copy of the cDNA, and the process repeated.

NASBA systems relies on the simultaneous activity of three enzymes (a reverse transcriptase, RNase H and RNA polymerase) to selectively amplify target mRNA sequences. The mRNA template is transcribed to cDNA by reverse transcription using an oligonucleotide that hybridises to the target sequence and comprises a RNA polymerase binding site at its 5′ end. The template RNA is digested with RNase H and double stranded DNA is synthesised. The RNA polymerase then produces multiple RNA copies of the cDNA and the process is repeated.

Clearly, the hybridisation to and/or amplification of a marker associated with a bipolar affective disorder using any of these methods is detectable using, for example, electrophoresis and/or mass spectrometry. In this regard, one or more of the probes/primers and/or one or more of the nucleotides used in an amplification reactions may be labelled with a detectable marker to facilitate rapid detection of a marker, for example, a fluorescent label (e.g. Cy5 or Cy3) or a radioisotope (e.g. ³²P).

Alternatively, amplification of a nucleic acid may be continuously monitored using a melting curve analysis method, such as that described in, for example, U.S. Pat. No. 6,174,670.

In a particularly preferred embodiment, a marker associated with a bipolar affective disorder comprises a single nucleotide polymorphism. As used herein, the term “single nucleotide polymorphism” shall be taken to mean that a specific nucleic acid in the genome of a subject or an expression product thereof (the SNP may also be transcribed) may be any of two possible nucleic acid bases, or any of three nucleic acid bases, or any of four nucleic acid bases. Within a population all (or both) forms of the polymorphism will be represented, however, it is preferable that individuals with a specific form of a polymorphism also develop and/or suffer from a bipolar effective disorder. In this regard, the individual may be homozygous or heterozygous for the specific polymorphism. Preferably, the subject is homozygous for one form of the SNP.

Additional methods for detecting SNPs are known in the art, and reviewed, for example, in Landegren et al., Genome Research 8: 769-776, 1998).

For example, a SNP that introduces or alters a sequence that is a recognition sequence for a restriction endonuclease is detected by digesting DNA with the endonuclease and detecting the fragment of interest using, for example, Southern blotting (described in Ausubel et al. (In: Current Protocols in Molecular Biology. Wiley Interscience, ISBN 047 150338, 1987) and Sambrook et al. (In: Molecular Cloning: Molecular Cloning: A Laboratory Manual, Cold Spring Harbor Laboratories, New York, Third Edition 2001)). Alternatively, a nucleic acid amplification method described supra, is used to amplify the region surrounding the SNP. The amplification product is then incubated with the endonuclease and any resulting fragments detected, for example, by electrophoresis, MALDI-TOF or PCR.

The direct analysis of the sequence of polymorphisms of the present invention can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)). For example, a region of the genomic FAT gene of a subject suspected of suffering from a bipolar affective disorder or at risk of suffering from a bipolar affective disorder is amplified using an amplification reaction, e.g., PCR. Following purification of the amplification of the amplification product, this is used in a sequencing reaction to determine the sequence of one or both alleles at the site of a SNP of interest.

Alternatively, a SNP is detected using single stranded conformational polymorphism (SSCP) analysis. SSCP analysis relies upon the formation of secondary structures in nucleic acids and the sequence dependent nature of these secondary structures. In one form of this analysis an amplification method, such as, for example, a method described supra, is used to amplify a nucleic acid that comprises a SNP. The amplified nucleic acids are then denatured, cooled and analysed using, for example, non-denaturing polyarcrylamide gel electrophoresis, mass spectrometry, or liquid chromatography (eg. HPLC or dHPLC). Regions that comprise different sequences form different secondary structures, and as a consequence migrate at different rates through, for example, a gel and/or a charged field. Clearly, a detectable marker may be incorporated into a probe/primer useful in SSCP analysis to facilitate rapid marker detection.

Alternatively, any nucleotide changes may be detected using, for example, mass spectrometry or capillary electrophoresis. For example, amplified products of a region of DNA comprising a SNP from a test sample are mixed with amplified products from a normal/healthy individual. The products are denatured and allowed to reanneal. Those samples that comprise a different nucleotide at the position of the SNP will not completely anneal to a nucleic acid molecule from a normal/healthy individual thereby changing the charge and/or conformation of the nucleic acid, when compared to a completely annealed nucleic acid. Such incorrect base pairing is detectable using, for example, mass spectrometry.

As exemplified herein, mass spectrometry is also useful for detecting the molecular weight of a short amplified product, wherein a nucleotide change causes a change in molecular weight of the nucleic acid molecule (such a method is also described, for example, in U.S. Pat. No. 6,574,700).

Allele specific PCR (as described, for example, In Liu et al., Genome Research, 7: 389-398, 1997) is also useful for determining the presence of one or other allele of a SNP. An oligonucleotide is designed, in which the most 3′ base of the oligonucleotide hybridizes to a specific form of the SNP of interest (i.e., allele). During a PCR reaction, if the 3′ end of the oligonucleotide does not hybridise to a target sequence, little or no PCR product is produced, indicating that a base other than that present in the oligonucleotide is present at the site of SNP in the sample. PCR products are then detected using, for example, gel or capillary electrophoresis or mass spectrometry.

Primer extension methods (described, for example, in Dieffenbach (ed) and Dveksler (ed) (In: PCR Primer: A Laboratory Manual, Cold Spring Harbour Laboratories, NY, 1995)) are also useful for the detection of a SNP. An oligonucleotide that hybridises to the region of a nucleic acid adjacent to the SNP. This oligonucleotide is then used in a primer extension protocol with a polymerase and a free nucleotide diphosphate that corresponds to either or any of the possible bases that occur at the SNP. Preferably the nucleotide-diphosphate is labelled with a detectable marker (e.g. a fluorophore). Following primer extension, unbound labelled nucleotide diphosphates are removed, e.g. using size exclusion chromatography or electrophoresis, or hydrolized, using for example, alkaline phosphatase, and the incorporation of the labelled nucleotide into the oligonucleotide is detected, indicating the base that is present at the site of the SNP. Alternatively, or in addition, as exemplified herein primer extension products are detected using mass spectrometry (eg. MALDI-TOF).

Clearly, the present invention extends to high-throughput forms of primer extension analysis, such as, for example, minisequencing (Sy Vämen et al., Genomics 9: 341-342, 1995). In such a method, a probe or primer (or multiple probes or primers) are immbolized on a solid support (e.g. a glass slide). A biological sample comprising nucleic acid is then brought into direct contact with the probe/s or primer/s, and a primer extension protocol performed with each of the free nucleotide bases labelled with a different detectable marker. The nucleotide present at a SNP or a number of SNPs is then determined by determining the detectable marker bound to each probe and/or primer.

Fluorescently labelled locked nucleic acid (LNA) molecules or fluorescently labelled protein-nucleic acid (PNA) molecules are useful for the detection of SNPs (as described in Simeonov and Nikiforov, Nucleic Acids Research, 30(17): 1-5, 2002). LNA and PNA molecules bind, with high affinity, to nucleic acid, in particular, DNA. Fluorophores (in particular, rhodomine or hexachlorofluorescein) conjugated to the LNA or PNA probe fluoresce at a significantly greater level upon hybridisation of the probe to target nucleic acid compared to a probe that has not hybridized to a target nucleic acid. However, the level of increase of fluorescence is not enhanced to the same level when even a single nucleotide mismatch occurs. Accordingly, the degree of fluorescence detected in a sample is indicative of the presence of a mismatch between the LNA or PNA probe and the target nucleic acid, such as, in the presence of a SNP. Preferably, fluorescently labelled LNA or PNA technology is used to detect a single base change in a nucleic acid that has been previously amplified using, for example, an amplification method described supra.

As will be apparent to the skilled artisan, LNA or PNA detection technology is amenable to a high-throughput detection of one or more markers immobilising an LNA or PNA probe to a solid support, as described in Orum et al., Clin. Chem. 45: 1898-1905, 1999.

Similarly, Molecular Beacons are useful for detecting SNPs directly in a sample or in an amplified product (see, for example, Mhlang and Malmberg, Methods 25: 463-471, 2001). Molecular beacons are single stranded nucleic acid molecules with a stem-and-loop structure. The loop structure is complementary to the region surrounding the SNP of interest. The stem structure is formed by annealing two “arms” complementary to each other, that are on either side of the probe (loop). A fluorescent moiety is bound to one arm and a quenching moiety that suppresses any detectable fluorescence when the molecular beacon is not bound to a target sequence bound to the other arm. Upon binding of the loop region to its target nucleic acid the arms are separated and fluorescence is detectable. However, even a single base mismatch significantly alters the level of fluorescence detected in a sample. Accordingly, the presence or absence of a particular base at the site of a SNP is determined by the level of fluorescence detected.

A single nucleotide polymorphism can also be identified by hybridisation to nucleic acid arrays, an example of which are described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

Clearly, the present invention encompasses other methods of detecting a SNP that is within a FAT gene and associated with a bipolar affective disorder, such as, for example, SNP microarrays (available from Affymetrix, or described, for example, in U.S. Pat. No. 6,468,743 or Hacia et al., Nature Genetics, 14: 441, 1996), Taqman assays (as described in Livak et al., Nature Genetics, 9: 341-342, 1995), solid phase minisequencing (as described in Syvamen et al., Genomics, 13: 1008-1017, 1992), minisequencing with FRET (as described in Chen and Kwok, Nucleic Acids Res. 25: 347-353, 1997) or pyrominisequencing (as reviewed in Landegren et al., Genome Res., 8(8): 769-776, 1998).

Protein Detection Methods

In another embodiment of the invention, the marker is a peptide, polypeptide or protein encoded by a nucleic acid that is within a FAT gene and associated with a bipolar affective disorder. In accordance with this embodiment, the marker is detected by contacting a biological sample derived from a subject with an antibody or ligand that specifically binds to said marker for a time and under conditions sufficient for an antibody/ligand-antigen complex to form and then detecting the complex wherein detection of the complex indicates that the subject being tested is predisposed to or suffers from a bipolar affective disorder.

As used herein the term “ligand” shall be taken in its broadest context to include any chemical compound, polynucleotide, peptide, protein, lipid, carbohydrate, small molecule, natural product, polymer, etc. that is capable of selectively binding, whether covalently or not, to one or more specific sites on a FAT polypeptide. The ligand may bind to its target via any means including hydrophobic interactions, hydrogen bonding, electrostatic interactions, van der Waals interactions, pi stacking, covalent bonding, or magnetic interactions amongst others. It is particularly preferred that a ligand is able to specifically bind to a specific form of a FAT polypeptide (e.g. a FAT polypeptide that comprises a marker that is linked with bipolar affective disorder).

As used herein, the term “antibody” refers to intact monoclonal or polyclonal antibodies, immunoglobulin (IgA, IgD, IgG, IgM, IgE) fractions, humanized antibodies, or recombinant single chain antibodies, as well as fragments thereof, such as, for example Fab, F(ab)2, and Fv fragments.

Clearly an antibody or ligand that is specifically capable of differentiating between a polypeptide or protein marker that is associated with a bipolar affective disorder and a peptide, polypeptide or protein that is not associated with a bipolar affective disorder is preferable. For example, the antibody or ligand may detect a single amino acid change in a peptide, polypeptide or protein (i.e. compared to a normal and/or healthy individual), or alternatively an insertion or deletion of two or more amino acids (compared to a normal and/or healthy individual). Antibodies and ligands that specifically bind to such alternate forms of a FAT polypeptide are clearly encompassed by the present invention.

Antibodies capable of specifically binding a FAT polypeptide, particularly a FAT peptide, polypeptide or protein that is a marker associated with a bipolar affective disorder are produced using methods known in the art. For example, a monoclonal antibody may be prepared using a hybridoma method (essentially as described by Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., (1981) pp. 563-681). In general, such procedures involve immunizing an animal (preferably a mouse) with a FAT polypeptide antigen or a FAT polypeptide-expressing cell. Splenocytes of such immunized mice are extracted and fused with a suitable myeloma cell line. Any suitable myeloma cell line may be employed in accordance with the present invention, e.g. the SP20 cell line. Following fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones that secrete antibodies capable of binding a form of a FAT polypeptide of interest and/or a fragment thereof.

Alternatively, a monoclonal antibody capable of binding to a form of a FAT polypeptide of interest or a fragment thereof is produced using a method such as, for example, a human B-cell hybridoma technique (Kozbar et al., Immunol. Today 4:72, 1983), a EBV-hybridoma technique to produce human monoclonal antibodies (Cole et al. Monoclonal Antibodies in Cancer Therapy, 1985 Allen R. Bliss, Inc., pages 77-96), or screening of combinatorial antibody libraries (Huse et al., Science 246: 1275, 1989).

A polyclonal or monoclonal antibody may be produced using a method essentially as described by Harlow and Lane (In: Antibodies: A Laboratory Manual, Cold Spring Harbor Laboratory, 1988). In one such technique, an immunogen comprising a FAT polypeptide or a fragment thereof is injected into any one of a variety of animals (e.g. a mouse, a rat, a rabbit, a sheep, a pig, a chicken or a goat). The immunogen is derived from a natural source, produced by recombinant expression means, or artificially generated, such as by chemical synthesis (e.g., BOC chemistry or FMOC chemistry). In this regard, a FAT polypeptide or a fragment thereof may serve as the immunogen without modification. Alternatively, a FAT polypeptide or a fragment thereof is joined to a carrier protein, such as, for example bovine serum albumin. The immunogen and optionally a carrier for the protein is injected into the animal host, preferably according to a predetermined schedule incorporating one or more booster immunizations, and blood collected from said the animals periodically. Optionally, the immunogen is injected in the presence of an adjuvant, such as, for example Freund's complete or incomplete adjuvant to enhance the immune response to the immunogen. Monoclonal or polyclonal antibodies specific for the polypeptide may then be purified from the blood isolated form an animal by, for example, affinity chromatography using the polypeptide coupled to a suitable solid support.

Methods for detecting a protein in a biological sample are known in the art and/or described herein.

For example, a standard solid-phase ELISA or FLISA format is particularly useful in determining the presence of a protein in a variety of samples.

In one form such an assay involves immobilising a biological sample onto a solid matrix, such as, for example a polystyrene or polycarbonate microwell or dipstick, a membrane, or a glass support (e.g. a glass slide). An antibody that specifically binds to a marker associated with a bipolar affective disorder is brought into direct contact with the immobilised biological sample, and forms a direct bond with any of its target protein present in said sample. This antibody is generally labelled with a detectable reporter molecule, such as, for example, a fluorescent label (e.g. FITC or Texas Red), a fluorescent semiconductor nanocrystal (as described in U.S. Pat. No. 6,306,610) in the case of a FLISA or an enzyme (e.g. horseradish peroxidase (HRP), alkaline phosphatase (AP) or β-galactosidase) in the case of an ELISA, or alternatively a second labelled antibody can be used that binds to the first antibody. Following washing to remove any unbound antibody the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal) in the case of an enzymatic label.

In another form, an ELISA or FLISA consists of immobilizing an antibody or ligand that specifically binds to a marker associated with a bipolar affective disorder on a solid matrix, such as, for example, a membrane, a polystyrene or polycarbonate microwell, a polystyrene or polycarbonate dipstick or a glass support. A sample is then brought into physical relation with said antibody, and said the marker associated with a bipolar affective disorder is bound or ‘captured’. The bound protein is then detected using a labelled secondary antibody, for example, an anti-human FAT antibody for the detection of human FAT. Alternatively, a third labelled antibody can be used that binds the second (detecting) antibody.

It will be apparent to the skilled person that the assay formats described herein are amenable to high throughput formats, such as, for example automation of screening processes, or a microarray format as described in Mendoza et al., Biotechniques 27(4): 778-788, 1999. Furthermore, variations of the above-described assay will be apparent to those skilled in the art, such as, for example, a competitive ELISA.

In another embodiment, Western blotting is used to determine the presence of a marker associated with a bipolar affective disorder in a sample. In such an assay, protein from a sample is separated using sodium doedecyl sulphate polyacrylamide gel electrophoresis (SDS-PAGE) using techniques well known in the art and described in, for example, Scopes (In: Protein Purification: Principles and Practice, Third Edition, Springer Verlag, 1994). Separated proteins are then transferred to a solid support, such as, for example, a membrane (e.g., a PVDF membrane), using methods known in the art, for example, electrotransfer. This membrane is then blocked and probed with a labeled antibody or ligand that specifically binds to the marker associated with a bipolar affective disorder. Alternatively, a labeled secondary, or even tertiary, antibody or ligand is used to detect the binding of a specific primary antibody. The presence of the label is then determined using an assay appropriate for the label used. An appropriate assay to detect the label will be apparent to the skilled artisan.

In another embodiment, a marker associated with a bipolar affective disorder is detected using immunohistochemistry or immunofluorescence. For example, a cell or tissue section that is to be analysed to determine the presence of a marker associated with a bipolar affective disorder is fixed to stabilize and protect both the cell and the proteins contained within the cell. Preferably, the method of fixation does not disrupt or destroy the antigenicity of the marker of bipolar affective disorder, thus rendering it undetectable. Methods of fixing a cell are known in the art and include for example, treatment with paraformaldehyde, treatment with alcohol, treatment with acetone, treatment with methanol, treatment with Bouin's fixative and treatment with glutaraldehyde. Following fixation a cell is incubated with a ligand or antibody capable of binding the marker associated with a bipolar affective disorder. The ligand or antibody may be labeled with a detectable marker, such as, for example, a fluorescent label (e.g. FITC or Texas Red), a fluorescent semiconductor nanocrystal (as described in U.S. Pat. No. 6,306,610) or an enzyme (e.g. horseradish peroxidase (HRP)), alkaline phosphatase (AP) or β-galactosidase. Alternatively, a second labeled antibody that binds to the first antibody can be used to detect the first antibody. Following washing to remove any unbound antibody, the marker of bipolar affective disorder bound to said labeled antibody is detected using the relevant detection means. Means of detecting a detectable marker will vary depending upon the type of label used and will be apparent to the skilled artisan.

Optionally, a method of detecting a marker associated with a bipolar affective disorder using immunofluorescence or immunohistochemistry will comprise additional steps such as, for example, cell permeabilization (using, for example, n-octyl-BD-glucopyranoside, deoxycholate, a non-ionic detergent such as Triton X-100 NP-40, low concentrations of ionic detergents, such as, for example SDS or saponin) and/or antigen retrieval (using, for example, heat).

Alternatively, a marker associated with a bipolar affective disorder is detected using a method such as, for example, mass spectrometry, matrix-assisted laser desorption/ionization time of flight (MALDI-TOF), electrospray ionisation (ESI), protein chip, biosensor technology, or fluorescence resonance energy transfer, is clearly contemplated in the present invention.

For example, biosensor devices generally employ an electrode surface in combination with current or impedance measuring elements to be integrated into a device in combination with the assay substrate (such as that described in U.S. Pat. No. 5,567,301). An antibody that specifically binds to a marker associated with a bipolar affective disorder is preferably incorporated onto the surface of a biosensor device and a biological sample contacted to said device. A change in the detected current or impedance by the biosensor device indicates protein binding to said antibody. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several proteins or peptides in a small amount of body fluids, such as, for example, biological samples obtained for prenatal testing.

Clearly other forms of biosensor are encompasses by the present invention, such as, for example, evanescent biosensors, microcantilever biosensors (as described in for example USSN 20030010097) or membrane biosensors as described in, for example, U.S. Pat. No. 5,234,566.

Biological Samples

In one embodiment, the biological sample is any sample that comprises a nucleated cell. More preferably, the biological sample comprises a cell that comprises a marker within a FAT gene or an expression product thereof that is associated with a bipolar affective disorder. Accordingly, preferred biological samples are samples derived from a human. Preferably, the biological sample is easily isolated, e.g. a sample selected from the group consisting of whole blood, serum, plasma, peripheral blood mononuclear cells (PBMC), a buffy coat fraction, saliva, semen, tears, sweat, hair, urine, a buccal cell and a skin cell.

Such samples are particularly preferred for use in assays that detect a marker within a FAT gene that is associated with a bipolar affective disorder that is detectable in genomic DNA.

In another embodiment, a biological sample comprises a cell or cell extract or mixture thereof derived from a tissue selected from the group consisting of a brain, a spinal cord, skin, a lung, a kidney and a pancreas.

Such samples are known to express the FAT gene at various stages of development i.e. while an embryo is developing. In particular tissue selected from the group consisting of a brain, a spinal cord, skin, a lung, a kidney and a pancreas express FAT during embryo development. Accordingly, a cell isolated from such a tissue are particularly useful in detecting a marker associated with a bipolar affective disorder in the developing embryo, ie in utero or prenatal testing.

Methods of isolating such a sample are known in the art and include, for example amniocentesis, chorionic villus sampling, fetal blood sampling (e.g. cordocentesis or percutaneous umbilical blood sampling) and other fetal tissue sampling (e.g., fetal skin biopsy).

As will be apparent from the preceding description, a method of detecting a marker associated with a bipolar affective disorder in an expression product of a FAT gene are particularly amenable to prenatal testing for a predisposition to a bipolar affective disorder.

In contrast, a method of detecting a marker associated with a bipolar affective disorder in genomic DNA is amenable to an assay for determining a bipolar affective disorder or a predisposition to bipolar affective disorder at any stage (i.e. pre-natal, post-natal and/or post-mortem).

Preferably, a biological sample has been isolated or derived previously from a subject by, for example, surgery, or using a syringe or swab.

Cell preparations or nucleic acid preparation derived from such tissues or cells are not to be excluded. The sample can be prepared on a solid matrix for histological analyses, or alternatively, in a suitable solution such as, for example, an extraction buffer or suspension buffer, and the present invention clearly extends to the testing of biological solutions thus prepared.

In one embodiment, a biological sample is derived or isolated from a subject suspected of suffering from a bipolar affective disorder. Such a subject may have been assessed by another method, such as, for example, by clinical assessment.

In another embodiment, a biological sample is derived or isolated from a subject at risk of developing a bipolar affective disorder, such as, for example, a subject with a family history of a bipolar affective disorder.

As will be apparent to the skilled artisan, detecting a marker that is associated with a bipolar affective disorder may also involve the step of detecting said marker (or another form of said marker) in a suitable control sample, eg. a normal individual or a typical population.

As used herein, the term “normal individual” shall be taken to mean that the subject is selected on the basis that they do not comprise or express a marker that is linked to bipolar affective disorder, nor do they suffer from a bipolar affective disorder. In particular said normal individual does not comprise or express a marker within a FAT gene that is associated with a bipolar affective disorder.

Furthermore, the normal subject has not been diagnosed with any form of bipolar affective disorder using the International Classification of Diseases of the World Health Organisation (10th Edition) and the Diagnostic and Statistic Manual (4th Edition). Preferably, said individual has not suffered from a major depressive or manic episode as defined in the International Classification of Diseases of the World Health Organisation (10th Edition) and the Diagnostic and Statistic Manual (4th Edition).

Alternatively, or in addition, a suitable control sample is a control data set comprising measurements of the marker being assayed for a typical population of subjects known not to suffer from a bipolar affective disorder.

In the present context, the term “typical population” with respect to subjects known not to suffer from a bipolar affective disorder and/or comprise or express a marker of a bipolar affective disorder shall be taken to refer to a population or sample of subjects tested using, for example, the International Classification of Diseases of the World Health Organisation (10th Edition) and the Diagnostic and Statistic Manual (4th Edition) and determined not to suffer from a bipolar affective disorder and/or tested to determine the presence or absence of a marker of bipolar affective disorder, wherein said subjects are representative of the spectrum of healthy subjects or subjects known not to suffer from bipolar affective disorder.

Given that bipolar affective disorder is a quantitative trait, a subject may suffer from bipolar affective disorder and not comprise or express a marker of bipolar affective disorder described herein. Alternatively, a subject may not suffer from a bipolar affective disorder, yet comprise or express a marker of bipolar affective disorder as described herein. However, a suitable control sample for the instant invention is a sample derived from a subject that does not suffer from a bipolar affective disorder and does not comprise or express a marker of a bipolar affective disorder.

In one embodiment, the method is performed using genomic DNA derived from a biological sample. In another embodiment, the method is performed using mRNA or cDNA derived from the biological sample.

Methods of Detecting a Marker Linked to Map Position 4q35.2 of the Human Genome

While determining a marker associated with a bipolar affective disorder, the present inventors found that the region of the genome linked to map position 4q35.2 of the human genome comprises a susceptibility locus for a bipolar affective disorder. Accordingly, another aspect of the present invention provides a method for determining a bipolar affective disorder or a predisposition to a bipolar affective disorder, said method comprising detecting a marker for a bipolar affective disorder which marker is linked to map position 4q35.2 of the human genome in a sample derived from a subject, wherein the detection is indicative of a bipolar affective disorder of a predisposition to a bipolar affective disorder in the subject.

In one embodiment, the marker linked to map position 4q35.2 is located between or comprises the microsatellite markers designated D4S1164 (SEQ ID NO: 21) and D4S1192 (SEQ ID NO: 27). Preferably, the marker linked to map position 4q35.2 is located between or comprises the microsatellite markers designated D4S910 (SEQ ID NO: 22) and D4S1374 (SEQ ID NO: 28). More preferably, the marker linked to map position 4q35.2 is located between or comprises the microsatellite markers designated D4S3173 (SEQ ID NO: 23) and D4S1375 (SEQ ID NO: 29). Even more preferably, the marker linked to map position 4q35.2 is located between or comprises the microsatellite markers designated D4S3236 (SEQ ID NO: 24) and D4S3051 (SEQ ID NO: 30). Still more preferably, the marker linked to map position 4q35.2 is located between or comprises the microsatellite markers designated D4S2827 (SEQ ID NO: 25) and D4S2643 (SEQ ID NO: 31)

As used herein, the term “map position 4q35.2 of the human genome” shall be taken to refer to the region of the human genome that comprises a genomic gene encoding a FAT gene. As will be apparent to those skilled in the art a “genomic gene” includes both protein-encoding regions (i.e. exons) and non-coding regions (i.e. 5′-upstream regulatory regions such as the promoter and 5′-untranslated region, intervening sequences or introns, and 3′-untranslated region). Accordingly, a genomic gene encoding FAT protein includes all such features and not merely the protein-encoding portion thereof.

In one embodiment, the marker linked to map position 4q35.2 comprises or consists of a microsatellite marker. Preferably, the microsatellite marker is selected from the group consisting of D4S1164 (SEQ ID NO: 21), D4S1192 (SEQ ID NO: 27), D4S910 (SEQ ID NO: 22), D4S1374 (SEQ ID NO: 28), D4S3173 (SEQ ID NO: 23), D4S1375 (SEQ ID NO: 29), D4S3236 (SEQ ID NO: 24), D4S3051 (SEQ ID NO: 30), D4S2827 (SEQ ID NO: 25) and D4S2643 (SEQ ID NO: 31).

In a preferred embodiment, the marker linked to map position 4q35.2 comprises, consists of or is within a nucleotide sequence at least about 80% identical to a sequence selected from the group consisting of:
(i) a sequence at least about 80% identical to a sequence selected from the group consisting of SEQ ID NO: 1, SEQ ID NO: 2 and SEQ ID NO: 4;
(ii) a sequence capable of encoding a polypeptide comprising an amino acid sequence at least 80% identical to the sequence set forth in SEQ ID NO: 3 and SEQ ID NO: 5; and
(iii) a sequence complementary to a sequence set forth in (i) or (ii).

In another preferred embodiment, the marker comprises, consists of or is located within the 3′ region of a FAT genomic gene, or the corresponding region of an expression product thereof. Preferably, the 3′ region of the FAT genomic gene comprises or consists of the region spanning from nucleotide position 139,260 to nucleotide position 170,001 of SEQ ID NO: 1 (ie a nucleic acid that comprises of consists of the sequence set forth in SEQ ID NO: 6).

In another preferred embodiment, the marker comprises, consists of or is located within the region of the FAT genomic gene corresponding to the region spanning from nucleotide position 146,012 to nucleotide position 170,001 of SEQ ID NO: 1, or the corresponding region of an expression product thereof.

In a further preferred embodiment, the marker comprises, consists of or is located within the region of the FAT genomic gene corresponding to the region spanning from nucleotide position 148,108 to nucleotide position 170,001 of SEQ ID NO: 1, or the corresponding region of an expression product thereof.

In a still further preferred embodiment, the marker comprises, consists of or is located within the region of the FAT genomic gene corresponding to the region spanning from nucleotide position 148,199 to nucleotide position 170,001 of SEQ ID NO: 1, or the corresponding region of an expression product thereof.

In another preferred embodiment, the marker comprises, consists of or is located within the region of the FAT genomic gene corresponding to the region spanning from nucleotide position 148,333 to nucleotide position 170,001 of SEQ ID NO: 1, or the corresponding region of an expression product thereof.

In a preferred embodiment, a marker that is associated with a bipolar affective disorder comprises or consists of a single nucleotide polymorphism (SNP). Methods of determining a SNP that is associated with a specific disorder are known in the art and/or described herein.

In a particularly preferred embodiment, a marker for a bipolar affective disorder which marker is linked to map position 4q35.2 of the human genome comprises or consists of a SNP. Preferably, the SNP is selected from the group consisting of a cytosine at a position corresponding to nucleotide 80,217 of SEQ ID NO: 1, a thymine at a position corresponding to nucleotide 130,625 of SEQ ID NO: 1, a thymine at a position corresponding to nucleotide 130,613 of SEQ ID NO: 1, a guanine at a position corresponding to nucleotide 139,968 of SEQ ID NO: 1, a thymine at a position corresponding to nucleotide 139,968 of SEQ ID NO: 1, a guanine at a position corresponding to nucleotide 142,199 of SEQ ID NO: 1, an adenine at a position corresponding to nucleotide 142,460 of SEQ ID NO: 1, a guanine at a position corresponding to nucleotide 145,782 of SEQ ID NO: 1, a guanine at a position corresponding to nucleotide 146,008 of SEQ ID NO: 1, a guanine at a position corresponding to nucleotide 146,012 of SEQ ID NO: 1, an adenine at a position corresponding to nucleotide 146,012 of SEQ ID NO: 1, a thymine at a position corresponding to nucleotide 148,108 of SEQ ID NO: 1, a cytosine at a position corresponding to nucleotide 148,108 of SEQ ID NO: 1, a thymine at a position corresponding to nucleotide 148,108 of SEQ ID NO: 1, an adenine at a position corresponding to nucleotide 148,199 of SEQ ID NO: 1, an adenine at a position corresponding to nucleotide 148,333 of SEQ ID NO: 1, a thymine at position 148,333 of SEQ ID NO: 1, a cytosine at a position corresponding to 148,333 of SEQ ID NO: 1, a cytosine at a position corresponding to nucleotide 151,403 of SEQ ID NO: 1 and a thymine at a position corresponding to nucleotide 153,127 of SEQ ID NO: 1.

As will be apparent to the skilled artisan, a marker that is linked to map position 4q35.2 of the human genome should be detectable using standard procedures, for example by nucleic acid hybridisation or antibody binding. Accordingly, a nucleic acid marker is preferably at least about 8 nucleotides in length (for example, for detection using a LNA probe). To provide more specific hybridisation a marker is preferably at least about 15 nucleotides in length or more preferably at least 20 to 30 nucleotides in length. Such markers are particularly amenable to detection by nucleic acid hybridisation-based assays, such as, for example, any known format of PCR or ligase chain reaction. A marker that is a microsatellite is at least about 60 nucleotides in length, preferably at least about 80 nucleotides in length, more preferably at least about 100 nucleotides in length. However, it will be apparent to the skilled artisan that such a marker is detectable with one or more probes of shorter length (e.g. at least about 8 to about 20 nucleotides in length, more preferably, about 12 to about 20 nucleotides in length, and, even more preferably, about 15 to about 20 nucleotides in length).

Preferably a protein marker is encoded by nucleic acid that is linked to map position 4q35.2 is suitable for antigen-based detection. As will be apparent to the skilled artisan, even a single amino acid change in a polypeptide is detectable in an antigen-based assay. However, for an antibody or ligand to detect such a change, a marker is preferably at least about 6 amino acids in length, more preferably at least about 8 to 10 amino acids in length, even more preferably at least about 14 amino acids in length. A protein marker may also be an entire protein, e.g. wherein the protein that is associated with a bipolar affective disorder is, for example, a conformation different to the protein in a normal or healthy individual.

Methods of detecting nucleic acid and proteinaceous markers associated with a bipolar affective disorder described supra shall be taken to apply mutatis mutandis to methods of detecting a marker linked to map position 4q35.2 of the human genome.

Clearly the present invention encompasses the use of a multiplex assay to determine the predisposition of a subject to a bipolar affective disorder or to diagnose a bipolar affective disorder. In this regard, such a multiplexed assay may detect two or more nucleic acid markers that are associated with bipolar affective disorder and/or linked to map position 4q35.2 of the human genome. Alternatively, or in addition, a multiplexed assay may detect two or more peptide, polypeptide or protein markers that are associated with bipolar affective disorder and or encoded by nucleic acid linked to map position 4q35.2 of the human genome. The combination of nucleic acid-based and antigen-based detection methods is also contemplated by the invention.

As a further alternative, a method of the present invention for determining a bipolar affective disorder or a predisposition to a bipolar affective disorder is multiplexed with an assay that detects and allele or polymorphism of another gene or expression product thereof associated with a bipolar affective disorder. For example, a method of the present invention is multiplexed with an assay that detects an allele, polymorphism or mutation of a gene (or an expression product thereof) selected from the group consisting of a tyrosine hydroxylase as described in Leboyer et al., Lancet, 335: 932, 1990), a serotonin transporter (5-HTT; hSERT) (as reported in Collier et al., Neuroreport, 7: 1675-1679, 1996 and Kirov et al., Psychol. Med., 29: 1249-1254, 1999), a catechol-o-methyl transferase (COMT) (as described, for example, in Lachman et al., Psych. Genet. 7: 13-17, 1997), a dopamine DRD3 receptor, a serotonin 5HT2A receptor (as described, for example, in Massat et al., Am. J. Hum. Genet. 96: 136-140, 2000) and a monoamine oxidase A (MAOA) (Furlong et al., Am. J. Med. Genet 88: 398-406, 1999).

Methods of Determining a Marker Associated with a Bipolar Affective Disorder

Given the tight association of the human FAT gene to a bipolar affective disorder, and the provision of numerous markers, the present invention further provides methods for identifying new markers for a bipolar affective disorder.

Another aspect of the present invention provides a method for identifying a marker that is associated with a bipolar affective disorder, said method comprising:
(i) identifying a polymorphism or allele within a FAT gene or an expression product thereof;
(ii) analyzing a panel of subjects to determine those that suffer from a bipolar affective disorder, wherein not all members of the panel comprise the polymorphism or allele; and
(iii) determining the variation in the development of a bipolar affective disorder wherein said variation indicates that the polymorphism or allele is associated with a subject's predisposition to a bipolar affective disorder.

Methods for determining the association between a marker and a disease, disorder and/or a phenotype are known in the art and reviewed, for example, in King (Ed) Rotter (Ed) and Motulski (Ed), The Genetic Basis of Common Disease, Oxford University Press, 2nd Edition, ISBN 0195125827, and Miller and Cronin (Eds), Genetic Polymorphisms and Susceptibility to Disease, Taylor and Francis, 1st Edition, ISBN 0748408223.

Generally, determining an association between a marker (eg a polymorphism and/or allele) and a disease, disorder or phenotype involves comparing the frequency of a polymorphism or allele at a specific locus between a sample of unrelated affected individuals (ie they comprise the phenotype of interest and/or suffer from the disease/disorder of interest) and an appropriate control that is representative of the allelic distribution in the normal population. Accordingly, association studies are performed at the population level rather than within families, as is the case for linkage studies.

Several methods are useful for determining an association between a marker and a disease, disorder and/or phenotype of interest. However, any such study should consider several parameters to avoid difficulties, such as, for example, population stratification, that may produce false positive results.

Population stratification occurs when there are multiple subgroups with different allele frequencies present within a population. The different underlying allele frequencies in the sampled subgroups may be independent of the disease, disorder and/or phenotype within each group, and, as a consequence, may produce erroneous conclusions of linkage disequilibrium or association.

Generally, problems of population stratification are avoided by using appropriate control samples. For example, case-comparison based design may be used in which a comparison between a group of unrelated probands with the disease, disorder and/or phenotype and a group of control (comparison) individuals who are unrelated to each other or to the probands, but who have been matched to the proband group on relevant variable (other than affection status) that may influence genotype (eg. sex, ethnicity and/or age).

Alternatively, controls are screened to exclude those subjects that have a personal history of the disease, disorder and/or phenotype of interest (and/or a family history of the disease, disorder and/or phenotype of interest). Such a “supernormal” control group is representative of the allele distribution of individuals unaffected by a disease, disorder and/or phenotype of interest.

Alternatively, a family-based association method may be used, in which non-transmitted alleles of the parents of a singly, ascertained proband are used as a random sample of alleles from which the proband was sampled. Such non-transmitted alleles are used to construct a matched control sample.

One extension of a family-based association method (the transmission disequilibrium test, TDT) uses a McNemar statistic to test for excess transmission of a marker allele to affected individuals above that expected by chance (Spielman et al., Am. J. Hum. Genet., 52: 506-516, 1993). Essentially, TDT considers parents who are heterozygous for an allele and/or polymorphism associated with a disease, disorder or phenotype and evaluates the frequency with which the allele and/or polymorphism or its alternate is transmitted to affected offspring. By only studying heterozygous parental genotypes TDT provides a test of association between linked loci and, as a consequence, avoids false associations between unlinked loci in the presence of population stratification.

The TDT method has been further refined to account for, for example multiallelic markers (Sham and Curtis Ann. Hum. Genet., 59: 323-326, 1995), multiple siblings in a family (Spielman and Ewens Am. J. Hum. Genet., 62:450-458, 1998), missing parental data (Curtis, Ann. Hum. Genet., 61: 319-333, 1997) and quantitative traits (Allison, Am. J. Hum. Genet., 60: 676-690, 1997 and Martin et al., Am. J. Hum. Genet., 67: 146-154, 2000).

In general, analysis of association is a test to detect non-random distribution of one or more alleles and/or polymorphisms within subjects affected by a disease/disorder and/or phenotype of interest. The comparison between the test population and a suitable control population is made under the null hypothesis assumption that the locus to which the alleles and/or polymorphisms are linked has no influence on phenotype, and from this a nominal p-value is produced. For analysis of a biallelic polymorphism (eg a SNP) using a case control study, a chi-square analysis (or equivalent test) of a 2×2 contingency table (for analysis of alleles) or a 3×2 contingency table (for analysis of genotypes) is used.

For analysis using a family-based association study, marker data from members of the family of each proband are used to estimate the expected null distributions and an appropriate statistical test performed that compares observed data with that expected under the null hypothesis.

Another method useful in the analysis of association of a marker with a disease, disorder and/or phenotype is the genomic control method (Devlin and Roeder, Biometrics, 55: 997-1004, 1999). For a case-control analysis of candidate allele/polymorphism the genetic control method computes chi-square test statistics for both null and candidate loci. The variability and/or magnitude of the test statistics abserved for the null loci are increased if population stratification and/or unmeasured genetic relationships among the subjects exists. This data is then used to derive a multiplier that is used to adjust the critical value for significance test for candidate loci. In this manner, genetic control permits analysis of stratified case-control data without an increased rate of false positives.

A structured association approach (Pritchard et al., Am. J. Hum. Genet., 67: 170-181, 2000) uses marker loci unlinked to a candidate marker to infer subpopulation membership. Latent class analysis is used to control for the effect of population substructure. Essentially, null loci are used to estimate the number of subpopulations and the probability of a subject's membership to each subpopulation. This method is then capable of accounting for a change in allele/polymorphism frequency as a result of population substructure.

Alternatively, or in addition, should a particular gene or gene product be likely to be involved in a disease, disorder or phenotype of interest a Bayesian statistical approach may be used to determine the significance of an association between an allele and/or polymorphism of that gene and the disease, disorder or phenotype of interest. Such an approach takes account of the prior probability that the locus under examination is involved in the disease, disorder or phenotype of interest (eg, Morris et al., Am. J. Hum. Genet., 67: 155-169, 2001).

Publicly available software are used to determine an association between an allele and/or polymorphism and a bipolar affective disorder or a predisposition to a bipolar affective disorder. Such software include, for example, the following:
(i) Analysis of Complex Traits (ACT), which includes methods for multivariate analysis of complex traits. ACT is based on the research reported in Amos, et al., Ann. Hum. Genet. 60:143-160, 1996 and Amos, Am. J. Hum. Genet., 54:535-543, 1994;
(ii) ADMIXMAP, a general-purpose program for modelling admixture using marker genotypes and trait data of individuals from an admixed population; useful for estimate individual and population level admixture, test for a relationship between disease risk and individual admixture in case-control, cross-sectional or cohort studies, localize genes underlying ethnic differences in disease risk by admixture mapping and control for population structure (variation in individual admixture) in genetic association studies so as to eliminate associations with unlinked genes;
(iii) ANALYZE, an accessory program for the LINKAGE program that facilitates both parametric and non-paramtetric tests for association;
(iii) BAMA (Bayesian analysis of multilocus association), useful for selecting a trait-associated subset of markers among many candidates; and
(iv) CLUMP, a Monte Carlo method for assessing significance of a case-control association study with multi-allelic marker;
(v) ET-TDT (evolutionary tree-transmission disequilibrium test) and ETTDT (extended transmission disequilibrium test), extensions of the TDT test; and
(vi) FBAT (family based association test), useful for testing for association/linkage between disease phenotypes and haplotypes by utilizing family-based controls

Preferably, a marker that is determined using any of the methods described supra is within a FAT gene and is associated with a bipolar affective disorder.

Methods of Determining a Marker for a Bipolar Affective Disorder Linked to Map Position 4q35.2 of the Human Genome

Another aspect of the present invention provides a method for identifying a marker for bipolar affective disorder comprising identifying a marker that is linked to chromosome position 4q35.2 of the human genome, wherein said marker is present in an individual suffering from bipolar affective disorder and said marker is not present in a suitable control sample, eg. a normal or healthy individual or a typical population of subjects that do not suffer from a bipolar affective disorder or the distribution of the marker is significantly associated with a bipolar affective disorder.

Preferably, the identified marker is present in a plurality of individuals suffering from bipolar affective disorder, and/or not present in a plurality of normal and/or healthy individuals.

In another embodiment, the present invention provides a method of identifying a marker that determines a subject's predisposition to a bipolar affective disorder and/or is diagnostic of a bipolar affective disorder comprising:
(i) identifying a locus that is associated with the genetic variation in a bipolar affective disorder;
(ii) identifying a marker that is linked to the locus at (i), wherein said marker associated with a subjects predisposition for a bipolar affective disorder and/or is diagnostic of a bipolar affective disorder;
(iii) analyzing a panel of subjects to determine those that suffer from bipolar affective disorder, wherein not all members of the panel comprise or express the marker; and
(iv) determining the variation in the presence or absence of the marker between the members of the panel wherein said variation indicates that the marker is involved in determining or is associated with a subjects predisposition for a bipolar affective disorder and/or is diagnostic of a bipolar affective disorder.

In a related embodiment, the present invention provides a method of identifying a marker that determines a subject's predisposition for a bipolar affective disorder and/or is diagnostic of a bipolar affective disorder comprising:
(i) identifying a locus that is associated with genetic variation in a bipolar affective disorder in a subject;
(ii) identifying an allele of a gene that is linked to the locus at (i), wherein said allele is a candidate allele that determines or is associated with a subjects predisposition for bipolar affective disorder and/or is diagnostic of a bipolar affective disorder;
(iii) analyzing a panel of subjects to determine those that suffer from bipolar affective disorder, wherein not all members of the panel comprise or express the allele and;
(iv) determining the variation in expression at (iii), wherein said variation in the presence or absence of the allele between the members of the panel indicates that the allele is a marker involved in determining or associated with a subjects predisposition for bipolar affective disorder and/or is diagnostic of a bipolar affective disorder.

In a further embodiment, the present invention provides a method of identifying a marker that determines a subjects predisposition for a bipolar affective disorder and/or is diagnostic of a bipolar affective disorder comprising:
(i) identifying a locus that is associated with genetic variation in a bipolar affective disorder in a subject;
(ii) identifying several alleles of a gene that are linked to the locus (i), wherein said gene is a candidate gene that determines a subjects predisposition for a bipolar affective disorder and/or is diagnostic of a bipolar affective disorder;
(iii) analyzing a panel of subjects to determine those that suffer from a bipolar affective disorder, wherein each of the members of the panel comprise or express at least one of the alleles; and
(iv) determining the variation at (iii) wherein said variation in the presence or expression of the alleles between the members of the panel indicates that the gene is a marker involved in determining or is associated with a subjects predisposition for a bipolar affective disorder and/or is diagnostic of a bipolar affective disorder.

In a preferred embodiment, the marker, allele or gene is linked to map position 4q35.2 of the human genome and is associated with a bipolar affective disorder.

In a preferred embodiment, the identified marker consists of a FAT gene, comprises a FAT gene or is contained within a FAT gene. In another preferred embodiment, the identified marker is an allele of a FAT gene.

A number of mapping methods for determining useful loci and estimating their effects have been described (e.g. as reviewed in, Broeckel and Schork, J Physiol, 2003, Zheng et al., World J. Gastroenterol. 9(8):1646-56, 2003, Tomer, Autoimmun Rev. August; 1(4):198-204, 2002, Haines and Pericack (Eds) In: Approaches to gene mapping in complex human diseases, John Wiley & Sons; 1 edition, 1998). In the present context, these methods are applied to identify the major component(s) of the total genetic variance that contribute(s) to the variation in the development of a bipolar affective disorder in a subject, such as, for example, determined by clinical assessment (e.g. using the International Classification of Diseases of the World Health Organisation (10th Edition) and the Diagnostic and Statistic Manual (4th Edition). More particularly, the segregation of known markers is used to map and/or characterize an underlying locus associated with a bipolar affective disorder. The locus method involves searching for associations between the segregating molecular markers and a bipolar affective disorder in a segregating population of subjects (i.e. by using pedigree studies), to identify the linkage of the marker to the locus.

As for statistical methods, Single Marker Analysis (Point Analysis) is used to detect a locus in the vicinity of a single marker. The number of subjects that develop a bipolar affective disorder in a population of subjects (e.g. humans) segregating for a particular marker, are compared according to the marker class. The difference in the number of subjects that develop a bipolar affective disorder provides an estimate of the phenotypic effect of substituting one allele for another allele at the locus. To determine whether or not the inferred phenotypic effect is significantly different from zero, a simple statistical test, such as t-test or F-test, is used. A significant value indicates that a locus is located in the vicinity of the marker. Single point analysis does not require a complete molecular linkage map. The further the locus is from the marker, the less likely it is to be detected statistically, as a consequence of recombination between the marker and the gene.

In the Anova, t-test or GLM approach, the association between marker genotype and development of a bipolar affective disorder comprises:
(i) classifying a number of subjects in a particular pedigree/s by marker genotype, such as for example, using microsatellite analyses or SNP analyses (using methods known in the art and/or described herein), thereby establishing classes of subjects;
(ii) determining the number of subjects that develop a bipolar affective disorder in each class of subjects in the pedigree, using a t-test, GLM or ANOVA; and
(iii) determining the significance of the differences in the mean at (ii), wherein a significant difference indicates that the marker is linked to the locus that determines a predisposition to or causes a bipolar affective disorder.

As will be known to those skilled in the art, the difference between the means of the classes provides an estimate of the effect of the locus in determining the predisposition of a subject to a bipolar affective disorder.

In the regression approach, the association between marker genotype and phenotype is determined by a process comprising:
(i) assigning numeric codes to marker genotypes; and
(ii) determining the regression value r for development of a bipolar affective disorder against the codes, wherein a significant value for r indicates that the marker is linked to the locus for a bipolar affective disorder, and wherein the regression slope gives an estimate of the effect of a particular locus on the development of a bipolar affective disorder.

For QTL interval mapping, the Mapmaker algorithm developed by Lincoln et al., Constructing genetic linkage maps with MAPMAKER/EXP version 3.0: A tutorial and reference manual. Whitehead Institute for Biomedical Research, Cambridge, Mass., USA, 1993, can be used. The principle behind interval mapping is to test a model for the presence of a QTL at many positions between two mapped marker loci. This model is a fit of a presumptive QTL to development of a bipolar affective disorder wherein the suitability of the fit is tested by determining the maximum likelihood that a QTL for a bipolar affective disorder lies between two segregating markers. For example, in the case of a QTL located between two segregating markers, the 2-loci marker genotypes of segregating subjects will each contain mixtures of QTL genotypes. Accordingly, it is possible to search for loci parameters that best approximate the distribution in the development of a bipolar affective disorder for each marker class. Models are evaluated by computing the likelihood of the observed distributions with and without fitting a QTL effect. The map position of a QTL is determined as the maximum likelihood from the distribution of likelihood values (LOD scores: ratio of likelihood that the effect occurs by linkage: likelihood that the effect occurs by chance), calculated for each locus.

Interval mapping by regression (Haley and Knott., Heredity 69, 315-324, 1992) is a simplification of the maximum likelihood method supra wherein basic QTL analysis or regression on coded marker genotypes is performed, except that phenotypes are regressed on the probability of a QTL genotype as determined from the linkage between development of a bipolar affective disorder and the nearest flanking markers. In most cases, regression mapping gives estimates of QTL position and effect that are almost identical to those given by the maximum likelihood method. The approximation deviates only at places where there are large gaps, or many missing genotypes.

In the composite interval mapping (CIM) method (Jansen and Stam, Genetics 136, 1447-1455, 1994; Utz and Melchinger, 1994, supra; Zeng, Genetics 136, 1457-1468, 1994), the analysis is performed in the usual way, except that the variance from other QTLs is accounted for by including partial regression giving more power and precision than simple interval mapping, because the effects of other QTLs are not present as residual variance. CIM can remove the bias that can be caused by the QTLs that are linked to the position being tested.

Publicly available software are used to map a locus for a bipolar affective disorder. Such software include, for example, the following:
(i) QTL Cartographer (http://statgen.mcsu.edu/qtlcart/cartographer.html) for single-marker regression, interval mapping, or composite interval mapping;
(ii) MapQTL (http://www.cpro.dlo.nl/cbw/); Qgene for performing either single-marker regression or interval regression to map loci; and
(iii) SAS for detecting a locus by identifying associations between marker genotype and a bipolar affective disorder by a single marker analysis approach such as ANOVA, t-test, GLM or REG.

Preferably, a marker that is determined using any of the methods described supra is in linkage disequilibrium with a locus associated with bipolar affective disorder. “Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage disequilibrium”. The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that comprise more than one allele, a first allelic pattern is in linkage disequilibrium with a second allelic pattern if all the alleles that comprise the first allelic pattern are in linkage disequilibrium with at least one of the alleles of the second allelic pattern

Clearly, the present invention extends to the use of a marker that is linked to map position 4q35.2 of the human genome and/or associated with a bipolar affective disorder in the manufacture of a diagnostic reagent for determining the predisposition of a subject to bipolar affective disorder or diagnosing bipolar affective disorder.

The present invention also provides a marker that is linked to map position 4q35.2 of the human genome and/or associated with a bipolar affective disorder when used to determine the predisposition of a subject to bipolar affective disorder or diagnose bipolar affective disorder.

Methods of Determining a Candidate Compound for Treatment of Bipolar Affective Disorder.

Currently, there are few known therapeutics for the treatment of bipolar affective disorder. Furthermore, the mode of action of those therapeutics that are currently in use is unknown. The identification of the involvement of the FAT gene in bipolar affective disorder provides an assay for use in determining candidate compounds for the treatment of a bipolar affective disorder.

The present inventors have shown that the level of FAT mRNA is reduced in mice that are treated with lithium, a compound that is a known therapeutic of a bipolar affective disorder and appears to be reduced in mice treated with the therapeutic compound valproate. Accordingly, another aspect of the present invention provides a method of determining a candidate compound for the treatment of a bipolar affective disorder comprising:
(i) administering a candidate compound to an animal or cell comprising or expressing a marker within a FAT gene that is associated with a bipolar affective disorder and determining the level of FAT expression in said cell or animal;
(ii) administering a candidate compound to an animal or cell that does not comprise or express a marker within a FAT gene that is associated with a bipolar affective disorder and determining the level of FAT expression in said cell or animal; and
(iii) comparing the level of FAT expression at (i) and (ii) wherein a decreased level of FAT expression at (i) relative to (ii) indicates that the compound is a candidate compound for the treatment of a bipolar affective disorder.

The inventors have shown that the level of mouse FAT is modulated by administration of lithium or valproate. Accordingly, the present invention additionally provides a method of determining a candidate compound for the treatment of a bipolar affective disorder comprising:
(i) administering a candidate compound to an animal or cell capable of expressing a FAT gene and determining the level of FAT expression in said cell or animal;
(ii) determining the level of FAT expression in an animal or cell capable of expressing a FAT gene in the absence of the candidate compound; and
(iii) comparing the level of FAT expression at (i) and (ii), wherein a decreased level of FAT expression at (i) relative to (ii) indicates that the compound is a candidate compound for the treatment of a bipolar affective disorder.

Preferably, the level of FAT expression is determined by determining the level of a FAT polypeptide in a cell or animal.

In a particularly preferred embodiment, the level of FAT expression is determined by determining the level of a FAT mRNA in a cell.

Methods of determining the level of a nucleic acid or polypeptide are known in the art and/or described herein.

For example, standard solid-phase ELISA assay formats are particularly useful for identifying the level fo expression of a FAT polypeptide. In accordance with this embodiment, an antibody that specifically binds to a FAT polypeptide is immobilized on a solid matrix, such as, for example an array of polymeric pins or a glass support. Lysate from a cell that has been incubated in the presence of a candidate compound or a cell that has not been incubated in the presence of a candidate compound is brought into physical relation with the immobilized antibody. The cell may also have been incubated in the presence of a compound that is a known therapeutic of a bipolar affective disorder, eg valproate or lithium, as a positive control. The level of captured FAT polypeptide is then detected with a suitable antibody, eg another anti-FAT antibody that binds to an epitope different to that of the capture antibody, or an anti-human antibody. The antibody is generally labeled with fluorescent molecules or conjugated to an enzyme (e.g. horseradish peroxidase), or alternatively, a second labeled antibody can be used that binds to the first antibody. The amount of FAT polypeptide is then determined using, for example, a standard curve determined using known amounts of, for example, recombinant FAT polypeptide. Alternatively, the level of a FAT polypeptide detected in the sample comprising a candidate compound may be directly compared to the level of FAT detected in a sample comprising no compound. Clearly, such an assay will require some degree of normalisation, eg., for total protein concentration.

In a particularly preferred embodiment, an amplification technique, such as, for example, real-time PCR is used to determine the level of FAT mRNA expression in a cell treated with a candidate compound. Methods of real-time PCR are known in the art and described, for example in Gibson et al., Genome Research 6: 995-1001, 1996; Heid et al., Genome Research 6:986-994, 1996). Real-time PCR is a technique that evaluates the level of PCR product accumulation during amplification. This technique permits quantitative evaluation of mRNA levels in multiple samples. Briefly, mRNA is extracted from a cell, tissue or organism and cDNA is prepared using standard techniques. Oligonucleotides and fluorescent probes are designed for specific detection of expression products of genes of interest using methods known in the art and/or described herein. To quantitate the amount of specific RNA in a sample, a standard curve is generated along using a known concentration of the nucleic acid of interest, e.g., a recombinant nucleic acid cloned into a plasmid or phagemid. Standard curves may be generated using the Ct values determined in the real-time PCR, which are related to the initial cDNA concentration used in the assay. In addition, a standard curve is generated for a positive control nucleic acid (eg. β-actin or HPRT). Such standard curves permit standardization of initial RNA content of a tissue sample to the amount of control for comparison purposes.

It will be apparent to the skilled person that the assay formats described herein are amenable to high throughput screening of samples.

As will be apparent to the skilled artisan, the method of the present aspect of the invention is preferably though not necessarily performed in an animal or cell expressing a human FAT gene. Preferably, the human FAT gene comprises one or more markers that are associated with bipolar affective disorder. More preferably, the human FAT gene comprises a cytosine at position 80,217 of SEQ ID NO: 1, a thymine at position 130,625 of SEQ ID NO: 1, a thymine at position 130,613 of SEQ ID NO: 1, a guanine at position 139,968 of SEQ ID NO: 1, a guanine at position 142,199 of SEQ ID NO: 1, an adenine at position 142,460 of SEQ ID NO: 1, a guanine at position 145,782 of SEQ ID NO: 1, a guanine at position 146,008 of SEQ ID NO: 1, a guanine at position 146,012 of SEQ ID NO: 1, a thymine at position 148,108 of SEQ ID NO: 1, an adenine at position 148,199 of SEQ ID NO: 1, an adenine at position 148,333 of SEQ ID NO: 1, a cytosine at position 151,403 of SEQ ID NO: 1 or a thymine at position 153,127 of SEQ ID NO: 1. More preferable, the human FAT gene comprises a guanine at position 139,968 of SEQ ID NO: 1, a guanine at position 146,012 of SEQ ID NO: 1, a thymine at position 148,108 of SEQ ID NO: 1 and an adenine at position 148,333 of SEQ ID NO: 1.

Preferably, the human FAT gene is a genomic gene, such as, for example, the sequence set forth in SEQ ID NO: 1. However, a human FAT cDNA is clearly encompassed by the present invention. As will be apparent to the skilled artisan, only those markers associated with bipolar affective disorder that are in a protein coding region or control the production of a protein coding region (eg a splice-donor or splice-acceptor site) of a human FAT gene are found in a human FAT cDNA.

In one embodiment, the human FAT gene is placed or is located in operable connection with the endogenous FAT promoter, thereby driving expression in those cells in which FAT is expressed in nature. In another embodiment, the human FAT gene is placed in operable connection with a heterologous promoter, for example to drive expression in a variety of tissues, or to produce inducible expression.

Placing a nucleic acid molecule under the regulatory control of, i.e., “in operable connection with”, a promoter sequence means positioning said molecule such that expression is controlled by the promoter sequence. Promoters are generally positioned 5′ (upstream) to the coding sequence that they control. To construct heterologous promoter/structural gene combinations, it is generally preferred to position the promoter at a distance from the gene transcription start site that is approximately the same as the distance between that promoter and the gene it controls in its natural setting, i.e., the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the gene from which it is derived. Again, as is known in the art, some variation in this distance can also occur.

Typical promoters suitable for expression in a or in a mammalian cell, mammalian tissue or intact mammal include, for example a promoter selected from the group consisting of, retroviral LTR elements, the SV40 early promoter, the SV40 late promoter, the cytomegalovirus (CMV) promoter, the CMV IE (cytomegalovirus immediate early) promoter, the EF_{1α} promoter (from human elongation factor 1α), the EM7 promoter, the UbC promoter (from human ubiquitin C), a neuron specific enolase (NSE) promoter, the human aromatic L-amino acid decarboxylase (Chatelin et al., Brain Res Mol Brain Res. 97(2):149-60, 2001).

Preferred mammalian cells for expression of the nucleic acid fragments include neuronal cell lines, such as, for example, a hNT2 cell, a GT1 cell, a LN18 cell, a Daoy cell, a NCIH250 cell, a DU145 cell, a SK-N-DZ cell or a PFSK cell.

Alternatively, a FAT encoding nucleic acid placed in operable connection with, for example, a constitutive promoter may be used to express FAT in any cell. For example a cell selected from the group consisting of a COS cell, a CHO cell, a murine 10T cell, a MEF cell, a NIH3T3 cell, a MDA-MB-231 cell, a MDCK cell, a HeLa cell, a K562 cell, a HEK 293 cell and a 293T cell.

Methods of producing expression constructs are known in the art and are described, for example, in Ausubel et al. (In: Current Protocols in Molecular Biology. Wiley Interscience, ISBN 047 150338, 1987) or Sambrook et al. (In: Molecular Cloning: Molecular Cloning: A Laboratory Manual, Cold Spring Harbor Laboratories, New York, Third Edition 2001).

Following production of a suitable gene construct, said construct is introduced into the relevant cell. Methods of introducing the gene constructs into a cell or organism for expression are well known to those skilled in the art and are described for example, in Ausubel et al. (In: Current Protocols in Molecular Biology. Wiley Interscience, ISBN 047 150338, 1987) and (Sambrook et al. (In: Molecular Cloning: Molecular Cloning: A Laboratory Manual, Cold Spring Harbor Laboratories, New York, Third Edition 2001). The method chosen to introduce the gene construct in depends upon the cell type in which the gene construct is to be expressed. Means for introducing recombinant DNA into a mammalian cell include, but are not limited to electroporation, PEG mediated transfection, microinjection, transfection mediated by DEAE-dextran, transfection mediated by calcium phosphate, transfection mediated by liposomes, such as, for example, Lipofectamine (Invitrogen) and/or cellfectin (Invitrogen), transduction by adenoviuses, herpesviruses, togaviruses or retroviruses and microparticle bombardment such as by using DNA-coated tungsten or gold particles (Agacetus Inc., WI, USA).

Alternatively, the gene construct is used to produce a transgenic animal, for example a transgenic mouse. Means of producing transgenic animals are known in the art and described, for example, in Hogan et al. (In: Manipulating the Mouse Embryo. A Laboratory Manual, 2^{nd} Edition. Cold Spring Harbour Laboratory. ISBN: 0879693843, 1994). For example, a gene construct comprising a human FAT cDNA or genomic gene is microinjected into the pronucleus of a fertilised mammalian oocyte. The oocyte is then microinjected into a uterus of a pseudopregnant recipient female mammal. Any offspring that are born are screened for presence of the transgene using in their genome, for example, PCR screening or Southern hybridisation using methods known in the art. Those mice that comprise the transgene are bred, and their offspring assayed for transgene expression, using, for example, Northern blotting, RT-PCR or Western blotting. Such mice are then useful for the screening assay of the present invention.

Transgenic animals are also produced by nuclear transfer technology as described in Schnieke, A. E. et al., 1997, Science, 278: 2130 and Cibelli, J. B. et al., 1998, Science, 280: 1256. Using this method, cells, e.g. fibroblasts, from donor animals are stably transfected with a plasmid incorporating the coding sequences for a form of a FAT polypeptide. Stable transfectants are then fused to enucleated oocytes, cultured and transferred into female recipients.

As will be apparent to the skilled artisan, it is preferable that a cell or animal only expresses the form of the FAT gene that comprises a marker associated with bipolar affective disorder. Accordingly, the endogenous form of a FAT gene may be disrupted using, for example, homologous recombination.

Methods of producing a non-human animal, preferably, a non-human mammal deficient in a gene (i.e. a knockout animal) are known in the art and described, for example in Hogan et al. (In: Manipulating the Mouse Embryo. A Laboratory Manual, 2^{nd} Edition. Cold Spring Harbour Laboratory. ISBN: 0879693843, 1994). For example, to produce a mutant mammal by homologous recombination an embryonic stem (ES) cell line capable of contributing to the germ line is transfected using an method known in the art and/or described herein with a targeting construct. A targeting construct typically comprises at least 2 regions that are almost identical to the region of the genome to be targeted that flank a detectable reporter molecule (such as, for example, a neomycin resistance gene or a β-galactosidase gene). Following transfection of ES cells, those that have successfully integrated the targeting construct into their genome (by homologous recombination) (as determined, for example, by PCR, Southern blotting, or negative selection) are microinjected into a wild-type blastocyst in order to produce a chimeric animal. As many ES cell lines retain the ability to differentiate into all cell types present in an animal, the chimera can have tissues, including the germ line, with contribution from both the normal blastocyst and the mutant ES cells. Germ-line chimeras are bred to yield animals that are heterozygous for the mutation introduced into the ES cell, and can be interbred to produce homozygous mutant mice.

Homologous recombination can also be used to produce homozygous mutant cell lines, for example as described by te Riele et al., 1990, Cruz et al., 1991; and Mortensen et al., 1991). Clearly, cell based assays are preferred for rapid determination of candidate compounds for the treatment of a bipolar affective disorder.

As will be apparent to the skilled artisan, a candidate compound for the treatment of a bipolar affective disorder that is determined using a cell based assay may be validated in an animal model. Such validation ensures that the compound only influences the onset or progression of a bipolar affective disorder, and is not, for example toxic to the animal.

A preferred model in which to validate a compound for the treatment of bipolar affective disorder is a FAT deficient animal produced using a method described supra that is transgenic for human FAT that comprises a marker that is associated with a bipolar disease. Methods of determining a bipolar affective disorder in an animal are described, for example, in Gogos et al., 95(17): 9991-9996. For example, an animal is studied to determine anxiety behaviour and prepulse inhibition. Preferably, a candidate compound for the treatment of bipolar affective disorder reduces anxiety behaviour and/or prepulse inhibition in a genetically modified animal subject to levels similar to that observed in a wild-type animal. As will be apparent to the skilled artisan, a candidate compound that is validated in an animal model is then further validated in clinical trials.

In one embodiment of the present invention, a modulator is a small molecule (e.g. a chemical compound, agonist, antagonist, allosteric modulator, competitive inhibitor, or non-competitive inhibitor of FAT activity).

In one embodiment, the compound administered comprises nucleic acid, For example, the nucleic acid is an antagonist of FAT expression, such as, for example, an antisense nucleic acid, peptide nucleic acid (PNA), ribozyme, or interfering RNA, which is complementary, in whole or in part, to a target molecule comprising a sense strand, and can hybridise with the target molecule. When introduced into a cell using suitable methods, such a nucleic acid inhibits the expression of the FAT gene encoded by the sense strand. Antisense nucleic acid, ribozymes (e.g. Cech et al., U.S. Pat. No. 4,987,071; Cech et al., U.S. Pat. No. 5,116,742; Bartel and Szostak, Science 261, 1411-1418, 1993), nucleic acid capable of forming a triple helix (e.g. Helene, Anticancer Drug Res. 6, 569-584, 1991), PNAs (Hyrup et al., Bioorganic & Med. Chem. 4, 5-23, 1996; O'Keefe et al., Proc. Natl. Acad. Sci. USA 93, 14670-14675, 1996), interfering RNAs (Elbashir et al., Nature 411, 494-498, 2001; Sharp, Genes Devel. 15, 485-490, 2001; Lipardi et al., Cell 107, 297-307, 2001; Nishikura, Cell 107, 415-418, 2001) or small interfering RNAs (siRNA) may be produced by standard techniques known to the skilled artisan, based upon the sequences disclosed herein.

Accordingly, the antisense nucleic acid comprises a nucleic acid that is complementary to at least about 15-20 contiguous nucleotides of a sequence having at least about 80% identity to SEQ ID NOs: 1, 2 or 4. Ribozyme, PNA, interfering RNA or siRNA comprises a sequence that is complementary to at least about 15-20 contiguous nucleotides of a sequence having at least about 80% identity to SEQ ID NOs: 2 or 4 or a sequence transcribed from the sequence set forth in SEQ ID NO: 1 and can hybridise thereto. Preferably, an antisense ribozyme, PNA, interfering RNA or siRNA is capable of specifically hybridising to a FAT gene that comprises a marker that is associated with bipolar affective disorder

Longer molecules, comprising a sequence that is complementary to at least about 25, or 30, or 35, or 40, or 45, or 50 contiguous nucleotides of mRNA encoding a human FAT polypeptide are also encompassed by the present invention.

In one embodiment, a construct comprising an antisense nucleic acid, ribozyme, PNA, interfering RNA or siRNA, can be introduced into a suitable cell to inhibit expression of FAT. In another embodiment, such a construct can be introduced into some or all of the cells of a mammal. The antisense nucleic acid, ribozyme, PNA, or interfering RNA, inhibits expression of the target gene.

An antisense oligonucleotide is also useful for altering the splicing of a mRNA, eg. a FAT mRNA. Accordingly, alternate splicing of a FAT mRNA to include an additional sequence and/or an additional sequence, is be suppressed using an antisense oligonucleotide that hybridises to the splice junction of the region of FAT that is to be excluded. Such an approach has been shown to be successful for the suppression of the inclusion of exon 10 into the tau protein (Kalbfuss et al., J. Biol. Chem. 276: 42986-42993, 2001.

The use of antibodies that can inhibit one or more functions characteristic of a FAT protein, such as a binding activity, is also encompassed by the present invention.

Furthermore, the use of ligands, such as, for example, peptides that modulate activity of a FAT polypeptide is also encompassed by the present invention. Preferably, such a peptide is expressed within the cell, although the peptide may also be introduced into the cell using methods known in the art.

As will be apparent to the skilled artisan the present invention encompasses peptides that modulate the interaction of a FAT polypeptide with another peptide, polypeptide or protein, wherein that interaction inhibits the development of bipolar affective disorder.

The modulators identified using the methods described herein are useful for the therapeutic or prophylactic treatment of a bipolar affective disorder.

The present invention clearly encompasses the use of any in silico analytical method and/or industrial process for carrying the screening methods described herein into a pilot scale production or industrial scale production of an inhibitory compound identified in such screens. This invention also provides for the provision of information for any such production. Accordingly, a further aspect of the present invention provides a process for identifying or determining a compound or modulator supra, said method comprising:
(i) performing a method as described herein to thereby identify or determine a compound for the treatment of a bipolar affective disorder;
(ii) optionally, determining the structure of the compound; and
(iii) providing the compound or the name or structure of the compound such as, for example, in a paper form, machine-readable form, or computer-readable form.

Naturally, for compounds that are known albeit not previously tested for their function using a screen provided by the present invention, determination of the structure of the compound is implicit in step (i) supra. This is because the skilled artisan will be aware of the name and/or structure of the compound at the time of performing the screen.

As used herein, the term “providing the compound” shall be taken to include any chemical or recombinant synthetic means for producing said compound or alternatively, the provision of a compound that has been previously synthesized by any person or means.

In a preferred embodiment, the compound or the name or structure of the compound is provided with an indication as to its use e.g., as determined by a screen described herein.

A further aspect of the present invention provides a process for producing a compound supra, said method comprising:

a process for identifying or determining a compound or modulator supra, said method comprising:

(i) performing a method as described herein to thereby identify or determine a compound for the treatment of a bipolar affective disorder;
(ii) optionally, determining the structure of the compound;
(iii) optionally, providing the name or structure of the compound such as, for example, in a paper form, machine-readable form, or computer-readable form; and
(iv) providing the compound.

In a preferred embodiment, the synthesized compound or the name or structure of the compound is provided with an indication as to its use e.g., as determined by a screen described herein.

A further aspect of the present invention provides a method of manufacturing a compound for the treatment of a bipolar affective disorder comprising:
(i) determining a candidate compound for the treatment of bipolar affective disorder; and
(ii) using the compound in the manufacture of a therapeutic or prophylactic for the treatment of bipolar affective disorder.

In one embodiment, the method comprises the additional step of isolating the candidate compound. Alternatively, a compound is identified and is produced for use in the manufacture of a compound for the treatment of a bipolar affective disorder.

Formulation of a compound to be administered will depend upon the route of administration selected (e.g. solution, emulsion, capsule). An appropriate composition or medicament comprising the compound can be prepared in a physiological carrier or vehicle (see, generally Remington's Pharmaceutical Sciences, 17th Edition, Mack Publishing Co., Pa., 1985).

Alternatively, or in addition the nucleic acid encoding the protein/peptide can be introduced into a host cell capable of expressing said protein/peptide for delivery. In accordance with this embodiment, the cells can be implanted (either alone or in a barrier device), injected or introduced by any other method known in the art.

Preferably, the compound is a pharmaceutical compound.

Formulation of a pharmaceutical compound will vary according to the route of administration selected (e.g., solution, emulsion, capsule). An appropriate composition comprising the identified modulator to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluid, nutrient or electrolyte replenishers and the like (See, generally, Remington's Pharmaceutical Sciences, 17th Edition, Mack Publishing Co., Pa., 1985). For inhalation, the agent can be solubilized and loaded into a suitable dispenser for administration (e.g., an atomizer, nebulizer or pressurized aerosol dispenser).

Furthermore, where the agent is a protein or peptide, the agent can be administered via in vivo expression of the recombinant protein. In vivo expression can be accomplished via somatic cell expression according to suitable methods (see, e.g. U.S. Pat. No. 5,399,346). In this embodiment, nucleic acid encoding the protein can be incorporated into a retroviral, adenoviral or other suitable vector (preferably, a replication deficient infectious vector) for delivery, or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

As will be apparent to a skilled artisan, a compound that is active in vivo is particular preferred. A compound that is active in a human subject is even more preferred. Accordingly, when manufacturing a compound that is for the treatment of a bipolar affective disorder it is preferable to ensure that any components added to the compound do not inhibit or modify the activity of said compound.

The present invention is described further in the following non-limiting examples.

### EXAMPLE 1

### Mapping a Bipolar Affective Disorder Locus to Map Position 4q35.2 of the Human Genome

1.1 Samples

The families used in the present study were ascertained as part of an ongoing bipolar genetics study via the Mood Disorders Unit, Prince of Wales Hospital/School of Psychiatry, University of New South Wales, Sydney, Australia. Medium to large multigenerational pedigrees with illness over at least two generations and containing a minimum of three affected individuals, at least two of whom were diagnosed with bipolar I (BPI), were recruited. Families were almost entirely of British or Irish descent. The families were assessed using the Diagnostic Interview for Genetic Studies (DIGS) (Numberger et al. Arch. Gen. Psychiatry, 51: 849-859, 1994), with interviews being undertaken by experienced psychologists and psychiatric nurses trained in this instrument. Best-estimate Research Diagnostic Criteria diagnoses were made by senior psychiatrists after independent evaluation of DIGS interviews, family informant data (using the FIGS instrument), and medical records. All marrying-in-individuals were routinely question about any family history of psychiatric illness to ensure unilateral descent of bipolar disorder in the pedigrees. All individuals who participated in the study provided appropriate informed written consent. The study was approved by the Ethics Committee of the University of New South Wales and complies with the guidelines of the National Health and Medical Research Council and the Helsinki Declaration.

The pedigree cohort in the present study comprises 55 pedigrees. The cohort is made up of 36 extended pedigrees, eight nuclear pedigrees with multiple affected offspring, and 11 affected sib pairs with parents. The 55 pedigrees contain 983 individuals with 674 individuals available for analysis including 214 affected individuals. In total, there are 108 individuals with BPI, 13 with bipolar II (BPII), 30 with schizoaffective disorder-manic type (SZ/MA), and 63 with recurrent unipolar depression (UP). The average number of individuals per pedigree is 11.8, with an average number of affected individuals per pedigree of 3.7.

Simulation analyses using SLINK and MSIM (Ott, Proc. Natl. Acad. Sci. USA, 86: 4175-4178, 1989; Weeks et al. Am. J. Hum. Genet. 47: A204, 1990) were performed on these pedigrees to determine the power of the cohort to detect linkage and the expected maximum LOD scores to occur under heterogeneity and under the assumption of no linkage. Under the assumption of linkage and homogeneity, using a broad disease definition, a dominant model with maximum 90% age-specific penetrance and a six allele marker, the expected average and maximum LOD scores for the entire pedigree cohort are 8.3 and 22, respectively. Under the assumption of linkage and 50% heterogeneity, using a broad disease definition, a dominant model with maximum 90% age-specific penetrance and a six allele marker, the expected average and maximum LOD scores for the entire pedigree cohort are 2.5 and 19.3, respectively. Using the same model, but under the assumption of no linkage, the expected average and maximum LOD scores for the entire pedigree cohort are 0.3 and 4.9, respectively.

1.2 Genotyping

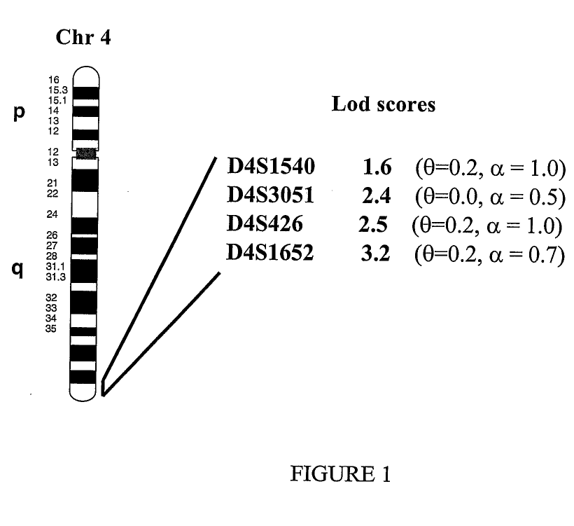

DNA was extracted from whole blood using a standard salting-out method. Several microsatellite markers spanning ˜50 cM on 4q35 were used in our analysis. The markers in the region were identified using the chromosome 4 summary map from The Genetic Location DataBase (LDB). Primer sequences, expected-size information, and known allele frequencies for most markers were obtained from The Genome Database. Several of the markers used in the present analysis and their relative location on Chromosome 4q are shown in FIG. 1.

PCR was performed in a 15 μl volume containing 60 ng DNA, 250 μM dNTPs, 0.33 μM of each primer (the forward primer labeled with 6-FAM fluorescent dye), 2.5 mM MgCl₂, 1×PCR buffer, and 0.6 U AmpliTaq Gold polymerase (Applied Biosystems). PCR reactions were carried out on a hybaid OmniGene thermal cycler (Middlesex, UK) using the following protocol; 95° C. for 12 min, 30 cycles of 95° C. for 30 s, either 58 or 60° C. for 30 s, 72° C. for 30 s, 72° C. for 5 min. PCR product (1.5 μl) was mixed with loading dye (2.5 μl formamide, 0.5 μl of 50 mg/ml blue dextran:50 mM EDTA, 0.5 μl GeneScan500 Tamra size standard). Samples were loaded onto a 4% polyacrylamide gel and electrophoresis was performed on an ABI PRISM 377 DNA Sequencer. Products were detected using the GeneScan Analysis program, version 3.1 and alleles were assigned using the Genotyper program, version 2.5 (Applied Biosystems).

There was an average of 383 genotypes determined per marker. The average number of informative meioses per marker was 290 and the average number of phase known meioses was 32.

1.3 Marker Ordering

CRIMAP (version 2.4) was used to construct multi-locus linkage maps of the 4q35 markers in our pedigree cohort. Markers were arranged according to informativeness (i.e., number of phase known meioses). The two most informative markers, D4S1554 and D4S171, were used as anchor markers. Each marker, in order of informativeness, was then added to the map using the ‘all’ function of CRIMAP and log₁₀ likelihoods were calculated. Alternative orders were tested using the ‘flips’ function, which calculates all permutations of adjacent markers, until only a single marker order was given. The ‘fixed’ function was then used to calculate the maximum likelihood recombination fractions and Kosambi cM distances for the genetic map.

1.4 Linkage Analysis

Two-point LOD score analysis was carried out using the ANALYZE package (Terwilliger, 1996). Three diseases and two penetrance models were used in analysis. In disease model I, individuals diagnosed with BPI or SZ/MA were classified as affected and all other family members were classified as unaffected. In model II, individuals diagnosed with BPI, SZ/MA, or BPII were classified as affected and all other family members were considered unaffected. In model III, individuals diagnosed with BPI, SZ/MA, and BPII or UP were classified as affected and all other family members were considered unaffected. Four liability classes (class 1, <20 years; class 2, 20-29 years; class 3, 30-39 years, and class 4, >40 years) were used in the analysis with maximum age-specific penetrance levels of either 60 or 90%. In the 90% model, liability classes were defined with penetrances of 0.18, 0.45, 0.68, and 0.9; those in the 60% model were defined with penetrances of 0.12, 0.30, 0.45, and 0.6. The data was analysed under both dominant and recessive inheritance models. The disease-allele frequencies was set at 0.035 for the dominant model and 0.2 for the recessive model, and the phenocopy rate of 5% was used in all analyses. Allele frequencies were calculated from the data using the DOWNFREQ program from the ANALYZE package (Terwilliger, 1996) using all individuals. Affected Sibpair and TDT analyses were performed using the ANALYZE package (Terwilliger, 1996).

1.5 Results

The expanded 55 pedigree cohort, comprising 674 individuals, of whom 214 are affected was genotyped with several markers spanning the 4q35 region, including D4S1540, D4S426, D4S3051, D4S2921, and D4S1652, to test for evidence of linkage to the 4q35 region.

Evidence for linkage to the 4q35 region was strengthened in the 55 pedigree cohort, with a maximum two-point LOD score of 3.19 (θ=0.18) at marker D4S1652, using diagnostic model III with a dominant mode of inheritance and 90% maximum age-specific penetrance. LOD scores greater than 1.5 were also observed for other markers genotyped in the 55 pedigrees including: D4S426, 2.49 (θ=0.2) under a dominant model and D4S3051, 2.38 (θ=0.16) and D4S1540, 1.57 (θ=0.18) under a recessive model (as shown in FIG. 1). No LOD scores greater than 1.5 were observed for any marker using the narrower disease models I (BPI and SZ/MA as affected) or II (BPI, SZ/MA, and BPII as affected).

### EXAMPLE 2

### SNP Analysis of Genes in the Susceptibility Locus on Chromosome 4q35

Following identification of the region of 4q35 of the human genome that is linked with bipolar affective disorder, SNPs located within this region and, in particular, SNPs located within the human FAT gene were analysed for an association with a bipolar affective diroder based upon a case-control study design. These were screened in an affected individual (case) and their spouse (control) from the seven bipolar kindreds previously reported by Badenhop et al. Am. J. Med. Genet. 117B: 23-32 2003 as showing evidence for linkage to the chromosome 4q35 locus using the methods described below.

2.1 SNP Detection by Direct Sequencing Following PCR Amplification

Oligonucleotide primers were designed from genomic DNA sequence to amplify and sequence coding regions of the FAT gene, as well as flanking intronic sequences.

The sequence of the oligonucleotides are listed in Table 1:

TABLE 1
Nucleotide sequences of oligonucleotides used to amplify and sequence regions
of the FAT gene comprising SNPs

| Gene | SNP | oligonucleotide sequence |
|---|---|---|
| FAT | rs172903 | TGAACGATGAAAGAGGCACGACAC (SEQ ID NO: 32) |
| | | GCTGAGTCACAGAAAGGCTGGTTA (SEQ ID NO: 33) |
| | rs2249916 & rs2249917 | CTGAATCTGGGGTGTGTTTTG (SEQ ID NO: 34) |
| | | CAAGTGAGTCCATTTGTTTTTG (SEQ ID NO: 35) |
| | rs2637777 | TGTGCTGGAAACAGAGAACAGGTT (SEQ ID NO: 36) |
| | | ACAGTGCCATAGACAAAGGTGATT (SEQ ID NO: 37) |
| | rs767168 | GCACCCCTTGAGACTGATTTAG (SEQ ID NO: 38) |
| | | ATCTGTGGAGATTGATGACATTTG (SEQ ID NO: 39) |
| | rs2289550 | CAACGCTGTGGAGAAACATACC (SEQ ID NO: 40) |
| | | TAAAACCCTAAATCAGTCTCAAGG (SEQ ID NO: 41) |
| | rs1280097 | CCTCTACACTGGCAAACAAAGC (SEQ ID NO: 42) |
| | | TTACAGACCTCGGAGAATAAAGAT (SEQ ID NO: 43) |
| | rs1280096 | ATGACGCTATGAAAAGGTATGAAC (SEQ ID NO: 44) |
| | | TTGTTTGCCAGTGTAGAGGATTAT (SEQ ID NO: 45) |
| | rs2306990 | ATGACGCTATGAAAAGGTATGAAC (SEQ ID NO: 46) |
| | | TTGTTTGCCAGTGTAGAGGATTAT (SEQ ID NO 47) |
| | rs1298865, rs2306988, & | TTCTTCGTTTGAGTTTGTAAGGTG (SEQ ID NO: 48) |
| | rs2306987 | GTTAAGAAAGTTGCGAGGAAGATT (SEQ ID NO: 49) |

PCR was performed in 25 μl volumes containing 10 mM Tris-Cl, pH 8.3; 50 mM KCl; 2.0 mM MgCl₂; 200 μM each dNTP; 20 ng genomic DNA; 20 pmole each primer, and 1 U AmpliTaq Gold DNA polymerase (Applied Biosystems).

Reactions were cycled with initial denaturation at 94° C. for 12 min, followed by 30 cycles of 94° C. for 30 s, 60° C. for 45 s, 72° C. for 1 min, and a final extension of 10 min at 72° C.

Amplified products were purified (QIAquick PCR purification, Qiagen) and subjected to automated sequence analysis using the ABI377 automated sequencer with Big-Dye terminator sequencing (Applied Biosystems Inc.). Sequencing primers were the same as used in PCR amplification.

2.2 SNP Analysis Using Mass Spectrometry

Oligonucleotide primers were designed from genomic DNA sequence to flank the SNPs of interest.

Nucleotide sequences of the oligonucleotides used in the analysis of SNPs from the FAT gene are shown in Table 2.

TABLE 2
Oligonucleotides used in the analysis of SNPs in the human FAT gene using mass spectrometry

| | | | PCR | |
|---|---|---|---|---|
| | | | product | |
| Gene | SNP | PCR oligonucleotide sequence | length | Extension oligonucleotide sequence |
| FAT | rs3775309 | GTTGGATGTTCCTGTACATACGAAGAAG | 118 | AAAATTACTTCTGAACTTTTTCTT |
| | | (SEQ ID NO: 50) | | (SEQ ID NO: 52) |
| | | ACGTTGGATGGAGAGCTTATCTGTAGGTGG | | |
| | | (SEQ ID NO: 51) | | |
| | rs1973352 | ACGTTGGATGTGTTTCTGACAATCACAGCC | 90 | AATCACAGCCTGAAAACACA |
| | | (SEQ ID NO: 53) | | (SEQ ID NO: 55) |
| | | ACGTTGGATGGAGCCTGTTTGTTTTGTTTTC | | |
| | | (SEQ ID NO: 54) | | |
| | rs2306987 | ACGTTGGATGTACTGACAAGTTCAAACACC | 89 | CAAGTTCAAACACCTATTCA |
| | | (SEQ ID NO: 56) | | (SEQ ID NO: 58) |
| | | ACGTTGGATGTTGCATGAATGTCTGCTGGG | | |
| | | (SEQ ID NO: 57) | | |

The PCR oligonucleotides were used to amplify the region of the FAT gene that comprises the SNP of interest (as indicated in Table 2). Genomic DNA (approximately 2.5 ng) was used in a 5 μl PCR reaction. Following amplification unincorporated nucleotides are dephosphorylated using arctic shrimp alkaline phosphatase, which is then heat inactivated.

Primer extension reactions were performed by using triple terminator mixes. Assays were grouped according to the SNP-specific requirements on the termination mixes (ddACG, ddACT, ddAGT, and ddCGT, respectively), ie. should one of the nucleotides present at the site of the SNP be a thymine (T), ddATP is used in the reaction. Accordingly, when the thymine residue is present at the sit of the SNP the extension oligonucleotide is extended by one nucleotide. When the thymine is not present at the site of the SNP, the extension oligonucleotide is extended by more than one nucleotide.

The resulting primer extension reactions are desalted and the molecular weight of the primer determined using MALDI-TOF.

2.3 Analysis of Genotyping

Analysis of the FAT SNPs was extended to our unrelated bipolar case-control cohort consisting of 71 bipolar cases and 66 control individuals. All individuals in the case-control cohort were genotyped for each identified SNP. While common alleles or genotypes are expected to be found in both cases and controls, a disease-associated allele or genotype will be found at greater frequency in cases. Association analysis was performed with all informative SNPs. χ² analysis was used to test for association between bipolar and individual SNP alleles and genotypes.

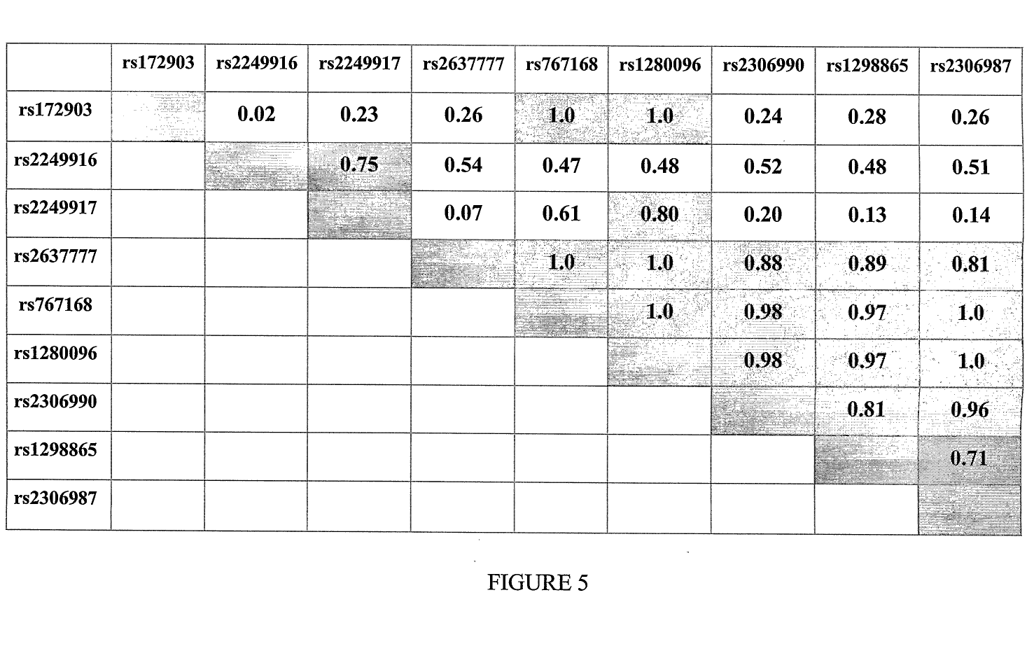

Results of analysis of SNPs located within the FAT gene are shown in Table 3 and FIG. 5.

TABLE 3
Results of analysis of association of SNPs within FAT with bipolar affective disorder.

| | | | Genotype | Allele |
|---|---|---|---|---|
| FAT | SNP position (including refSNP ID) | SNP | p-value | p-value |
| | Intron 2, rs172903, acceptor −4 | G/C | 0.23 | 0.43 |
| | Exon 13, rs2249916, codon 3117 | Synonymous, GAT [symbol] GAC | 0.56 | 0.31 |
| | Exon 13, rs2249917, codon 3121 | Synonymous, AAC [symbol] AAT | 0.38 | 0.31 |
| | Exon 19, rs2637777, codon 3554 | Non-synonymous, TCT [symbol] GCT | 0.09 | 0.12 |
| | Intron 20, rs767168, acceptor −204 | A/G | 0.97 | 0.998 |
| | Exon 21, rs2289550, codon 3847 | Synonymous, ACG [symbol] ACA | 0.46 | 0.18 |
| | Exon 23, rs1280097, codon 4061 | Non-synonymous, AAC [symbol] AAG | 0.5 | 0.61 |
| | Intron 23, rs1280096, donor +143 | A/G | 0.86 | 0.61 |
| | Intron 23, rs2306990, donor +147 | A/G | 0.09 | 0.09 |
| | Exon 26, rs1298865, codon 4367 | Synonymous, TCC [symbol] TCT | 0.04 | 0.15 |
| | Intron 26, rs2306988, donor +54 | T/A | | |
| | Intron 26, rs2306987, donor +188 | T/A | 0.016 | 0.04 |
| | Intron 26, rs3775309, acceptor −3211 | A/C | | |
| | Intron 26, rs1973352, acceptor −1487 | C/T | | |

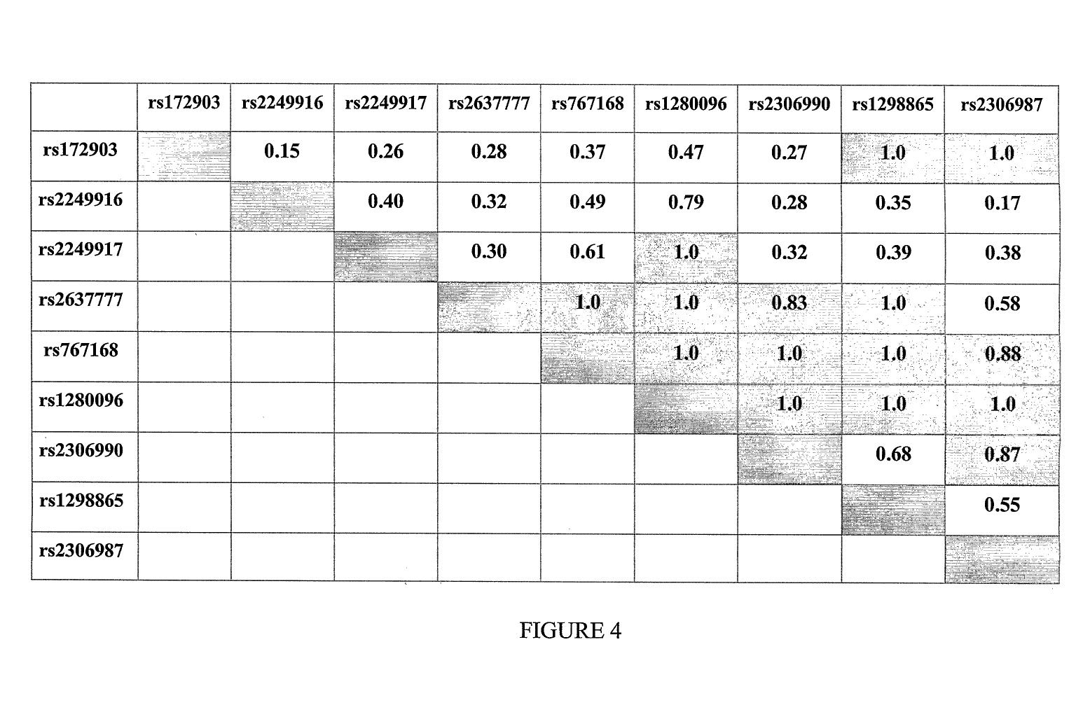

As shown in Table 3 and FIGS. 4 to 6, SNPs that show a significant association with bipolar affective disorder are located between exon 19 and the 3′ end of the FAT gene. The most significant associations are observed for the SNPs located in exon 26 and intron 26 of FAT. These data indicate that subjects that are homozygous for specific nucleotides at the SNPs located at the 3′ end of the FAT gene have a higher incidence of developing a bipolar affective disorder, than those subjects that carry another nucleotide.

Genotype frequencies for each SNP were tested using χ² analysis and all were found to be in Hardy-Weinberg equilibrium.

Analysis of linkage disequilibrium (LD) among all tested SNPs, determined that the associated SNPs are in high LD and comprise a haplotype block that spans 8.7 kb, which is located wholly within the FAT gene (FIGS. 4 to 6). No LD was detected between FAT and any adjacent gene.

### EXAMPLE 3

### Analysis SNPs in the FAT Gene are Associated with Bipolar Affective Disorder in a Number of Populations

Using the methods described in Example 2 the association of several SNPs with bipolar affective disorder was assessed in independently ascertained bipolar disorder cohorts. These cohorts included (i) 333 New Zealand cases and controls (173 case, 160 controls), (ii) 1348 UK cases and controls (669 cases, 679 controls), (iii) 90 UK parent-proband trios, and (iv) 173 Bulgarian parent-proband trios. Given the high LD between putatively associated SNPs observed in the Australian case-control cohort, three representative tagSNPs were selected for analysis among subsequent cohorts (Table 4). In addition to total cohort analysis, association was also tested for the presence or absence of a known family history of psychiatric illness (in first or second degree relative), and for the presence or absence of psychosis.

Statistically significant evidence for association with bipolar affective disorder was observed in the UK case-control cohort for those cases with a family history of psychiatric illness (allelic P=0.007, genotypic P=0.025). This result is consistent with that observed for the familial biased Australian case-control cohort, with the same alleles over represented. Statistically significant evidence for association was also observed in the UK case-control cohort for those cases without a family history of mental illness (allelic P=0.050, genotypic P=0.020), however, the observed bias was for the alternate allele to that over-represented among cases with a family history.

Both UK and Bulgarian trio cohorts showed statistically significant evidence for association among those bipolar cases who had exhibited psychosis (UK allelic P=0.003, Bulgarian allelic P=0.024). This data provides compelling evidence for association between FAT genotype and bipolar disorder among unrelated individuals from four independent cohorts.

The significant but opposing shifts in genotype frequencies observed between cases with a family history of psychiatric illness, and those without a family history appear to be an example of molecular heterosis, a common phenomenon that may be seen in as many as 50% of association studies (Comings and MacMurray, Mol. Genet. Metab. 71: 19-31, 2000).

TABLE 3
Association analysis of chromosome 4q35 tagSNPs

| | rs2304865 | | rs1298865 | | rs2306987 | |
|---|---|---|---|---|---|---|
| | Allele | geno | allele | geno | allele | geno |
| Overall | | | | | | |
| Aust case-control | 0.007 | 0.009 | 0.072 | 0.044 | 0.020 | 0.005 |
| UK case-control | 0.274 | 0.542 | 0.838 | 0.147 | 0.463 | 0.439 |
| UK trios | | | | | 0.084 | |
| Bul trios | | | | | >0.5 | |
| FH | | | | | | |
| Aust case-control | 0.007 | 0.006 | 0.010 | 0.007 | 0.010 | 0.004 |
| UK case-control | 0.007 | 0.025 | 0.038 | 0.078 | 0.365 | 0.592 |
| UK trios | | | | | 0.060 | |
| Bul trios | | | | | 0.128 | |
| No FH | | | | | | |
| Aust case-control | 0.310 | 0.528 | 0.980 | 0.639 | 0.384 | 0.228 |
| UK case-control | 0.841 | 0.975 | 0.082 | 0.020 | 0.050 | 0.022 |
| UK trios | | | | | n/a | |
| Bul trios | | | | | n/a | |
| Psych | | | | | | |
| Aust case-control | 0.092 | 0.176 | 0.197 | 0.382 | 0.178 | 0.261 |
| UK case-control | 0.466 | 0.487 | 0.512 | 0.194 | 0.329 | 0.484 |
| UK trios | | | | | 0.003 | |
| Bul trios | | | | | 0.024 | |
| No psych | | | | | | |
| Aust case-control | 0.012 | 0.011 | 0.029 | 0.009 | 0.013 | 0.005 |
| UK case-control | 0.085 | 0.079 | 0.729 | 0.272 | 0.947 | 0.298 |
| UK trios | | | | | >0.5 | |
| Bul trios | | | | | 0.058 | |

geno, genotype.
Aust, Australian.
UK, United Kingdom.
Bul, Bulgarian.
FH, family history of psychiatric illness.
Psych, psychosis.

### EXAMPLE 4

### Polymorphisms Associated with Bipolar Affective Disorder do not Modulate FAT Expression

4.1 Real Time PCR Analysis of FAT mRNA Expression

Total RNA was prepared from human cerebellum with TRI Reagent (Sigma-Aldrich) and reverse transcribed using Superscript™ III M-MLV reverse transcriptase (Invitrogen) according to the manufacturer's instructions.

RT-PCR was carried out on reverse transcribed RNA derived from human cerebellum, as well as DNA from the following cDNA libraries (all from Clontech Laboratories Inc.); human brain-hypothalamus 5′ Stretch cDNA library, human hippocampus cDNA library, human brain-amygdala 5′ Stretch Plus cDNA library. RT-PCR was carried out as previously described (Blair et al., Mol. Psychiatry. 7: 867-873 2002.

4.2 Results

To determine whether the bipolar associated FAT genotype is correlated with FAT mRNA levels in vivo, we undertook quantitative real-time RT-PCR in neural tissue. No correlation was observed among an Australian population cohort of 115 normal individuals.

### EXAMPLE 5

### Lithium and Valproate Modulate FAT Expression

5.1 Treatment of Mice with Lithium or Valproate

The animals used in the experiments to determine the effect of lithium and valproiate on FAT expression were in-bred Mus musculus, C57B16 strain, between the ages of 8-16 weeks.

Control mice (matched littermates of the test mice) received intraperitoneal injections once daily of 500 μL of sterile 0.9% saline solution for a period of 7 days.

Mice treated with lithium were administered lithium chloride (Sigma-Aldrich) by intraperitoneal injection at a concentration of 340 mg lithium/kg/day (8 mmol/kg/day) for 7 days (a single injection per day). Lithium chloride was prepared in water, and NaOH solution was added to adjust the pH to 7.5.

Mice treated with valproate were administered 2-propylpentanoic acid (Sigma-Aldrich) by intraperitoneal injection at a concentration of 350 mg/kg/day for 7 days (a single injection per day). Valproate was prepared in 0.9% saline solution.

Total volume of each injection did not exceed 500 μL.

5.2 Tissue Collection

Four hours after the final injection on the seventh day of treatment, mice were euthanised using 4% halothane anaesthesia. The whole brain was immediately removed and snap-frozen in liquid nitrogen. In addition, a cardiac puncture was performed to collect 500 μL of blood for drug serum quantitation. Whole mouse brains were stored at −80° C., until required.

5.3 Assessment of Drug Concentration in Treated Mice

To quantitate the circulating drug level in treated mice, cardiac puncture blood sample from each mouse were assayed for drug concentration by the St. Vincent's Hospital diagnostic testing facility.

Mouse brains were selected for further analysis if the serum concentration of individual mice were equivalent to that used to treat humans (i.e., 0.6-1.0 mmol/L of lithium or 45-125 mg/ml (315-885 μmol/L) of valproate).

5.4 Total RNA Isolation

Total RNA was prepared by extracting whole mouse brain tissue in TRI Reagent (Sigma-Aldrich, Sydney, NSW). Thawed whole brains were homogenised in TR1 Reagent solution (1 mL of TRI Reagent used per 100 mg of brain tissue) for 10 min using a polypropylene hand homogeniser (Sigma-Aldrich). After homogenisation, 200 μL of chloroform per 1 mL of TRI Reagent (Sigma-Aldrich) was added and mixed by vortexing for 30 sec. After a 10 min incubation, the upper inorganic phase was separated by centrifugation at 12,000×g (10,500 rpm) for 10 min at 4° C. Total RNA was precipitated (from the inorganic phase) by adding 500 μL of isopropanol per mL of TRI Reagent. RNA pellets were washed with 2 mL of 75% ethanol, air dried in a fume hood and resuspended in 100 μL of RNase-free water. The concentration of total RNA samples were determined by measuring the absorbance at 260 nm using a GeneQuant UV spectrophotometer (Pharmacia Biotech, Sydney, NSW).

5.5 Reverse Transcription

Reverse transcription was performed using 2 μg of total RNA. Prior to cDNA synthesis, total RNA was incubated for 15 min at room temperature with DNase I (2 units, Invitrogen, La Jolla, Calif., USA) in DNase I reaction buffer (200 mM Tris-HCl (pH 8.4), 20 mM MgCl₂, 500 mM KCl) to remove any DNA contamination. DNase I treatment was terminated by adding 2 μL of 25 mM EDTA (pH 8.0) and incubated at 65° C. for 10 min.

Total RNA was then denatured for 10 min at 65° C. in the presence of oligo dT₁₂₋₁₈ (500 ng; Invitrogen). After denaturing, cDNA was synthesised by adding first strand buffer (50 mM Tris-HCl (pH 8.3), 75 mM KCl, 3 mM MgCl₂; Invitrogen), dNTP (400 μM each, Promega), DTT (10 mM), RNaseOUT™ ribonuclease inhibitor (80 units, Invitrogen), Superscript™ III M-MLV reverse transcriptase (400 units, Invitrogen) and incubated for 60 min at 50° C. The reaction was terminated by incubation at 70° C. for 15 min and stored at −20° C.

5.6 Quantitative Real-Time PCR Analysis

5.6.1 PCR Primers

Forward and reverse primers were designed using MacVector v6.5.3 software (Oxford Molecular Group) and synthesised by Sigma Genosys Australia. Details of the oligonucleotides are shown in Table 4.

TABLE 4
Details of the oligonucleotides used in the analysis of FAT mRNA expression levels

| | | | PCR | |
|---|---|---|---|---|
| Gene | Forward primer | Reverse primer | productssize(bp) | An.(° C.) |
| FAT | CCGCCGTTGTGTTGTTCTTTTG (SEQ | AACGACGCTAACACCACCACAC (SEQ | 426 | 65 |
| | ID NO: 59) | ID NO: 60) | | |
| GAPDH | AACTTTGGCATTGTGGAAGGG (SEQ | TCATCATACTTGGCAGGTTTCTCC | 272 | 58 |
| | ID NO: 61) | (SEQ ID NO: 62) | | |
| β-actin | TGGGAATGGGTCAGAAGGACTC | GGTCATCTTTTCACGGTTGGC (SEQ ID | 227 | 58 |
| | (SEQ ID NO: 63) | NO: 64) | | |
| HPRT | GCTGGTGAAAAGGACCTCTCG (SEQ | CTGGCAACATCAACAGGACTCC (SEQ | 221 | 52 |
| | ID NO: 65) | ID NO: 66) | | |

An, annealing temperature for primer pair.

5.6.2 Real-Time PCR Amplification

Real-time PCR amplification was performed using 1 μL of 2/5 diluted cDNA in a 10 μL PCR reaction containing 1× Platinum® SYBR® Green qPCR SuperMix-UDG (SYBR® Green I, 30 U/mL Platinum® Taq DNA polymerase, 20 mM Tris-HCl (pH 8.4), 50 mM KCl, 3 mM MgCl₂, 200 μM dATP, 200 μM dCTP, 200 μM dGTP, 200 μM dUTP, 20 U/mL UDG, and stablisiers, Invitrogen) and forward and reverse primers (200 nM each). Amplification conditions were as follows; uracil-DNA glycosylase treatment at 50° C. for 2 min, denaturation at 95° C. for 2 min, and 35 cycles of amplification (denaturation at 95° C. for 5 sec, annealing for 15 sec, and extension for 15 sec at 72° C.). SYBR Green I fluorescence was measured at the end of annealing and extension steps. Following amplification, samples were dissociated by incremental heating between 72° C. and 99° C., at a rate of 1° C./5 sec. During this dissociation, SYBR Green I fluorescence was constantly measured. Amplification was performed in 100 μL tubes (Corbett Research, Mortlake, NSW, Australia) using a Rotor-Gene 3000 PCR machine (Corbett Research).

5.6.3 PCR Quantitation and Statistical Analysis

PCR amplification was performed simultaneously on reverse transcribed RNA prepared from the whole brains of 7 control mice, 8 mice treated with lithium, and 6 mice treated with valproate. Quantitative PCR critical threshold values for each sample were determined using Rotor-Gene v5.0.37 computer program (Corbett Research). mRNA copy number was determined relative to DNA standards of known concentration. The relative expression for mfatl was expressed as a ratio of mfatl mRNA concentration, to that of the housekeeping gene gapdh. The mean ratio and standard deviation for control, lithium, and valproate cDNAs were calculated using the StatView software v5.47 (Abacus Concepts). Unpaired Student's t-tests were performed, with a two-tailed α value of 0.05.

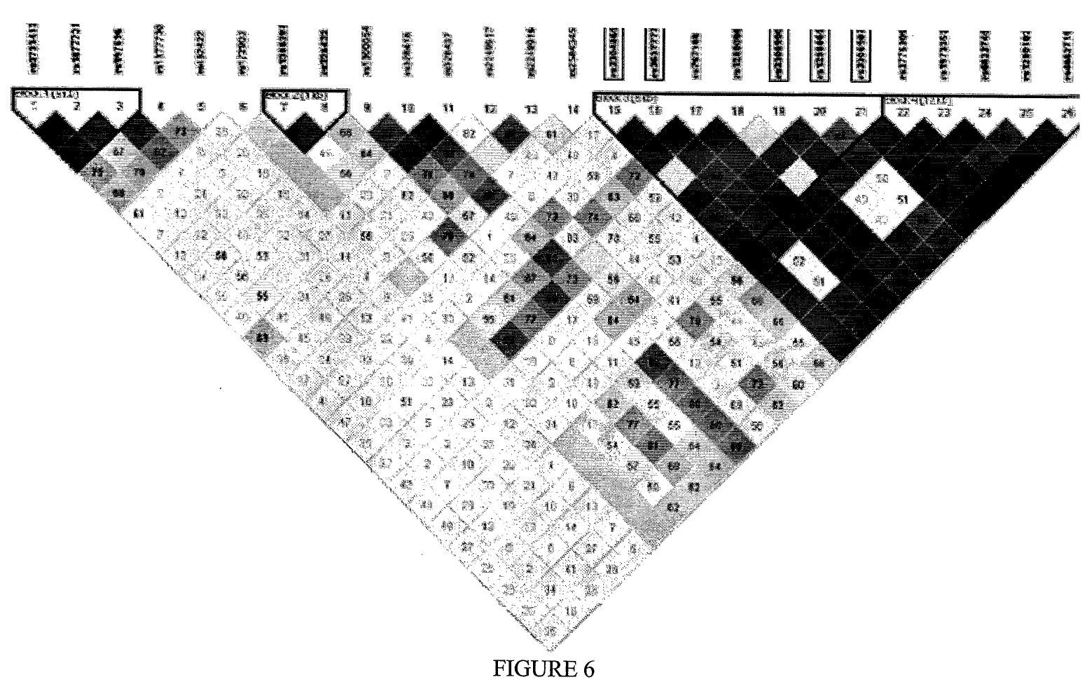

As shown in FIG. 6 the relative mRNA expression levels for mouse FAT were reduced following administration of either lithium or valproate. In particular, administration of lithium alone significantly reduced mfatl expression by 2.7 fold (p=0.027).

### EXAMPLE 6

### Exon Trapping to Determine the Effect of Polymorphisms on the Splicing of FAT

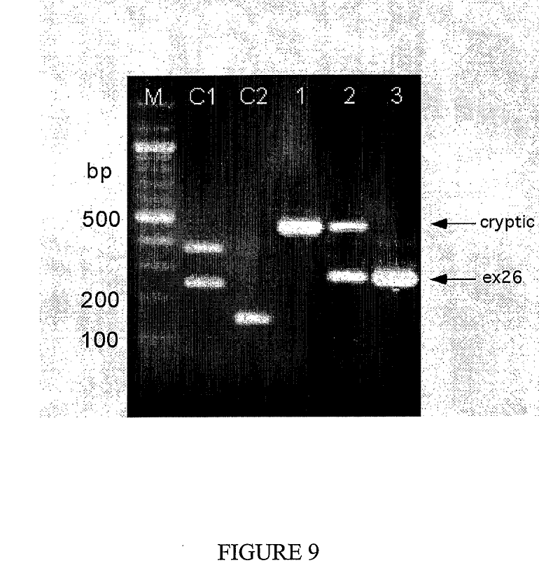

To study the effect of the SNPs on splicing, and in particular the polymorphisms that occurs at positions 148,129 and 148,333 of SEQ ID NO: 1, an exon-trapping system (Life Technologies, Inc., Frederick, Mass.) was used. Genomic DNA from bipolar affective disorder subjects and from a normal individual containing exon 26, and flanking sequences from introns 25 and 26, was amplified using PCR. Clone 1 (which comprises a thymine at a position corresponding to nucleotide position 148,129 of SEQ ID NO: 1 and an adenine at a position corresponding to nucleotide position 148,333 of SEQ ID NO: 1) and clone 2 (which comprises a cytosine at a position corresponding to nucleotide position 148,129 of SEQ ID NO: 1 and a thymine at a position corresponding to nucleotide position 148,333 of SEQ ID NO: 1) were amplified with oligonucleotides comprising the sequences set forth in SEQ ID NO: 67 (5-TTCTTCGTTTGAGTTTGTAAGGTG-3′) and SEQ ID NO: 68 (5-GTTAAGAAAGTTGCGAGGAAGATT-3′). Clone 3 (which comprises a thymine at a position corresponding to nucleotide position 148,129 of SEQ ID NO: 1) was amplified with oligonucleotides comprising the sequence set forth in SEQ ID NO: 69 (5′-TTCTTCGTTTGAGTTTGTAAGGTG-3′) and SEQ ID NO: 70 (5′-CTATTTTCCCCAGCAGACATTC-3′). The PCR products were electrophoresed on an agarose gel, stained with ethidium bromide, visualized under UV light, gel-purified (gel extraction kit, QIAGEN), and then subcloned into the pGEM-T-easy vector (Promega Corp., Madison Wis.). After the correct sequence is confirmed by DNA sequencing, the insert was cloned into the EcoRI site of the pSPL3 vector (Life Technologies, Inc.). Regions of the resulting vectors are shown in FIG. 9. The plasmid DNA was then amplified in Escherichia coli, extracted, purified, and used for transfection.

HEK-293 cells were grown in 10-cm dishes in DMEM (Life Technologies, Inc., Gaithersburg, Md.) supplemented with 10% bovine calf serum (Life Technologies, Inc.) and 50 mg/liter gentamicin in a 10% CO₂ atmosphere at 37° C. When cells reached approximately 50% confluence, they were transfected with plasmid DNA (normal, mutant, or control pSPL3, containing an exon of known size)/10-cm dish. Twenty-four hours later cells were harvested, and total RNA extracted with TRIzol (Life Technologies, Inc.). First strand synthesis of cDNA was catalyzed by the Superscript II ribonuclease H—reverse transcriptase (RT). RT-PCR was performed according to the manufacturer's instructions supplemented with the exon-trapping system (Life Technologies, Inc.). The products of the RT-PCR were electrophoresed on an agarose gel, stained with ethidium bromide, and visualized under UV light (FIG. 10). RT-PCR products were gel purified, cloned, and then sequenced (PE Applied Biosystems). Differences in the sequence of the mRNA produced by the nucleic acid isolated from a bipolar affective disorder subject compared to a normal subject indicates that a change in the nucleotide sequence of the FAT gene alters the splicing of this gene.

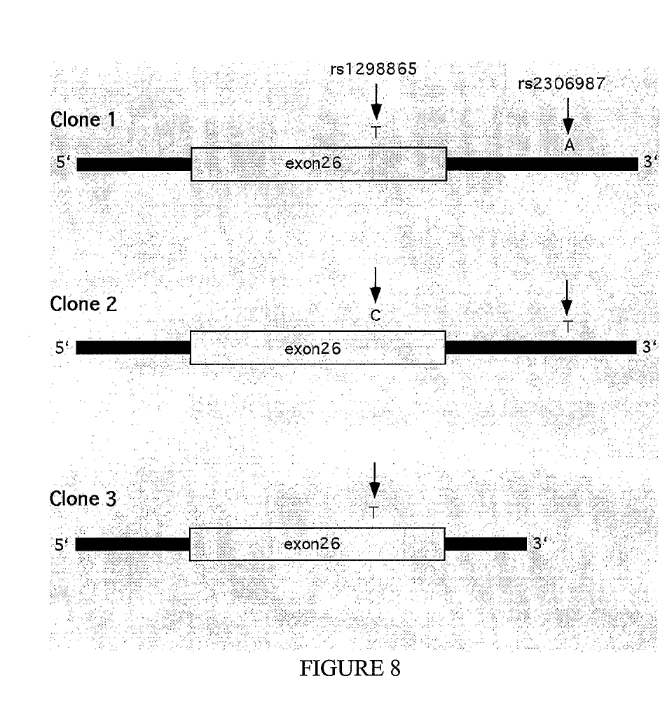

Analysis by RT-PCR of spliced mRNA derived from exon-trap clones following transfection in HEK293 cells indicates that cells comprising the clone 1 exon trap vector (ie. comprising the SNPs significantly associated with a bipolar affective disorder) preferentially spliced to a cryptic splice acceptor site that lies 93 bp downstream of rs2306987. This resulted in an additional 55 bp of FAT genomic DNA and flanking vector sequence being included in the PCR product (FIG. 8) A similar splicing event was observed in cells comprising the clone 2 exon trap vector, however, to a dramatically reduced degree.

A search of the EST database (dbEST) did not identify any entries that contained the 55 bp FAT genomic sequence present in the cryptically spliced product shown in FIG. 8. Without being bound by theory this observation suggests that transcription of the endogenous FAT gene which utilises this cryptic splice site does not produce mature mRNA (ie. the mRNA is degraded).

### EXAMPLE 7

### Northern Blotting Analysis of FAT Expression in the Brain

Northern blotting to determine FAT expression is performed essentially as described in Dunne et al., Genomics, 30: 207-223, 1995. Essentially RNA is isolated from brain tissue samples from bipolar affective disorder subjects or control subjects using the methods of Favaloro et al., Methods Enzymol., 65: 718, 1980. Poly(A)+ RNA is then selected using oligo(dT)-cellulose essentially as described in Aviv and Leder, Proc. Natl. Acad. Sci. USA, 69: 1408, 1972. mRNA is denatured and electrophoresed in a 1% formaldehyde gel and transferred to a Hybond-N+ filter (Amersham) using methods known in the art.

Probes that span the entire coding region of the FAT gene are prepared essentially as described in Dunne et al., Genomics, 30: 207-223, 1995 and labelled with ³²P. The use of a variety of probes determines the presence of multiple splice forms of FAT in the brain.

Probes are hybridised to the filters overnight at 42° C., before washing and finally hybridisation is detected using X-ray film.

### EXAMPLE 8

### Localisation of FAT Gene Expression in the Brain

In situ hybridisation to detect FAT mRNA expression is performed to determine the localisation of FAT mRNA in the brain of bipolar affective disorder subjects and control subjects. Briefly, probes that correspond to the 5′ end and 3′ end of the FAT cDNA, in addition to probes that detect alternative splice forms of FAT observed in bipolar affective disorder subjects (Example 6) are amplified using PCR and cloned into the pGEM-T Easy vector (Promega). Vectors are then linearized with ScaI and single stranded RNA probes are synthesised using T7 or Sp6 polymerase as appropriate to produce both sense and antisense probes. The probes are synthesized with a DIG RNA labelling kit (Boehringer Mannheim GmbH, Mannheim, Germany).

Sections from various regions of the brain of bipolar affective disorder subjects and control subjects are fixed for 5 min in 2×SSC containing 4% formaldehyde, washed twice in 2×SSC for 1 min, and then rinsed in 0.1 M triethanolamine containing 0.25% acetic anhydride for 5 min at room temperature (RT). The sections are washed in 2×SSC for 5 min, dehydrated, air-dried and hybridised in 50 μl hybridisation solution (50% formamide, 4×SSPE, 1× Denhardt's reagent, 10% dextran sulfate, 100 mM DTT, 0.1% SDS, 100 μg/ml salmon sperm DNA, 100 μg/ml yeast tRNA; the pH of the solution is adjusted to 8.5) containing 200 fmol/ml DIG-labeled probe. Hybridisation is performed under parafilm coverslips in a humidified chamber at 55° C. for 24 h. The sections are rinsed in 2×SSC at RT and 55° C. for 5 and 10 min, respectively, then treated with RNase A (16 μg/ml) at 37° C. for 30 min. The sections are then washed in 2×SSC/50% formamide at 55° C. for 2×10 min, and in 2×SSC at 55° C. and RT for 10 min and 5 min, respectively. After post-hybridisation, the sections are washed in buffer B1 (100 mM Tris-HCl pH 7.5 and 150 mM NaCl) for 5 min, blocked in 5% heat-inactivated sheep serum in B1 for 2 h and incubated in sheep anti-DIG-alkaline phosphatase conjugate (Boehringer Mannheim; 1:1,000 dilution) in 5% sheep serum in B1 at 4° C. for 24 h. Sections are washed in B1 for 3×5 min, and then in buffer B2 (100 mM Tris-HCl pH 9.5, 100 mM NaCl and 50 mM MgCl₂) for 10 min, and are developed in B2 containing 340 μg/ml nitroblue tetrazolium (NBT) and 180 μg/ml 5-bromo-4-chloro-3-indolyl phosphate (BCIP) for 24 h under darkroom conditions. The color reaction iss stopped by rinsing the sections in a mixture of 10 mM Tris-HCl pH 8.0 and 1 mM EDTA for 5 min in RT, and the sections are then dehydrated and covered with Entellan (Merck, Darmstadt, Germany).

Stained sections are then viewed under a standard light microscope.

### EXAMPLE 9

### Preparation of a Monoclonal Antibody that Recognizes Human FAT

A monoclonal antibody that specifically binds human FAT1 is produced using methods known in the art. Briefly, a peptide antigen that corresponds to the N-terminus of human FAT (QQHTEV, SEQ ID NO: 31) is synthesised essentially using the methods described in Bodanszky, M. (1984) Principles of Peptide Synthesis, Springer-Verlag, Heidelberg and Bodanszky, M. & Bodanszky, A. (1984) The Practice of Peptide Synthesis, Springer-Verlag, Heidelberg.

A peptide comprising the sequence SKPKPSSEPISFEESVFSC (SEQ ID NO: 72) was also synthesized for production of a monoclonal antibody.

Peptides are purified using HPLC and purity assessed by amino acid analysis.

Female BalB/c mice are immunized with a purified form of the peptide. Initially mice are sensitised by intraperitoneal injection of Hunter's Titermax adjuvant (CytRx Corp., Norcross, Ga.,). Three boosts of the peptide are administered at 2, 5.5 and 6.5 months post initial sensitisation. The first of these boosts is a subcutaneous injection while the remaining are administered by intraperitoneal injection. The final boost is administered 3 days prior to fusion.

The splenocytes of one of the immunized BALB/c mice is fused to X63-Ag8.653 mouse myeloma cells using PEG 1500. Following exposure to the PEG 1500 cells are incubated at 37° C. for 1 hour in heat inactivated foetal bovine serum. Fused cells are then transferred to RPMI 1640 medium and incubated overnight at 37° C. with 10% CO₂. The following day cells are plated using RPMI 1640 media that has been supplemented with macrophage culture supernatants.

Two weeks after fusion, hybridoma cells are screened for antibody production by solid phase ELISA assay. Standard microtitre plates are coated with the synthetic peptide in a carbonate based buffer. Plates are then blocked with BSA, washed and then the test samples (i.e. supernatant from the fused cells) is added, in addition to control samples, (i.e. supernatant from an unfused cell). Antigen-antibody binding is detected by incubating the plates with goat-anti-mouse HRP conjugate (Jackson ImmunoResearch Laboratories) and ABTS peroxidase substrate system (Vector Laboratories, Burlingame, Calif. 94010, USA). Absorbance is read on an automatic plate reader at a wavelength of 405 nm.

Any colonies that are identified as positive by these screens continue to be grown and screened for several further weeks. Stable colonies are then isolated and stored at 80° C.

Positive stable hybridomas are then cloned by growing in culture for a short period of time and diluting the cells to a final concentration of 0.1 cells/well of a 96 well tissue culture plate. These clones are then screened using the previously described assay. This procedure is then repeated in order to ensure the purity of the clone.

Four different dilutions, 5 cells/well, 2 cells/well, 1 cell/well, 0.5 cells/well of the primary clone are prepared in 96-wells microtiter plates to start the secondary cloning. Cells are diluted in IMDM tissue culture media containing the following additives: 20% fetal bovine serum (FBS), 2 mM L-glutamine, 100 units/ml of penicillin, 100 μg/ml of streptomycin, 1% GMS-S, 0.075% NaHCO₃. To determine clones that secrete anti-human FAT antibody, supernatants from individual wells of the 0.2 cells/well microtiter plate are withdrawn after two weeks of growth and tested for the presence of antibody by ELISA assay as described above.

All positive clones are then adapted and expanded in RPMI media containing the following additives: 10% FBS, 2 mM L-glutamine, 100 units/ml of penicillin, 100 μg/ml of streptomycin, 1% GMS-S, 0.075% NaHCO₃, and 0.013 mg/ml of oxalaacetic acid. A specific antibody is purified by Protein A affinity chromatography from the supernatant of cell culture.

The titer of the antibodies produced using this method are determined using the Easy Titer kit available from Pierce (Rockford, Ill., USA). This kit utilises beads that specifically bind mouse antibodies, and following binding of such an antibody these beads aggregate and no longer absorb light to the same degree as unassociated beads. Accordingly, the amount of an antibody in the supernatant of a hybridoma is assessed by comparing the OD measurement obtained from this sample to the amount detected in a standard, such as for example mouse IgG.

### EXAMPLE 10

### FAT Protein Expression in the Brain

Four-micron frozen sections isolated from human brain sections are postfixed in 4% paraformaldehyde and 20% glucose for 5 min at 4° C. Following washing sections are incubated for 30 min with CAS Block (Zymed, South San Francisco, Calif., USA). Sections are then incubated overnight at 4° C. with the mouse monoclonal antibody described in Example 9. Sections are incubated with 1:10 NGS instead of the primary antiserum as the negative control. After thorough washing with PBS (3×5 min changes), the sections are incubated with goat anti-mouse Alexa Fluor 568 (Molecular Probes), diluted 1:200 with PBS for 1 h at room temperature. Sections are rinsed with PBS (2×5 min), rinsed in tap water for 5 min, and mounted for microscope viewing.

### EXAMPLE 11

### Analysis of SNPs Using Fluorescent Single Nucleotide Primer Extension Analysis

11.1 PCR

In the first round of PCR, a fragment is amplified from genomic DNA using 0.5 μM each two primers comprising the sequences set forth in SEQ ID NOs: 8 and 9 or SEQ ID NOs: 11 and 12 and PCR supermix high fidelity (Life Technologies, Rockville, Md., USA) in a final volume of 50 μL containing 10 U/mL DNA polymerase in 60 mM Tris-SO₄ (pH 9.1), 18 mM (NH₄)₂SO₄, 2 mM MgSO₄, and 200 μM each of the four dNTPs. Thermocycling conditions: 3 min initial denaturation at 96° C. and 25 cycles of 1 min 96° C., 30 s 58° C., 3 min 72° C.

11.2 Primer Extension

PCR products are purified using Centri-sep columns (Princepton Separations, Adelphia, N.J., USA); 25-500 ng of this 422 bp fragment then serves as a template for subsequent primer extension using Cy5 labeled primer comprising the sequence set forth in SEQ ID NO: 7 (for the PCR product generated using the primers set forth in SEQ ID NO: 8 and 9) (300 nM) and 4 U of Thermo Sequenase, 0.2 mM of the three dNTPs (DATP, dCTP and dGTP) and 0.2 mM of ddTTP in a buffer of 26 mM Tris-HCl (pH 9.5) and 6.5 mM MgCl₂ in a final volume of 20 μL. For the reaction using the PCR product generated using the primer set forth in SEQ ID NO: 11 and 12 a Cy5 labeled primer (300 nM) comprising the sequence set forth in SEQ ID NO: 10 is used in a reaction comprising 4 U of Thermo Sequenase, 0.2 mM of the three dNTPs (dTTP, dCTP and dGTP) and 0.2 mM of ddATP in a buffer of 26 mM Tris-HCl (pH 9.5) and 6.5 mM MgCl₂ in a final volume of 20 μL. Thermocycling conditions: 3 min initial denaturation at 94° C. and 40 cycles of 30 s 94° C., 30 s 58° C., 30 s 72° C., followed by a 2 min final extension at 72° C. dNTPs are from Life Technologies, ddTTP and ddATP are from Sigma (St. Louis, Mo., USA), Thermo Sequenase and Sequenase reaction buffer are from Amersham (Cleveland, Ohio, USA). 10 μL formamide is added to 2 μL primer extension product and incubated at 95° C. for 2 min prior to CE analysis for denaturation.

11.3 Capillary Electrophoresis

For all experiments the P/ACE-MDQ (Beckman Coulter Inc., Fullerton, Calif., USA) instrument is used in normal polarity separations mode (cathode at the detection site). The separation is monitored on-column by laser-induced fluorescence detection (LIF) using a red diode laser (635 nm) and a narrow band pass emission filter (670 nm). The temperature of the cartridge holding the separation capillary column is thermostated at 30±0.1° C. by the active liquid cooling of the device. A 10 cm effective separation length capillary (30 cm total) with an ID of 75 μm is used for separation of the denatured primer extension products. 10% PVP (Mᵣ 1 300 000 from Aldrich) solution is used as sieving and wall coating matrix dissolved in 1×TBE buffer (89 mM Tris, 89 mM boric acid, 2 mM EDTA.Na₂, pH 8.4). Bodipy FL Hydrazide is used as neutral marker for electroosmotic flow (EOF) measurement (Molecular Probes, Eugene, Oreg., USA). The primer extension samples are electrokinetically injected at 10 kV for 30 s and separated at 20 kV. The data is acquired and evaluated by the P/ACE System MDQ software.

### EXAMPLE 12

### Analysis of SNPs Using MALDI-TOF Mass Spectrometry

12.1 Amplification of Regions Containing a Single Nucleotide Polymorphism

The bipolar affective disorder markers at position 148,108 or 148,333 of SEQ ID NO: 1 are amplified under universal conditions in a 25-μL reaction containing 50-100 ng DNA, 0.2-0.4 μM each primer (comprising the sequence set forth in SEQ ID NO: 8 and 9 for 148,108 and SEQ ID NO: 11 and 12 for 148,333), 2.5 mM MgCl₂, 200 μM each dNTP and 1 U HotStarTaq DNA Polymerase (Qiagen) in reaction buffer supplied by the manufacturer. PCR The thermal cycling conditions are: 15 min at 95° C. followed by 15 cycles of 94° C. for 30 s, 58° C. for 1 min and 72° C. for 1 min, then 30 cycles of 94° C. for 30 s, 58° C. for 1 min and 72° C. for 1 min, with a final 10 min incubation at 72° C.

12.2 Single Nucleotide Primer Extension Assay

Following PCR, 1 U of shrimp alkaline phosphatase (SAP; Roche Molecular Biochemicals) is added to 20 μL of PCR product to hydrolyze residual dNTPs. The mixture is incubated for 60 min at 37° C. followed by enzyme inactivation for 20 min at 95° C. Primer extension reactions are performed in 10-μL volumes and contained 5 μL of ‘SAP-treated’ PCR product, 30 μM each ddNTP, 1-3 μM genotyping primer (SEQ ID NO: 7 for 148,108 and SEQ ID NO: 10 for 148,333) and 1-2 U ThermoSequenase DNA polymerase (Amersham Pharmacia Biotech). Thermal cycling conditions consisted of denaturation at 94° C. for 2 min, followed by 50 cycles of 94° C. for 30 s and 37° C. for 90 s. Primer concentrations are balanced empirically to ensure that some unextended primer remained to act as an internal mass standard.

12.3 Sample Preparation and MALDI-TOFMS Analysis

Prior to analysis, samples are desalted by dialysis through 96-well membrane filter plates with 0.05 μm pore size (MAVM N05, Millipore) which are floated on deionized water for approximately 2 h. The MALDI plate is spotted with 1 μL of 3-hydroxypicolinic acid (3-HPA) matrix (50 mg/mL in 50% acetonitrile and 25 mM diammonium citrate) and allowed to dry before deposition of 1 μL of desalted primer extension sample. Mass spectra are collected using a Voyager-DE™ PRO MALDI-TOF instrument (PerSeptive Biosystems, Framingham, Mass., USA) equipped with a 337 nm nitrogen laser. Measurements are taken manually in linear, positive ion mode at a 20-24 kV acceleration voltage and 200 ns delayed ion extraction. To avoid saturation of the detector from matrix ions, masses <1000 Da are suppressed by an ion deflector. Unextended primers are used as internal standards for mass calibration. The nucleotide incorporated at the variant site is determined manually by calculating the mass difference between the unextended primer and the extension product. In the case of a heterozygote, two mass-resolved extension products are produced.

### EXAMPLE 13

### Detection of SNPs Using Fluorescence-Adapted SSCP

13.1 PCR

The primers described in Examples 11 and 12 are then adapted for fluorescence-adapted SSCP analysis, four primers are prepared as follows: the sequence-specific forward primer (SEQ ID NO: 8 or SEQ ID NO: 11) is conjugated with 5-TGA CCG GCA GCA AAA TTG-3′ (SEQ ID NO: 13) tail at its 5′ end (to form oligonucleotides that comprise the nucleotide sequence set forth in SEQ ID NOs: 15 and 17; the sequence-specific reverse primer (SEQ ID NOs: 9 or SEQ ID NO: 12) conjugated with 5-TGT AAA ACG ACG GCC AGT-3′ (SEQ ID NO: 14) tail at its 5′ end to form oligonucleotides comprising the nucleotide sequence set forth in SEQ ID NO: 16 and 18; the Cy-5 labeled 5-TGA CCG GCA GCA AAA TTG-3′ primer (SEQ ID NO: 19) (Amersham Bioscience, NJ, USA); and the Cy-5 labeled 5-TGT AAA ACG ACG GCC AGT-3′ (SEQ ID NO: 20) primer (Amersham Biosciences).

PCR is performed using a PCR mix containing 50-100 ng of Genomic DNA (from normal or bipolar subjects, or a subject being tested)<2 μmol of the conjugated sequence-specific forward primer (SEQ ID NO: 15 or 17), 2 μmol of the conjugated sequence-specific reverse primer (SEQ ID NO: 16 or 18), 10 μmol of Cy-5 labeled primer comprising the sequence set forth in SEQ ID NO: 19, 10 μmol of Cy-5 labeled primer comprising the sequence set forth in SEQ ID NO: 20, 200 μM of dNTP, 2 μL of 10× reaction buffer, 1 U of rTaq polymerase (Takara Bio, Shiga, Japan) in a final 20 μL reaction volume. The PCR amplification procedure entails an initial denaturation cycle (94° C., 4 min); 25 cycles of 1 min each at 94° C., 58° C. and 72° C., and 10 cycles of 1 min each at 94° C., 58° C. and 72° C. This was followed by 7 min at 72° C. for final extension in a GeneAmp PCR System 9600 (Applied Biosystems, CA, USA).

13.2 Gel Electrophoresis

The fluorescence-labeled DNA fragments are diluted 2-5 times with a loading buffer consisting of formamide and EDTA (100% formamide:50 mM EDTA=5:1). The DNA samples are heated at 94° C. for 5 min and cooled immediately on ice. This solution (1 to 5 μL) is applied to a non-denaturing polyacrylamide gel using 0.5×MDE gel solution (bioWhittaker Molecular Applications, ME, USA) with 1×TBE buffer (100 mM Tris-borate, pH 8.3, 1 mM EDTA). The short gel plate is applied to the Amersham Biosciences Model ALF Express DNA sequencer. The gel temperature is maintained at 15° C. Electrophoresis is performed in 0.5×TBE buffer at 30 W for 5 h, and the data is collected and analysed using Fragment Manager software (Amersham Biosciences).

### EXAMPLE 14

### Production of a Transgenic Mouse Expressing Human FAT

To produce a transgenic mouse expressing a human FAT gene a yeast artificial chromosome (YAC) that comprised the entire genomic FAT gene is isolated. LYS2 gene is incorporated into the YAC by retrofitting with a 9-kb insert from pRV1, which also contains a neomycin-resistance gene (Srivastava et al., Gene, 103: 53-59, 1991).

14.1 Mutagenesis of the FAT YAC

To mutate the FAT YAC, the “pop-in, pop-out” gene-targeting strategy is used (Duff et al., Gene Ther., 1: 70-75, 1994). The gene-targeting vector is constructed by cloning a fragment spanning from exon 26 to exon 27 of the human FAT gene (from a bipolar affective disorder subject, including either a cytosine at a position corresponding to position 148,108 of SEQ ID NO: 1 or an adenosine at a position corresponding to position 148333 of SEQ ID NO: 1) previously amplified by PCR into the yeast integrating vector pRS406, which contains the URA3 gene (Stratagene). Following DNA sequencing, the gene-targeting vector is linearized and introduced into AB1380 spheroplasts containing the FAT YAC. Transformants are initially selected on plates lacking uracil and later grown on plates lacking uracil, and Lys. To identify targeted clones (the pop-in step), yeast colonies were analyzed by pulsed-field gel electrophoresis and Southern blot analysis. For the pop-out step, yeast harbouring a targeted YAC were grown in medium lacking Lys overnight and then plated onto 5-fluo-roorotic acid plates (Rothstein, Methods Enzymol., 194: 281-301, 1991). The colonies were subsequently plated onto plates lacking Lys and examined by pulsed-field gel electrophoresis, Southern blot, and PCR analyses.

14.2 Analysis of the Gene-Targeting Events in the YACs

High molecular weight yeast DNA is electrophoresed on a 1% pulsed-field agarose gel in 0.5×TBE (1×=90 mM Tris/90 mM boric acid/2 mM EDTA, pH 8.3) at 6 V/cm at 14° C., with initial and final switching times of 0.22 and 21.79 sec, respectively. To identify gene-targeting events, fluorescent single nucleotide primer extension is performed essentially as described in Example 9.

14.3 Generation of Transgenic Mice

To generate transgenic mice expressing the mutant FAT genes, each of the YACs are purified from pulsed-field agarose gels as described (Peterson et al., Proc. Natl. Acad. Sci. USA., 90: 7593-7597, 1993), adjusted to a concentration of 3 ng/μl, and microinjected into C57BL/6×SJL mouse zygotes. Transgenic founders are identified by analysis of tail DNA for the mutant sequence. Founders are then bred to generate lines expressing human FAT mutated at position 148,108 (tghFAT148,108) or at position 148333 (tghFAT148,333). Samples isolated from the brain of offspring of the founders mice are analysed for the presence of human FAT using specific Western blots using the monoclonal antibody described in Example 7 to determine those mice that express human FAT.

### EXAMPLE 15

### Generation of a Mouse Deficient in Mouse FAT that Expresses Human FAT

The transgenic lines described in Example 14 are mated with the mFAT1^{+/-} mice (Clani et al., Molecular and Cellular Biology, 23(10): 3575-3582) to produce mFAT^{+/-} tghFAT148,108^{+/-} mice and mFAT^{+/-} tghFAT148,108^{+/-} mice. Each of these lines are then interbred to produce mFAT^{-/-} tghFAT148,108+/−mice and mFAT^{-/-} tghFAT148,108^{-/-} mice and mFAT^{-/-} tghFAT148,108+/−mice and mFAT^{-/-} tghFAT148,108^{-/-} mice. Accordingly, hemizygous and homozygous mice that express human FAT (either with a cytosine at a position corresponding to position 148,108 of SEQ ID NO: 1 or an adenosine at a position corresponding to position 148333 of SEQ ID NO: 1) in the absence of mouse FAT are produced.

### EXAMPLE 16

### Analysis of FAT Transgenic Mice for a Bipolar Affective Disorder Phenotype

The effect of the human FAT gene comprising either of the SNPs incorporated into the transgenes on anxiety in transgenic mice (Examples 14 and 15) is recorded in a dark/light exploratory model in a two-compartment light/dark box. The apparatus and conditions used are a clear acrylic chamber (40.5×40.5×30 cm) with an enclosed black acrylic box (10×20.5×20.5 cm) inserted into the right half of the chamber with an opening (13×5 cm) allowing for passage between the two compartments (movement between the compartments is monitored by an infrared beam). The apparatus is equipped with infrared sensors for the automatic recording of horizontal activity (Digiscan Model RXYZCM, Accuscan Instruments, Columbus, Ohio). The open compartment is directly illuminated by a 60-W bulb placed 40 cm above the floor of the compartment. Animals initially are placed in the centre of the dark compartment, and data collection commenced immediately for 10 min. Variables measured include latency to emerge from the dark compartment into the more aversive brightly lit compartment and the amount of time spent ambulating in each of the compartments. PPI is assayed several weeks after the dark/light test. Each of two startle chambers (SR-Lab, San Diego Instruments) contains a transparent acrylic cylinder (4 cm in diameter) mounted on a frame to which a motion sensor is attached for the detection and transduction of movement, and a sound generation system is used for the delivery of background white noise and acoustic stimuli. A CompuAdd 386 microprocessor and San Diego Instruments interface board and software are used for the delivery of stimuli and response recording (100 l-ms readings beginning at startle stimulus onset). Response amplitude is calculated as the maximum response level occurring during the 100-ms recording. Both chambers are calibrated for equivalent stimulus intensities and response sensitivities, and experimental groups are balanced across chambers. Immediately after placement in the chamber, the animal is given a 4-min acclimation period during which background noise (65 dB) is continually present, and then received four no-stimulus trials, four startle stimulus alone trials, and then 10 sets of the following four trial types counter balanced to control for order: 40-ms, 115-dB noise burst alone (startle stimulus); startle stimulus preceded 100 ms by a 20-ms, 71-dB, or 77-dB noise burst; and no stimulus. Intertrial interval is variable (average 15 sec). At the end of this block of 40 trials, the animal again receive four startle stimulus alone trials followed by four no-stimulus trials. Mice that show attenuation of the startle response have reduced sensorimotor gating, a phenotype also observed in subjects suffering from bipolar affective disorder.

1. A method for determining a bipolar affective disorder or a predisposition to a bipolar affective disorder, said method comprising detecting a marker that is linked to map position 4q35.2 of the human genome in a sample derived from a subject, wherein the detection is indicative of a bipolar affective disorder or a predisposition to a bipolar affective disorder in the subject.

2. (canceled)

3. The method according to claim 1 wherein the marker linked to map position 4q35.2 is located within or comprises the FAT gene.

4. A method for determining a bipolar affective disorder or a predisposition to a bipolar affective disorder, said method comprising detecting a marker within a FAT gene or an expression product thereof that is associated with a bipolar affective disorder in a sample derived from a subject, wherein a presence of the marker is indicative of a bipolar affective disorder or a predisposition to a bipolar affective disorder in the subject and wherein the FAT gene comprises a nucleotide sequence selected from the group consisting of:
(i) a nucleotide sequence at least 80% identical to the nucleotide sequence set forth in SEQ ID NO: 1;
(ii) a nucleotide sequence that encodes a mRNA at least 80% identical to the nucleotide sequence set forth in SEQ ID NO: 2 or 4; and
(iii) a nucleotide sequence that encodes a polypeptide comprising an amino acid sequence at least 80% identical to the amino acid sequence set forth in SEQ ID NO: 3 or 5.

5. (canceled)

6. The method according to claim 4 wherein the marker is located within the 3′ region of the FAT gene that comprises a nucleotide sequence corresponding to the region spanning from nucleotide position 139,260 to nucleotide position 170,001 of SEQ ID NO: 1.

7. (canceled)

8. (canceled)

9. (canceled)

10. (canceled)

11. (canceled)

12. (canceled)

13. The method according to claim 4 wherein the marker comprises a single nucleotide polymorphism (SNP).

14. (canceled)

15. (canceled)

16. (canceled)

17. (canceled)

18. The method according to claim 4 wherein the marker comprises a nucleic acid comprising a nucleotide sequence at least about 80% identical to at least about 20 contiguous nucleotides in a sequence selected from the group consisting of:
(i) a sequence selected from the group consisting of SEQ ID NO: 1, SEQ ID NO: 2 and SEQ ID NO: 4;
(ii) a sequence capable of encoding a polypeptide comprising an amino acid sequence at least 80% homologous to the sequence set forth in SEQ ID NO: 3 and SEQ ID NO: 5; and
(iii) a sequence complementary to a sequence set forth in (i) or (ii).

19. The method according to claim 4 wherein the marker is detected by hybridizing a nucleic acid probe or primer comprising the sequence of the marker to a marker linked to nucleic acid in a biological sample derived from a subject under moderate to high stringency hybridization conditions and detecting the hybridization using a detection means, wherein hybridization of the probe to the sample nucleic acid indicates that the subject being tested is predisposed to or suffers from a bipolar affective disorder.

20. The method according to claim 4 wherein the marker is detected by hybridizing a nucleic acid probe or primer comprising the sequence of the marker to a nucleic acid that is linked to the marker in nucleic acid in a biological sample derived from a subject under moderate to high stringency hybridization conditions and detecting the hybridization by a detection means, wherein hybridization of the probe to the sample nucleic acid indicates that the subject being tested is predisposed to or suffers from a bipolar affective disorder.

21. The method according to claim 19 wherein the detection means is a nucleic acid hybridization reaction or a nucleic acid amplification reaction.

22. (canceled)

23. (canceled)

24. The method according to claim 4 wherein the marker is detected by contacting a biological sample derived from the subject with an antibody capable of specifically binding to said marker for a time and under conditions sufficient for an antibody-ligand complex to form and then detecting the complex wherein detection of the complex indicates that the subject being tested is predisposed to or suffers from a bipolar affective disorder.

25. The method according to claim 4 wherein the biological sample comprises a nucleated cell.

26. (canceled)

27. (canceled)

28. (canceled)

29. (canceled)

30. (canceled)

31. (canceled)

32. (canceled)

33. (canceled)

34. A probe or primer comprising at least about 20 nucleotides that is capable of selectively hybridizing to the sequence set forth in SEQ ID NO: 1 and detecting a marker in a FAT gene that is associated with a bipolar affective disorder or a predisposition to a bipolar affective disorder.

35. (canceled)

36. A method for determining a subject that carries a gene or allele of a gene or a polymorphism that is associated with a bipolar affective disorder comprising detecting a marker within a FAT gene that is associated with a bipolar affective disorder in a sample derived from the subject, wherein detection of said marker indicates that the subject is a carrier of a gene or allele of a gene or a polymorphism is associated with a bipolar affective disorder.

37. A method of treatment or prophylaxis of a bipolar affective disorder comprising:
(i) performing the method of claim 1 for determining a bipolar affective disorder or a predisposition to a bipolar affective disorder; and
(ii) administering or recommending a therapeutic for the treatment of bipolar affective disorder.

38. A method for identifying a marker that is associated with a bipolar affective disorder, said method comprising:
(i) identifying a polymorphism or allele within a FAT gene or an expression product thereof;
(ii) analyzing a panel of subjects to determine those that suffer from a bipolar affective disorder, wherein not all members of the panel comprise the polymorphism or allele; and
(iii) determining the variation in the development of a bipolar affective disorder wherein said variation indicates that the polymorphism or allele is associated with a subject's predisposition to a bipolar affective disorder.

39. A method for determining a candidate compound for the treatment of a bipolar affective disorder comprising:
(i) administering a candidate compound to an animal or cell comprising or expressing a marker within a FAT gene that is associated with a bipolar affective disorder and determining the level of FAT expression in said cell or animal;
(ii) administering a candidate compound to an animal or cell that does not comprise or express a marker within a FAT gene that is associated with a bipolar affective disorder and determining the level of FAT expression in said cell or animal; and
(iii) comparing the level of FAT expression at (i) and (ii),
wherein a decreased level of FAT expression at (i) relative to (ii) indicates that the compound is a candidate compound for the treatment of a bipolar affective disorder.

40. A method for determining a candidate compound for the treatment of a bipolar affective disorder comprising:
(i) administering a candidate compound to an animal or cell capable of expressing a FAT gene and determining the level of FAT expression in said cell or animal;
(ii) determining the level of FAT expression in an animal or cell capable of expressing a FAT gene in the absence of the candidate compound; and
(iii) comparing the level of FAT expression at (i) and (ii),
wherein a decreased level of FAT expression at (i) relative to (ii) indicates that the compound is a candidate compound for the treatment of a bipolar affective disorder.

41. (canceled)

42. A process for identifying or determining a compound or modulator for the treatment of a bipolar affective disorder said method comprising:
(i) performing the method according to claim 39 to thereby identify or determine a compound for the treatment of a bipolar affective disorder;
(ii) optionally, determining the structure of the compound;
(iii) optionally, providing the name or structure of the compound; and
(iv) providing the compound.

43. A process of manufacturing a compound for the treatment of a bipolar affective disorder comprising:
(i) determining a candidate compound for the treatment of a bipolar affective disorder by performing the method according to claim 39; and
(ii) using the compound in the manufacture of a therapeutic or prophylactic for the treatment of bipolar affective disorder.
